SEQUENCE LISTING (151 sequences)

SEQ ID NO: 1 - protein, 19 aa, synthetic construct
  REGION 1..19: note = Description of Artificial Sequence: Synthetic peptide
GGGGSGGGGSGGGSGGGGS

SEQ ID NO: 2 - protein, 15 aa, synthetic construct
  REGION 1..15: note = Description of Artificial Sequence: Synthetic peptide
GGGGSGGGGSGGGGS

SEQ ID NO: 3 - protein, 24 aa, synthetic construct
  REGION 1..24: note = Description of Artificial Sequence: Synthetic peptide
GGGGSGGGGSGGGGSGGGSGGGGS

SEQ ID NO: 4 - protein, 29 aa, synthetic construct
  REGION 1..29: note = Description of Artificial Sequence: Synthetic peptide
GGGGSGGGGSGGGGSGGGGSGGGSGGGGS

SEQ ID NO: 5 - protein, 5 aa, synthetic construct
  REGION 1..5: note = Description of Artificial Sequence: Synthetic peptide
GGGGS

SEQ ID NO: 6 - protein, 10 aa, synthetic construct
  REGION 1..10: note = Description of Artificial Sequence: Synthetic peptide
GGGGSGGGGS

SEQ ID NO: 7 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 8 - DNA, 213 bp, Homo sapiens
  source 1..213: mol_type = other DNA
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 9 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 10 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 11 - DNA, 81 bp, Homo sapiens
  source 1..81: mol_type = other DNA
ttttgggtgctggtggtggttggtggagtcctggcttgctatagcttgctagtaacagtggcctttattattttctgggtg

SEQ ID NO: 12 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 13 - DNA, 117 bp, synthetic construct
  misc_feature 1..117: note = Description of Artificial Sequence: Synthetic polynucleotide
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 14 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - protein, 22 aa, Homo sapiens
QNQLYNELNLGRREEYDVLDKR

SEQ ID NO: 16 - DNA, 66 bp, Homo sapiens
  source 1..66: mol_type = other DNA
cagaaccagctctataacgagctcaatctaggacgaagagaggagtacgatgttttggacaagaga

SEQ ID NO: 17 - protein, 22 aa, synthetic construct
  REGION 1..22: note = Description of Artificial Sequence: Synthetic peptide
QNQLFNELNLGRREEFDVLDKR

SEQ ID NO: 18 - DNA, 66 bp, synthetic construct
  misc_feature 1..66: note = Description of Artificial Sequence: Synthetic oligonucleotide
  source 1..66: mol_type = other DNA
cagaaccagctctttaacgagctcaatctaggacgaagagaggagttcgatgttttggacaagaga

SEQ ID NO: 19 - protein, 23 aa, synthetic construct
  REGION 1..23: note = Description of Artificial Sequence: Synthetic peptide
QEGLYNELQKDKMAEAYSEIGMK

SEQ ID NO: 20 - DNA, 69 bp, synthetic construct
  misc_feature 1..69: note = Description of Artificial Sequence: Synthetic oligonucleotide
  source 1..69: mol_type = other DNA
caggaaggcctgtacaatgaactgcagaaagataagatggcggaggcctacagtgagattgggatgaaa

SEQ ID NO: 21 - protein, 23 aa, synthetic construct
  REGION 1..23: note = Description of Artificial Sequence: Synthetic peptide
QEGLFNELQKDKMAEAFSEIGMK

SEQ ID NO: 22 - DNA, 69 bp, synthetic construct
  misc_feature 1..69: note = Description of Artificial Sequence: Synthetic oligonucleotide
  source 1..69: mol_type = other DNA
caggaaggcctgttcaatgaactgcagaaagataagatggcggaggccttcagtgagattgggatgaaa

SEQ ID NO: 23 - protein, 22 aa, synthetic construct
  REGION 1..22: note = Description of Artificial Sequence: Synthetic peptide
HDGLYQGLSTATKDTYDALHMQ

SEQ ID NO: 24 - DNA, 66 bp, synthetic construct
  misc_feature 1..66: note = Description of Artificial Sequence: Synthetic oligonucleotide
  source 1..66: mol_type = other DNA
cacgatggcctttaccagggtctcagtacagccaccaaggacacctacgacgcccttcacatgcag

SEQ ID NO: 25 - protein, 22 aa, synthetic construct
  REGION 1..22: note = Description of Artificial Sequence: Synthetic peptide
HDGLFQGLSTATKDTFDALHMQ

SEQ ID NO: 26 - DNA, 66 bp, synthetic construct
  misc_feature 1..66: note = Description of Artificial Sequence: Synthetic oligonucleotide
  source 1..66: mol_type = other DNA
cacgatggccttttccagggtctcagtacagccaccaaggacaccttcgacgcccttcacatgcag

SEQ ID NO: 27 - protein, 112 aa, synthetic construct
  REGION 1..112: note = Description of Artificial Sequence: Synthetic polypeptide
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 28 - DNA, 336 bp, synthetic construct
  misc_feature 1..336: note = Description of Artificial Sequence: Synthetic polynucleotide
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 29 - DNA, 123 bp, synthetic construct
  misc_feature 1..123: note = Description of Artificial Sequence: Synthetic polynucleotide
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 30 - protein, 255 aa, synthetic construct
  REGION 1..255: note = Description of Artificial Sequence: Synthetic polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 31 - DNA, 126 bp, synthetic construct
  misc_feature 1..126: note = Description of Artificial Sequence: Synthetic polynucleotide
  source 1..126: mol_type = other DNA
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 32 - protein, 823 aa, Homo sapiens
MGGRVFLVFLAFCVWLTLPGAETQDSRGCARWCPQDSSCVNATACRCNPGFSSFSEIITTPMETCDDINECATLSKVSCGKFSDCWNTEGSYDCVCSPGYEPVSGAKTFKNESENTCQDVDECQQNPRLCKSYGTCVNTLGSYTCQCLPGFKLKPEDPKLCTDVNECTSGQNPCHSSTHCLNNVGSYQCRCRPGWQPIPGSPNGPNNTVCEDVDECSSGQHQCDSSTVCFNTVGSYSCRCRPGWKPRHGIPNNQKDTVCEDMTFSTWTPPPGVHSQTLSRFFDKVQDLGRDYKPGLANNTIQSILQALDELLEAPGDLETLPRLQQHCVASHLLDGLEDVLRGLSKNLSNGLLNFSYPAGTELSLEVQKQVDRSVTLRQNQAVMQLDWNQAQKSGDPGPSVVGLVSIPGMGKLLAEAPLVLEPEKQMLLHETHQGLLQDGSPILLSDVISAFLSNNDTQNLSSPVTFTFSHRSVIPRQKVLCVFWEHGQNGCGHWATTGCSTIGTRDTSTICRCTHLSSFAVLMAHYDVQEEDPVLTVITYMGLSVSLLCLLLAALTFLLCKAIQNTSTSLHLQLSLCLFLAHLLFLVAIDQTGHKVLCSIIAGTLHYLYLATLTWMLLEALYLFLTARNLTVVNYSSINRFMKKLMFPVGYGVPAVTVAISAASRPHLYGTPSRCWLQPEKGFIWGFLGPVCAIFSVNLVLFLVTLWILKNRLSSLNSEVSTLRNTRMLAFKATAQLFILGCTWCLGILQVGPAARVMAYLFTIINSLQGVFIFLVYCLLSQQVREQYGKWSKGIRKLKTESEMHTLSSSAKADTSKPSTVN

SEQ ID NO: 33 - protein, 8 aa, synthetic construct
  REGION 1..8: note = Description of Artificial Sequence: Synthetic peptide
GYTFTNYW

SEQ ID NO: 34 - protein, 8 aa, synthetic construct
  REGION 1..8: note = Description of Artificial Sequence: Synthetic peptide
VYPGDGDT

SEQ ID NO: 35 - protein, 11 aa, synthetic construct
  REGION 1..11: note = Description of Artificial Sequence: Synthetic peptide
ARGFTAYGMDY

SEQ ID NO: 36 - protein, 5 aa, synthetic construct
  REGION 1..5: note = Description of Artificial Sequence: Synthetic peptide
SSVSY

SEQ ID NO: 37 - skipped

SEQ ID NO: 38 - protein, 9 aa, synthetic construct
  REGION 1..9: note = Description of Artificial Sequence: Synthetic peptide
QQWSSNPLT

SEQ ID NO: 39 - protein, 118 aa, synthetic construct
  REGION 1..118: note = Description of Artificial Sequence: Synthetic polypeptide
QVQLQQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSS

SEQ ID NO: 40 - protein, 106 aa, synthetic construct
  REGION 1..106: note = Description of Artificial Sequence: Synthetic polypeptide
EIVLTQSPATMSASPGERVTMSCSASSSVSYMHWYQQKSGQSPKRWIYDTSKLASGVPARFSGSGSGTDYTFTISSMEPEDFATYYCQQWSSNPLTFGGGTKLEIK

SEQ ID NO: 41 - protein, 239 aa, synthetic construct
  REGION 1..239: note = Description of Artificial Sequence: Synthetic polypeptide
QVQLQQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSSGGGGSGGGGSGGGGSEIVLTQSPATMSASPGERVTMSCSASSSVSYMHWYQQKSGQSPKRWIYDTSKLASGVPARFSGSGSGTDYTFTISSMEPEDFATYYCQQWSSNPLTFGGGTKLEIK

SEQ ID NO: 42 - DNA, 717 bp, synthetic construct
  misc_feature 1..717: note = Description of Artificial Sequence: Synthetic polynucleotide
  source 1..717: mol_type = other DNA
caagttcagctccagcagagcggcgccgaagtggcaaagcctggagcgtcagtcaagctgtcctgcaaagcgagtggctatacgttcacgaactactggatgcagtggataaagcaggctcccgggcagggtctggagtggattggagccgtctacccaggggacggcgacacccggcacactcaaaagttcaagggcaaggccaccctgaccgctgacaagagcacaagcacagcgtacatggaggtgtcctctttgagatccgaagataccgctgtgtattattgtgcccggggcttcactgcatacgggatggattactggggacaaggcactaccgtgactgtcagctccgggggtggaggctcaggcggggggggttcaggaggggggggatctgaaattgtgctgacacagagccctgccacaatgtctgctagccctggcgagcgcgtgaccatgtcttgtagcgccagcagcagcgtgtcctacatgcattggtatcaacagaagtccggccagtctcccaagcggtggatctacgatacaagcaagctggcctccggcgtgcccgccagattttctggcagcggctctggaacagattacaccttcaccatctctagcatggaacctgaggattttgccacctactattgccagcagtggtccagcaatcccctgacatttggaggaggcaccaagctggaaattaag

SEQ ID NO: 43 - protein, 118 aa, synthetic construct
  REGION 1..118: note = Description of Artificial Sequence: Synthetic polypeptide
QVQLQQSGAEVKKPGASVKLSCKASGYTFTNYWMQWIRQAPGQGLEWIGAVYPGDGDTRYTQKFQGRATLTADTSISTAYMEVSRLRSDDTAVYYCARGFTAYGMDYWGQGTTVTVSS

SEQ ID NO: 44 - protein, 106 aa, synthetic construct
  REGION 1..106: note = Description of Artificial Sequence: Synthetic polypeptide
EIVLTQSPATMSASPGERVTMSCSASSSVSYMHWYQQKSGLSPKRWIYDTSKLASGVPDRFSGSGSGTDYTFTISRMEPEDFATYYCQQWSSNPLTFGGGTKLEIK

SEQ ID NO: 45 - protein, 239 aa, synthetic construct
  REGION 1..239: note = Description of Artificial Sequence: Synthetic polypeptide
QVQLQQSGAEVKKPGASVKLSCKASGYTFTNYWMQWIRQAPGQGLEWIGAVYPGDGDTRYTQKFQGRATLTADTSISTAYMEVSRLRSDDTAVYYCARGFTAYGMDYWGQGTTVTVSSGGGGSGGGGSGGGGSEIVLTQSPATMSASPGERVTMSCSASSSVSYMHWYQQKSGLSPKRWIYDTSKLASGVPDRFSGSGSGTDYTFTISRMEPEDFATYYCQQWSSNPLTFGGGTKLEIK

SEQ ID NO: 46 - protein, 118 aa, synthetic construct
  REGION 1..118: note = Description of Artificial Sequence: Synthetic polypeptide
QVQLQQSGAEVKKPGASVKLSCKASGYTFTNYWMQWVRQAPGQGLEWIGAVYPGDGDTRYTQKFQGRATLTADTSTSTVYMEVSSLRSEDTAVYYCARGFTAYGMDYWGQGTTVTVSS

SEQ ID NO: 47 - protein, 106 aa, synthetic construct; identical to SEQ ID NO: 44
  REGION 1..106: note = Description of Artificial Sequence: Synthetic polypeptide

SEQ ID NO: 48 - protein, 239 aa, synthetic construct
  REGION 1..239: note = Description of Artificial Sequence: Synthetic polypeptide
QVQLQQSGAEVKKPGASVKLSCKASGYTFTNYWMQWVRQAPGQGLEWIGAVYPGDGDTRYTQKFQGRATLTADTSTSTVYMEVSSLRSEDTAVYYCARGFTAYGMDYWGQGTTVTVSSGGGGSGGGGSGGGGSEIVLTQSPATMSASPGERVTMSCSASSSVSYMHWYQQKSGLSPKRWIYDTSKLASGVPDRFSGSGSGTDYTFTISRMEPEDFATYYCQQWSSNPLTFGGGTKLEIK

SEQ ID NO: 49 - protein, 118 aa, synthetic construct
  REGION 1..118: note = Description of Artificial Sequence: Synthetic polypeptide
QVQLQQSGAEVKKPGASVKVSCKASGYTFTNYWMQWVRQAPGQGLEWMGAVYPGDGDTRHTQKFKGRVTMTRDTSTSTVYMELSSLRSEDTAVYYCARGFTAYGMDYWGQGTLVTVSS

SEQ ID NO: 50 - protein, 106 aa, synthetic construct
  REGION 1..106: note = Description of Artificial Sequence: Synthetic polypeptide
EIVLTQSPATLSLSPGERATLSCSASSSVSYMHWYQQKPGLAPRLLIYDTSKLASGIPDRFSGSGSGTDFTLTISRLEPEDFAVYYCQQWSSNPLTFGQGTKVEIK

SEQ ID NO: 51 - protein, 242 aa, synthetic construct
  REGION 1..242: note = Description of Artificial Sequence: Synthetic polypeptide
QVQLQQSGAEVKKPGASVKVSCKASGYTFTNYWMQWVRQAPGQGLEWMGAVYPGDGDTRHTQKFKGRVTMTRDTSTSTVYMELSSLRSEDTAVYYCARGFTAYGMDYWGQGTLVTVSSASTGGGGSGGGGSGGGGSEIVLTQSPATLSLSPGERATLSCSASSSVSYMHWYQQKPGLAPRLLIYDTSKLASGIPDRFSGSGSGTDFTLTISRLEPEDFAVYYCQQWSSNPLTFGQGTKVEIK

SEQ ID NO: 52 - protein, 118 aa, synthetic construct
  REGION 1..118: note = Description of Artificial Sequence: Synthetic polypeptide
QVQLQQSGAEVKKPGASVKVSCKASGYTFTNYWMQWVRQAPGQGLEWIGAVYPGDGDTRHTQKFKGRVTMTADKSTSTVYMELSSLRSEDTAVYYCARGFTAYGMDYWGQGTLVTVSS

SEQ ID NO: 53 - protein, 106 aa, synthetic construct
  REGION 1..106: note = Description of Artificial Sequence: Synthetic polypeptide
QIVLTQSPATLSLSPGERATLTCSASSSVSYMHWYQQKPGLSPKRWIYDTSKLASGVPDRFSGSGSGTDYTFTIRRLEPEDFATYYCQQWSSNPLTFGQGTKVEIK

SEQ ID NO: 54 - protein, 242 aa, synthetic construct
  REGION 1..242: note = Description of Artificial Sequence: Synthetic polypeptide
QVQLQQSGAEVKKPGASVKVSCKASGYTFTNYWMQWVRQAPGQGLEWIGAVYPGDGDTRHTQKFKGRVTMTADKSTSTVYMELSSLRSEDTAVYYCARGFTAYGMDYWGQGTLVTVSSASTGGGGSGGGGSGGGGSQIVLTQSPATLSLSPGERATLTCSASSSVSYMHWYQQKPGLSPKRWIYDTSKLASGVPDRFSGSGSGTDYTFTIRRLEPEDFATYYCQQWSSNPLTFGQGTKVEIK

SEQ ID NO: 55 - protein, 118 aa, synthetic construct
  REGION 1..118: note = Description of Artificial Sequence: Synthetic polypeptide
QVQLQQSGAEVKKPGASVKLSCKASGYTFTNYWMQWIRQAPGQGLEWIGAVYPGDGDTRYTQKFQGRATLTADTSTSTAYMEVSSLRSEDTAVYYCARGFTAYGMDYWGQGTTVTVSS

SEQ ID NO: 56 - protein, 106 aa, synthetic construct
  REGION 1..106: note = Description of Artificial Sequence: Synthetic polypeptide
EIVLTQSPATLSASPGERVTMSCSASSSVSYMHWYQQKPGLAPRRWIYDTSKLASGVPDRFSGSGSGTDYTFTISRMEPEDFATYYCQQWSSNPLTFGGGTKLEIK

SEQ ID NO: 57 - protein, 239 aa, synthetic construct
  REGION 1..239: note = Description of Artificial Sequence: Synthetic polypeptide
QVQLQQSGAEVKKPGASVKLSCKASGYTFTNYWMQWIRQAPGQGLEWIGAVYPGDGDTRYTQKFQGRATLTADTSTSTAYMEVSSLRSEDTAVYYCARGFTAYGMDYWGQGTTVTVSSGGGGSGGGGSGGGGSEIVLTQSPATLSASPGERVTMSCSASSSVSYMHWYQQKPGLAPRRWIYDTSKLASGVPDRFSGSGSGTDYTFTISRMEPEDFATYYCQQWSSNPLTFGGGTKLEIK

SEQ ID NO: 58 - protein, 20 aa, synthetic construct
  REGION 1..20: note = Description of Artificial Sequence: Synthetic peptide
MYRMQLLSCIALSLALVTNS

SEQ ID NO: 59 - protein, 20 aa, synthetic construct
  REGION 1..20: note = Description of Artificial Sequence: Synthetic peptide
MYSMQLASCVTLTLVLLVNS

SEQ ID NO: 60 - protein, 20 aa, synthetic construct
  REGION 1..20: note = Description of Artificial Sequence: Synthetic peptide
METPAQLLFLLLLWLPDTTG

SEQ ID NO: 61 - protein, 20 aa, synthetic construct
  REGION 1..20: note = Description of Artificial Sequence: Synthetic peptide
METDTLLLWVLLLWVPGSTG

SEQ ID NO: 62 - protein, 21 aa, synthetic construct
  REGION 1..21: note = Description of Artificial Sequence: Synthetic peptide
MALPVTALLLPLALLLHAARP

SEQ ID NO: 63 - protein, 18 aa, synthetic construct
  REGION 1..18: note = Description of Artificial Sequence: Synthetic peptide
MALPVTALLLPLALLLHA

SEQ ID NO: 64 - protein, 16 aa, synthetic construct
  REGION 1..16: note = Description of Artificial Sequence: Synthetic peptide
MKWVTFISLLFSSAYS

SEQ ID NO: 65 - protein, 30 aa, synthetic construct
  REGION 1..30: note = Description of Artificial Sequence: Synthetic polypeptide
MDSKGSSQKGSRLLLLLVVSNLLLCQGVVS

SEQ ID NO: 66 - protein, 462 aa, synthetic construct
  REGION 1..462: note = Description of Artificial Sequence: Synthetic polypeptide
QVQLQQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSSGGGGSGGGGSGGGGSEIVLTQSPATMSASPGERVTMSCSASSSVSYMHWYQQKSGQSPKRWIYDTSKLASGVPARFSGSGSGTDYTFTISSMEPEDFATYYCQQWSSNPLTFGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 67 - DNA, 1386 bp, synthetic construct
  misc_feature 1..1386: note = Description of Artificial Sequence: Synthetic polynucleotide
  source 1..1386: mol_type = other DNA
caagttcagctccagcagagcggcgccgaagtggcaaagcctggagcgtcagtcaagctgtcctgcaaagcgagtggctatacgttcacgaactactggatgcagtggataaagcaggctcccgggcagggtctggagtggattggagccgtctacccaggggacggcgacacccggcacactcaaaagttcaagggcaaggccaccctgaccgctgacaagagcacaagcacagcgtacatggaggtgtcctctttgagatccgaagataccgctgtgtattattgtgcccggggcttcactgcatacgggatggattactggggacaaggcactaccgtgactgtcagctccgggggtggaggctcaggcggggggggttcaggaggggggggatctgaaattgtgctgacacagagccctgccacaatgtctgctagccctggcgagcgcgtgaccatgtcttgtagcgccagcagcagcgtgtcctacatgcattggtatcaacagaagtccggccagtctcccaagcggtggatctacgatacaagcaagctggcctccggcgtgcccgccagattttctggcagcggctctggaacagattacaccttcaccatctctagcatggaacctgaggattttgccacctactattgccagcagtggtccagcaatcccctgacatttggaggaggcaccaagctggaaattaagagagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 68 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 69 - protein, 10 aa, synthetic construct
  REGION 1..10: note = Description of Artificial Sequence: Synthetic peptide
GGSISSSTYY

SEQ ID NO: 70 - protein, 7 aa, synthetic construct
  REGION 1..7: note = Description of Artificial Sequence: Synthetic peptide
THYRGST

SEQ ID NO: 71 - protein, 11 aa, synthetic construct
  REGION 1..11: note = Description of Artificial Sequence: Synthetic peptide
ARELTGEVFDY

SEQ ID NO: 72 - protein, 6 aa, synthetic construct
  REGION 1..6: note = Description of Artificial Sequence: Synthetic peptide
QSISSY

SEQ ID NO: 73 - skipped

SEQ ID NO: 74 - protein, 9 aa, synthetic construct
  REGION 1..9: note = Description of Artificial Sequence: Synthetic peptide
QQSYSTPFT

SEQ ID NO: 75 - protein, 119 aa, synthetic construct
  REGION 1..119: note = Description of Artificial Sequence: Synthetic polypeptide
QLQLQESGPGLVKPSETLSLTCTVSGGSISSSTYYWGWIRQPPRKGLEWIGSTHYRGSTYYNPSLKSRVTISVDTSKNQFSLKVSSVTAADTAVYYCARELTGEVFDYWGQGTLVTVSS

SEQ ID NO: 76 - protein, 107 aa, synthetic construct
  REGION 1..107: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 77 - DNA, 357 bp, synthetic construct
  misc_feature 1..357: note = Description of Artificial Sequence: Synthetic polynucleotide
  source 1..357: mol_type = other DNA
cagctccagctccaagagtcagggccaggtctcgtgaaaccgagtgagaccctgtccctgacctgcacagtgagtggtggatcaatctcaagctctacctactattgggggtggattcggcagccccctagaaaggggcttgagtggattggcagcactcattatcgaggatctacctattataatccttctctgaaaagcagagttaccatctctgtggatacgtccaaaaatcagttcagtctgaaggtatcatccgtgactgctgccgacacggccgtgtactattgcgcgagggagctgacaggtgaggtctttgactactggggccagggcacactcgtgaccgtgtcttct

SEQ ID NO: 78 - DNA, 321 bp, synthetic construct
  misc_feature 1..321: note = Description of Artificial Sequence: Synthetic polynucleotide
  source 1..321: mol_type = other DNA
gacatccagatgacgcagtccccttccagcttgtccgcatctgtgggtgatagggtcacgattacatgtagggctagtcagagtatttctagttacctgaattggtaccagcagaaaccaggcaaggcaccaaagttgctcatctatgcggcctcctctctgcaatctggcgtgccgtccagatttagtggatcaggctccggaaccgatttcacccttacgatctcctcacttcaacccgaggatttcgccacatattactgtcaacaaagctattctacaccgttcaccttcggaccggggacaaaagtggatattaaa

SEQ ID NO: 79 - protein, 244 aa, synthetic construct
  REGION 1..244: note = Description of Artificial Sequence: Synthetic polypeptide
QLQLQESGPGLVKPSETLSLTCTVSGGSISSSTYYWGWIRQPPRKGLEWIGSTHYRGSTYYNPSLKSRVTISVDTSKNQFSLKVSSVTAADTAVYYCARELTGEVFDYWGQGTLVTVSSASTGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIK

SEQ ID NO: 80 - DNA, 732 bp, synthetic construct
  misc_feature 1..732: note = Description of Artificial Sequence: Synthetic polynucleotide
  source 1..732: mol_type = other DNA
cagctccagctccaagagtcagggccaggtctcgtgaaaccgagtgagaccctgtccctgacctgcacagtgagtggtggatcaatctcaagctctacctactattgggggtggattcggcagccccctagaaaggggcttgagtggattggcagcactcattatcgaggatctacctattataatccttctctgaaaagcagagttaccatctctgtggatacgtccaaaaatcagttcagtctgaaggtatcatccgtgactgctgccgacacggccgtgtactattgcgcgagggagctgacaggtgaggtctttgactactggggccagggcacactcgtgaccgtgtcttctgcctcaacaggagggggtgggagtggaggcggtggatcagggggaggagggagtgacatccagatgacgcagtccccttccagcttgtccgcatctgtgggtgatagggtcacgattacatgtagggctagtcagagtatttctagttacctgaattggtaccagcagaaaccaggcaaggcaccaaagttgctcatctatgcggcctcctctctgcaatctggcgtgccgtccagatttagtggatcaggctccggaaccgatttcacccttacgatctcctcacttcaacccgaggatttcgccacatattactgtcaacaaagctattctacaccgttcaccttcggaccggggacaaaagtggatattaaa

SEQ ID NO: 81 - protein, 8 aa, synthetic construct
  REGION 1..8: note = Description of Artificial Sequence: Synthetic peptide
GGSISTYY

SEQ ID NO: 82 - protein, 7 aa, synthetic construct
  REGION 1..7: note = Description of Artificial Sequence: Synthetic peptide
IYYSGST

SEQ ID NO: 83 - protein, 14 aa, synthetic construct
  REGION 1..14: note = Description of Artificial Sequence: Synthetic peptide
AREDYYGSGSPFDY

SEQ ID NO: 84 - protein, 6 aa, synthetic construct
  REGION 1..6: note = Description of Artificial Sequence: Synthetic peptide
QGIRYD

SEQ ID NO: 85 - protein, 9 aa, synthetic construct
  REGION 1..9: note = Description of Artificial Sequence: Synthetic peptide
LQDYNFPRT

SEQ ID NO: 86 - protein, 120 aa, synthetic construct
  REGION 1..120: note = Description of Artificial Sequence: Synthetic polypeptide
QVQLQESGPGLVKPSETLSLTCTVSGGSISTYYWSWIRQPPGKGLEWIGYIYYSGSTKYNPSLKSRVTISVDTSKNLFSLKLSSVTAADTAVYYCAREDYYGSGSPFDYWGQGTLVTVSS

SEQ ID NO: 87 - protein, 107 aa, synthetic construct
  REGION 1..107: note = Description of Artificial Sequence: Synthetic polypeptide
AIQMTQSPSSLSASVGDRVTITCRASQGIRYDLGWYQQKPGKAPKLLIYAASSLQSGVPSRFSGSGSGTDFTLTISSLQPEDFATYYCLQDYNFPRTFGQGTKVEIK

SEQ ID NO: 88 - protein, 245 aa, synthetic construct
  REGION 1..245: note = Description of Artificial Sequence: Synthetic polypeptide
QVQLQESGPGLVKPSETLSLTCTVSGGSISTYYWSWIRQPPGKGLEWIGYIYYSGSTKYNPSLKSRVTISVDTSKNLFSLKLSSVTAADTAVYYCAREDYYGSGSPFDYWGQGTLVTVSSASTGGGGSGGGGSGGGGSAIQMTQSPSSLSASVGDRVTITCRASQGIRYDLGWYQQKPGKAPKLLIYAASSLQSGVPSRFSGSGSGTDFTLTISSLQPEDFATYYCLQDYNFPRTFGQGTKVEIK

SEQ ID NO: 89 - protein, 8 aa, synthetic construct
  REGION 1..8: note = Description of Artificial Sequence: Synthetic peptide
GFTFSSYG

SEQ ID NO: 90 - protein, 8 aa, synthetic construct
  REGION 1..8: note = Description of Artificial Sequence: Synthetic peptide
ISYDGSDK

SEQ ID NO: 91 - protein, 10 aa, synthetic construct
  REGION 1..10: note = Description of Artificial Sequence: Synthetic peptide
ARDKGYYFDY

SEQ ID NO: 92 - protein, 7 aa, synthetic construct
  REGION 1..7: note = Description of Artificial Sequence: Synthetic peptide
QSVGNRY

SEQ ID NO: 93 - skipped

SEQ ID NO: 94 - protein, 9 aa, synthetic construct
  REGION 1..9: note = Description of Artificial Sequence: Synthetic peptide
QQDYNLPLT

SEQ ID NO: 95 - protein, 117 aa, synthetic construct
  REGION 1..117: note = Description of Artificial Sequence: Synthetic polypeptide
QVQLVESGGGVVQPGRSLRLSCAASGFTFSSYGMHWVRQAPGKGLEWVAVISYDGSDKYYVDSVKGRFTISRDNSKNTLYLHMNSLRAEDTAVYYCARDKGYYFDYWGQGTLVTVSS

SEQ ID NO: 96 - protein, 108 aa, synthetic construct
  REGION 1..108: note = Description of Artificial Sequence: Synthetic polypeptide
EIVMTQSPATLSLSPGERATLSCRASQSVGNRYLSWYQQKPGQAPRLLIYGASTRATGIPARFSGSGSGTDFTLTISSLQPEDFAVYYCQQDYNLPLTFGGGTKVEIK

SEQ ID NO: 97 - protein, 243 aa, synthetic construct
  REGION 1..243: note = Description of Artificial Sequence: Synthetic polypeptide
QVQLVESGGGVVQPGRSLRLSCAASGFTFSSYGMHWVRQAPGKGLEWVAVISYDGSDKYYVDSVKGRFTISRDNSKNTLYLHMNSLRAEDTAVYYCARDKGYYFDYWGQGTLVTVSSASTGGGGSGGGGSGGGGSEIVMTQSPATLSLSPGERATLSCRASQSVGNRYLSWYQQKPGQAPRLLIYGASTRATGIPARFSGSGSGTDFTLTISSLQPEDFAVYYCQQDYNLPLTFGGGTKVEIK

SEQ ID NO: 98 - protein, 10 aa, synthetic construct
  REGION 1..10: note = Description of Artificial Sequence: Synthetic peptide
ARDGSRYFDY

SEQ ID NO: 99 - protein, 7 aa, synthetic construct
  REGION 1..7: note = Description of Artificial Sequence: Synthetic peptide
QSVHSKY

SEQ ID NO: 100 - protein, 117 aa, synthetic construct
  REGION 1..117: note = Description of Artificial Sequence: Synthetic polypeptide
QVQLVESGGGVVQPGRSLRLSCAASGFTFSSYGMHWVRQAPGKGLEWVAVISYDGSDKYSADSVKGRFNISRDNSKNTLYLQMNSLRAEDTAVYYCARDGSRYFDYWGQGTLVTVSS

SEQ ID NO: 101 - protein, 108 aa, synthetic construct
  REGION 1..108: note = Description of Artificial Sequence: Synthetic polypeptide
EIFMTQSPATLSLSPGERATLSCRASQSVHSKYLSWYQQKPGQAPSLLIYGASTRATGIPARFSGSGSGTDFTLTISSLQPEDFAVYYCQQDYNLPITFGQGTRLEIK

SEQ ID NO: 102 - protein, 243 aa, synthetic construct
  REGION 1..243: note = Description of Artificial Sequence: Synthetic polypeptide
QVQLVESGGGVVQPGRSLRLSCAASGFTFSSYGMHWVRQAPGKGLEWVAVISYDGSDKYSADSVKGRFNISRDNSKNTLYLQMNSLRAEDTAVYYCARDGSRYFDYWGQGTLVTVSSASTGGGGSGGGGSGGGGSEIFMTQSPATLSLSPGERATLSCRASQSVHSKYLSWYQQKPGQAPSLLIYGASTRATGIPARFSGSGSGTDFTLTISSLQPEDFAVYYCQQDYNLPITFGQGTRLEIK

SEQ ID NO: 103 - protein, 7 aa, synthetic construct
  REGION 1..7: note = Description of Artificial Sequence: Synthetic peptide
IYFSGST

SEQ ID NO: 104 - protein, 6 aa, synthetic construct
  REGION 1..6: note = Description of Artificial Sequence: Synthetic peptide
QGIRND

SEQ ID NO: 105 - protein, 9 aa, synthetic construct
  REGION 1..9: note = Description of Artificial Sequence: Synthetic peptide
LQDYNYPRT

SEQ ID NO: 106 - protein, 120 aa, synthetic construct
  REGION 1..120: note = Description of Artificial Sequence: Synthetic polypeptide
QVQLQESGPGLVKPSETLSLTCTVSGGSISTYYWSWIRQPPGKGLEWLGYIYFSGSTNYNPSLKSRLTISVAASKSQFSLKLSSVTAADTAVYYCAREDYYGSGSPFDYWGQGTLVTVSS

SEQ ID NO: 107 - protein, 107 aa, synthetic construct
  REGION 1..107: note = Description of Artificial Sequence: Synthetic polypeptide
AIQMTQSPSSLSASVGDRVTITCRASQGIRNDLGWFQQKPGKAPKLLIYAASSLQSGVPSRFSGSGSGTYFTLTISSLQPEDSATYYCLQDYNYPRTFGQGTKVEIK

SEQ ID NO: 108 - protein, 245 aa, synthetic construct
  REGION 1..245: note = Description of Artificial Sequence: Synthetic polypeptide
QVQLQESGPGLVKPSETLSLTCTVSGGSISTYYWSWIRQPPGKGLEWLGYIYFSGSTNYNPSLKSRLTISVAASKSQFSLKLSSVTAADTAVYYCAREDYYGSGSPFDYWGQGTLVTVSSASTGGGGSGGGGSGGGGSAIQMTQSPSSLSASVGDRVTITCRASQGIRNDLGWFQQKPGKAPKLLIYAASSLQSGVPSRFSGSGSGTYFTLTISSLQPEDSATYYCLQDYNYPRTFGQGTKVEIK

SEQ ID NO: 109 - protein, 8 aa, synthetic construct
  REGION 1..8: note = Description of Artificial Sequence: Synthetic peptide
GGSISTDY

SEQ ID NO: 110 - protein, 6 aa, synthetic construct
  REGION 1..6: note = Description of Artificial Sequence: Synthetic peptide
QDIRND

SEQ ID NO: 111 - protein, 120 aa, synthetic construct
  REGION 1..120: note = Description of Artificial Sequence: Synthetic polypeptide
QVQLQESGPGLVKPSETLSLTCTVSGGSISTDYWSWIRQPPGKGLEWIGYIYFSGSTKYNPSLKSRVTISVDTSKNQFSLKLSSVTAADTAVYYCAREDYYGSGSPFDYWGQGTLVTVSS

SEQ ID NO: 112 - protein, 107 aa, synthetic construct
  REGION 1..107: note = Description of Artificial Sequence: Synthetic polypeptide
AIQMTQSPSSLSASVGDRVTITCRASQDIRNDLGWFQQKPGKAPKLLIYAASSLQSGVPSRFSGSGSGTDFTLTISSLQPEDFATYYCLQDYNFPRTFGQGTKVEIK

SEQ ID NO: 113 - protein, 245 aa, synthetic construct
  REGION 1..245: note = Description of Artificial Sequence: Synthetic polypeptide
QVQLQESGPGLVKPSETLSLTCTVSGGSISTDYWSWIRQPPGKGLEWIGYIYFSGSTKYNPSLKSRVTISVDTSKNQFSLKLSSVTAADTAVYYCAREDYYGSGSPFDYWGQGTLVTVSSASTGGGGSGGGGSGGGGSAIQMTQSPSSLSASVGDRVTITCRASQDIRNDLGWFQQKPGKAPKLLIYAASSLQSGVPSRFSGSGSGTDFTLTISSLQPEDFATYYCLQDYNFPRTFGQGTKVEIK

SEQ ID NO: 114 - protein, 10 aa, synthetic construct
  REGION 1..10: note = Description of Artificial Sequence: Synthetic peptide
ARDGQFYFDY

SEQ ID NO: 115 - protein, 7 aa, synthetic construct
  REGION 1..7: note = Description of Artificial Sequence: Synthetic peptide
QSVTSRY

SEQ ID NO: 116 - protein, 9 aa, synthetic construct
  REGION 1..9: note = Description of Artificial Sequence: Synthetic peptide
QQDYNLPLT

SEQ ID NO: 117 - protein, 117 aa, synthetic construct
  REGION 1..117: note = Description of Artificial Sequence: Synthetic polypeptide
QVQLVESGGGVVQPGRSLRLSCAASGFTFSSYGMHWVRQAPGEGLEWVTVISYDGSDKYYADSVKGRFTISRDNSKSTLFLQMNSLRAEDTAVYYCARDGQFYFDYWGQGTLVTVSS

SEQ ID NO: 118 - protein, 108 aa, synthetic construct
  REGION 1..108: note = Description of Artificial Sequence: Synthetic polypeptide
EIVMTQSPATLSLSPGESATLSCRASQSVTSRYLSWYQQKPGQAPRLLMYGASTRPTGIPARFSGSGSGTDFTLTISSLQPEDFAVYYCQQDYNLPLTFGGGTKVEIK

SEQ ID NO: 119 - protein, 243 aa, synthetic construct
  REGION 1..243: note = Description of Artificial Sequence: Synthetic polypeptide
QVQLVESGGGVVQPGRSLRLSCAASGFTFSSYGMHWVRQAPGEGLEWVTVISYDGSDKYYADSVKGRFTISRDNSKSTLFLQMNSLRAEDTAVYYCARDGQFYFDYWGQGTLVTVSSASTGGGGSGGGGSGGGGSEIVMTQSPATLSLSPGESATLSCRASQSVTSRYLSWYQQKPGQAPRLLMYGASTRPTGIPARFSGSGSGTDFTLTISSLQPEDFAVYYCQQDYNLPLTFGGGTKVEIK

SEQ ID NO: 120 - protein, 8 aa, synthetic construct
  REGION 1..8: note = Description of Artificial Sequence: Synthetic peptide
GFTFSNYG

SEQ ID NO: 121 - protein, 8 aa, synthetic construct
  REGION 1..8: note = Description of Artificial Sequence: Synthetic peptide
ISYDGSDK

SEQ ID NO: 122 - protein, 11 aa, synthetic construct
  REGION 1..11: note = Description of Artificial Sequence: Synthetic peptide
ARDSGRYFFDY

SEQ ID NO: 123 - protein, 7 aa, synthetic construct
  REGION 1..7: note = Description of Artificial Sequence: Synthetic peptide
QSVSSRS

SEQ ID NO: 124 - skipped

SEQ ID NO: 125 - protein, 9 aa, synthetic construct
  REGION 1..9: note = Description of Artificial Sequence: Synthetic peptide
HQDYNLPLT

SEQ ID NO: 126 - protein, 118 aa, synthetic construct
  REGION 1..118: note = Description of Artificial Sequence: Synthetic polypeptide
QVQLVESGGGVVQPGRSLRLSCAASGFTFSNYGMHWVRQAPGKGLEWVAVISYDGSDKSYKDSVKGRFTIARDNSKNTLYLQMNSLRAEDTAVYYCARDSGRYFFDYWGQGTLVTVSS

SEQ ID NO: 127 - protein, 108 aa, synthetic construct
  REGION 1..108: note = Description of Artificial Sequence: Synthetic polypeptide
EIIMTQSPATLSLSPGERATLSCRASQSVSSRSLSWYQHKPGQAPRLLIYGPSTRATGIPARFSGSGSGTDFTLTISSLQPEDFAVYYCHQDYNLPLTFGGGTKVEIK

SEQ ID NO: 128 - protein, 244 aa, synthetic construct
  REGION 1..244: note = Description of Artificial Sequence: Synthetic polypeptide
QVQLVESGGGVVQPGRSLRLSCAASGFTFSNYGMHWVRQAPGKGLEWVAVISYDGSDKSYKDSVKGRFTIARDNSKNTLYLQMNSLRAEDTAVYYCARDSGRYFFDYWGQGTLVTVSSASTGGGGSGGGGSGGGGSEIIMTQSPATLSLSPGERATLSCRASQSVSSRSLSWYQHKPGQAPRLLIYGPSTRATGIPARFSGSGSGTDFTLTISSLQPEDFAVYYCHQDYNLPLTFGGGTKVEIK

SEQ ID NO: 129 - protein, 8 aa, synthetic construct
  REGION 1..8: note = Description of Artificial Sequence: Synthetic peptide
GFTFSKYG

SEQ ID NO: 130 - protein, 8 aa, synthetic construct
  REGION 1..8: note = Description of Artificial Sequence: Synthetic peptide
IWYDGSIK

SEQ ID NO: 131 - protein, 14 aa, synthetic construct
  REGION 1..14: note = Description of Artificial Sequence: Synthetic peptide
ARGSLWFGEFYFDY

SEQ ID NO: 132 - protein, 6 aa, synthetic construct
  REGION 1..6: note = Description of Artificial Sequence: Synthetic peptide
QGISSA

SEQ ID NO: 133 - skipped

SEQ ID NO: 134 - protein, 9 aa, synthetic construct
  REGION 1..9: note = Description of Artificial Sequence: Synthetic peptide
QQFNNYPRT

SEQ ID NO: 135 - protein, 121 aa, synthetic construct
  REGION 1..121: note = Description of Artificial Sequence: Synthetic polypeptide
QVKLVESGGGVVQPGRSLRLSCAASGFTFSKYGMHWVRQAPGKGLEWVAFIWYDGSIKNYADSVKGRFTTSRDNSKNTLYLQMNSLRAEDTAVYYCARGSLWFGEFYFDYWGQGTLVTVSS

SEQ ID NO: 136 - protein, 107 aa, synthetic construct
  REGION 1..107: note = Description of Artificial Sequence: Synthetic polypeptide
AIQLTQSPSSLSASVGDRVTITCRTSQGISSALAWYQQKPGKTPKLLIYDASSLESGVPSRFSGSGSGTDFTLTISSLQPEDFATYYCQQFNNYPRTFGQGTKVEIK

SEQ ID NO: 137 - protein, 246 aa, synthetic construct
  REGION 1..246: note = Description of Artificial Sequence: Synthetic polypeptide
QVKLVESGGGVVQPGRSLRLSCAASGFTFSKYGMHWVRQAPGKGLEWVAFIWYDGSIKNYADSVKGRFTTSRDNSKNTLYLQMNSLRAEDTAVYYCARGSLWFGEFYFDYWGQGTLVTVSSASTGGGGSGGGGSGGGGSAIQLTQSPSSLSASVGDRVTITCRTSQGISSALAWYQQKPGKTPKLLIYDASSLESGVPSRFSGSGSGTDFTLTISSLQPEDFATYYCQQFNNYPRTFGQGTKVEIK

SEQ ID NO: 138 - protein, 361 aa, synthetic construct
  REGION 1..361: note = Description of Artificial Sequence: Synthetic polypeptide
QLQLQESGPGLVKPSETLSLTCTVSGGSISSSTYYWGWIRQPPRKGLEWIGSTHYRGSTYYNPSLKSRVTISVDTSKNQFSLKVSSVTAADTAVYYCARELTGEVFDYWGQGTLVTVSSASTGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCEL

SEQ ID NO: 139 - DNA, 1083 bp, synthetic construct
  misc_feature 1..1083: note = Description of Artificial Sequence: Synthetic polynucleotide
  source 1..1083: mol_type = other DNA
cagctccagctccaagagtcagggccaggtctcgtgaaaccgagtgagaccctgtccctgacctgcacagtgagtggtggatcaatctcaagctctacctactattgggggtggattcggcagccccctagaaaggggcttgagtggattggcagcactcattatcgaggatctacctattataatccttctctgaaaagcagagttaccatctctgtggatacgtccaaaaatcagttcagtctgaaggtatcatccgtgactgctgccgacacggccgtgtactattgcgcgagggagctgacaggtgaggtctttgactactggggccagggcacactcgtgaccgtgtcttctgcctcaacaggagggggtgggagtggaggcggtggatcagggggaggagggagtgacatccagatgacgcagtccccttccagcttgtccgcatctgtgggtgatagggtcacgattacatgtagggctagtcagagtatttctagttacctgaattggtaccagcagaaaccaggcaaggcaccaaagttgctcatctatgcggcctcctctctgcaatctggcgtgccgtccagatttagtggatcaggctccggaaccgatttcacccttacgatctcctcacttcaacccgaggatttcgccacatattactgtcaacaaagctattctacaccgttcaccttcggaccggggacaaaagtggatattaaacgggcggccgcccccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 140 - protein, 881 aa, synthetic construct
  REGION 1..881: note = Description of Artificial Sequence: Synthetic polypeptide
MALPVTALLLPLALLLHAQVQLQQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSSGGGGSGGGGSGGGGSEIVLTQSPATMSASPGERVTMSCSASSSVSYMHWYQQKSGQSPKRWIYDTSKLASGVPARFSGSGSGTDYTFTISSMEPEDFATYYCQQWSSNPLTFGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPRGSGATNFSLLKQAGDVEENPGPMALPVTALLLPLALLLHAQLQLQESGPGLVKPSETLSLTCTVSGGSISSSTYYWGWIRQPPRKGLEWIGSTHYRGSTYYNPSLKSRVTISVDTSKNQFSLKVSSVTAADTAVYYCARELTGEVFDYWGQGTLVTVSSASTGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKVDIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCEL

SEQ ID NO: 141 - DNA, 8940 bp, synthetic construct
  misc_feature 1..8940: note = Description of Artificial Sequence: Synthetic polynucleotide
  source 1..8940: mol_type = other DNA
ggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttttaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagaagccagtttgcatctgtcaggatcaatttcccattatgccagtcatattaattactagtcaattagttgatttttatttttgacatatacatgtgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggctctcccagtgactgccctactgcttcccctagcgcttctcctgcatgcacaagttcagctccagcagagcggcgccgaagtggcaaagcctggagcgtcagtcaagctgtcctgcaaagcgagtggctatacgttcacgaactactggatgcagtggataaagcaggctcccgggcagggtctggagtggattggagccgtctacccaggggacggcgacacccggcacactcaaaagttcaagggcaaggccaccctgaccgctgacaagagcacaagcacagcgtacatggaggtgtcctctttgagatccgaagataccgctgtgtattattgtgcccggggcttcactgcatacgggatggattactggggacaaggcactaccgtgactgtcagctccgggggtggaggctcaggcggggggggttcaggaggggggggatctgaaattgtgctgacacagagccctgccacaatgtctgctagccctggcgagcgcgtgaccatgtcttgtagcgccagcagcagcgtgtcctacatgcattggtatcaacagaagtccggccagtctcccaagcggtggatctacgatacaagcaagctggcctccggcgtgcccgccagattttctggcagcggctctggaacagattacaccttcaccatctctagcatggaacctgaggattttgccacctactattgccagcagtggtccagcaatcccctgacatttggaggaggcaccaagctggaaattaagagagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgcggaagcggagctactaacttcagcctgctgaagcaggctggagacgtggaggagaaccctggacccatggccctgcccgtcaccgctttgcttctgccactggccttgctgctccacgctcagctccagctccaagagtcagggccaggtctcgtgaaaccgagtgagaccctgtccctgacctgcacagtgagtggtggatcaatctcaagctctacctactattgggggtggattcggcagccccctagaaaggggcttgagtggattggcagcactcattatcgaggatctacctattataatccttctctgaaaagcagagttaccatctctgtggatacgtccaaaaatcagttcagtctgaaggtatcatccgtgactgctgccgacacggccgtgtactattgcgcgagggagctgacaggtgaggtctttgactactggggccagggcacactcgtgaccgtgtcttctgcctcaacaggagggggtgggagtggaggcggtggatcagggggaggagggagtgacatccagatgacgcagtccccttccagcttgtccgcatctgtgggtgatagggtcacgattacatgtagggctagtcagagtatttctagttacctgaattggtaccagcagaaaccaggcaaggcaccaaagttgctcatctatgcggcctcctctctgcaatctggcgtgccgtccagatttagtggatcaggctccggaaccgatttcacccttacgatctcctcacttcaacccgaggatttcgccacatattactgtcaacaaagctattctacaccgttcaccttcggaccggggacaaaagtggatattaaacgggcggccgcccccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaacaaacggggcagaaagaagctcctgtatatattcaaacaaccatttatgagaccagtacaaactactcaagaggaagatggctgtagctgccgatttccagaagaagaagaaggaggatgtgaactgtaacagccactcgaggatcc

SEQ ID NO: 142 - protein, 22 aa, synthetic construct
  REGION 1..22: note = Description of Artificial Sequence: Synthetic peptide
GSGATNFSLLKQAGDVEENPGP

SEQ ID NO: 143 - DNA, 66 bp, synthetic construct
  misc_feature 1..66: note = Description of Artificial Sequence: Synthetic oligonucleotide
  source 1..66: mol_type = other DNA
ggaagcggagctactaacttcagcctgctgaagcaggctggagacgtggaggagaaccctggaccc

SEQ ID NO: 144 - DNA, 354 bp, synthetic construct
  misc_feature 1..354: note = Description of Artificial Sequence: Synthetic polynucleotide
  source 1..354: mol_type = other DNA
caagttcagctccagcagagcggcgccgaagtggcaaagcctggagcgtcagtcaagctgtcctgcaaagcgagtggctatacgttcacgaactactggatgcagtggataaagcaggctcccgggcagggtctggagtggattggagccgtctacccaggggacggcgacacccggcacactcaaaagttcaagggcaaggccaccctgaccgctgacaagagcacaagcacagcgtacatggaggtgtcctctttgagatccgaagataccgctgtgtattattgtgcccggggcttcactgcatacgggatggattactggggacaaggcactaccgtgactgtcagctcc

SEQ ID NO: 145 - DNA, 318 bp, synthetic construct
  misc_feature 1..318: note = Description of Artificial Sequence: Synthetic polynucleotide
  source 1..318: mol_type = other DNA
gaaattgtgctgacacagagccctgccacaatgtctgctagccctggcgagcgcgtgaccatgtcttgtagcgccagcagcagcgtgtcctacatgcattggtatcaacagaagtccggccagtctcccaagcggtggatctacgatacaagcaagctggcctccggcgtgcccgccagattttctggcagcggctctggaacagattacaccttcaccatctctagcatggaacctgaggattttgccacctactattgccagcagtggtccagcaatcccctgacatttggaggaggcaccaagctggaaattaag

SEQ ID NO: 146 - protein, 118 aa, synthetic construct
  REGION 1..118: note = Description of Artificial Sequence: Synthetic polypeptide
QVQLVQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSS

SEQ ID NO: 147 - protein, 106 aa, synthetic construct; identical to SEQ ID NO: 40
  REGION 1..106: note = Description of Artificial Sequence: Synthetic polypeptide

SEQ ID NO: 148 - protein, 239 aa, synthetic construct
  REGION 1..239: note = Description of Artificial Sequence: Synthetic polypeptide
QVQLVQSGAEVAKPGASVKLSCKASGYTFTNYWMQWIKQAPGQGLEWIGAVYPGDGDTRHTQKFKGKATLTADKSTSTAYMEVSSLRSEDTAVYYCARGFTAYGMDYWGQGTTVTVSSGGGGSGGGGSGGGGSEIVLTQSPATMSASPGERVTMSCSASSSVSYMHWYQQKSGQSPKRWIYDTSKLASGVPARFSGSGSGTDYTFTISSMEPEDFATYYCQQWSSNPLTFGGGTKLEIK

SEQ ID NO: 149 - protein, 18 aa, synthetic construct
  REGION 1..18: note = Description of Artificial Sequence: Synthetic peptide
ASTGGGGSGGGGSGGGGS

SEQ ID NO: 150 - protein, 4 aa, synthetic construct
  REGION 1..4: note = Description of Artificial Sequence: Synthetic peptide
RAAA

SEQ ID NO: 151 - protein, 9 aa, synthetic construct
  REGION 1..9: note = Description of Artificial Sequence: Synthetic peptide
QQDYNLPIT

(19) United States
(12) Patent Application Publication
(10) Pub. No.: US 2025/0250356 A1
(43) Pub. Date: Aug. 7, 2025

(54) CHIMERIC RECEPTORS TARGETING ADGRE2 AND/OR CLEC12A AND USES THEREOF

(71) Applicants: Memorial Sloan-Kettering Cancer Center, New York, NY (US); Millennium Pharmaceuticals, Inc., Cambridge, MA (US); Sloan-Kettering Institute for Cancer Research, New York, NY (US); Memorial Hospital for Cancer and Allied Diseases, New York, NY (US)

(72) Inventors: Michel Sadelain, New York, NY (US); Sascha P. Haubner, New York, NY (US); Jorge Mansilla-Soto, Tampa, FL (US); Xingyue He, Cambridge, MA (US); Gary Shapiro, Cambridge, MA (US)

(73) Assignees: Memorial Sloan-Kettering Cancer Center, New York, NY (US); Millennium Pharmaceuticals, Inc., Cambridge, MA (US); Sloan-Kettering Institute for Cancer Research, New York, NY (US); Memorial Hospital for Cancer and Allied Diseases, New York, NY (US)

(21) Appl. No.: 19/185,375

(22) Filed: Apr. 22, 2025

Related U.S. Application Data
(62) Division of application No. 17/728,183, filed on Apr. 25, 2022, now Pat. No. 12,331,129.
(60) Provisional application No. 63/287,655, filed on Dec. 9, 2021.
(60) Provisional application No. 63/179,799, filed on Apr. 26, 2021.

(51) Int. Cl. C07K 16/28; A61K 38/00; A61K 39/00; A61K 40/11; A61K 40/31; A61K 40/42; A61P 35/00; A61P 35/02; C07K 14/705; C07K 14/725

(52) U.S. Cl. CPC C07K 16/2896; A61K 40/11; A61K 40/31; A61K 40/4202; A61K 40/4211; A61K 40/4224; A61P 35/00; A61P 35/02; C07K 14/7051; C07K 14/70517; C07K 14/70521; C07K 14/70578; C07K 16/2851; A61K 38/00; A61K 2039/505; A61K 2239/29; A61K 2239/31; A61K 2239/38; A61K 2239/48; C07K 2317/24; C07K 2317/565; C07K 2317/622; C07K 2317/92; C07K 2319/02; C07K 2319/03; C07K 2319/33

(57) ABSTRACT

The presently disclosed subject matter provides for chimeric receptors that target ADGRE2 and chimeric receptors that target CLEC12A. The presently disclosed subject matter also provides for cells comprising the ADGRE2-targeted chimeric receptors, cells comprising the CLEC12A-targeted chimeric receptors, and cells comprising the ADGRE2-targeted chimeric receptors and the CLEC12A-targeted chimeric receptors. The presently disclosed subject matter further provides uses of such cells for treating tumors, e.g., AML.

### CROSS-REFERENCE TO RELATED APPLICATIONS

This application is a Divisional application of U.S. patent application Ser. No. 17/728,183, filed Apr. 25, 2022, now allowed, which claims priority to U.S. Provisional Patent Application No. 63/179,799, filed Apr. 26, 2021, and U.S. Provisional Patent Application No. 63/287,655, filed Dec. 9, 2021, the contents of each of which are incorporated by reference in their entirety, and to each of which priority is claimed.

### SEQUENCE LISTING

The specification further incorporates by reference the Sequence Listing submitted herewith via Patent Center on Apr. 22, 2025. The Sequence Listing.xml file, identified as 0871080165.xml, is 211,649 bytes in size and was created on Apr. 17, 2025. The Sequence Listing, electronically filed herewith, does not extend beyond the scope of the specification and thus does not contain new matter.

### 1. INTRODUCTION

The presently disclosed subject matter provides methods and compositions for immunotherapies. It relates to chimeric receptors that target ADGRE2, and chimeric receptors that target CLEC12A, cells comprising such chimeric receptors, and methods of using such cells for treatments, e.g., for treating acute myeloid leukemia (AML).

### 2. BACKGROUND OF THE INVENTION

Cell-based immunotherapy is a therapy with curative potential for the treatment of cancer. T cells and other immune cells may be modified to target tumor antigens through the introduction of genetic material coding for artificial or synthetic receptors for antigen, termed Chimeric Antigen Receptors (CARs), specific to selected antigens. Targeted T cell therapy using CARs has shown recent clinical success in treating hematologic malignancies.

Relapsed and refractory acute myeloid leukemia (R/RAML) has a very poor prognosis. The only curative option is allogeneic hematopoietic stem cell transplantation, which is often associated with treatment failure and significant therapy-related toxicity and mortality. Novel therapeutic approaches are therefore direly needed for R/RAML. Over the past few years, autologous T cells genetically modified to express a chimeric antigen receptor (CAR) targeting CD19 have revolutionized the treatment and improved the outcomes of patients with R/R B-cell hematologic malignancies, leading to the approval by the FDA of three CD19 CARS (tisagenlecleucel, axicabtagene ciloleucel, and brexucabtagene autoleucel) for R/R acute lymphoblastic leukemia and/or certain B-cell non-Hodgkin lymphomas. In the case of AML, the clinical investigation of CAR T cell therapy is still in an early phase, and clinical results mainly with CD33 and CD123 CAR T cells suggest challenges both in terms of safety and efficacy, which are due to abundant expression of CD33 and CD123 in normal hematopoiesis and phenotypic heterogeneity in AML tumor cells. Accordingly, there are needs for a novel combinatorial CAR format for R/RAML that has the potential to provide improved safety and efficacy relative to alternative CAR therapies currently under clinical investigation.

### 3. SUMMARY OF THE INVENTION

The presently disclosed subject matter provides chimeric receptors that target ADGRE2, and chimeric receptors that target CLEC12A, cells comprising such chimeric receptors, and methods of using such cells for treatments, e.g., for treating acute myeloid leukemia (AML).

In one aspect, the presently disclosed subject matter provides chimeric receptors that target ADGRE2. In certain embodiments, the ADGRE2-targeted chimeric receptor comprises an extracellular antigen-binding domain that binds to ADGRE2, a transmembrane domain, and an intracellular domain. In certain embodiments, the extracellular antigen-binding domain comprises: a) a heavy chain variable region comprising a CDR1 comprising the amino acid sequence set forth in SEQ ID NO: 33 or a conservative modification thereof, a CDR2 comprising the amino acid sequence set forth in SEQ ID NO: 34 or a conservative modification thereof, and a CDR3 comprising the amino acid sequence set forth in SEQ ID NO: 35 or a conservative modification thereof; and/or b) a light chain variable region comprising a CDR1 comprising the amino acid sequence set forth in SEQ ID NO: 36 or a conservative modification thereof, a CDR2 comprising the amino acid sequence set forth in SEQ ID NO: 37 or a conservative modification thereof, and a CDR3 comprising the amino acid sequence set forth in SEQ ID NO: 38 or a conservative modification thereof.

In certain embodiments, the extracellular antigen-binding domain comprises a single-chain variable fragment (scFv), a Fab, or a F(ab)₂. In certain embodiments, the extracellular antigen-binding domain comprises an scFv. In certain embodiments, the scFv is a humanized scFv.

In certain embodiments, the heavy chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35. In certain embodiments, the light chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38. In certain embodiments, the heavy chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and the light chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38.

In certain embodiments, the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 39, SEQ ID NO:43, SEQ ID NO: 46, SEQ ID NO:49, SEQ ID NO: 52, SEQ ID NO: 55, or SEQ ID NO: 146. In certain embodiments, the heavy chain variable region comprises the amino acid sequence set forth in SEQ ID NO: 39, SEQ ID NO:43, SEQ ID NO:46, or SEQ ID NO:49. In certain embodiments, the heavy chain variable region comprises the amino acid sequence set forth in SEQ ID NO: 39.

In certain embodiments, the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 40, SEQ ID NO: 44, SEQ ID NO: 47, SEQ ID NO: 50, SEQ ID NO: 53, SEQ ID NO: 56, or SEQ ID NO: 147. In certain embodiments, the light chain variable region comprises the amino acid sequence set forth in SEQ ID NO: 40, SEQ ID NO: 44, SEQ ID NO: 47, or SEQ ID NO: 50. In certain embodiments, the light chain variable region comprises the amino acid sequence set forth in SEQ ID NO: 40.

In certain embodiments, the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 39, SEQ ID NO:43, SEQ ID NO: 46, SEQ ID NO:49, SEQ ID NO: 52, or SEQ ID NO: 55; and the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 40, SEQ ID NO: 44, SEQ ID NO: 47, SEQ ID NO: 50, SEQ ID NO: 53, or SEQ ID NO: 56. In certain embodiments, the heavy chain variable region comprises the amino acid sequence set forth in SEQ ID NO: 39, SEQ ID NO: 43, SEQ ID NO: 46, or SEQ ID NO:49; and the light chain variable region comprises the amino acid sequence set forth in SEQ ID NO: 40, SEQ ID NO: 44, SEQ ID NO: 47, or SEQ ID NO: 50.

In certain embodiments,
a) the heavy chain variable region comprises the amino acid sequence set forth in SEQ ID NO: 39; and the light chain variable region comprises the amino acid sequence set forth in SEQ ID NO: 40;
b) the heavy chain variable region comprises the amino acid sequence set forth in SEQ ID NO: 43; and the light chain variable region comprises the amino acid sequence set forth in SEQ ID NO: 44;
c) the heavy chain variable region comprises the amino acid sequence set forth in SEQ ID NO: 46; and the light chain variable region comprises the amino acid sequence set forth in SEQ ID NO: 47;
d) the heavy chain variable region comprises the amino acid sequence set forth in SEQ ID NO: 49; and the light chain variable region comprises the amino acid sequence set forth in SEQ ID NO: 50;
e) the heavy chain variable region comprises the amino acid sequence set forth in SEQ ID NO: 52; and the light chain variable region comprises the amino acid sequence set forth in SEQ ID NO: 53;
f) the heavy chain variable region comprises the amino acid sequence set forth in SEQ ID NO: 55; and the light chain variable region comprises the amino acid sequence set forth in SEQ ID NO: 56; or
g) the heavy chain variable region comprises the amino acid sequence set forth in SEQ ID NO: 146; and the light chain variable region comprises the amino acid sequence set forth in SEQ ID NO: 147.

In certain embodiments,
a) the heavy chain variable region comprises the amino acid sequence set forth in SEQ ID NO: 39; and the light chain variable region comprises the amino acid sequence set forth in SEQ ID NO: 40;
b) the heavy chain variable region comprises the amino acid sequence set forth in SEQ ID NO: 43; and the light chain variable region comprises the amino acid sequence set forth in SEQ ID NO: 44;
c) the heavy chain variable region comprises the amino acid sequence set forth in SEQ ID NO: 46; and the light chain variable region comprises the amino acid sequence set forth in SEQ ID NO: 47; or
d) the heavy chain variable region comprises the amino acid sequence set forth in SEQ ID NO: 49; and the light chain variable region comprises the amino acid sequence set forth in SEQ ID NO: 50.

In certain embodiments, the heavy chain variable region comprises the amino acid sequence set forth in SEQ ID NO: 39; and the light chain variable region comprises the amino acid sequence set forth in SEQ ID NO: 40.

In certain embodiments, the extracellular antigen-binding domain comprises a linker between the heavy chain variable region and the light chain variable region. In certain embodiments, the linker consists of the amino acid sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 149.

In certain embodiments, the heavy chain variable region and the light chain variable region are positioned from the N- to the C-terminus: V_{H}-V_{L}. In certain embodiments, the extracellular antigen-binding domain comprises or is an scFv, which comprises or consists of the amino acid sequence set forth in SEQ ID NO: 41, SEQ ID NO: 45, SEQ ID NO: 48, SEQ ID NO: 51, SEQ ID NO: 54, SEQ ID NO: 57, SEQ ID NO: 148. In certain embodiments, the extracellular antigen-binding domain comprises or is an scFv, which comprises or consists of the amino acid sequence set forth in SEQ ID NO: 41, SEQ ID NO: 45, SEQ ID NO: 48, or SEQ ID NO: 51. In certain embodiments, the extracellular antigen-binding domain comprises or is an scFv, which comprises or consists of the amino acid sequence set forth in SEQ ID NO: 41.

In certain embodiments, the extracellular antigen-binding domain binds to ADGRE2 with a dissociation constant (K_{D}) of less than about 10-8 M, less than about 10-9 M, less than about 10-10 M, less than about 10-11 M, less than about 10-12 M, or less than about 10-13 M. In certain embodiments, the extracellular antigen-binding domain binds to ADGRE2 with an EC50 of between about 1 and about 100 nM. In certain embodiments, the EC50 is between about 10 and about 95 nM. In certain embodiments, the EC50 is between about 25 and about 75 nM.

In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B 4 polypeptide, or a BTLA polypeptide. In certain embodiments, the transmembrane domain comprises a CD28 polypeptide.

In certain embodiments, the intracellular domain comprises a CD3% polypeptide. In certain embodiments, the CD3ζ polypeptide is a modified CD3ζ polypeptide. In certain embodiments, the modified CD3ζ polypeptide comprises a native ITAM1, an ITAM2 variant consisting of two loss-of-function mutations, and an ITAM3 variant consisting of two loss-of-function mutations. In certain embodiments, the native ITAM1 consists of the amino acid sequence set forth in SEQ ID NO: 15. In certain embodiments, the ITAM2 variant consists of the amino acid sequence set forth in SEQ ID NO: 21. In certain embodiments, the ITAM3 variant consists of the amino acid sequence set forth in SEQ ID NO: 25. In certain embodiments, the modified CD3ζ polypeptide comprises or consists of the amino acid sequence set forth in SEQ ID NO: 27.

In certain embodiments, the intracellular domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide.

In certain embodiments, the ADGRE2-targeted chimeric receptor is a chimeric antigen receptor (CAR), a chimeric co-stimulating receptor (CCR), or a TCR like fusion molecule. In certain embodiments, the ADGRE2-targeted chimeric receptor is a CAR.

In one aspect, the presently disclosed subject matter provides chimeric receptors that target CLEC12A. In certain embodiments, the CLEC12A-targeted chimeric receptor comprises an extracellular antigen-binding domain that binds to CLEC12A, a transmembrane domain, and an intracellular domain. In certain embodiments, the extracellular antigen-binding domain comprises a heavy chain variable region comprising:
i) a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof;
ii) a CDR1 comprising the amino acid sequence set forth in SEQ ID NO: 81 or a conservative modification thereof, a CDR2 comprising the amino acid sequence set forth in SEQ ID NO: 82 or a conservative modification thereof, and a CDR3 comprising the amino acid sequence set forth in SEQ ID NO: 83 or a conservative modification thereof;
iii) a CDR1 comprising the amino acid sequence set forth in SEQ ID NO: 89 or a conservative modification thereof, a CDR2 comprising the amino acid sequence set forth in SEQ ID NO: 90 or a conservative modification thereof, and a CDR3 comprising the amino acid sequence set forth in SEQ ID NO: 91 or a conservative modification thereof;
iv) a CDR1 comprising the amino acid sequence set forth in SEQ ID NO: 89 or a conservative modification thereof, a CDR2 comprising the amino acid sequence set forth in SEQ ID NO: 90 or a conservative modification thereof, and a CDR3 comprising the amino acid sequence set forth in SEQ ID NO: 98 or a conservative modification thereof;
v) a CDR1 comprising the amino acid sequence set forth in SEQ ID NO: 81 or a conservative modification thereof, a CDR2 comprising the amino acid sequence set forth in SEQ ID NO: 103 or a conservative modification thereof, and a CDR3 comprising the amino acid sequence set forth in SEQ ID NO: 83 or a conservative modification thereof;
vi) a CDR1 comprising the amino acid sequence set forth in SEQ ID NO: 109 or a conservative modification thereof, a CDR2 comprising the amino acid sequence set forth in SEQ ID NO: 103 or a conservative modification thereof, and a CDR3 comprising the amino acid sequence set forth in SEQ ID NO: 83 or a conservative modification thereof;
vii) a CDR1 comprising the amino acid sequence set forth in SEQ ID NO: 89 or a conservative modification thereof, a CDR2 comprising the amino acid sequence set forth in SEQ ID NO: 90 or a conservative modification thereof, and a CDR3 comprising the amino acid sequence set forth in SEQ ID NO: 114 or a conservative modification thereof;
viii) a CDR1 comprising the amino acid sequence set forth in SEQ ID NO: 120 or a conservative modification thereof, a CDR2 comprising the amino acid sequence set forth in SEQ ID NO: 121 or a conservative modification thereof, and a CDR3 comprising the amino acid sequence set forth in SEQ ID NO: 122 or a conservative modification thereof; or
ix) a CDR1 comprising the amino acid sequence set forth in SEQ ID NO: 129 or a conservative modification thereof, a CDR2 comprising the amino acid sequence set forth in SEQ ID NO: 130 or a conservative modification thereof, and a CDR3 comprising the amino acid sequence set forth in SEQ ID NO: 131 or a conservative modification thereof; and/or
   a light chain variable region comprising:
i) a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof;
ii) a CDR1 comprising the amino acid sequence set forth in SEQ ID NO: 84 or a conservative modification thereof, a CDR2 comprising the amino acid sequence set forth in SEQ ID NO: 73 or a conservative modification thereof, and a CDR3 comprising the amino acid sequence set forth in SEQ ID NO: 85 or a conservative modification thereof;
iii) a CDR1 comprising the amino acid sequence set forth in SEQ ID NO: 92 or a conservative modification thereof, a CDR2 comprising the amino acid sequence set forth in SEQ ID NO: 93 or a conservative modification thereof, and a CDR3 comprising the amino acid sequence set forth in SEQ ID NO: 94 or a conservative modification thereof;
iv) a CDR1 comprising the amino acid sequence set forth in SEQ ID NO: 99 or a conservative modification thereof, a CDR2 comprising the amino acid sequence set forth in SEQ ID NO: 93 or a conservative modification thereof, and a CDR3 comprising the amino acid sequence set forth in SEQ ID NO: 151 or a conservative modification thereof;
v) a CDR1 comprising the amino acid sequence set forth in SEQ ID NO: 104 or a conservative modification thereof, a CDR2 comprising the amino acid sequence set forth in SEQ ID NO: 73 or a conservative modification thereof, and a CDR3 comprising the amino acid sequence set forth in SEQ ID NO: 105 or a conservative modification thereof;
vi) a CDR1 comprising the amino acid sequence set forth in SEQ ID NO: 110 or a conservative modification thereof, a CDR2 comprising the amino acid sequence set forth in SEQ ID NO: 73 or a conservative modification thereof, and a CDR3 comprising the amino acid sequence set forth in SEQ ID NO: 85 or a conservative modification thereof;
vii) a CDR1 comprising the amino acid sequence set forth in SEQ ID NO: 115 or a conservative modification thereof, a CDR2 comprising the amino acid sequence set forth in SEQ ID NO: 93 or a conservative modification thereof, and a CDR3 comprising the amino acid sequence set forth in SEQ ID NO: 116 or a conservative modification thereof;
viii) a CDR1 comprising the amino acid sequence set forth in SEQ ID NO: 123 or a conservative modification thereof, a CDR2 comprising the amino acid sequence set forth in SEQ ID NO: 124 or a conservative modification thereof, and a CDR3 comprising the amino acid sequence set forth in SEQ ID NO: 125 or a conservative modification thereof; or
ix) a CDR1 comprising the amino acid sequence set forth in SEQ ID NO: 132 or a conservative modification thereof, a CDR2 comprising the amino acid sequence set forth in SEQ ID NO: 133 or a conservative modification thereof, and a CDR3 comprising the amino acid sequence set forth in SEQ ID NO: 134 or a conservative modification thereof.

In certain embodiments, the extracellular antigen-binding domain comprises a single-chain variable fragment (scFv), a Fab, or a F(ab)₂. In certain embodiments, the extracellular antigen-binding domain comprises an scFv. In certain embodiments, the scFv is a human scFv.

In certain embodiments, the heavy chain variable region comprises:
a) a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
b) a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83;
c) a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 91;
d) a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 98;
e) a CDR1 comprising the amino acid sequence set forth in SEQ ID NO: 81, a CDR2 comprising the amino acid sequence set forth in SEQ ID NO: 103, and a CDR3 comprising the amino acid sequence set forth in SEQ ID NO: 83;
f) a CDR1 comprising the amino acid sequence set forth in SEQ ID NO: 109, a CDR2 comprising the amino acid sequence set forth in SEQ ID NO: 103, and a CDR3 comprising the amino acid sequence set forth in SEQ ID NO: 83;
g) a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 114;
h) a CDR1 comprising the amino acid sequence set forth in SEQ ID NO: 120, a CDR2 comprising the amino acid sequence set forth in SEQ ID NO: 121, and a CDR3 comprising the amino acid sequence set forth in SEQ ID NO: 122; or
i) a CDR1 comprising the amino acid sequence set forth in SEQ ID NO: 129, a CDR2 comprising the amino acid sequence set forth in SEQ ID NO: 130, and a CDR3 comprising the amino acid sequence set forth in SEQ ID NO: 131.

In certain embodiments, the heavy chain variable region comprises:
a) a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71;
b) a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83;
c) a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 91; or
d) a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 98.

In certain embodiments, the heavy chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71.

In certain embodiments, the light chain variable region comprises:
a) a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
b) a CDR1 comprising the amino acid sequence set forth in SEQ ID NO: 84, a CDR2 comprising the amino acid sequence set forth in SEQ ID NO: 73, and a CDR3 comprising the amino acid sequence set forth in SEQ ID NO: 85;
c) a CDR1 comprising the amino acid sequence set forth in SEQ ID NO: 92, a CDR2 comprising the amino acid sequence set forth in SEQ ID NO: 93, and a CDR3 comprising the amino acid sequence set forth in SEQ ID NO: 94;
d) a CDR1 comprising the amino acid sequence set forth in SEQ ID NO: 99, a CDR2 comprising the amino acid sequence set forth in SEQ ID NO: 93, and a CDR3 comprising the amino acid sequence set forth in SEQ ID NO: 151;
e) a CDR1 comprising the amino acid sequence set forth in SEQ ID NO: 104, a CDR2 comprising the amino acid sequence set forth in SEQ ID NO: 73, and a CDR3 comprising the amino acid sequence set forth in SEQ ID NO: 105;
f) a CDR1 comprising the amino acid sequence set forth in SEQ ID NO: 110, a CDR2 comprising the amino acid sequence set forth in SEQ ID NO: 73, and a CDR3 comprising the amino acid sequence set forth in SEQ ID NO: 85;
g) a CDR1 comprising the amino acid sequence set forth in SEQ ID NO: 115, a CDR2 comprising the amino acid sequence set forth in SEQ ID NO: 93, and a CDR3 comprising the amino acid sequence set forth in SEQ ID NO: 116;
h) a CDR1 comprising the amino acid sequence set forth in SEQ ID NO: 123, a CDR2 comprising the amino acid sequence set forth in SEQ ID NO: 124, and a CDR3 comprising the amino acid sequence set forth in SEQ ID NO: 125; or
i) a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134.

In certain embodiments, the light chain variable region comprises:
a) a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
b) a CDR1 comprising the amino acid sequence set forth in SEQ ID NO: 84, a CDR2 comprising the amino acid sequence set forth in SEQ ID NO: 73, and a CDR3 comprising the amino acid sequence set forth in SEQ ID NO: 85;
c) a CDR1 comprising the amino acid sequence set forth in SEQ ID NO: 92, a CDR2 comprising the amino acid sequence set forth in SEQ ID NO: 93, and a CDR3 comprising the amino acid sequence set forth in SEQ ID NO: 94; or
d) a CDR1 comprising the amino acid sequence set forth in SEQ ID NO: 99, a CDR2 comprising the amino acid sequence set forth in SEQ ID NO: 93, and a CDR3 comprising the amino acid sequence set forth in SEQ ID NO: 151.

In certain embodiments, the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

In certain embodiments, the chimeric receptor comprises:
a) the heavy chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
b) the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 73, and a CDR3 comprising the amino acid sequence set forth in SEQ ID NO: 85;
c) the heavy chain variable region comprises a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 91; and the light chain variable region comprises a CDR1 comprising the amino acid sequence set forth in SEQ ID NO: 92, a CDR2 comprising the amino acid sequence set forth in SEQ ID NO: 93, and a CDR3 comprising the amino acid sequence set forth in SEQ ID NO: 94;
d) the heavy chain variable region comprises a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 98; and the light chain variable region comprises a CDR1 comprising the amino acid sequence set forth in SEQ ID NO: 99, a CDR2 comprising the amino acid sequence set forth in SEQ ID NO: 93, and a CDR3 comprising the amino acid sequence set forth in SEQ ID NO: 151;
e) the heavy chain variable region comprises a CDR1 comprising the amino acid sequence set forth in SEQ ID NO: 81, a CDR2 comprising the amino acid sequence set forth in SEQ ID NO: 103, and a CDR3 comprising the amino acid sequence set forth in SEQ ID NO: 83; and the light chain variable region comprises a CDR1 comprising the amino acid sequence set forth in SEQ ID NO: 104, a CDR2 comprising the amino acid sequence set forth in SEQ ID NO: 73, and a CDR3 comprising the amino acid sequence set forth in SEQ ID NO: 105;
f) the heavy chain variable region comprises a CDR1 comprising the amino acid sequence set forth in SEQ ID NO: 109, a CDR2 comprising the amino acid sequence set forth in SEQ ID NO: 103, and a CDR3 comprising the amino acid sequence set forth in SEQ ID NO: 83; and the light chain variable region comprises a CDR1 comprising the amino acid sequence set forth in SEQ ID NO: 110, a CDR2 comprising the amino acid sequence set forth in SEQ ID NO: 73, and a CDR3 comprising the amino acid sequence set forth in SEQ ID NO: 85;
g) the heavy chain variable region comprises a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 114; and the light chain variable region comprises a CDR1 comprising the amino acid sequence set forth in SEQ ID NO: 115, a CDR2 comprising the amino acid sequence set forth in SEQ ID NO: 93, and a CDR3 comprising the amino acid sequence set forth in SEQ ID NO: 116;
h) the heavy chain variable region comprises a CDR1 comprising the amino acid sequence set forth in SEQ ID NO: 120, a CDR2 comprising the amino acid sequence set forth in SEQ ID NO: 121, and a CDR3 comprising the amino acid sequence set forth in SEQ ID NO: 122; and the light chain variable region comprises a CDR1 comprising the amino acid sequence set forth in SEQ ID NO: 123, a CDR2 comprising the amino acid sequence set forth in SEQ ID NO: 124, and a CDR3 comprising the amino acid sequence set forth in SEQ ID NO: 125; or
i) the heavy chain variable region comprises a CDR1 comprising the amino acid sequence set forth in SEQ ID NO: 129, a CDR2 comprising the amino acid sequence set forth in SEQ ID NO: 130, and a CDR3 comprising the amino acid sequence set forth in SEQ ID NO: 131; and the light chain variable region comprises a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134.

In certain embodiments,
a) the heavy chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
b) the heavy chain variable region comprises a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and the light chain variable region comprises a CDR1 comprising the amino acid sequence set forth in SEQ ID NO: 84, a CDR2 comprising the amino acid sequence set forth in SEQ ID NO: 73, and a CDR3 comprising the amino acid sequence set forth in SEQ ID NO: 85;
c) the heavy chain variable region comprises a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 91; and the light chain variable region comprises a CDR1 comprising the amino acid sequence set forth in SEQ ID NO: 92, a CDR2 comprising the amino acid sequence set forth in SEQ ID NO: 93, and a CDR3 comprising the amino acid sequence set forth in SEQ ID NO: 94; or
d) the heavy chain variable region comprises a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 98; and the light chain variable region comprises a CDR1 comprising the amino acid sequence set forth in SEQ ID NO: 99, a CDR2 comprising the amino acid sequence set forth in SEQ ID NO: 93, and a CDR3 comprising the amino acid sequence set forth in SEQ ID NO: 151.

In certain embodiments, the heavy chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

In certain embodiments, the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 75, SEQ ID NO: 86, SEQ ID NO: 95, SEQ ID NO: 100, SEQ ID NO: 106, SEQ ID NO: 111, SEQ ID NO: 117, SEQ ID NO: 126, or SEQ ID NO: 135. In certain embodiments, the heavy chain variable region comprises the amino acid sequence set forth in SEQ ID NO: 75, SEQ ID NO: 86, SEQ ID NO: 95, SEQ ID NO: 100, SEQ ID NO: 106, SEQ ID NO: 111, SEQ ID NO: 117, SEQ ID NO: 126, or SEQ ID NO: 135. In certain embodiments, the heavy chain variable region comprises the amino acid sequence set forth in SEQ ID NO: 75, SEQ ID NO: 86, SEQ ID NO: 95, or SEQ ID NO: 100. In certain embodiments, the heavy chain variable region comprises the amino acid sequence set forth in SEQ ID NO: 75.

In certain embodiments, the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 76, SEQ ID NO: 87, SEQ ID NO: 96, SEQ ID NO: 101, SEQ ID NO: 107, SEQ ID NO: 112, SEQ ID NO: 118, SEQ ID NO: 127, or SEQ ID NO: 136. In certain embodiments, the light chain variable region comprises the amino acid sequence set forth in SEQ ID NO: 76, SEQ ID NO: 87, SEQ ID NO: 96, SEQ ID NO: 101, SEQ ID NO: 107, SEQ ID NO: 112, SEQ ID NO: 118, SEQ ID NO: 127, or SEQ ID NO: 136. In certain embodiments, the light chain variable region comprises the amino acid sequence set forth in SEQ ID NO: 76, SEQ ID NO: 87, SEQ ID NO: 96, or SEQ ID NO: 101. In certain embodiments, the light chain variable region comprises the amino acid sequence set forth in SEQ ID NO: 76.

In certain embodiments,
a) the heavy chain variable region comprises the amino acid sequence set forth in SEQ ID NO: 75; and the light chain variable region comprises the amino acid sequence set forth in SEQ ID NO: 76;
b) the heavy chain variable region comprises the amino acid sequence set forth in SEQ ID NO: 86; and the light chain variable region comprises the amino acid sequence set forth in SEQ ID NO: 87;
c) the heavy chain variable region comprises the amino acid sequence set forth in SEQ ID NO: 95; and the light chain variable region comprises the amino acid sequence set forth in SEQ ID NO: 96;
d) the heavy chain variable region comprises the amino acid sequence set forth in SEQ ID NO: 100; and the light chain variable region comprises the amino acid sequence set forth in SEQ ID NO: 101;
e) the heavy chain variable region comprises the amino acid sequence set forth in SEQ ID NO: 106; and the light chain variable region comprises the amino acid sequence set forth in SEQ ID NO: 107;
f) the heavy chain variable region comprises the amino acid sequence set forth in SEQ ID NO: 111; and the light chain variable region comprises the amino acid sequence set forth in SEQ ID NO: 112;
g) the heavy chain variable region comprises the amino acid sequence set forth in SEQ ID NO: 117; and the light chain variable region comprises the amino acid sequence set forth in SEQ ID NO: 118;
h) the heavy chain variable region comprises the amino acid sequence set forth in SEQ ID NO: 126; and the light chain variable region comprises the amino acid sequence set forth in SEQ ID NO: 127; or
i) the heavy chain variable region comprises the amino acid sequence set forth in SEQ ID NO: 135; and the light chain variable region comprises the amino acid sequence set forth in SEQ ID NO: 136.

In certain embodiments, the chimeric receptor comprises:
a) the heavy chain variable region comprises the amino acid sequence set forth in SEQ ID NO: 75; and the light chain variable region comprises the amino acid sequence set forth in SEQ ID NO: 76;
b) the heavy chain variable region comprises the amino acid sequence set forth in SEQ ID NO: 86; and the light chain variable region comprises the amino acid sequence set forth in SEQ ID NO: 87;
c) the heavy chain variable region comprises the amino acid sequence set forth in SEQ ID NO: 95; and the light chain variable region comprises the amino acid sequence set forth in SEQ ID NO: 96; or
d) the heavy chain variable region comprises the amino acid sequence set forth in SEQ ID NO: 100; and the light chain variable region comprises the amino acid sequence set forth in SEQ ID NO: 101.

In certain embodiments, the heavy chain variable region comprises the amino acid sequence set forth in SEQ ID NO: 75; and the light chain variable region comprises the amino acid sequence set forth in SEQ ID NO: 76.

In certain embodiments, the extracellular antigen-binding domain comprises a linker between the heavy chain variable region and the light chain variable region. In certain embodiments, the linker consists of the amino acid sequence set forth in SEQ ID NO: 1, SEQ ID NO: 2, SEQ ID NO: 3, SEQ ID NO: 4, SEQ ID NO: 5, SEQ ID NO: 6, or SEQ ID NO: 149.

In certain embodiments, the heavy chain variable region and the light chain variable region are positioned from the N- to the C-terminus: V_{H}-V_{L}. In certain embodiments, the extracellular antigen-binding domain comprises or is an scFv, which comprises or consists of the amino acid sequence set forth in SEQ ID NO: 79, SEQ ID NO: 88, SEQ ID NO: 97, SEQ ID NO: 102, SEQ ID NO: 108, SEQ ID NO: 113, SEQ ID NO: 119, SEQ ID NO: 128, or SEQ ID NO: 137. In certain embodiments, the extracellular antigen-binding domain comprises or is an scFv, which comprises or consists of the amino acid sequence set forth in SEQ ID NO: 79, SEQ ID NO: 88, SEQ ID NO: 97, or SEQ ID NO: 102. In certain embodiments, the extracellular antigen-binding domain comprises or is an scFv, which comprises or consists of the amino acid sequence set forth in SEQ ID NO: 79.

In certain embodiments, the extracellular antigen-binding domain comprises binds to CLEC12A with a disassociation constant (K_{D}) of less than about 10-8 M, less than about 10-9 M, less than about 10-10 M, less than about 10-11 M, less than about 10-12 M, or less than about 10-13 M. In certain embodiments, the K_{D} is about 0.1 pM or below. In certain embodiments, the K_{D} is between about 0.05 PM and about 0.5 pM. In certain embodiments, the K_{D} is between about 0.1 nM and about 5.0 nM. In certain embodiments, the K_{D} is between about 0.3 nM and about 3.5 nM. In certain embodiments, the extracellular antigen-binding domain binds to CLEC12A with an EC50 of between about 1 nM and about 100 nM.

In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B 4 polypeptide, or a BTLA polypeptide. In certain embodiments, the transmembrane domain comprises a CD8 polypeptide.

In certain embodiments, the CLEC12A-targeted chimeric receptor is a chimeric antigen receptor (CAR), a chimeric co-stimulating receptor (CCR), or a TCR like fusion molecule. In certain embodiments, the CLEC12A-targeted chimeric receptor is a chimeric co-stimulating receptor (CCR).

In certain embodiments, the intracellular domain does not comprise a CD3% polypeptide. In certain embodiments, the intracellular domain comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a 4-1BB polypeptide.

In certain embodiments, the chimeric receptor is expressed from a vector. In certain embodiments, the vector is a viral vector. In certain embodiments, the viral vector is a retroviral vector.

The presently disclosed subject matter further provides cells comprising the chimeric receptor disclosed herein. In certain embodiments, the cell comprises an ADGRE2-targeted chimeric receptor disclosed herein. In certain embodiments, the cell comprises a CLEC12A-targeted chimeric receptor disclosed herein. In certain embodiments, the cell comprises a) an ADGRE2-targeted chimeric receptor disclosed herein, and b) a CLEC12A-targeted chimeric receptor disclosed herein. In certain embodiments, the ADGRE2-targeted chimeric receptor is a chimeric antigen receptor (CAR) and the CLEC12A-targeted chimeric receptor is a a chimeric co-stimulating receptor (CCR).

In certain embodiments, the CAR comprises an extracellular antigen-binding domain that binds to ADGRE2 and comprises:
a) a heavy chain variable region comprising a CDR1 comprising the amino acid sequence set forth in SEQ ID NO: 33 or a conservative modification thereof, a CDR2 comprising the amino acid sequence set forth in SEQ ID NO: 34 or a conservative modification thereof, and a CDR3 comprising the amino acid sequence set forth in SEQ ID NO: 35 or a conservative modification thereof; and/or
b) a light chain variable region comprising a CDR1 comprising the amino acid sequence set forth in SEQ ID NO: 36 or a conservative modification thereof, a CDR2 comprising the amino acid sequence set forth in SEQ ID NO: 37 or a conservative modification thereof, and a CDR3 comprising the amino acid sequence set forth in SEQ ID NO: 38 or a conservative modification thereof.

In certain embodiments, the heavy chain variable region comprises a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and the light chain variable region comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38.

In certain embodiments,
a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 39; and/or
b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 40.

In certain embodiments, the heavy chain variable region comprises the amino acid sequence set forth in SEQ ID NO: 39; and the light chain variable region comprises the amino acid sequence set forth in SEQ ID NO: 40.

In certain embodiments, the CCR comprises an extracellular antigen-binding domain that binds to CLEC12A and comprises:
a) a heavy chain variable region comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof; and/or
b) a light chain variable region comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof.

In certain embodiments, the heavy chain variable region comprises a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and the light chain variable region comprises a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74.

In certain embodiments,
a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 75; and/or
b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical or homologous to the amino acid sequence set forth in SEQ ID NO: 76.

In certain embodiments, the heavy chain variable region comprises the amino acid sequence set forth in SEQ ID NO: 75; and the light chain variable region comprises the amino acid sequence set forth in SEQ ID NO: 76.

In certain embodiments, the cell is transduced with the ADGRE2-targeted chimeric receptor and/or the CLEC12A-targeted chimeric receptor. In certain embodiments, the ADGRE2-targeted chimeric receptor and/or the CLEC12A-targeted chimeric receptor is constitutively expressed on the surface of the cell. In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, a stem cell from which a lymphoid cell may be differentiated, and a stem cell from which a myeloid cell may be differentiated. In certain embodiments, the cell is a T cell. In certain embodiments, the T cell is selected from the group consisting of helper T cells, cytotoxic T cells, memory T cells, regulatory T cells, tumor-infiltrating lymphocyte (TIL), Natural Killer T cells, mucosal associated invariant T cells, and γδ T cells. In certain embodiments, the cell is a Natural Killer (NK) cell. In certain embodiments, the NK cell is derived from a stem cell. In certain embodiments, the stem cell is a pluripotent stem cell. In certain embodiments, the pluripotent stem cell is an embryoid stem cell or an induced pluripotent stem cell.

Furthermore, the presently disclosed subject matter provides nucleic acid molecules encoding the chimeric receptors disclosed herein. In certain embodiments, the nucleic acid molecule encodes an ADGRE2-targeted chimeric receptor disclosed herein. In certain embodiments, the nucleic acid molecule encodes a CLEC12A-targeted chimeric receptor disclosed herein.

In certain embodiments, the nucleic acid molecule further comprises a promoter that is operably linked to the chimeric receptor. In certain embodiments, the promoter is endogenous or exogenous. In certain embodiments, the exogenous promoter is selected from the group consisting of an elongation factor (EF)-1 promoter, a cytomegalovirus immediate-early promoter (CMV) promoter, a simian virus 40 early promoter (SV40) promoter, a phosphoglycerate kinase (PGK) promoter, a metallothionein promoter, and Ubiquitin C promoter. In certain embodiments, the promoter is an inducible promoter. In certain embodiments, the inducible promoter is selected from the group consisting of a NFAT transcriptional response element (TRE) promoter, a CD69 promoter, a CD25 promoter, an IL-2 promoter, a 4-1BB promoter, a PD 1 promoter, and a LA G 3 promoter. In certain embodiments, the promoter is an endogenous promoter. In certain embodiments, the endogenous promoter is selected from a TCR alpha promoter, a TCR beta promoter, and a beta 2-microglobulin promoter.

The presently disclosed subject matter also provides a nucleic acid composition comprising a first nucleic acid molecule encoding an ADGRE2-targeted chimeric receptor disclosed herein, and a second nucleic acid molecule encoding a CLEC12A-targeted chimeric receptor disclosed herein.

The presently disclosed subject matter also provides vectors comprising the nucleic acid molecule disclosed herein or the nucleic acid composition disclosed herein. In certain embodiments, the vector is a viral vector. In certain embodiments, the vector is a retroviral vector.

The presently disclosed subject matter further provides cells expressing the nucleic acid molecule disclosed herein or the nucleic acid composition disclosed herein. In certain embodiments, the cell is a T cell.

The presently disclosed subject matter provides compositions comprising the cell disclosed herein. In certain embodiments, the composition is a pharmaceutical composition further comprising a pharmaceutically acceptable carrier. In certain embodiments, the composition comprises between about 25×10⁶ and about 150×10⁶ cells. In certain embodiments, the composition comprises between about 25×10⁶ and about 50×10⁶ cells. In certain embodiments, the composition comprises about 2.5×10⁶ cells.

The presently disclosed subject matter further provides various methods of using the presently disclosed cells. The presently disclosed subject matter provides methods of reducing tumor burden in a subject. In certain embodiments, the method comprises administering to the subject the cells or the composition disclosed herein. In certain embodiments, the method reduces the number of tumor cells, reduces tumor size, and/or eradicates the tumor in the subject.

The presently disclosed subject matter provides methods of increasing or lengthening survival of a subject having a tumor. In certain embodiments, the methods comprise administering to the subject the cells or the composition disclosed herein.

The presently disclosed subject matter provides methods of treating and/or preventing a tumor in a subject. In certain embodiments, the methods comprise administering to the subject the cells or the composition disclosed herein.

In certain embodiments, the tumor expresses ADGRE2 and/or CLEC12A. In certain embodiments, the tumor is cancer. In certain embodiments, the tumor is blood cancer. In certain embodiments, the tumor is selected from the group consisting of multiple myeloma, leukemia, lymphomas, and myeloid malignancies. In certain embodiments, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. In certain embodiments, the leukemia is acute myeloid leukemia (AML). In certain embodiments, the AML is relapsed/refractory acute myeloid leukemia (R/RAML). In certain embodiments, the myeloid malignancies are selected from the group consisting of myelodysplastic syndromes (MDS), myeloproliferative neoplasms (MPN), myeloid/lymphoid neoplasms (e.g., myeloid/lymphoid neoplasms with eosinophilia and rearrangement of Platelet Derived Growth Factor Receptor Alpha (PDGFRA), Platelet Derived Growth Factor Receptor Beta (PDGFRB), or Fibroblast Growth Factor Receptor 1 (FGFR1), or with PCM 1-JAK 2), acute myeloid leukemia (AML), blastic plasmacytoid dendritic cell neoplasm, B-lymphoblastic leukemia/lymphoma, and T-lymphoblastic leukemia/lymphoma. In certain embodiments, the myeloid malignancies comprise myelodysplastic syndromes (MDS). In certain embodiments, the subject is a human subject.

Furthermore, the presently disclosed subject matter provides methods for producing cells comprising the chimeric receptors disclosed herein. In certain embodiments, the method comprises introducing into the cell a nucleic acid molecule that encodes the chimeric receptor disclosed herein.

Furthermore, the presently disclosed subject matter provides methods for producing a cell comprising an ADGRE2-targeted chimeric receptor disclosed herein, and a CLEC12A-targeted chimeric receptor disclosed herein. In certain embodiments, the method comprises introducing into the cell a nucleic acid molecule that encodes the ADGRE2-targeted chimeric receptor and a nucleic acid molecule that encodes the CLEC12A-targeted chimeric receptor disclosed herein.

### 4. BRIEF DESCRIPTION OF THE FIGURES

The following Detailed Description, given by way of example, but not intended to limit the invention to specific embodiments described, may be understood in conjunction with the accompanying drawings.

FIGS. 1A and 1B illustrate target antigen expression profiles of ADGRE2, CD33, and CD123 evaluated via flow cytometry of fresh AML patient samples (peripheral blood or bone marrow); n=16 r/r adult AML patients with the morphological disease. Target expression was compared between bulk AML cells (CD45_{dim}/SSC_{low} gate) and leukemic stem cells [LSC; Lin-CD34+CD38-(CD45RA+) within bulk AML cell gate]. Gates for target-positivity were set based on the same-sample negative control population (T or B cells). FIG. 1A shows the percentage of target-positive AML cells within the bulk or LSC population of an individual patient sample represented by each dot. In the majority of evaluated AML patients >90% of AML cells were positive for ADGRE2, both for bulk and LSC. FIG. 1B shows the percentage of evaluated AML patient population with >70% target-positive AML cells. All evaluated AML patients had >70% ADGRE2-positive AML cells, both for bulk and LSC.

FIG. 2 illustrates a schematic of CAR, CCR, and CAR+CCR.

FIG. 3 depicts the rationale for ADCLEC.syn1 approach for improved anti-leukemic efficacy compared to CD33 and CD123-CAR. ADGRE2-CAR alone provides some anti-leukemic activity but may be limited due to ADGRE2-low escape mechanisms. CLEC12A-CCR alone does not mediate any cell lysis. ADCLEC.syn1 combines a low-affinity ADGRE2-CAR with a high-affinity CLEC12A-CCR, thereby increasing AML-directed avidity and reducing the risk of ADGRE2-low AML escape. In addition, CLEC12A-CCR-dependent trans-co-stimulation via 4-1BB further enhances T cell functionality. In comparison, single-targeting CD33-CAR or CD123-CAR approaches could be limited in efficacy due to antigen-low escape and phenotypic heterogeneity (see target profiles in FIG. 1).

FIGS. 4A-4C illustrate protein expression profiles of ADGRE2, CLEC12A (CD371), CD33, and CD123 in normal hematopoiesis and non-hematopoietic tissues, showing largely non-overlapping expression profile of ADGRE2 and CLEC12A. FIGS. 4A and 4B show flow cytometric co-expression profiles of ADGRE2/CLEC12A vs CD33/CD123 on representative adult normal donor bone marrow cell populations (gating: monocytes—CD45ₕᵢ/SSC_{med}/CD14+, granulocytes—CD45_{dim}/SSCₕᵢ, HSC—CD45_{dim}/SSC_{low}/CD34+/CD38−/CD45RA−/CD90+, T cells—CD45ₕᵢ/SSC_{low}/CD3+, B cells—CD45ₕᵢ/SSC_{low}/CD19+). FIG. 4C shows a heatmap depicting the summary of immunohistochemistry co-staining for ADGRE2 and CLEC12A in formalin-fixed paraffin-embedded normal human tissues. Staining intensity refers to cellular positivity, excluding fluids with a high probability for unspecific staining. ADGRE2/CLEC12A had expected co-expression on myeloid cells in immune-related tissues (mostly monocytic lineage), but no or restricted overlapping expression was found in other evaluated tissues.

FIG. 5 depicts the rationale for ADCLEC.syn1 approach for improved safety profile compared to CD33- and CD123-CAR. ADGRE2-CAR with optimized affinity and fine-turned CD3ζ-signaling strength alone spares cells with low or very-low levels of ADGRE2 (HSC or granulocytes, respectively). High-affinity CLEC12A-CCR alone does not mediate any cell lysis. ADCLEC.syn1 combines a low-affinity ADGRE2-CAR with a high-affinity CLEC12A-CCR. Normal hematopoietic cells have a largely non-overlapping expression profile of ADGRE2 and CLEC12A, and CLEC12A is negative on HSC. Therefore, the addition of a CLEC12A-CCR does not increase the risk of HSC toxicity.

FIG. 6 illustrates the ADGRE2 scFv binder selection scheme.

FIGS. 7A-7C illustrate in vitro 18 h CAR cytotoxicity assay in the context of different ADGRE2 target expression levels. A favorable profile for leading humanized ADGRE2 scFvs, with maximum cytotoxicity at high ADGRE2 levels and minimal cytotoxicity at very-low ADGRE2 levels was observed. Different humanized ADGRE2 scFv candidates and the original 2A 1 scFv were tested in SFG-retroviral 28z1XX CAR vector backbone; for each ADGRE2 scFv, 2 signal peptides were studied, one containing our established CD8a signal peptide and the other containing an alternative IgHV 1-4 signal peptide. Shown in colored lines are 6 scFvs with cytotoxicity features. The remaining scFv candidates (not included in the legend) are shown in grey. CAR T cells were cocultured for 18 h with MOLM 13 AML cell lines expressing different levels of ADGRE2: high (WT, FIG. 7A), low (clone 1E8, FIG. 7B), and very-low (clone 9D6, FIG. 7C). Cytotoxicity was measured based on the Luciferase signal released from MOLM 13 cells.

FIG. 8 illustrates in vivo antitumor CAR efficacy of six (6) humanized ADGRE2 scFvs in the context of different ADGRE2 target expression levels. In vivo antitumor CAR efficacy model showing a favorable profile for leading humanized ADGRE2 scFvs, with potent efficacy at high ADGRE2 levels, reduced cytotoxicity at low ADGRE2 levels, and absent cytotoxicity at very-low ADGRE2 levels. Six different humanized ADGRE2 scFv candidates and the original 2A 1 scFv were tested in the SFG-based retroviral 28z1XX CAR backbone. In vivo MOLM 13 AML cell line xenograft experiment with 6-8 week-old NSG mice. On day-5, mice were injected via tail-vein with the indicated MOLM 13 cell line clone (dose: 1E6 cells per mouse). On day-1, AML engraftment was confirmed via ffLuc-based in vivo bioluminescence imaging. On day 0, mice were injected via tail-vein with CAR T cells (dose: 3E5 CAR-positive cells per mouse). Subsequently, the AML burden was quantified via bioluminescence imaging and is represented via total flux (p/s).

FIG. 9 illustrates CLEC12A scFv binder selection scheme.

FIGS. 10A and 10B illustrate in vitro CAR cytotoxicity of CLEC12A scFvs in TRAC CAR-28z1XX format in the context of AML cell lines U937 and MOLM 13. In vitro 18 h CAR cytotoxicity assay showing a favorable profile for leading CLEC12A scFvs, with potent efficacy at high and low CLEC12A levels. Different CLEC12A scFv candidates were tested in TRAC-AAV 28z1XX CAR backbone. Shown in colored lines are the nine (9) scFv with the highest efficacy in the context of different CLEC12A levels. FIG. 10A shows cytotoxicity with CLEC12A-high cell line U 937. FIG. 10B shows cytotoxicity with CLEC12A-low cell line MOLM 13. The remaining scFv candidates are shown in grey. T cells were cocultured for 18 h with AML cell lines. Cytotoxicity was measured based on Luciferase signal from AML cell lines.

FIG. 11 illustrates in vivo antitumor CAR efficacy model showing high efficacy for leading CLEC12A scFvs. Nine (9) different CLEC12A scFv candidates were tested in the TRAC-AAV 28z1XX CAR backbone. In vivo U937 AML cell line xenograft experiment with 6-8 week-old NSG mice. On day-4, mice were injected via tail-vein with the indicated U 937 cell line clone (dose: 1E6 cells per mouse). On day-1, AML engraftment was confirmed via ffLuc-based in vivo bioluminescence imaging. On day 0, mice were injected via tail-vein with CAR T cells (dose: 4E5 CAR-positive cells per mouse). Subsequently, the AML burden was quantified via bioluminescence imaging and is represented via total flux (p/s).

FIGS. 12A-12C illustrate in vitro and in vivo CAR efficacy assays validating the ADCLEC.syn1 concept (as outlined as a schematic in FIG. 3). FIG. 12A shows in vitro 18 h CAR cytotoxicity assay. ADCLEC.syn1-transduced T cells were cocultured with EL 4 murine lymphoma cell line expressing no target (ADGRE2−/CLEC12A−) or overexpressing either CAR target alone (ADGRE2+/CLEC12A−), CCR target alone (ADGRE2−/CLEC12A+), or both CAR and CCR target (ADGRE2+/CLEC12A+). CD19-targeting 1928z1XX CAR construct was used as a negative control. % Cytotoxicity indicates target-specific killing efficacy at a given effector:target (E:T) ratio. FIGS. 12B and 12C show in vivo MOLM 13 AML cell line xenograft experiment with 6-8 week-old NSG mice. On day-5, mice were injected via tail-vein with the indicated M OLM 13 cell line clone (dose: 1×10⁶ cells per mouse). On day-1, AML engraftment was confirmed via ffLuc-based in vivo bioluminescence imaging. On day 0, mice were injected via tail-vein with CAR T cells (dose: 5×10⁵ CAR-positive cells per mouse). Subsequently, the AML burden was quantified via bioluminescence imaging and is represented via total flux (p/s).

FIG. 13 illustrates a pSFG-ADCLEC.syn1 restriction map (8940 bp).

FIG. 14 illustrates the gammaretroviral vector design for ADCLEC.syn1 bicistronic construct including ADGRE2ADGRE2-A-CAR and CLEC12ACLEC12A-A-CCR. Abbreviations: LTR=long terminal repeat, SD=splice donor site, SA=splice acceptor site, SP =signal peptide, scFv=single-chain variable fragment, H=hinge, TM=transmembrane, C=costimulatory domain, S=stimulatory domain.

FIG. 15 illustrates a representative ADCLEC.syn1 CAR T cell manufacturing plan.

FIG. 16 illustrates an algorithm to guide treatment course following D 30 disease assessment.

FIGS. 17A and 17B illustrate solubilized membrane protein (SM P) assays.

FIGS. 18A and 18B illustrate combinatorial gating strategies. FIG. 18A shows previously described combinatorial CAR gating strategies. FIG. 18B shows the “IF-BETTER” gating strategy.

FIGS. 19A and 19B illustrate in vitro CAR efficacy assays validating the “IF-BETTER” gating strategy in the context of ADGRE2/CLEC12A co-targeting via ADCLEC.syn1. FIG. 19A shows cytotoxicity induced by ADCLEC.syn1-transduced T cells and cocultured with EL4 murine lymphoma cell line expressing no target (ADGRE2⁻/CLEC12A⁻) or overexpressing either CAR target alone (ADGRE2⁺/CLEC12A⁻), CCR target alone (ADGRE2⁻/CLEC12A⁺), or both CAR and CCR target (ADGRE2⁺/CLEC12A⁺). 1928z1XX CAR was used as a negative control. FIG. 19B shows cytotoxicity induced by untransduced, ADGRE2−CAR−, CLEC12A−CCR−, or ADCLEC.syn1-transduced T cells cocultured with MOLM 13 target cells that were modified to express different ADGRE2 levels (i.e., ADGRE2-high (WT), ADGRE2-low, and ADGRE2-very-low).

FIG. 20 illustrates in vivo validation of the “IF-BETTER” gating strategy using ADCLEC.syn1 T cells. Tumor growth and survival were determined in NSG AML xenograft models receiving an injection of untransduced, ADGRE2−CAR−, ADGRE2−CAR+CLEC12A−CAR−, CLEC12A−CCR−, or ADCLEC.syn1-transduced T cells.

FIG. 21 illustrates in vivo stress test of ADCLEC.syn1 via T cell dose-titration and AML re-challenge. Tumor growth and survival were determined in NSG AML xenograft models receiving an injection of ADCLEC.syn1-transduced T cells at different doses.

### 5. DETAILED DESCRIPTION OF THE INVENTION

The presently disclosed subject matter provides chimeric receptors that target ADGRE2, and chimeric receptors that target CLEC12A. The presently disclosed subject matter further provides cells comprising the presently disclosed A DGRE2-targeted chimeric receptor, cells comprising the presently disclosed CLEC12A-targeted chimeric receptor, and cells comprising the presently disclosed ADGRE2-targeted chimeric receptor, and the presently disclosed CLEC12A-targeted chimeric receptor. The cells can be immunoresponsive cells, e.g., genetically modified immunoresponsive cells (e.g., T cells or NK cells). The presently disclosed subject matter also provides methods of using such cells for treatments, e.g., for treating and/or preventing a tumor associated with ADGRE2 and/or CLEC12A (e.g., AML).

Non-limiting embodiments of the presently disclosed subject matter are described by the present specification and Examples.

For purposes of clarity of disclosure and not by way of limitation, the detailed description is divided into the following subsections:
5.1. Definitions;
5.2. Chimeric Receptors;
5.3. Extracellular antigen-binding domains of ADGRE2-Targeted Chimeric Receptors;
5.4 Exemplified ADGRE2-Targeted Chimeric Receptors;
5.5. Extracellular antigen-binding domains of CLEC12A-Targeted Chimeric Receptors;
5.6. Exemplified CLEC12A-Targeted Chimeric Receptors;
5.7. Cells;
5.8. Nucleic Acid Molecules, Vector and Genetic Modifications;
5.9. Formulations and Administration; and
5.10. Methods of Treatment.

5.1. Definitions

Unless defined otherwise, all technical and scientific terms used herein have the meaning commonly understood by a person skilled in the art to which the presently disclosed subject matter belongs. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-IBB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IM GT numbering system (Lefranc, The Immunologist (1999); 7:132-136; Lefranc et al., Dev. Comp. Immunol. (2003); 27:55-77). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the IM GT numbering system. In certain embodiments, the CDR regions are delineated using the IM GT numbering system accessible at http://www.imgt.org/IMGT_vquest/input.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80 (6): 1910-1917 (2008) and WO 2014/087010, the contents of which are hereby incorporated by reference in their entireties. In certain embodiments, the linker is a G4S linker.

In certain embodiments, the linker comprises or consists of the amino acid sequence set forth in SEQ ID NO: 1, which is provided below:

[SEQ ID NO: 1]
GGGGSGGGGSGGGSGGGGS

In certain embodiments, the linker comprise or consists of the amino acid sequence set forth in SEQ ID NO: 2, which is provided below:

[SEQ ID NO: 2]
GGGGSGGGGSGGGGS

In certain embodiments, the linker comprises or consists of the amino acid sequence set forth in SEQ ID NO: 3, which is provided below:

[SEQ ID NO: 3]
GGGGSGGGGSGGGGSGGGSGGGGS

In certain embodiments, the linker comprises or consists of the amino acid sequence set forth in SEQ ID NO: 4, which is provided below:

[SEQ ID NO: 4]
GGGGSGGGGSGGGGSGGGGSGGGSGGGGS

In certain embodiments, the linker comprises or consists of the amino acid sequence set forth in SEQ ID NO: 5, which is provided below:

[SEQ ID NO: 5]
GGGGS

In certain embodiments, the linker comprises or consists of the amino acid sequence set forth in SEQ ID NO: 6, which is provided below:

[SEQ ID NO: 6]
GGGGSGGGGS

In certain embodiments, the linker comprises the first three amino acids of the heavy chain constant region. In certain embodiments, the linker comprises or consists of the amino acid sequence set forth in SEQ ID NO: 149, which is provided below:

[SEQ ID NO: 149]
ASTGGGGSGGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. Proc. Nat. Acad. Sci. USA, (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) (2008); 27 (6): 445-51; Peter et al., J Cachexia Sarcopenia Muscle (2012); August 12; Shieh et al., J Imunol (2009); 183 (4): 2277-85; Giomarelli et al., Thromb Haemost (2007); 97 (6): 955-63; Fife eta., J Clin Invst (2006); 116 (8): 2252-61; Brocks et al., Immunotechnology 1997 3 (3): 173-84; Moosmayer et al., Ther Immunol 1995 2 (10:31-40). Agonistic scFvs having stimulatory activity have been described (Peter et al., J Biol Chem (2003); 25278 (38): 36740-7; Xie et al., Nat Biotech 1997 15 (8): 768-71; Ledbetter et al., Crit Rev Immunol (1997); 17 (5-6): 427-55; Ho et al., BioChim Biophys Acta (2003); 1638 (3): 257-66).

The term “chimeric antigen receptor” or “CAR” as used herein refers to a molecule comprising an extracellular antigen-binding domain that is fused to an intracellular signaling domain that is capable of activating or stimulating an immunoresponsive cell. In certain embodiments, the CAR also comprises a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain.

The term “chimeric co-stimulating receptor” or “CCR” refers to a chimeric receptor that binds to an antigen and provides co-stimulatory signals, but does not alone provide an activation signal. CCR is described in Krause, et al., J. Exp. Med. (1998); 188 (4): 619-626, and US20020018783, the contents of which are incorporated by reference in their entireties. CCRs mimic co-stimulatory signals, but unlike, CARs, do not alone provide an activation signal, e.g., CCRs lack a CD3ζ polypeptide.

By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTY BOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM 120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PA M 250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215: 403-10. BLAST protein searches can be performed with the X BLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., X BLA ST and NBLAST) can be used. An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “receptor” is meant a polypeptide, or portion thereof, present on a cell membrane that selectively binds one or more ligand.

By “signal sequence” or “leader sequence” is meant a peptide sequence (e.g., 5, 10, 15, 20, 25 or 30 amino acids) present at the N-terminus of newly synthesized proteins that directs their entry to the secretory pathway

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “a conservative sequence modification” refers to an amino acid modification that does not significantly affect or alter the binding characteristics of the presently disclosed chimeric receptors comprising the amino acid sequence. Conservative modifications can include amino acid substitutions, additions and deletions. Modifications can be introduced into the extracellular antigen-binding domain of the presently disclosed chimeric receptors by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (I) above) using the functional assays described herein. In certain embodiments, no more than one, no more than two, no more than three, no more than four, no more than five residues within a specified sequence or a CDR region are altered.

Other aspects of the presently disclosed subject matter are described in the following disclosure and are within the ambit of the presently disclosed subject matter.

### 5.2. Chimeric Receptors

In certain embodiments, the presently disclosed chimeric receptor comprises an extracellular antigen-binding domain that binds to ADGRE2 or CLEC12A. The extracellular antigen-binding domain can be an antigen-binding fragment of an antibody, an antigen-binding fragment of a heavy chain variable region (V_{H}) of an antibody, an antigen-binding fragment of a light chain variable region (V_{L}) of an antibody, a single chain variable fragment (scFv), a Fab, or F(ab)₂. In certain embodiments, the extracellular antigen-binding fragment is a single chain variable fragment (scFv). In certain embodiments, the scFv is a human scFv. In certain embodiments, the scFv is a humanized scFv. In certain embodiments, the scFv is a murine scFv. In certain embodiments, the Fab is crosslinked.

In certain embodiments, the presently disclosed chimeric receptor is a chimeric antigen receptor (CAR). In certain embodiments, the presently disclosed chimeric receptor is a chimeric co-stimulating receptor (CCR). In certain embodiments, the chimeric receptor is a TCR like fusion molecule.

### 5.2.1. Chimeric Antigen Receptor (CAR)

In certain embodiments, the chimeric receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD35). “Third generation” CARs comprise those that provide multiple co-stimulation (e.g., CD28 and 4-1BB) and activation (CD35). In certain embodiments, the chimeric receptor is a second generation CAR. In certain embodiments, the chimeric receptor is a CAR that comprises an intracellular domain of a co-stimulatory molecule or a fragment thereof.

### 5.2.1.1. Extracellular Antigen-Binding Domain of a CAR

In certain embodiments, the extracellular antigen-binding domain is a single chain variable fragment (scFv). In certain embodiments, the scFv is a human scFv. In certain embodiments, the scFv is a humanized scFv. In certain embodiments, the scFv is a murine scFv. In certain embodiments, the scFv is identified by screening scFv phage library with an antigen-Fc fusion protein.

In certain embodiments, the extracellular antigen-binding domain is a Fab. In certain embodiments, the Fab is crosslinked. In certain embodiments, the extracellular antigen-binding domain is a F(ab)₂. Any of the foregoing molecules may be comprised in a fusion protein with a heterologous sequence to form the extracellular antigen-binding domain.

Binding of the extracellular antigen-binding domain of a chimeric receptor, e.g., a CAR, can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassay, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and Y Pet).

### 5.2.1.2. Transmembrane Domain of a CAR

In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICA M-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of the CAR comprises a CD8 polypeptide (e.g., a transmembrane domain of CD8 or a fragment thereof).

In certain embodiments, the transmembrane domain of the CAR comprises a CD8 polypeptide (e.g., a transmembrane domain of CD8 or a fragment thereof). In certain embodiments, the transmembrane domain of the CAR comprises a CD8 polypeptide (e.g., a transmembrane domain of human CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 7) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 7, which is at least about 20, or at least about 30, or at least about 40, or at least about 50, at least about 60, at least about 70, and up to about 235 amino acids in length. In certain embodiments, the CD8 polypeptide comprises or consists of amino acids 1 to 235, 1 to 50, 50 to 100, 100 to 150, 150 to 200, 137 to 207, or 200 to 235 of SEQ ID NO: 7. In certain embodiments, the transmembrane domain of the CAR comprises a CD8 polypeptide comprising or consisting of amino acids 137 to 207 of SEQ ID NO: 7. SEQ ID NO: 7 is provided below.

[SEQ ID NO: 7]
MALPVTALLLPLALLLHAARPSQFRVSPLDRTWNLGETVELKCQVLLSNPT
SGCSWLFQPRGAAASPTFLLYLSQNKPKAAEGLDTQRFSGKRLGDTFVLTL
SDFRRENEGYYFCSALSNSIMYFSHFVPVFLPAKPTTTPAPRPPTPAPTIA
SQPLSLRPEACRPAAGGAVHTRGLDFACDIYIWAPLAGTCGVLLLSLVITL
YCNHRNRRRVCKCPRPVVKSGDKPSLSARYV

An exemplary nucleotide sequence encoding amino acids 137 to 207 of SEQ ID NO: 7 is set forth in SEQ ID NO: 8, which is provided below.

[SEQ ID NO: 8]
Cccaccacgacgccagcgccgcgaccaccaaccccggcgcccacgatcgcg
tcgcagcccctgtccctgcgcccagaggcgtgccggccagcggcggggggc
gcagtgcacacgagggggctggacttcgcctgtgatatctacatctgggcg
cccctggccgggacttgtggggtccttctcctgtcactggttatcaccctt
tactgcaac

In certain embodiments, the transmembrane domain of the CAR comprises a CD8 polypeptide (e.g., a transmembrane domain of mouse CD8 or a fragment thereof). In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: AAA 92533.1 (SEQ ID NO: 9) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 9, which is at least about 20, or at least about 30, or at least about 40, or at least about 50, or at least about 60, or at least about 70, or at least about 100, or at least about 200, and up to about 247 amino acids in length. In certain embodiments, the CD8 polypeptide comprises or consists of amino acids 1 to 247, 1 to 50, 50 to 100, 100 to 150, 150 to 200, 151 to 219, or 200 to 247 of SEQ ID NO: 9. In certain embodiments, the transmembrane domain of the CAR comprises a CD8 polypeptide comprising or consisting of amino acids 151 to 219 of SEQ ID NO: 9. SEQ ID NO: 9 is provided below.

| [SEQ ID NO: 9] | |
|---|---|
| 1 | MASPLTRFLS LNLLLMGESI ILGSGEAKPQ APELRIFPKK MDAELGQKVD LVCEVLGSVS |
| 61 | QGCSWLFQNS SSKLPQPTFV VYMASSHNKI TWDEKLNSSK LFSAVRDTNN KYVLTLNKFS |
| 121 | KENEGYYFCS VISNSVMYFS SVVPVLQKVN STTTKPVLRT PSPVHPTGTS QPQRPEDCRP |
| 181 | RGSVKGTGLD FACDIYIWAP LAGICVAPLL SLIITLICYH RSRKRVCKCP RPLVRQEGKP |
| 241 | RPSEKIV |

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a CD28 polypeptide (e.g., a transmembrane domain of human CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_006130 (SEQ ID NO: 10) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 10, which is at least about 20, or at least about 30, or at least about 40, or at least about 50, and up to about 220 amino acids in length. In certain embodiments, the CD28 polypeptide comprises or consists of amino acids 1 to 220, 1 to 50, 50 to 100, 100 to 150, 150 to 200, 153 to 179, or 200 to 220 of SEQ ID NO: 9. In certain embodiments, the transmembrane domain of the CAR comprises a CD28 polypeptide comprising or consisting of amino acids 153 to 179 of SEQ ID NO: 10. SEQ ID NO: 10 is provided below:

| [SEQ ID NO: 10] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

An exemplary nucleotide sequence encoding amino acid 153 to 179 of SEQ ID NO: 10 is set forth in SEQ ID NO: 11, which is provided below.

[SEQ ID NO: 11]
ttttgggtgctggtggtggttggtggagtcctggcttgctatagcttgcta
gtaacagtggcctttattattttctgggtg

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_031668.3 (SEQ ID NO: 12) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 12, which is at least about 20, or at least about 30, or at least about 40, or at least about 50, and up to about 218 amino acids in length. In certain embodiments, the CD28 polypeptide comprises or consists of amino acids 1 to 220, 1 to 50, 50 to 100, 100 to 150, 150 to 200, 151 to 177, or 200 to 218 of SEQ ID NO: 12. In certain embodiments, the transmembrane domain of the CAR comprises a CD28 polypeptide comprising or consisting of amino acids 151 to 177 of SEQ ID NO: 12. SEQ ID NO: 12 is provided below:

| [SEQ ID NO: 12] | |
|---|---|
| 1 | MTLRLLFLAL NFFSVQVTEN KILVKQSPLL VVDSNEVSLS CRYSYNLLAK EFRASLYKGV |
| 61 | NSDVEVCVGN GNFTYQPQFR SNAEFNCDGD FDNETVTFRL WNLHVNHTDI YFCKIEFMYP |
| 121 | PPYLDNERSN GTIIHIKEKH LCHTQSSPKL FWALVVVAGV LFCYGLLVTV ALCVIWTNSR |
| 181 | RNRLLQSDYM NMTPRRPGLT RKPYQPYAPA RDFAAYRP |

In certain embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH2CH3 region of immunoglobulin and portions of CD3, a portion of a CD28 polypeptide (e.g., a portion of SEQ ID NO: 10 or 12), a portion of a CD8 polypeptide (e.g., a portion of SEQ ID NO: 7 or 9), a variation of any of the foregoing which is at least about 80%, at least about 85%, at least about 90%, at least about 95%, or at least about 100% homologous or identical thereto, or a synthetic spacer sequence.

In certain embodiments, the hinge/spacer region of the CAR comprises a CD28 polypeptide. In certain embodiments, the hinge/spacer region of the CAR comprises a CD28 polypeptide comprising or consisting of amino acids 114 to 152 of SEQ ID NO: 10.

An exemplary nucleotide sequence encoding amino acid 114 to 152 of SEQ ID NO: 10 is set forth in SEQ ID NO: 13, which is provided below.

[SEQ ID NO: 13]
attgaagttatgtatcctcctccttacctagacaatgagaagagcaatgga
accattatccatgtgaaagggaaacacctttgtccaagtcccctatttccc
ggaccttctaagccc

### 5.2.1.3. Intracellular Signaling Domain of a CAR

In certain embodiments, the CAR comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAM s), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3-chain is the primary transmitter of signals from endogenous TCRs.

In certain embodiments, the intracellular signaling domain of the CAR comprises a native CD3ζ. In certain embodiments, the native CD3ζ polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_932170 (SEQ ID NO: 14) or a fragment thereof, and/or may optionally comprise up to one or up to two or up to three conservative amino acid substitutions. In certain embodiments, the native CD3ζ polypeptide comprises or consists of an amino acid sequence that is a consecutive portion of SEQ ID NO: 14, which is at least about 20, or at least about 30, or at least about 40, or at least about 50, at least about 100, at least about 110, and up to about 164 amino acids in length. In certain embodiments, the native CD34 polypeptide comprises or consists of amino acids 1 to 164, 1 to 50, 50 to 100, 52 to 164, 100 to 150, or 150 to 164 of SEQ ID NO: 14. In certain embodiments, the intracellular signaling domain of the CAR comprises a CD3ζ polypeptide comprising or consisting of amino acids 52 to 164 of SEQ ID NO: 14. SEQ ID NO: 14 is provided below:

| [SEQ ID NO: 14] | |
|---|---|
| 1 | MKWKALFTAA ILQAQLPITE AQSFGLLDPK LCYLLDGILF IYGVILTALF LRVKFSRSAD |
| 61 | APAYQQGQNQ LYNELNLGRR EEYDVLDKRR GRDPEMGGKP QRRKNPQEGL YNELQKDKMA |
| 121 | EAYSEIGMKG ERRRGKGHDG LYQGLSTATK DTYDALHMQA LPPR |

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD34 polypeptide comprises a native ITAM1. In certain embodiments, the native ITAM1 comprises or consists of the amino acid sequence set forth in SEQ ID NO: 15.

[SEQ ID NO: 15]
QNQLYNELNLGRREEYDVLDKR

An exemplary nucleotide sequence encoding the amino acid sequence of SEQ ID NO: 15 is set forth in SEQ ID NO: 16, which is provided below.

[SEQ ID NO: 16]
Cagaaccagctctataacgagctcaatctaggacgaagagaggagtacgat
gttttggacaagaga

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 17, which is provided below.

[SEQ ID NO: 17]
QNQLFNELNLGRREEFDVLDKR

An exemplary nucleotide sequence encoding the amino acid sequence of SEQ ID NO: 17 is set forth in SEQ ID NO: 18, which is provided below.

[SEQ ID NO: 18]
CAGAACCAGCTCTTTAACGAGCTCAATCTAGGACGAAGAGAGGAGTTCGAT
GTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 19, which is provided below.

[SEQ ID NO: 19]
QEGLYNELQKDKMAEAYSEIGMK

An exemplary nucleotide sequence encoding the amino acid sequence of SEQ ID NO: 19 is set forth in SEQ ID NO: 20, which is provided below.

[SEQ ID NO: 20]
CAGGAAGGCCTGTACAATGAACTGCAGAAAGATAAGATGGCGGAGGCCTAC
AGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM2 variant consists of two loss-of-function mutations. In certain embodiments, the ITAM2 variant comprises or consists of the amino acid sequence set forth in SEQ ID NO: 21, which is provided below.

[SEQ ID NO: 21]
QEGLFNELQKDKMAEAFSEIGMK

An exemplary nucleotide sequence encoding the amino acid sequence of SEQ ID NO: 21 is set forth in SEQ ID NO: 22, which is provided below.

[SEQ ID NO: 22]
Caggaaggcctgttcaatgaactgcagaaagataagatggcggaggccttc
agtgagattgggatgaaa

In certain embodiments, the modified CD3ζ polypeptide comprises a native ITAM3. In certain embodiments, the native ITAM3 comprises or consists of the amino acid sequence set forth in SEQ ID NO: 23, which is provided below.

[SEQ ID NO: 23]
HDGLYQGLSTATKDTYDALHMQ

An exemplary nucleotide sequence encoding the amino acid sequence of SEQ ID NO: 23 is set forth in SEQ ID NO: 24, which is provided below.

[SEQ ID NO: 24]
CACGATGGCCTTTACCAGGGTCTCAGTACAGCCACCAAGGACACCTACGAC
GCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 25, which is provided below.

[SEQ ID NO: 25]
HDGLFQGLSTATKDTFDALHMQ

An exemplary nucleotide sequence encoding the amino acid sequence of SEQ ID NO: 25 is set forth in SEQ ID NO: 26, which is provided below.

[SEQ ID NO: 26]
Cacgatggccttttccagggtctcagtacagccaccaaggacaccttcgac
gcccttcacatgcag

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 15, an ITAM2 variant consisting of the amino acid sequence set forth in SEQ ID NO: 21, and an ITAM3 variant consisting of the amino acid sequence set forth in SEQ ID NO: 25. In certain embodiments, the modified CD3ζ polypeptide is designated as “1XX”. In certain embodiments, the modified CD3ζ polypeptide comprises or consists of the amino acid sequence set forth in SEQ ID NO: 27. SEQ ID NO: 27 is provided below.

[SEQ ID NO: 27]
RVKFSRSADA PAYQQGQNQL YNELNLGRRE EYDVLDKRRG
RDPEMGGKPR RKNPQEGLFN ELQKDKMAEA FSEIGMKGER
RRGKGHDGLF QGLSTATKDT FDALHMQALP PR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 27 or a fragment thereof, and/or may optionally comprise up to one or up to two or up to three conservative amino acid substitutions.

An exemplary nucleotide sequence encoding the amino acid sequence of SEQ ID NO: 27 is set forth in SEQ ID NO: 28, which is provided below.

[SEQ ID NO: 28]
agagtgaagttcagcaggagcgcagacgcccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgttcaatgaactgcagaaagataagat
ggcggaggccttcagtgagattgggatgaaaggcgagcgccggaggggca
aggggcacgatggccttttccagggtctcagtacagccaccaaggacacc
ttcgacgcccttcacatgcaggccctgccccctcgc

In certain embodiments, the CAR is a second-generation CAR. In certain embodiments, the intracellular signaling domain of the CAR further comprises at least a co-stimulatory signaling region. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, and NKGD2. The co-stimulatory molecule can bind to a co-stimulatory ligand, which is a protein expressed on cell surface that upon binding to its receptor produces a co-stimulatory response, i.e., an intracellular response that effects the stimulation provided when an chimeric receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 10 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 10, which is at least about 20, or at least about 30, or at least about 40, or at least about 50, and up to about 220 amino acids in length. In certain embodiments, the CD28 polypeptide comprises or consists of amino acids 1 to 220, 1 to 50, 50 to 100, 100 to 150, 114 to 220, 150 to 200, 180 to 220, or 200 to 220 of SEQ ID NO: 10. In certain embodiments, the intracellular signaling domain of the CAR comprises a co-stimulatory signaling region that comprises a CD28 polypeptide comprising or consisting of amino acids 180 to 220 of SEQ ID NO: 10.

An exemplary nucleic acid sequence encoding amino acids 180 to 220 of SEQ ID NO: 10 is set forth in SEQ ID NO: 29, which is provided below.

[SEQ ID NO: 29]
Aggagtaagaggagcaggctcctgcacagtgactacatgaacatgactcc
ccgccgccccgggcccacccgcaagcattaccagccctatgccccaccac
gcgacttcgcagcctatcgctcc

In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 12 or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 12, which is at least about 20, or at least about 30, or at least about 40, or at least about 50, and up to 218 amino acids in length. In certain embodiments, the CD28 polypeptide comprises or consists of amino acids 1 to 218, 1 to 50, 50 to 100, 100 to 150, 150 to 218, 178 to 218, or 200 to 218 of SEQ ID NO: 12. In certain embodiments, the co-stimulatory signaling region of a presently disclosed CAR comprises a CD28 polypeptide that comprises or consists of the amino acids 178 to 218 of SEQ ID NO: 12.

In certain embodiments, the intracellular signaling domain of the CAR comprises a co-stimulatory signaling region that comprises a 4-1BB polypeptide, e.g., an intracellular domain of 4-1BB or a fragment thereof (e.g., an intracellular domain of human 4-1BB or a fragment thereof). The 4-1BB polypeptide can comprise or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No.: NP_001552 (SEQ ID NO: 30) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 30, which is at least about 20, or at least about 30, or at least about 40, or at least about 50, or at least about 100, or at least about 150, or at least about 150, and up to about 255 amino acids in length. In certain embodiments, the 4-1BB polypeptide comprises or consists of amino acids 1 to 255, 1 to 50, 50 to 100, 100 to 150, 150 to 200, 200 to 255, or 214 to 255 of SEQ ID NO: 30. In certain embodiments, the intracellular signaling domain of the CAR comprises a co-stimulatory signaling region that comprises a 4-1BB polypeptide comprising or consisting of amino acids 214 to 255 of SEQ ID NO: 30. SEQ ID NO: 30 is provided below.

| [SEQ ID NO: 30] | |
|---|---|
| 1 | MGNSCYNIVA TLLLVLNFER TRSLQDPCSN CPAGTFCDNN RNQICSPCPP NSFSSAGGQR |
| 61 | TCDICRQCKG VFRTRKECSS TSNAECDCTP GFHCLGAGCS MCEQDCKQGQ ELTKKGCKDC |
| 121 | CFGTFNDQKR GICRPWTNCS LDGKSVLVNG TKERDVVCGP SPADLSPGAS SVTPPAPARE |
| 181 | PGHSPQIISF FLALTSTALL FLLFFLTLRF SVVKRGRKKL LYIFKQPFMR PVQTTQEEDG |
| 241 | CSCRFPEEEE GGCEL |

An exemplary nucleic acid sequence encoding amino acids 214 to 255 of SEQ ID NO: 30 is set forth in SEQ ID NO: 31, which is provided below.

[SEQ ID NO: 31]
aaacggggcagaaagaagctcctgtatatattcaaacaaccatttatgag
accagtacaaactactcaagaggaagatggctgtagctgccgatttccag
aagaagaagaaggaggatgtgaactg

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

### 5.2.2. Chimeric Co-Stimulatory Receptor (CCR)

In certain embodiments, the chimeric receptor is a CCR. The presently disclosed CCR binds to an antigen (e.g., ADGRE2 or CLEC12A) and provides co-stimulatory signals, but does not alone provide an activation signal. In certain embodiments, the CCR does not comprise a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (K loss et al., Nature Biotechnology (2013); 31 (1): 71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to an antigen (e.g., ADGRE2 or CLEC12A), a transmembrane domain, and a co-stimulatory signaling region that comprises an intracellular domain of at least one co-stimulatory molecule or a fragment thereof. In certain embodiments, the CCR does not alone deliver an activation signal to an immunoresponsive cell. Non-limiting examples of co-stimulatory molecules include CD28, 4-1BB, OX40, ICOS, DAP-10, CD27, CD40, and NKGD2. In certain embodiments, the co-stimulatory signaling region of the CCR comprises an intracellular domain of a co-stimulatory signaling molecule or a fragment thereof. In certain embodiments, the one co-stimulatory signaling molecule is CD28. In certain embodiments, the one co-stimulatory signaling molecule is 4-1BB. In certain embodiments, the co-stimulatory signaling region of the CCR comprises an intracellular domain of a first co-stimulatory signaling molecule or a fragment thereof and an intracellular domain of a second co-stimulatory signaling molecule or a fragment thereof. In certain embodiments, the first and second co-stimulatory signaling molecules are CD28 and 4-1BB.

Similar to a CAR, the extracellular antigen-binding domain of the CCR can be an scFv, a Fab, a F(ab)₂, or a fusion protein with a heterologous sequence to form the extracellular antigen-binding domain of the CCR.

5.2.3. TCR like Fusion Molecules

In certain embodiments, the chimeric receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety), synthetic T cell receptor and antigen receptor (STAR) (e.g., those disclosed in Liu et al. Science Translational Medicine (2021); 13 (586): eabb5191, which is incorporated by reference in its entirety), antibody-T-cell receptor (AbTCR) (e.g., those disclosed in X u et al. Cell Discovery (2018) 4:62, which is incorporated by reference in its entirety), and T cell antigen coupler (TAC) (e.g., those disclosed in Helsen et al. Nature Communications (2018); 9:3049, which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC polypeptide, a native or modified TR B C polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen (e.g., ADGRE2 or CLEC12A), is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

### 5.3. Extracellular Antigen-Binding Domain of ADGRE2-Targeted Chimeric Receptors

In certain embodiments, the presently disclosed chimeric receptor targets ADGRE2. In certain embodiments, the presently disclosed chimeric receptor comprises an extracellular antigen-binding domain that binds to ADGRE2.

Adhesion G Protein-Coupled Receptor E2 (ADGRE2), also known as EMR2, CD312, VBU or CD97, is a member of the adhesion GPCR family. It is expressed by monocytes/macrophages, dendritic cells and all types of granulocytes. ADGRE2 is a cell surface receptor that binds to the chondroitin sulfate moiety of glycosaminoglycan chains and promotes cell attachment. It promotes granulocyte chemotaxis, degranulation and adhesion. In macrophages, ADGRE2 promotes the release of inflammatory cytokines, including IL 8 and TNF. Signals probably through G-proteins.

In certain embodiments, the presently disclosed chimeric receptor targets human ADGRE2. In certain embodiments, the presently disclosed chimeric receptor comprises an extracellular antigen-binding domain that binds to human ADGRE2. In certain embodiments, the human ADGRE2 comprises or consists of the amino acid sequence with a Uniprot Reference No: Q9UHX3-1 (SEQ ID NO: 32), or a fragment thereof. SEQ ID NO: 32 is provided below:

[SEQ ID NO: 32]
MGGRVFLVFL AFCVWLTLPG AETQDSRGCA RWCPQDSSCV
NATACRCNPG FSSFSEIITT PMETCDDINE CATLSKVSCG
KFSDCWNTEG SYDCVCSPGY EPVSGAKTFK NESENTCQDV
DECQQNPRLC KSYGTCVNTL GSYTCQCLPG FKLKPEDPKL
CTDVNECTSG QNPCHSSTHC LNNVGSYQCR CRPGWQPIPG
SPNGPNNTVC EDVDECSSGQ HQCDSSTVCF NTVGSYSCRC
RPGWKPRHGI PNNQKDTVCE DMTFSTWTPP PGVHSQTLSR
FFDKVQDLGR DYKPGLANNT IQSILQALDE LLEAPGDLET
LPRLQQHCVA SHLLDGLEDV LRGLSKNLSN GLLNFSYPAG
TELSLEVQKQ VDRSVTLRQN QAVMQLDWNQ AQKSGDPGPS
VVGLVSIPGM GKLLAEAPLV LEPEKQMLLH ETHQGLLQDG
SPILLSDVIS AFLSNNDTQN LSSPVTFTFS HRSVIPRQKV
LCVFWEHGQN GCGHWATTGC STIGTRDTST ICRCTHLSSF
AVLMAHYDVQ EEDPVLTVIT YMGLSVSLLC LLLAALTFLL
CKAIQNTSTS LHLQLSLCLF LAHLLFLVAI DQTGHKVLCS
IIAGTLHYLY LATLTWMLLE ALYLFLTARN LTVVNYSSIN
RFMKKLMFPV GYGVPAVTVA ISAASRPHLY GTPSRCWLQP
EKGFIWGFLG PVCAIFSVNL VLFLVTLWIL KNRLSSLNSE
VSTLRNTRML AFKATAQLFI LGCTWCLGIL QVGPAARVMA
YLFTIINSLQ GVFIFLVYCL LSQQVREQYG KWSKGIRKLK
TESEMHTLSS SAKADTSKPS TVN

Human ADGRE2 comprises an EGF-like 1 domain, an EGF-like 2 domain, an EGF-like 3 domain, an EGF-like 4 domain, an EGF-like 5 domain, and a GPS domain. In certain embodiments, the EGF-like 1 domain comprises or consists of amino acids 25 to 66 of SEQ ID NO: 32. In certain embodiments, the EGF-like 2 domain comprises or consists of amino acids 67 to 118 of SEQ ID NO: 32. In certain embodiments, the EGF-like 3 domain comprises or consists of amino acids 119 to 162 of SEQ ID NO: 32. In certain embodiments, the EGF-like 4 domain comprises or consists of amino acids163 to 211 of SEQ ID NO: 32. In certain embodiments, the EGF-like 5 domain comprises or consists of amino acids 212 to 260 of SEQ ID NO: 32. In certain embodiments, the GPS domain comprises or consists of amino acids 479 to 529 of SEQ ID NO: 32.

In certain embodiments, the presently disclosed chimeric receptor targets an ADGRE2 polypeptide comprising or consisting of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 32 or a fragment thereof.

In certain embodiments, the extracellular antigen-binding domain of a presently disclosed ADGRE2-targeted chimeric receptor binds to the stalk region of ADGRE2. In certain embodiments, the extracellular antigen-binding domain of a presently disclosed ADGRE2-targeted chimeric receptor binds to the GPS domain of ADGRE2. In certain embodiments, the extracellular antigen-binding domain of a presently disclosed ADGRE2-targeted chimeric receptor binds to the EGF-like 5 domain of ADGRE2.

In certain embodiments, the ADGRE2-targeted chimeric receptor is a chimeric antigen receptor (CAR). In certain embodiments, the ADGRE2-targeted CAR has the structure disclosed in Section 5.2.1. In certain embodiments, the ADGRE2-targeted CAR comprises an extracellular antigen-binding domain that binds to ADGRE2, a transmembrane domain, and an intracellular signaling domain.

In certain embodiments, the ADGRE2-targeted chimeric receptor is a Chimeric Co-Stimulatory Receptor (CCR). In certain embodiments, the ADGRE2-targeted CCR has the structure disclosed in Section 5.2.2. In certain embodiments, the ADGRE2-targeted CCR comprises an extracellular antigen-binding domain that binds to ADGRE2, a transmembrane domain, and an intracellular signaling domain that does not provide an activation signal to an immunoresponsive cell, e.g., the intracellular signaling domain does not comprise a CD3ζ polypeptide.

In certain embodiments, the ADGRE2-targeted chimeric receptor is a TCR like Fusion Molecules. In certain embodiments, the ADGRE2-targeted TCR like Fusion Molecules has the structure disclosed in Section 5.2.3.

In certain embodiments, the extracellular antigen-binding domain of the ADGRE2-targeted chimeric receptor (e.g., an ADGRE2-targeted scFv) binds to ADGRE2 (e.g., human ADGRE2) with a dissociation constant (K_{D}) of at least about 1×10⁻⁶ M, at least about 1×10⁻⁷ M, at least about 1×10⁻⁸ M, at least about 1×10⁻⁹ M, or at least about 1×10⁻¹⁰ M. In certain embodiments, the extracellular antigen-binding domain of the ADGRE2-targeted chimeric receptor (e.g., an ADGRE2-targeted scFv) binds to ADGRE2 (e.g., human ADGRE2) with a dissociation constant (K_{D}) of at least about 2×10⁻⁸ M. In certain embodiments, the extracellular antigen-binding domain of the ADGRE2-targeted chimeric receptor (e.g., an ADGRE2-targeted scFv) to ADGRE2 (e.g., human ADGRE2) with a dissociation constant (K_{D}) of between about 2×10⁻⁸ M and about 8×10⁻⁹ M.

In certain embodiments, the extracellular antigen-binding domain of the ADGRE2-targeted chimeric receptor (e.g., an ADGRE2-targeted scFv) binds to ADGRE2 (e.g., human ADGRE2) with a dissociation constant (K_{D}) between about 1 nM and about 50 nM, between about 5 nM and about 30 nM, between about 5 nM and about 25 nM, or between about 8 nM and about 20 nM. In certain embodiments, the extracellular antigen-binding domain of the ADGRE2-targeted chimeric receptor (e.g., an ADGRE2-targeted scFv) binds to ADGRE2 (e.g., human ADGRE2) with a dissociation constant (K_{D}) of at least about 50 nM, at least about 40 nM, at least about 35 nM, at least about 30 nM, at least about 25 nM, at least about 20 nM, at least about 19 nM, at least about 18 nM, at least about 17 nM, at least about 16 nM, at least about 15 nM, at least about 14 nM, at least about 13 nM, at least about 12 nM, at least about 11 nM, at least about 10 nM, at least about 9 nM, at least about 8 nM, at least about 7 nM, at least about 6 nM, or at least about 5 nM.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, and a V_{H} CDR3 comprising the amino acid sequence set forth in SEQ ID NO: 35 or a conservative modification thereof. SEQ ID NOs: 33-35 are provided in Table 1.

In certain embodiments, the extracellular antigen-binding domain of the ADGRE2-targeted chimeric receptor (e.g., an ADGRE2-targeted scFv) comprises a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof. SEQ ID NOs: 36-38 are provided in Table 1.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, a V_{H} CDR3 comprising the amino acid sequence set forth in SEQ ID NO: 35 or a conservative modification thereof, a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification, and a V_{L} CDR3 comprising the amino acid sequence set forth in SEQ ID NO: 38 or a conservative modification thereof.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33, a V_{H} CDR2 comprising the amino acid sequence set forth in SEQ ID NO: 34, a V_{H} CDR3 comprising the amino acid sequence set forth in SEQ ID NO: 35, a V_{L} CDR1 comprising the amino acid sequence set forth in SEQ ID NO: 36, a V_{L} CDR2 comprising the amino acid sequence set forth in SEQ ID NO: 37, and a V_{L} CDR3 comprising the amino acid sequence set forth in SEQ ID NO: 38.

In certain embodiments, the extracellular antigen-binding domain of the ADGRE2-targeted chimeric receptor (e.g., an ADGRE2-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 39. For example, the extracellular antigen-binding domain of the ADGRE2-targeted chimeric receptor (e.g., an ADGRE2-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 39. In certain embodiments, the extracellular antigen-binding domain of the ADGRE2-targeted chimeric receptor (e.g., an ADGRE2-targeted scFv) comprises a V_{H} comprising the amino acid sequence set forth in SEQ ID NO: 39. SEQ ID NO: 39 is provided in Table 1 below.

In certain embodiments, the extracellular antigen-binding domain of the ADGRE2-targeted chimeric receptor (e.g., an ADGRE2-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 40. For example, the extracellular antigen-binding domain of the ADGRE2-targeted chimeric receptor (e.g., an ADGRE2-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 40. In certain embodiments, the extracellular antigen-binding domain of the ADGRE2-targeted chimeric receptor (e.g., an ADGRE2-targeted scFv) comprises a V_{L} comprising the amino acid sequence set forth in SEQ ID NO: 40. SEQ ID NO: 40 is provided in Table 1 below.

In certain embodiments, the extracellular antigen-binding domain of the ADGRE2-targeted chimeric receptor (e.g., an ADGRE2-targeted scFv) comprises a V_{H} comprising the amino acid sequence set forth in SEQ ID NO: 39, and a V_{L} comprising the amino acid sequence set forth in SEQ ID NO: 40. In certain embodiments, the V_{H} and V_{L} are linked via a linker. In certain embodiments, the linker comprises the amino acid sequence set forth in SEQ ID NO: 2. In certain embodiments, the linker comprises the amino acid sequence set forth in SEQ ID NO: 149.

In certain embodiments, the variable regions within the extracellular antigen-binding domain of the ADGRE2-targeted chimeric receptor have to be linked one after another such that at the N-terminus of the extracellular antigen-binding domain, a heavy chain variable region (V_{H}) is positioned. In certain embodiments, if the extracellular antigen-binding domain of the ADGRE2-targeted chimeric receptor is an scFv, the variable regions are positioned from the N- to the C-terminus: V_{H}-V_{L}. In certain embodiments, the ADGRE2-targeted scFv comprises or consists of the amino acid sequence set forth in SEQ ID NO: 41. In certain embodiments, the ADGRE2-targeted scFv is designated as “ADGRE2-A”. An exemplary nucleotide sequence encoding the amino acid sequence of SEQ ID NO: 41 is set forth in SEQ ID NO: 42. SEQ ID NOs: 41 and 42 are provided in Table 1. The CDRs provided in Table 1 are identified according to the IMGT numbering system.

TABLE 1
(ADGRE2-A)

| | CDRs | | |
|---|---|---|---|
| | 1 | 2 | 3 |
| V_{H} | GYTFTNYW [SEQ ID | VYPGDGDT [SEQ ID | ARGFTAYGMDY [SEQ |
| | NO: 33] | NO: 34] | ID NO: 35] |
| V_{L} | SSVSY [SEQ ID NO: | DTS [SEQ ID NO: | QQWSSNPLT [SEQ ID |
| | 36] | 37] | NO: 38] |

| | |
|---|---|
| Full V_{H} | QVQLQQSGAEVAKPGASVKLSCKASGYTFTNYWMQWIKQAPGQGLEWIGAVYPGDGDTR |
| | HTQKFKGKATLTADKSTSTAYMEVSSLRSEDTAVYYCARGFTAYGMDYWGQGTTVTVSS |
| | [SEQ ID NO: 39] |
| Full V_{L} | EIVLTQSPATMSASPGERVTMSCSASSSVSYMHWYQQKSGQSPKRWIYDTSKLASGVPA |
| | RFSGSGSGTDYTFTISSMEPEDFATYYCQQWSSNPLTFGGGTKLEIK [SEQ ID NO: |
| | 40] |
| DNA | caagttcagctccagcagagcggcgccgaagtggcaaagcctggagcgtcagtcaagct |
| Full V_{H} | gtcctgcaaagcgagtggctatacgttcacgaactactggatgcagtggataaagcagg |
| | ctcccgggcagggtctggagtggattggagccgtctacccaggggacggcgacacccgg |
| | cacactcaaaagttcaagggcaaggccaccctgaccgctgacaagagcacaagcacagc |
| | gtacatggaggtgtcctctttgagatccgaagataccgctgtgtattattgtgcccggg |
| | gcttcactgcatacgggatggattactggggacaaggcactaccgtgactgtcagctcc |
| | [SEQ ID NO: 144] |
| DNA | gaaattgtgctgacacagagccctgccacaatgtctgctagccctggcgagcgcgtgac |
| Full V_{L} | catgtcttgtagcgccagcagcagcgtgtcctacatgcattggtatcaacagaagtccg |
| | gccagtctcccaagcggtggatctacgatacaagcaagctggcctccggcgtgcccgcc |
| | agattttctggcagcggctctggaacagattacaccttcaccatctctagcatggaacc |
| | tgaggattttgccacctactattgccagcagtggtccagcaatcccctgacatttggag |
| | gaggcaccaagctggaaattaag [SEQ ID NO: 145] |
| V_{H}-V_{L} | QVQLQQSGAEVAKPGASVKLSCKASGYTFTNYWMQWIKQAPGQGLEWIGAVYPGDGDTR |
| scFv | HTQKFKGKATLTADKSTSTAYMEVSSLRSEDTAVYYCARGFTAYGMDYWGQGTTVTVSS |
| | GGGGSGGGGSGGGGSEIVLTQSPATMSASPGERVTMSCSASSSVSYMHWYQQKSGQSPK |
| | RWIYDTSKLASGVPARFSGSGSGTDYTFTISSMEPEDFATYYCQQWSSNPLTFGGGTKL |
| | EIK [SEQ ID NO: 41] |
| DNA for | caagttcagctccagcagagcggcgccgaagtggcaaagcctggagcgtcagtcaagct |
| V_{H}-V_{L} | gtcctgcaaagcgagtggctatacgttcacgaactactggatgcagtggataaagcagg |
| scFv | ctcccgggcagggtctggagtggattggagccgtctacccaggggacggcgacacccgg |
| | cacactcaaaagttcaagggcaaggccaccctgaccgctgacaagagcacaagcacagc |
| | gtacatggaggtgtcctctttgagatccgaagataccgctgtgtattattgtgcccggg |
| | gcttcactgcatacgggatggattactggggacaaggcactaccgtgactgtcagctcc |
| | gggggtggaggctcaggcggggggggttcaggaggggggggatctgaaattgtgctgac |
| | acagagccctgccacaatgtctgctagccctggcgagcgcgtgaccatgtcttgtagcg |
| | ccagcagcagcgtgtcctacatgcattggtatcaacagaagtccggccagtctcccaag |
| | cggtggatctacgatacaagcaagctggcctccggcgtgcccgccagattttctggcag |
| | cggctctggaacagattacaccttcaccatctctagcatggaacctgaggattttgcca |
| | cctactattgccagcagtggtccagcaatcccctgacatttggaggaggcaccaagctg |
| | gaaattaag [SEQ ID NO: 42] |

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, and a V_{H} CDR3 comprising the amino acid sequence set forth in SEQ ID NO: 35 or a conservative modification thereof. SEQ ID NOs: 33-35 are provided in Tables 1 and 2.

In certain embodiments, the extracellular antigen-binding domain of the ADGRE2-targeted chimeric receptor (e.g., an ADGRE2-targeted scFv) comprises a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof. SEQ ID NOs: 36-38 are provided in Tables 1 and 2.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, a V_{H} CDR3 comprising the amino acid sequence set forth in SEQ ID NO: 35 or a conservative modification thereof, a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33, a V_{H} CDR2 comprising the amino acid sequence set forth in SEQ ID NO: 34, a V_{H} CDR3 comprising the amino acid sequence set forth in SEQ ID NO: 35, a V_{L} CDR1 comprising the amino acid sequence set forth in SEQ ID NO: 36, a V_{L} CDR2 comprising the amino acid sequence set forth in SEQ ID NO: 37, and a V_{L} CDR3 comprising the amino acid sequence set forth in SEQ ID NO: 38.

In certain embodiments, the extracellular antigen-binding domain of the ADGRE2-targeted chimeric receptor (e.g., an ADGRE2-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 43. For example, the extracellular antigen-binding domain of the ADGRE2-targeted chimeric receptor (e.g., an ADGRE2-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 43. In certain embodiments, the extracellular antigen-binding domain of the ADGRE2-targeted chimeric receptor comprises a V_{H} comprising the amino acid sequence set forth in SEQ ID NO: 43. SEQ ID NO: 43 is provided in Table 2 below.

In certain embodiments, the extracellular antigen-binding domain of the ADGRE2-targeted chimeric receptor (e.g., an ADGRE2-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 44. For example, the extracellular antigen-binding domain of the chimeric receptor (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 44. In certain embodiments, the extracellular antigen-binding domain of the ADGRE2-targeted chimeric receptor comprises a V_{L} comprising the amino acid sequence set forth in SEQ ID NO: 44. SEQ ID NO: 44 is provided in Table 2 below.

In certain embodiments, the extracellular antigen-binding domain of the ADGRE2-targeted chimeric receptor (e.g., an ADGRE2-targeted scFv) comprises a V_{H} comprising the amino acid sequence set forth in SEQ ID NO: 43, and a V_{L} comprising the amino acid sequence set forth in SEQ ID NO: 44. In certain embodiments, the V_{H} and V_{L} are linked via a linker. In certain embodiments, the linker comprises the amino acid sequence set forth in SEQ ID NO: 2. In certain embodiments, the linker comprises the amino acid sequence set forth in SEQ ID NO: 149.

In certain embodiments, the variable regions within the extracellular antigen-binding domain of the ADGRE2-targeted chimeric receptor have to be linked one after another such that at the N-terminus of the extracellular antigen-binding domain, a heavy chain variable region (V_{H}) is positioned. In certain embodiments, if the extracellular antigen-binding domain of the ADGRE2-targeted chimeric receptor is an scFv, the variable regions are positioned from the N- to the C-terminus: V_{H}-V_{L}. In certain embodiments, the ADGRE2-targeted scFv comprises or consists of the amino acid sequence set forth in SEQ ID NO: 45, which is provided in Table 2. In certain embodiments, the ADGRE2-targeted scFv is designated as “ADGRE2-B”. The CDRs provided in Table 2 are identified according to the IM GT numbering system.

TABLE 2
(ADGRE2-B)

| | CDRs | | |
|---|---|---|---|
| | 1 | 2 | 3 |
| V_{H} | GYTFTNYW [SEQ ID | VYPGDGDT [SEQ ID | ARGFTAYGMDY [SEQ ID NO: |
| | NO: 33] | NO: 34] | 35] |
| V_{L} | SSVSY [SEQ ID | DTS [SEQ ID NO: | QQWSSNPLT [SEQ ID NO: |
| | NO: 36] | 37] | 38] |

| | |
|---|---|
| Full V_{H} | QVQLQQSGAEVKKPGASVKLSCKASGYTFTNYWMQWIRQAPGQGLEWIGAVYPGDGDTRYT |
| | QKFQGRATLTADTSISTAYMEVSRLRSDDTAVYYCARGFTAYGMDYWGQGTTVTVSS |
| | [SEQ ID NO: 43] |
| Full V_{L} | EIVLTQSPATMSASPGERVTMSCSASSSVSYMHWYQQKSGLSPKRWIYDTSKLASGVPDRF |
| | SGSGSGTDYTFTISRMEPEDFATYYCQQWSSNPLTFGGGTKLEIK [SEQ ID NO: 44] |
| V_{H}-V_{L} | QVQLQQSGAEVKKPGASVKLSCKASGYTFTNYWMQWIRQAPGQGLEWIGAVYPGDGDTRYT |
| scFv | QKFQGRATLTADTSISTAYMEVSRLRSDDTAVYYCARGFTAYGMDYWGQGTTVTVSSGGGG |
| | SGGGGSGGGGSEIVLTQSPATMSASPGERVTMSCSASSSVSYMHWYQQKSGLSPKRWIYDT |
| | SKLASGVPDRFSGSGSGTDYTFTISRMEPEDFATYYCQQWSSNPLTFGGGTKLEIK [SEQ |
| | ID NO: 45] |

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 345 or a conservative modification thereof, and a V_{H} CDR3 comprising the amino acid sequence set forth in SEQ ID NO: 35 or a conservative modification thereof. SEQ ID NOS: 33-35 are provided in Tables 1-3.

In certain embodiments, the extracellular antigen-binding domain of the ADGRE2-targeted chimeric receptor (e.g., an ADGRE2-targeted scFv) comprises a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof. SEQ ID NOs: 36-38 are provided in Tables 1-3.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, a V_{H} CDR3 comprising the amino acid sequence set forth in SEQ ID NO: 35 or a conservative modification thereof, a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33, a V_{H} CDR2 comprising the amino acid sequence set forth in SEQ ID NO: 34, a V_{H} CDR3 comprising the amino acid sequence set forth in SEQ ID NO: 35, a V_{L} CDR1 comprising the amino acid sequence set forth in SEQ ID NO: 36, a V_{L} CDR2 comprising the amino acid sequence set forth in SEQ ID NO: 37, and a V_{L} CDR3 comprising the amino acid sequence set forth in SEQ ID NO: 38.

In certain embodiments, the extracellular antigen-binding domain of the ADGRE2-targeted chimeric receptor (e.g., an ADGRE2-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 46. For example, the extracellular antigen-binding domain of the chimeric receptor (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 46. In certain embodiments, the extracellular antigen-binding domain of the ADGRE2-targeted chimeric receptor comprises a V_{H} comprising the amino acid sequence set forth in SEQ ID NO: 46. SEQ ID NO: 46 is provided in Table 3 below.

In certain embodiments, the extracellular antigen-binding domain of the ADGRE2-targeted chimeric receptor (e.g., an ADGRE2-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 47. For example, the extracellular antigen-binding domain of the ADGRE2-targeted chimeric receptor (e.g., an ADGRE2-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 47. In certain embodiments, the extracellular antigen-binding domain of the ADGRE2-targeted chimeric receptor comprises a V_{L} comprising the amino acid sequence set forth in SEQ ID NO: 47. SEQ ID NO: 47 is provided in Table 3 below.

In certain embodiments, the extracellular antigen-binding domain of the ADGRE2-targeted chimeric receptor (e.g., an ADGRE2-targeted scFv) comprises a V_{H} comprising the amino acid sequence set forth in SEQ ID NO: 46, and a V_{L} comprising the amino acid sequence set forth in SEQ ID NO: 47. In certain embodiments, the V_{H} and V_{L} are linked via a linker. In certain embodiments, the linker comprises the amino acid sequence set forth in SEQ ID NO: 2. In certain embodiments, the linker comprises the amino acid sequence set forth in SEQ ID NO: 149.

In certain embodiments, the variable regions within the extracellular antigen-binding domain of the ADGRE2-targeted chimeric receptor have to be linked one after another such that at the N-terminus of the extracellular antigen-binding domain, a heavy chain variable region (V_{H}) is positioned. In certain embodiments, if the extracellular antigen-binding domain of the ADGRE2-targeted chimeric receptor is an scFv, the variable regions are positioned from the N- to the C-terminus: V_{H}-V_{L}. In certain embodiments, the ADGRE2-targeted scFv comprises or consists of the amino acid sequence set forth in SEQ ID NO: 48, which is provided in Table 3. In certain embodiments, the ADGRE2-targeted scFv is designated as “ADGRE2-C”. The CDRs provided in Table 3 are identified according to the IM GT numbering system.

TABLE 3
(ADGRE2-C)

| | CDRs | | |
|---|---|---|---|
| | 1 | 2 | 3 |
| V_{H} | GYTFTNYW [SEQ ID | VYPGDGDT [SEQ ID | ARGFTAYGMDY [SEQ ID NO: |
| | NO: 33] | NO: 34] | 35] |
| V_{L} | SSVSY [SEQ ID | DTS [SEQ ID NO: | QQWSSNPLT [SEQ ID NO: |
| | NO: 36] | 37] | 38] |

| | |
|---|---|
| Full V_{H} | QVQLQQSGAEVKKPGASVKLSCKASGYTFTNYWMQWVRQAPGQGLEWIGAVYPGDGDTRYT |
| | QKFQGRATLTADTSTSTVYMEVSSLRSEDTAVYYCARGFTAYGMDYWGQGTTVTVSS |
| | [SEQ ID NO: 46] |
| Full V_{L} | EIVLTQSPATMSASPGERVTMSCSASSSVSYMHWYQQKSGLSPKRWIYDTSKLASGVPDRF |
| | SGSGSGTDYTFTISRMEPEDFATYYCQQWSSNPLTFGGGTKLEIK |
| | [SEQ ID NO: 47] |
| V_{H}-V_{L} | QVQLQQSGAEVKKPGASVKLSCKASGYTFTNYWMQWVRQAPGQGLEWIGAVYPGDGDTRYT |
| scFv | QKFQGRATLTADTSTSTVYMEVSSLRSEDTAVYYCARGFTAYGMDYWGQGTTVTVSS |
| | GGGGSGGGGSGGGGSEIVLTQSPATMSASPGERVTMSCSASSSVSYMHWYQQKSGLSPKRW |
| | IYDTSKLASGVPDRFSGSGSGTDYTFTISRMEPEDFATYYCQQWSSNPLTFGGGTKLEIK |
| | [SEQ ID NO: 48] |

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, and a V_{H} CDR3 comprising the amino acid sequence set forth in SEQ ID NO: 35 or a conservative modification thereof. SEQ ID NOS: 33-35 are provided in Tables 1-4.

In certain embodiments, the extracellular antigen-binding domain of the ADGRE2-targeted chimeric receptor (e.g., an ADGRE2-targeted scFv) comprises a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof. SEQ ID NOs: 36-38 are provided in Tables 1-4.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, a V_{H} CDR3 comprising the amino acid sequence set forth in SEQ ID NO: 35 or a conservative modification thereof, a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33, a V_{H} CDR2 comprising the amino acid sequence set forth in SEQ ID NO: 34, a V_{H} CDR3 comprising the amino acid sequence set forth in SEQ ID NO: 35, a V_{L} CDR1 comprising the amino acid sequence set forth in SEQ ID NO: 36, a V_{L} CDR2 comprising the amino acid sequence set forth in SEQ ID NO: 37, and a V_{L} CDR3 comprising the amino acid sequence set forth in SEQ ID NO: 38.

In certain embodiments, the extracellular antigen-binding domain of the ADGRE2-targeted chimeric receptor (e.g., an ADGRE2-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 49. For example, the extracellular antigen-binding domain of the ADGRE2-targeted chimeric receptor (e.g., an ADGRE2-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 49. In certain embodiments, the extracellular antigen-binding domain of ADGRE2-targeted chimeric receptor comprises a V_{H} comprising the amino acid sequence set forth in SEQ ID NO: 49. SEQ ID NO: 49 is provided in Table 4 below.

In certain embodiments, the extracellular antigen-binding domain of the ADGRE2-targeted chimeric receptor (e.g., an ADGRE2-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 50. For example, the extracellular antigen-binding domain of the ADGRE2-targeted chimeric receptor (e.g., an ADGRE2-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 50. In certain embodiments, the extracellular antigen-binding domain of the ADGRE2-targeted chimeric receptor comprises a V_{L} comprising the amino acid sequence set forth in SEQ ID NO: 50. SEQ ID NO: 50 is provided in Table 4 below.

In certain embodiments, the extracellular antigen-binding domain of the ADGRE2-targeted chimeric receptor (e.g., an ADGRE2-targeted scFv) comprises a V_{H} comprising the amino acid sequence set forth in SEQ ID NO: 49, and a V_{L} comprising the amino acid sequence set forth in SEQ ID NO: 50. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the ADGRE2-targeted chimeric receptor have to be linked one after another such that at the N-terminus of the extracellular antigen-binding domain, a heavy chain variable region (V_{H}) is positioned. In certain embodiments, if the extracellular antigen-binding domain of the ADGRE2-targeted chimeric receptor is an scFv, the variable regions are positioned from the N- to the C-terminus: V_{H}-V_{L}. In certain embodiments, the ADGRE2-targeted scFv comprises or consists of the amino acid sequence set forth in SEQ ID NO: 51, which is provided in Table 4. In certain embodiments, the ADGRE2-targeted scFv is designated as “ADGRE2-D”. The CDRs provided in Table 4 are identified according to the IM GT numbering system.

TABLE 4
(ADGRE2-D)

| | CDRs | | |
|---|---|---|---|
| | 1 | 2 | 3 |
| V_{H} | GYTFTNYW [SEQ ID | VYPGDGDT [SEQ ID | ARGFTAYGMDY [SEQ ID |
| | NO: 33] | NO: 34] | NO: 35] |
| V_{L} | SSVSY [SEQ ID | DTS [SEQ ID NO: 37] | QQWSSNPLT [SEQ ID NO: |
| | NO: 36] | | 38] |

| | |
|---|---|
| Full V_{H} | QVQLQQSGAEVKKPGASVKVSCKASGYTFTNYWMQWVRQAPGQGLEWMGAVYPGDGDTRHT |
| | QKFKGRVTMTRDTSTSTVYMELSSLRSEDTAVYYCARGFTAYGMDYWGQGTLVTVSS |
| | [SEQ ID NO: 49] |
| Full V_{L} | EIVLTQSPATLSLSPGERATLSCSASSSVSYMHWYQQKPGLAPRLLIYDTSKLASGIPDRF |
| | SGSGSGTDFTLTISRLEPEDFAVYYCQQWSSNPLTFGQGTKVEIK |
| | [SEQ ID NO: 50] |
| V_{H}-V_{L} | QVQLQQSGAEVKKPGASVKVSCKASGYTFTNYWMQWVRQAPGQGLEWMGAVYPGDGDTRHT |
| scFv | QKFKGRVTMTRDTSTSTVYMELSSLRSEDTAVYYCARGFTAYGMDYWGQGTLVTVSSASTG |
| | GGGSGGGGSGGGGSEIVLTQSPATLSLSPGERATLSCSASSSVSYMHWYQQKPGLAPRLLI |
| | YDTSKLASGIPDRFSGSGSGTDFTLTISRLEPEDFAVYYCQQWSSNPLTFGQGTKVEIK |
| | [SEQ ID NO: 51] |

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, and a V_{H} CDR3 comprising the amino acid sequence set forth in SEQ ID NO: 35 or a conservative modification thereof. SEQ ID NOS: 34-36 are provided in Tables 1-5.

In certain embodiments, the extracellular antigen-binding domain of the ADGRE2-targeted chimeric receptor (e.g., an ADGRE2-targeted scFv) comprises a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof. SEQ ID NOs: 36-38 are provided in Tables 1-5.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, a V_{H} CDR3 comprising the amino acid sequence set forth in SEQ ID NO: 5 or a conservative modification thereof, a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33, a V_{H} CDR2 comprising the amino acid sequence set forth in SEQ ID NO: 34, a V_{H} CDR3 comprising the amino acid sequence set forth in SEQ ID NO: 35, a V_{L} CDR1 comprising the amino acid sequence set forth in SEQ ID NO: 36, a V_{L} CDR2 comprising the amino acid sequence set forth in SEQ ID NO: 37, and a V_{L} CDR3 comprising the amino acid sequence set forth in SEQ ID NO: 38.

In certain embodiments, the extracellular antigen-binding domain of the ADGRE2-targeted chimeric receptor (e.g., an ADGRE2-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 52. For example, the extracellular antigen-binding domain of the ADGRE2-targeted chimeric receptor (e.g., an ADGRE2-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 52. In certain embodiments, the extracellular antigen-binding domain of the ADGRE2-targeted chimeric receptor comprises a V_{H} comprising the amino acid sequence set forth in SEQ ID NO: 52. SEQ ID NO: 52 is provided in Table 5 below.

In certain embodiments, the extracellular antigen-binding domain of the ADGRE2-targeted chimeric receptor (e.g., an ADGRE2-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 53. For example, the extracellular antigen-binding domain of the ADGRE2-targeted chimeric receptor (e.g., an ADGRE2-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 53. In certain embodiments, the extracellular antigen-binding domain of the ADGRE2-targeted chimeric receptor comprises a V_{L} comprising the amino acid sequence set forth in SEQ ID NO: 53. SEQ ID NO: 53 is provided in Table 5 below.

In certain embodiments, the extracellular antigen-binding domain of the ADGRE2-targeted chimeric receptor (e.g., an ADGRE2-targeted scFv) comprises a V_{H} comprising the amino acid sequence set forth in SEQ ID NO: 52, and a V_{L} comprising the amino acid sequence set forth in SEQ ID NO: 53. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the ADGRE2-targeted chimeric receptor have to be linked one after another such that at the N-terminus of the extracellular antigen-binding domain, a heavy chain variable region (V_{H}) is positioned. In certain embodiments, if the extracellular antigen-binding domain of the ADGRE2-targeted chimeric receptor is an scFv, the variable regions are positioned from the N- to the C-terminus: V_{H}-V_{L}. In certain embodiments, the ADGRE2-targeted scFv comprises or consists of the amino acid sequence set forth in EQ ID NO: 54, which is provided in Table 5. In certain embodiments, the ADGRE2-targeted scFv is designated as “ADGRE2-E”. The CDRs provided in Table 5 are identified according to the IM GT numbering system.

TABLE 5
(ADGRE2-E)

| | CDRs | | |
|---|---|---|---|
| | 1 | 2 | 3 |
| V_{H} | GYTFTNYW [SEQ ID | VYPGDGDT [SEQ ID | ARGFTAYGMDY [SEQ ID |
| | NO: 33] | NO: 34] | NO: 35] |
| V_{L} | SSVSY [SEQ ID NO: | DTS [SEQ ID NO: 37] | QQWSSNPLT [SEQ ID NO: |
| | 36] | | 38] |

| | |
|---|---|
| Full V_{H} | QVQLQQSGAEVKKPGASVKVSCKASGYTFTNYWMQWVRQAPGQGLEWIGAVYPGDGDTRHT |
| | QKFKGRVTMTADKSTSTVYMELSSLRSEDTAVYYCARGFTAYGMDYWGQGTLVTVSS |
| | [SEQ ID NO: 52] |
| Full V_{L} | QIVLTQSPATLSLSPGERATLTCSASSSVSYMHWYQQKPGLSPKRWIYDTSKLASGVPDRF |
| | SGSGSGTDYTFTIRRLEPEDFATYYCQQWSSNPLTFGQGTKVEIK [SEQ ID NO: 53] |
| V_{H}-V_{L} | QVQLQQSGAEVKKPGASVKVSCKASGYTFTNYWMQWVRQAPGQGLEWIGAVYPGDGDTRHT |
| scFv | QKFKGRVTMTADKSTSTVYMELSSLRSEDTAVYYCARGFTAYGMDYWGQGTLVTVSSASTG |
| | GGGSGGGGSGGGGSQIVLTQSPATLSLSPGERATLTCSASSSVSYMHWYQQKPGLSPKRWI |
| | YDTSKLASGVPDRFSGSGSGTDYTFTIRRLEPEDFATYYCQQWSSNPLTFGQGTKVEIK |
| | [SEQ ID NO: 54] |

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, and a V_{H} CDR3 comprising the amino acid sequence set forth in SEQ ID NO: 35 or a conservative modification thereof. SEQ ID NOS: 33-35 are provided in Tables 1-6.

In certain embodiments, the extracellular antigen-binding domain of the ADGRE2-targeted chimeric receptor (e.g., an ADGRE2-targeted scFv) comprises a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof. SEQ ID NOs: 36-38 are provided in Tables 1-6.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, a V_{H} CDR3 comprising the amino acid sequence set forth in SEQ ID NO: 35 or a conservative modification thereof, a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33, a V_{H} CDR2 comprising the amino acid sequence set forth in SEQ ID NO: 34, a V_{H} CDR3 comprising the amino acid sequence set forth in SEQ ID NO: 35, a V_{L} CDR1 comprising the amino acid sequence set forth in SEQ ID NO: 36, a V_{L} CDR2 comprising the amino acid sequence set forth in SEQ ID NO: 37, and a V_{L} CDR3 comprising the amino acid sequence set forth in SEQ ID NO: 38.

In certain embodiments, the extracellular antigen-binding domain of the ADGRE2-targeted chimeric receptor (e.g., an ADGRE2-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 55. For example, the extracellular antigen-binding domain of the ADGRE2-targeted chimeric receptor (e.g., an ADGRE2-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 55. In certain embodiments, the extracellular antigen-binding domain of the ADGRE2-targeted chimeric receptor comprises a V_{H} comprising the amino acid sequence set forth in SEQ ID NO: 55. SEQ ID NO: 55 is provided in Table 6 below.

In certain embodiments, the extracellular antigen-binding domain of the ADGRE2-targeted chimeric receptor (e.g., an ADGRE2-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 56. For example, the extracellular antigen-binding domain of the ADGRE2-targeted chimeric receptor (e.g., an ADGRE2-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 56. In certain embodiments, the extracellular antigen-binding domain of the ADGRE2-targeted chimeric receptor comprises a V_{L} comprising the amino acid sequence set forth in SEQ ID NO: 56. SEQ ID NO: 56 is provided in Table 6 below.

In certain embodiments, the extracellular antigen-binding domain of the ADGRE2-targeted chimeric receptor (e.g., an ADGRE2-targeted scFv) comprises a V_{H} comprising the amino acid sequence set forth in SEQ ID NO: 55, and a V_{L} comprising the amino acid sequence set forth in SEQ ID NO: 56. In certain embodiments, the V_{H} and V_{L} are linked via a linker. In certain embodiments, the linker comprises the amino acid sequence set forth in SEQ ID NO: 2. In certain embodiments, the linker comprises the amino acid sequence set forth in SEQ ID NO: 149.

In certain embodiments, the variable regions within the extracellular antigen-binding domain of the ADGRE2-targeted chimeric receptor have to be linked one after another such that at the N-terminus of the extracellular antigen-binding domain, a heavy chain variable region (V_{H}) is positioned. In certain embodiments, if the extracellular antigen-binding domain of the ADGRE2-targeted chimeric receptor is an scFv, the variable regions are positioned from the N- to the C-terminus: V_{H}-V_{L}. In certain embodiments, the ADGRE2-targeted scFv comprises or consists of the amino acid sequence set forth in in SEQ ID NO: 57, which is provided in Table 6. In certain embodiments, the ADGRE2-targeted scFv is designated as “ADGRE2-F”. The CDRs provided in Table 6 are identified according to the IM GT numbering system.

TABLE 6
(ADGRE2-F)

| | CDRs | | |
|---|---|---|---|
| | 1 | 2 | 3 |
| V_{H} | GYTFTNYW [SEQ ID | VYPGDGDT [SEQ ID | ARGFTAYGMDY [SEQ ID |
| | NO: 33] | NO: 34] | NO: 35] |
| V_{L} | SSVSY [SEQ ID NO: | DTS [SEQ ID NO: 37] | QQWSSNPLT [SEQ ID |
| | 36] | | NO: 38] |

| | |
|---|---|
| Full V_{H} | QVQLQQSGAEVKKPGASVKLSCKASGYTFTNYWMQWIRQAPGQGLEWIGAVYPGDGDTRYTQ |
| | KFQGRATLTADTSTSTAYMEVSSLRSEDTAVYYCARGFTAYGMDYWGQGTTVTVSS [SEQ |
| | ID NO: 55] |
| Full V_{L} | EIVLTQSPATLSASPGERVTMSCSASSSVSYMHWYQQKPGLAPRRWIYDTSKLASGVPDRFS |
| | GSGSGTDYTFTISRMEPEDFATYYCQQWSSNPLTFGGGTKLEIK [SEQ ID NO: 56] |
| V_{H}-V_{L} | QVQLQQSGAEVKKPGASVKLSCKASGYTFTNYWMQWIRQAPGQGLEWIGAVYPGDGDTRYTQ |
| scFv | KFQGRATLTADTSTSTAYMEVSSLRSEDTAVYYCARGFTAYGMDYWGQGTTVTVSSGGGGSG |
| | GGGSGGGGSEIVLTQSPATLSASPGERVTMSCSASSSVSYMHWYQQKPGLAPRRWIYDTSKL |
| | ASGVPDRFSGSGSGTDYTFTISRMEPEDFATYYCQQWSSNPLTFGGGTKLEIK [SEQ ID |
| | NO: 57] |

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, and a V_{H} CDR3 comprising the amino acid sequence set forth in SEQ ID NO: 35 or a conservative modification thereof. SEQ ID NOS: 33-35 are provided in Tables 1-7.

In certain embodiments, the extracellular antigen-binding domain of the ADGRE2-targeted chimeric receptor (e.g., an ADGRE2-targeted scFv) comprises a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof. SEQ ID NOs: 36-38 are provided in Tables 1-7.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33 or a conservative modification thereof, a V_{H} CDR2 comprising the amino acid sequence set forth in SEQ ID NO: 34 or a conservative modification thereof, a V_{H} CDR3 comprising the amino acid sequence set forth in SEQ ID NO: 35 or a conservative modification thereof, a V_{L} CDR1 comprising the amino acid sequence set forth in SEQ ID NO: 36 or a conservative modification thereof, a V_{L} CDR2 comprising the amino acid sequence set forth in SEQ ID NO: 37 or a conservative modification thereof, and a V_{L} CDR3 comprising the amino acid sequence set forth in SEQ ID NO: 38 or a conservative modification thereof.

In certain embodiments, the extracellular antigen-binding domain of the ADGRE2-targeted chimeric receptor (e.g., an ADGRE2-targeted scFv) comprises a V_{H} CDR1 comprising the amino acid sequence set forth in SEQ ID NO: 33, a V_{H} CDR2 comprising the amino acid sequence set forth in SEQ ID NO: 34, a V_{H} CDR3 comprising the amino acid sequence set forth in SEQ ID NO: 35, a V_{L} CDR1 comprising the amino acid sequence set forth in SEQ ID NO: 36, a V_{L} CDR2 comprising the amino acid sequence set forth in SEQ ID NO: 37, and a V_{L} CDR3 comprising the amino acid sequence set forth in SEQ ID NO: 38.

In certain embodiments, the extracellular antigen-binding domain of the ADGRE2-targeted chimeric receptor (e.g., an ADGRE2-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 146. For example, the extracellular antigen-binding domain of the ADGRE2-targeted chimeric receptor (e.g., an ADGRE2-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 146. In certain embodiments, the extracellular antigen-binding domain of the ADGRE2-targeted chimeric receptor comprises a V_{H} comprising the amino acid sequence set forth in SEQ ID NO: 146. SEQ ID NO: 146 is provided in Table 7 below.

In certain embodiments, the extracellular antigen-binding domain of the ADGRE2-targeted chimeric receptor (e.g., an ADGRE2-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 147. For example, the extracellular antigen-binding domain of the ADGRE2-targeted chimeric receptor (e.g., an ADGRE2-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino acid sequence set forth in SEQ ID NO: 147. In certain embodiments, the extracellular antigen-binding domain of the ADGRE2-targeted chimeric receptor comprises a V_{L} comprising the amino acid sequence set forth in SEQ ID NO: 147. SEQ ID NO: 147 is provided in Table 7 below.

In certain embodiments, the extracellular antigen-binding domain of the ADGRE2-targeted chimeric receptor (e.g., an ADGRE2-targeted scFv) comprises a V_{H} comprising the amino acid sequence set forth in SEQ ID NO: 146, and a V_{L} comprising the amino acid sequence set forth in SEQ ID NO: 147. In certain embodiments, the V_{H} and V_{L} are linked via a linker. In certain embodiments, the linker comprises the amino acid sequence set forth in SEQ ID NO: 2. In certain embodiments, the linker comprises the amino acid sequence set forth in SEQ ID NO: 149.

In certain embodiments, the variable regions within the extracellular antigen-binding domain of the ADGRE2-targeted chimeric receptor have to be linked one after another such that at the N-terminus of the extracellular antigen-binding domain, a heavy chain variable region (V_{H}) is positioned. In certain embodiments, if the extracellular antigen-binding domain of the ADGRE2-targeted chimeric receptor is an scFv, the variable regions are positioned from the N- to the C-terminus: V_{H}-V_{L}. In certain embodiments, the ADGRE2-targeted scFv comprises or consists of the amino acid sequence set forth in in SEQ ID NO: 148, which is provided in Table 7. In certain embodiments, the ADGRE2-targeted scFv is designated as “ADGRE2-G”. The CDRs provided in Table 7 are identified according to the IM GT numbering system.

TABLE 7
(ADGRE2-G)

| | CDRs | | |
|---|---|---|---|
| | 1 | 2 | 3 |
| V_{H} | GYTFTNYW [SEQ ID | VYPGDGDT [SEQ ID | ARGFTAYGMDY [SEQ ID |
| | NO: 33] | NO: 34] | NO: 35] |
| V_{L} | SSVSY [SEQ ID NO: | DTS [SEQ ID NO: 37] | QQWSSNPLT [SEQ ID NO: |
| | 36] | | 38] |

| | |
|---|---|
| Full V_{H} | QVQLVQSGAEVAKPGASVKLSCKASGYTFTNYWMQWIKQAPGQGLEWIGAVYPGDGDTRHTQ |
| | KFKGKATLTADKSTSTAYMEVSSLRSEDTAVYYCARGFTAYGMDYWGQGTTVTVSS [SEQ |
| | ID NO: 146] |
| Full V_{L} | EIVLTQSPATMSASPGERVTMSCSASSSVSYMHWYQQKSGQSPKRWIYDTSKLASGVPARFS |
| | GSGSGTDYTFTISSMEPEDFATYYCQQWSSNPLTFGGGTKLEIK [SEQ ID NO: 147] |
| V_{H}-V_{L} | QVQLVQSGAEVAKPGASVKLSCKASGYTFTNYWMQWIKQAPGQGLEWIGAVYPGDGDTRHTQ |
| scFv | KFKGKATLTADKSTSTAYMEVSSLRSEDTAVYYCARGFTAYGMDYWGQGTTVTVSSGGGGSG |
| | GGGSGGGGSEIVLTQSPATMSASPGERVTMSCSASSSVSYMHWYQQKSGQSPKRWIYDTSKL |
| | ASGVPARFSGSGSGTDYTFTISSMEPEDFATYYCQQWSSNPLTFGGGTKLEIK [SEQ ID |
| | NO: 148] |

The V_{H} and/or V_{L} amino acid sequences comprising or consisting of at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 39, SEQ ID NO: 40, SEQ ID NO: 43, SEQ ID NO: 44, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 146, or SEQ ID NO: 147) may contain substitutions (e.g., conservative substitutions), insertions, or deletions relative to the specified sequence(s), but retain the ability to bind to a target antigen (e.g., ADGRE2). In certain embodiments, a total of 1 to 10 amino acids are substituted, inserted and/or deleted in a specific sequence (e.g., SEQ ID NO: 39, SEQ ID NO: 40, SEQ ID NO: 43, SEQ ID NO: 44, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 146, or SEQ ID NO: 147). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NO: 39, SEQ ID NO: 40, SEQ ID NO: 43, SEQ ID NO: 44, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 146, or SEQ ID NO: 147, including post-translational modifications of that sequence (SEQ ID NO: 39, SEQ ID NO: 40, SEQ ID NO: 43, SEQ ID NO: 44, SEQ ID NO: 46, SEQ ID NO: 47, SEQ ID NO: 49, SEQ ID NO: 50, SEQ ID NO: 52, SEQ ID NO: 53, SEQ ID NO: 55, SEQ ID NO: 56, SEQ ID NO: 146, or SEQ ID NO: 147).

In certain embodiments, the extracellular antigen-binding domain of a presently disclosed ADGRE2-targeted chimeric receptor cross-competes for binding to ADGRE2 with a reference antibody or an antigen-binding portion thereof comprising the a V_{H} CDR1 comprising the amino acid sequence set forth in SEQ ID NO: 33, a V_{H} CDR2 comprising the amino acid sequence set forth in SEQ ID NO: 34; a V_{H} CDR3 comprising the amino acid sequence set forth in SEQ ID NO: 35; a V_{L} CDR1 comprising the amino acid sequence set forth in SEQ ID NO: 36; a V_{L} CDR2 comprising the amino acid sequence set forth in SEQ ID NO: 37; and a V_{L} CDR3 comprising the amino acid sequence set forth in SEQ ID NO: 38. In certain embodiments, the extracellular antigen-binding domain of a presently disclosed ADGRE2-targeted chimeric receptor cross-competes for binding to ADGRE2 with a reference antibody or an antigen-binding portion thereof comprising the V_{H} and V_{L} sequences of, for example, any one of the presently disclosed scFvs (e.g., ADGRE2-A, ADGRE2-B, ADGRE2-C, ADGRE2-D, ADGRE2-E, ADGRE2-F, and ADGRE2-G).

In certain embodiments, the extracellular antigen-binding domain of a presently disclosed ADGRE2-targeted chimeric receptor binds to the same or substantially the same epitope region on ADGRE2 with a reference antibody or an antigen-binding portion thereof comprising the a V_{H} CDR1 comprising the amino acid sequence set forth in SEQ ID NO: 33, a V_{H} CDR2 comprising the amino acid sequence set forth in SEQ ID NO: 34; a V_{H} CDR3 comprising the amino acid sequence set forth in SEQ ID NO: 35; a V_{L} CDR1 comprising the amino acid sequence set forth in SEQ ID NO: 36; a V_{L} CDR2 comprising the amino acid sequence set forth in SEQ ID NO: 37; and a V_{L} CDR3 comprising the amino acid sequence set forth in SEQ ID NO: 38. In certain embodiments, the extracellular antigen-binding domain of a presently disclosed ADGRE2-targeted chimeric receptor binds to the same or substantially the same epitope region on ADGRE2 with a reference antibody or an antigen-binding portion thereof comprising the V_{H} and V_{L} sequences of, for example, any one of the presently disclosed scFvs (e.g., ADGRE2-A, ADGRE2-B, ADGRE2-C, ADGRE2-D, ADGRE2-E, ADGRE2-F, and ADGRE2-G).

Extracellular antigen-binding domains of the presently disclosed ADGRE2-targeted chimeric receptors that cross-compete or compete with the reference antibody or antigen-binding portions thereof for binding to ADGRE2 can be identified by using routine methods known in the art, including, but not limited to, ELISAs, radioimmunoassay (RIA s), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody to an ADGRE2, admixing the reference antibody with a test extracellular antigen-binding domain, measuring a second binding of the reference antibody to the ADGRE2 in the presence of the test extracellular antigen-binding domain, and comparing the initial binding with the second binding of the reference antibody, wherein a decreased second binding of the reference antibody to the ADGRE2 in comparison to the initial binding indicates that the test extracellular antigen-binding domain cross-competes with the reference antibody for binding to ADGRE2, e.g., one that recognizes the same or substantially the same epitope, an overlapping epitope, or an adjacent epitope. In certain embodiments, the reference antibody is labeled, e.g., with a fluorochrome, biotin, or peroxidase. In certain embodiments, the ADGRE2 is expressed in cells, e.g., in a flow cytometry test. In certain embodiments, the ADGRE2 is immobilized onto a surface, including a Biacore ship (e.g., in a Biacore test), or other media suitable for surface plasmon resonance analysis. The binding of the reference antibody in the presence of a completely irrelevant antibody (that does not bind to ADGRE2) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody to ADGRE2 by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody for binding to ADGRE2. In certain embodiments, the assays are performed at room temperature.

In certain embodiments, the antibody-binding assay comprises measuring an initial binding of a test extracellular antigen-binding domain to ADGRE2, admixing the test extracellular antigen-binding domain with a reference antibody, measuring a second binding of the test extracellular antigen-binding domain to ADGRE2 in the presence of the reference antibody, and comparing the initial binding with the second binding of the test extracellular antigen-binding domain, where a decreased second binding of the test extracellular antigen-binding domain to ADGRE2 in comparison to the initial binding indicates that the test extracellular antigen-binding domain cross-competes with the reference antibody for binding to ADGRE2, e.g., one that recognizes the same or substantially the same epitope, an overlapping epitope, or an adjacent epitope. In certain embodiments, the test extracellular antigen-binding domain is labeled, e.g., with a fluorochrome, biotin, or peroxidase. In certain embodiments, the ADGRE2 is expressed in cells, e.g., in a flow cytometry test. In certain embodiments, the ADGRE2 is immobilized onto a surface, including a Biacore ship (e.g., in a Biacore test), or other media suitable for surface plasmon resonance analysis. The binding of the test extracellular antigen-binding domain in the presence of a completely irrelevant antibody (that does not bind to ADGRE2) can serve as the control high value. The control low value can be obtained by incubating a labeled test extracellular antigen-binding domain with an unlabeled test extracellular antigen-binding domain, where competition and reduced binding of the labeled test extracellular antigen-binding domain would occur. In certain embodiments, a test extracellular antigen-binding domain, whose binding to ADGRE2 is decreased by at least about 20%, at least about 30%, at least about 40%, at least about 50%, at least about 60%, at least about 70%, at least about 80%, at least about 90%, or at least about 95% in the presence of a reference antibody, is considered to be an extracellular antigen-binding domain that cross-competes with the reference antibody for binding to ADGRE2. In certain embodiments, the assays are performed at room temperature.

In certain embodiments, the extracellular antigen-binding domain of the presently disclosed ADGRE2-targeted chimeric receptor comprises a linker connecting the heavy chain variable region and light chain variable region of the extracellular antigen-binding domain. In certain embodiments, the linker comprises the amino acid sequence set forth in SEQ ID NO: 1. In certain embodiments, the linker comprises the amino acid sequence set forth in SEQ ID NO: 2. In certain embodiments, the linker comprises the amino acid sequence set forth in SEQ ID NO: 3. In certain embodiments, the linker comprises the amino acid sequence set forth in SEQ ID NO: 4. In certain embodiments, the linker comprises the amino acid sequence set forth in SEQ ID NO: 5. In certain embodiments, the linker comprises the amino acid sequence set forth in SEQ ID NO: 6. In certain embodiments, the linker comprises the amino acid sequence set forth in SEQ ID NO: 149.

In certain embodiments, the variable regions within the extracellular antigen-binding domain of the ADGRE2-targeted chimeric receptor have to be linked one after another such that at the N-terminus of the extracellular antigen-binding domain, a heavy chain variable region (V_{H}) is positioned. In certain embodiments, if the extracellular antigen-binding domain of the ADGRE2-targeted chimeric receptor is an scFv, the variable regions are positioned from the N- to the C-terminus: V_{H}-V_{L}.

In certain embodiments, the variable regions within the extracellular antigen-binding domain of the ADGRE2-targeted chimeric receptor have to be linked one after another such that at the N-terminus of the extracellular antigen-binding domain, a light chain variable region (V_{L}) is positioned. In certain embodiments, if the extracellular antigen-binding domain of the ADGRE2-targeted chimeric receptor is an scFv, the variable regions are positioned from the N- to the C-terminus: V_{L}-V_{H}.

In addition, the ADGRE2-targeted chimeric receptor can comprise a leader or a signal peptide that directs the nascent protein into the endoplasmic reticulum. In certain embodiments, the leader or signal peptide is positioned at (e.g., covalently joined to) the N-terminus of the extracellular antigen-binding domain of the ADGRE2-targeted chimeric receptor. Signal peptide or leader can be essential if the chimeric receptor is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. Non-limiting examples of signal peptides or leader sequences include an IL-2 signal sequence, a CD8 leader sequence, a kappa leader sequence, an albumin leader sequence, and a prolactin leader sequence. In certain embodiments, the IL-2 signal sequence comprises or consists of the amino acid sequence set forth in SEQ ID NO. 58 or SEQ ID NO: 59. In certain embodiments, the kappa leader sequence comprises or consists of the amino acid sequence set forth in SEQ ID NO. 60 or SEQ ID NO: 61. In certain embodiments, the CD8 signal sequence comprises or consists of the amino acid sequence set forth in SEQ ID NO. 62 or SEQ ID NO: 63. In certain embodiments, the albumin leader sequence comprises or consists of the amino acid sequence set forth in SEQ ID NO: 64. In certain embodiments, the prolactin leader sequence comprises or consists of the amino acid sequence set forth in SEQ ID NO: 65. In certain embodiments, the ADGRE2-targeted chimeric receptor comprises a signal peptide that comprises a CD8 polypeptide. In certain embodiments, the ADGRE2-targeted chimeric receptor comprises a signal peptide that comprises a CD8 polypeptide comprising or consisting of the amino acid sequence set forth in SEQ ID NO: 63.

[SEQ ID NO: 58]
MYRMQLLSCIALSLALVTNS
[SEQ ID NO: 59]
MYSMQLASCVTLTLVLLVNS
[SEQ ID NO: 60]
METPAQLLFLLLLWLPDTTG
[SEQ ID NO: 61]
METDTLLLWVLLLWVPGSTG
[SEQ ID NO: 62]
MALPVTALLLPLALLLHAARP
[SEQ ID NO: 63]
MALPVTALLLPLALLLHA
[SEQ ID NO: 64]
MKWVTFISLLESSAYS
[SEQ ID NO: 65]
MDSKGSSQKGSRLLLLLVVSNLLLCQGVVS

### 5.4. Exemplified ADGRE2-Targeted Chimeric Receptors

In certain embodiments, the ADGRE2-targeted chimeric receptor is an ADGRE2-targeted CAR. In certain embodiments, the ADGRE2-targeted CAR comprises (a) an extracellular antigen-binding domain comprising (i) a V_{H} that comprises a CDR1 comprising the amino acid sequence set forth in SEQ ID NO: 33, a CDR2 comprising the amino acid sequence set forth in SEQ ID NO: 34, and a V_{H} CDR3 comprising the amino acid sequence set forth in SEQ ID NO: 35, and (ii) a V_{L} that comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; (b) a hinge/spacer region comprising a CD28 polypeptide, (c) a transmembrane domain comprising a CD28 polypeptide (e.g., a transmembrane domain of human CD28 or a fragment thereof), and (d) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 2. In certain embodiments, the V_{H} and V_{L} are positioned from the N- to the C-terminus: V_{H}-V_{L}. In certain embodiments, the transmembrane domain comprises a CD28 polypeptide comprising or consisting of the amino acids 153 to 179 of SEQ ID NO: 10. In certain embodiments, the co-stimulatory signaling region comprises a CD28 polypeptide comprising or consisting of the amino acids 180 to 220 of SEQ ID NO: 10. In certain embodiments, the hinge/spacer region comprises a CD28 polypeptide comprising or consisting of the amino acids 114 to 152 of SEQ ID NO: 10. In certain embodiments, the extracellular antigen-binding domain and transmembrane domain are linked via a linker. In certain embodiments, the linker consists of the amino acid sequence set forth in SEQ ID NO: 150. SEQ ID NO: 150 is provided below. In certain embodiments, the ADGRE2-targeted CAR comprises or consists of the amino acid sequence set forth in SEQ ID NO: 66, which is provided below.

[SEQ ID NO: 66]
QVQLQQSGAEVAKPGASVKLSCKASGYTFTNYWMQWIKQAPGQGLEWIGA
VYPGDGDTRHTQKFKGKATLTADKSTSTAYMEVSSLRSEDTAVYYCARGF
TAYGMDYWGQGTTVTVSSGGGGSGGGGSGGGGSEIVLTQSPATMSASPGE
RVTMSCSASSSVSYMHWYQQKSGQSPKRWIYDTSKLASGVPARFSGSGSG
TDYTFTISSMEPEDFATYYCQQWSSNPLTFGGGTKLEIKRAAAIEVMYPP
PYLDNEKSNGTIIHVKGKHLCPSPLFPGPSKPFWVLVVVGGVLACYSLLV
TVAFIIEWVRSKRSRLLHSDYMNMTPRRPGPTRKHYQPYAPPRDFAAYRS
RVKFSRSADAPAYQQGQNQLYNELNLGRREEYDVLDKRRGRDPEMGGKPR
RKNPQEGLFNELQKDKMAEAFSEIGMKGERRRGKGHDGLFQGLSTATKDT
FDALHMQALPPR
[SEQ ID NO: 150]
RAAA

An exemplary nucleic acid sequence the amino acid sequence of SEQ ID NO: 66 is set forth in SEQ ID NO: 67, which is provided below.

[SEQ ID NO: 67]
caagttcagctccagcagagcggcgccgaagtggcaaagcctggagcgtc
agtcaagctgtcctgcaaagcgagtggctatacgttcacgaactactgga
tgcagtggataaagcaggctcccgggcagggtctggagtggattggagcc
gtctacccaggggacggcgacacccggcacactcaaaagttcaagggcaa
ggccaccctgaccgctgacaagagcacaagcacagcgtacatggaggtgt
cctctttgagatccgaagataccgctgtgtattattgtgcccggggcttc
actgcatacgggatggattactggggacaaggcactaccgtgactgtcag
ctccgggggtggaggctcaggcggggggggttcaggaggggggggatctg
aaattgtgctgacacagagccctgccacaatgtctgctagccctggcgag
cgcgtgaccatgtcttgtagcgccagcagcagcgtgtcctacatgcattg
gtatcaacagaagtccggccagtctcccaagcggtggatctacgatacaa
gcaagctggcctccggcgtgcccgccagattttctggcagcggctctgga
acagattacaccttcaccatctctagcatggaacctgaggattttgccac
ctactattgccagcagtggtccagcaatcccctgacatttggaggaggca
ccaagctggaaattaagagagcggccgcaattgaagttatgtatcctcct
ccttacctagacaatgagaagagcaatggaaccattatccatgtgaaagg
gaaacacctttgtccaagtcccctatttcccggaccttctaagccctttt
gggtgctggtggtggttggtggagtcctggcttgctatagcttgctagta
acagtggcctttattattttctgggtgaggagtaagaggagcaggctcct
gcacagtgactacatgaacatgactccccgccgccccgggcccacccgca
agcattaccagccctatgccccaccacgcgacttcgcagcctatcgctcc
agagtgaagttcagcaggagcgcagacgcccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgttcaatgaactgcagaaagataagat
ggcggaggccttcagtgagattgggatgaaaggcgagcgccggaggggca
aggggcacgatggccttttccagggtctcagtacagccaccaaggacacc
ttcgacgcccttcacatgcaggccctgccccctcgc

In certain embodiments, the ADGRE2-targeted chimeric receptor comprises a signal peptide at the N-terminus of the extracellular antigen-binding domain. In certain embodiments, the signal peptide comprises a CD8 polypeptide comprising or consisting of the amino acid sequence set forth in SEQ ID NO: 63.

### 5.5. Extracellular Antigen-binding Domain of CLEC12A-Targeted Chimeric Receptors

In certain embodiments, the presently disclosed chimeric receptor targets CLEC12A. In certain embodiments, the presently disclosed chimeric receptor comprises an extracellular antigen-binding domain that binds to CLEC12A.

C-type lectin domain family 12 member A (CLEC12A), also known as CLL-1, CLL1, DCAL-2, MICL, CD371, is a 30 kD C-type lectin transmembrane glycoprotein. It is expressed on monocytes, granulocytes, natural killer (NK) cells, and basophils. CLEC12A is an immunoinhibitory receptor that recruits Src homology phosphatases SHP-1 and SHP-2 to its phosphorylated cytoplasmic immunoreceptor tyrosine-based inhibitory motif (ITIM) (Sancho et al., Annu Rev. Immunol (2012); 30:491-529; Y an et al., Front Immunol (2015); 6:408; Lahoud et al., J Immunol (2011); 187:842). CLEC12A has been implicated as a negative regulatory uric acid crystals (monosodium urate, MSU) receptor that controls autoimmunity and inflammatory disease (Neumann et al., Immunity (2014); 40:389-99). CLEC12A is a negative regulator of granulocyte and monocyte function (Marshall et al., J Biol Chem (2004); 279 (15): 14792-802; Pyz et al., Eur J Immunol (2008); 38 (4): 1157-63).

In certain embodiments, the presently disclosed chimeric receptor targets human CLEC12A. In certain embodiments, the presently disclosed chimeric receptor comprises an extracellular antigen-binding domain that binds to human CLEC12A. In certain embodiments, the human CLEC12A comprises or consists of the amino acid sequence with a UniProt Reference No: Q5QGZ9-2 (SEQ ID NO: 68), or a fragment thereof. SEQ ID NO: 68 is provided below:

[SEQ ID NO: 68]
MSEEVTYADLQFQNSSEMEKIPEIGKFGEKAPPAPSHVWRPAALFLTLLC
LLLLIGLGVLASMEHVTLKIEMKKMNKLQNISEELQRNISLQLMSNMNIS
NKIRNLSTTLQTIATKLCRELYSKEQEHKCKPCPRRWIWHKDSCYFLSDD
VQTWQESKMACAAQNASLLKINNKNALEFIKSQSRSYDYWLGLSPEEDST
RGMRVDNIINSSAWVIRNAPDLNNMYCGYINRLYVQYYHCTYKKRMICEK
MANPVQLGSTYFREA

Human CLEC12A comprises a cytoplasmic domain, a transmembrane domain, and an extracellular domain. In certain embodiments, the cytoplasmic domain comprises or consists of amino acids 1 to 43 of SEQ ID NO: 68. In certain embodiments, the transmembrane domain comprises or consists of amino acid 44 to 64 of SEQ ID NO: 68. In certain embodiments, the extracellular domain comprises or consists of amino acid 65 to 265 of SEQ ID NO: 68.

In certain embodiments, the presently disclosed chimeric receptor targets a CLECL 12A polypeptide comprising or consisting of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 68 or a fragment thereof.

In certain embodiments, the extracellular antigen-binding domain of a presently disclosed CLEC12A-targeted chimeric receptor binds to the extracellular domain of CLEC12A.

In certain embodiments, the CLEC12A-targeted chimeric receptor is a chimeric antigen receptor (CAR). In certain embodiments, the CLEC12A-targeted CAR has the structure disclosed in Section 5.2.1. In certain embodiments, the CLEC12A-targeted CAR comprises an extracellular antigen-binding domain that binds to CLEC12A, a transmembrane domain, and an intracellular signaling domain.

In certain embodiments, the CLEC12A-targeted chimeric receptor is a Chimeric Co-Stimulatory Receptor (CCR). In certain embodiments, the CLEC12A-targeted CCR has the structure disclosed in Section 5.2.2. In certain embodiments, the CLEC12A-targeted CCR comprises an extracellular antigen-binding domain that binds to CLEC12A, a transmembrane domain, and an intracellular signaling domain that does not provide an activation signal to an immunoresponsive cell, e.g., the intracellular signaling domain does not comprise a CD3ζ polypeptide.

In certain embodiments, the CLEC12A-targeted chimeric receptor is a TCR like Fusion Molecules. In certain embodiments, the CLEC12A-targeted TCR like Fusion Molecules has the structure disclosed in Section 5.2.3.

In certain embodiments, the extracellular antigen-binding domain of the CLEC12A-targeted chimeric receptor (e.g., a CLEC12A-targeted scFv) binds to CLEC12A (e.g., human CLEC12A) with a dissociation constant (K_{D}) of at least about 1×10⁻⁶ M, at least about 1×10⁻⁷ M, at least about 1×10⁻⁸ M, at least about 1×10⁻⁹ M, or at least about 1×10⁻¹⁰ M. In certain embodiments, the extracellular antigen-binding domain of the CLEC12A-targeted chimeric receptor (e.g., a CLEC12A-targeted scFv) binds to CLEC12A (e.g., human CLEC12A) with a dissociation constant (K_{D}) of at least about 2×10⁻⁸ M. In certain embodiments, the extracellular antigen-binding domain of the CLEC12A-targeted chimeric receptor (e.g., a CLEC12A-targeted scFv) binds to CLEC12A (e.g., human CLEC12A) with a dissociation constant (K_{D}) of between about 2×10⁻⁸ M and about 8×10⁻⁹ M.

In certain embodiments, the extracellular antigen-binding domain of the CLEC12A-targeted chimeric receptor (e.g., a CLEC12A-targeted scFv) binds to CLEC12A (e.g., human CLEC12A) with a dissociation constant (K_{D}) between about 1 nM and about 50 nM, between about 5 nM and 30 nM, between about 5 nM and about 25 nM, or between about 8 nM and about 20 nM. In certain embodiments, the extracellular antigen-binding domain of the CLEC12A-targeted chimeric receptor (e.g., a CLEC12A-targeted scFv) binds to CLEC12A (e.g., human CLEC12A) with a dissociation constant (K_{D}) of at least about 50 nM, at least about 40 nM, at least about 35 nM, at least about 30 nM, at least about 25 nM, at least about 20 nM, at least about 19 nM, at least about 18 nM, at least about 17 nM, at least about 16 nM, at least about 15 nM, at least about 14 nM, at least about 13 nM, at least about 12 nM, at least about 11 nM, at least about 10 nM, at least about 9 nM, at least about 8 nM, at least about 7 nM, at least about 6 nM, at least about 5 nM.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 69 or a conservative modification thereof, a V_{H} CDR2 comprising the amino acid sequence set forth in SEQ ID NO: 70 or a conservative modification thereof, and a V_{H} CDR3 comprising the amino acid sequence set forth in SEQ ID NO: 71 or a conservative modification thereof. SEQ ID NOs: 69-71 are provided in Table 8.

In certain embodiments, the extracellular antigen-binding domain of the CLEC12A-targeted chimeric receptor (e.g., a CLEC12A-targeted scFv) comprises a V_{L} CDR1 comprising the amino acid sequence set forth in SEQ ID NO: 72 or a conservative modification thereof, a V_{L} CDR2 comprising the amino acid sequence set forth in SEQ ID NO: 73 or a conservative modification thereof, and a V_{L} CDR3 comprising the amino acid sequence set forth in SEQ ID NO: 74 or a conservative modification thereof. SEQ ID NOs: 72-74 are provided in Table 8.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 69 or a conservative modification thereof, a V_{H} CDR2 comprising the amino acid sequence set forth in SEQ ID NO: 70 or a conservative modification thereof, a V_{H} CDR3 comprising the amino acid sequence set forth in SEQ ID NO: 71 or a conservative modification thereof, a V_{L} CDR1 comprising the amino acid sequence set forth in SEQ ID NO: 72 or a conservative modification thereof, a V_{L} CDR2 comprising the amino acid sequence set forth in SEQ ID NO: 73 or a conservative modification, and a V_{L} CDR3 comprising the amino acid sequence set forth in SEQ ID NO: 74 or a conservative modification thereof.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 69, a V_{H} CDR2 comprising the amino acid sequence set forth in SEQ ID NO: 70, a V_{H} CDR3 comprising the amino acid sequence set forth in SEQ ID NO: 71, a V_{L} CDR1 comprising the amino acid sequence set forth in SEQ ID NO: 72, a V_{L} CDR2 comprising the amino acid sequence set forth in SEQ ID NO: 73, and a V_{L} CDR3 comprising the amino acid sequence set forth in SEQ ID NO: 74.

In certain embodiments, the extracellular antigen-binding domain of the CLEC12A-targeted chimeric receptor (e.g., a CLEC12A-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 75. For example, the extracellular antigen-binding domain of the CLEC12A-targeted chimeric receptor (e.g., a CLEC12A-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 108. In certain embodiments, the extracellular antigen-binding domain of the CLEC12A-targeted chimeric receptor comprises a V_{H} comprising the amino acid sequence set forth in SEQ ID NO: 75. SEQ ID NO: 75 is provided in Table 8 below.

In certain embodiments, the extracellular antigen-binding domain of the CLEC12A-targeted chimeric receptor (e.g., a CLEC12A-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 76. For example, the extracellular antigen-binding domain of the CLEC12A-targeted chimeric receptor (e.g., a CLEC12A-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 109. In certain embodiments, the extracellular antigen-binding domain of the CLEC12A-targeted chimeric receptor comprises a V_{L} comprising the amino acid sequence set forth in SEQ ID NO: 76. SEQ ID NO: 76 is provided in Table 8 below.

In certain embodiments, the extracellular antigen-binding domain of the CLEC12A-targeted chimeric receptor (e.g., a CLEC12A-targeted scFv) comprises a V_{H} comprising the amino acid sequence set forth in SEQ ID NO: 75, and a V_{L} comprising the amino acid sequence set forth in SEQ ID NO: 76. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}. In certain embodiments, the CLEC12A-targeted scFv comprises or consists of the amino acid sequence set forth in SEQ ID NO: 79. In certain embodiments, the CLEC12A-targeted scFv is designated as “CLEC12A-A”. An exemplary nucleotide sequence encoding the amino acid sequence of SEQ ID NO: 79 is set forth in SEQ ID NO: 80. SEQ ID NOs: 79 and 80 are provided in Table 8. The CDRs provided in Table 8 are identified according to the IM GT numbering system.

TABLE 8
(CLEC12A-A)

| CDRs | 1 | 2 | 3 |
|---|---|---|---|
| V_{H} | GGSISSSTYY [SEQ ID | THYRGST [SEQ ID | ARELTGEVFDY [SEQ |
| | NO: 69] | NO: 70] | ID NO: 71] |
| V_{L} | QSISSY [SEQ ID NO: | AAS [SEQ ID NO: | QQSYSTPFT [SEQ ID |
| | 72] | 73] | NO: 74] |

| | |
|---|---|
| Full V_{H} | QLQLQESGPGLVKPSETLSLTCTVSGGSISSSTYYWGWIRQPPRKGLEWIGSTHYRGST |
| | YYNPSLKSRVTISVDTSKNQFSLKVSSVTAADTAVYYCARELTGEVFDYWGQGTLVTVS |
| | S [SEQ ID NO: 75] |
| Full V_{L} | DIQMTQSPSSLSASVGDRVTITCRASQSISSYLNWYQQKPGKAPKLLIYAASSLQSGVP |
| | SRFSGSGSGTDFTLTISSLQPEDFATYYCQQSYSTPFTFGPGTKVDIK [SEQ ID |
| | NO: 76] |
| DNA for | Cagctccagctccaagagtcagggccaggtctcgtgaaaccgagtgagaccctgtccct |
| Full V_{H} | gacctgcacagtgagtggtggatcaatctcaagctctacctactattgggggtggattc |
| | ggcagccccctagaaaggggcttgagtggattggcagcactcattatcgaggatctacc |
| | tattataatccttctctgaaaagcagagttaccatctctgtggatacgtccaaaaatca |
| | gttcagtctgaaggtatcatccgtgactgctgccgacacggccgtgtactattgcgcga |
| | gggagctgacaggtgaggtctttgactactggggccagggcacactcgtgaccgtgtct |
| | tct [SEQ ID NO: 77] |
| DNA for | Gacatccagatgacgcagtccccttccagcttgtccgcatctgtgggtgatagggtcac |
| Full V_{L} | gattacatgtagggctagtcagagtatttctagttacctgaattggtaccagcagaaac |
| | caggcaaggcaccaaagttgctcatctatgcggcctcctctctgcaatctggcgtgccg |
| | tccagatttagtggatcaggctccggaaccgatttcacccttacgatctcctcacttca |
| | acccgaggatttcgccacatattactgtcaacaaagctattctacaccgttcaccttcg |
| | gaccggggacaaaagtggatattaaa [SEQ ID NO: 78] |
| V_{H}-V_{L} | QLQLQESGPGLVKPSETLSLTCTVSGGSISSSTYYWGWIRQPPRKGLEWIGSTHYRGST |
| scFv | YYNPSLKSRVTISVDTSKNQFSLKVSSVTAADTAVYYCARELTGEVFDYWGQGTLVTVS |
| | SASTGGGGSGGGGSGGGGSDIQMTQSPSSLSASVGDRVTITCRASQSISSYLNWYQQKP |
| | GKAPKLLIYAASSLQSGVPSRFSGSGSGTDFTLTISSLQPEDFATYYCQQSYSTPFTFG |
| | PGTKVDIK [SEQ ID NO: 79] |
| DNA for | Cagctccagctccaagagtcagggccaggtctcgtgaaaccgagtgagaccctgtccct |
| V_{H}-V_{L} | gacctgcacagtgagtggtggatcaatctcaagctctacctactattgggggtggattc |
| scFv | ggcagccccctagaaaggggcttgagtggattggcagcactcattatcgaggatctacc |
| | tattataatccttctctgaaaagcagagttaccatctctgtggatacgtccaaaaatca |
| | gttcagtctgaaggtatcatccgtgactgctgccgacacggccgtgtactattgcgcga |
| | gggagctgacaggtgaggtctttgactactggggccagggcacactcgtgaccgtgtct |
| | tctgcctcaacaggagggggtgggagtggaggcggtggatcagggggaggagggagtga |
| | catccagatgacgcagtccccttccagcttgtccgcatctgtgggtgatagggtcacga |
| | ttacatgtagggctagtcagagtatttctagttacctgaattggtaccagcagaaacca |
| | ggcaaggcaccaaagttgctcatctatgcggcctcctctctgcaatctggcgtgccgtc |
| | cagatttagtggatcaggctccggaaccgatttcacccttacgatctcctcacttcaac |
| | ccgaggatttcgccacatattactgtcaacaaagctattctacaccgttcaccttcgga |
| | ccggggacaaaagtggatattaaa [SEQ ID NO: 80] |

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 81 or a conservative modification thereof, a V_{H} CDR2 comprising the amino acid sequence set forth in SEQ ID NO: 82 or a conservative modification thereof, and a V_{H} CDR3 comprising the amino acid sequence set forth in SEQ ID NO: 83 or a conservative modification thereof. SEQ ID NOs: 81-83 are provided in Table 9.

In certain embodiments, the extracellular antigen-binding domain of the CLEC12A-targeted chimeric receptor (e.g., a CLEC12A-targeted scFv) comprises a V_{L} CDR1 comprising the amino acid sequence set forth in SEQ ID NO: 84 or a conservative modification thereof, a V_{L} CDR2 comprising the amino acid sequence set forth in SEQ ID NO: 73 or a conservative modification thereof, and a V_{L} CDR3 comprising the amino acid sequence set forth in SEQ ID NO: 85 or a conservative modification thereof. SEQ ID NOs: 73, 84 and 85 are provided in Table 9.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 81 or a conservative modification thereof, a V_{H} CDR2 comprising the amino acid sequence set forth in SEQ ID NO: 82 or a conservative modification thereof, a V_{H} CDR3 comprising the amino acid sequence set forth in SEQ ID NO: 83 or a conservative modification thereof, a V_{L} CDR1 comprising the amino acid sequence set forth in SEQ ID NO: 84 or a conservative modification thereof, a V_{L} CDR2 comprising the amino acid sequence set forth in SEQ ID NO: 73 or a conservative modification, and a V_{L} CDR3 comprising the amino acid sequence set forth in SEQ ID NO: 85 or a conservative modification thereof.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 81, a V_{H} CDR2 comprising the amino acid sequence set forth in SEQ ID NO: 82, a V_{H} CDR3 comprising the amino acid sequence set forth in SEQ ID NO: 83, a V_{L} CDR1 comprising the amino acid sequence set forth in SEQ ID NO: 84, a V_{L} CDR2 comprising the amino acid sequence set forth in SEQ ID NO: 73, and a V_{L} CDR3 comprising the amino acid sequence set forth in SEQ ID NO: 85.

In certain embodiments, the extracellular antigen-binding domain of the CLEC12A-targeted chimeric receptor (e.g., a CLEC12A-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 86. For example, the extracellular antigen-binding domain of the CLEC12A-targeted chimeric receptor (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 86. In certain embodiments, the extracellular antigen-binding domain of the CLEC12A-targeted chimeric receptor comprises a V_{H} comprising the amino acid sequence set forth in SEQ ID NO: 86. SEQ ID NO: 86 is provided in Table 9 below.

In certain embodiments, the extracellular antigen-binding domain of the CLEC12A-targeted chimeric receptor (e.g., a CLEC12A-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 87. For example, the extracellular antigen-binding domain of the CLEC12A-targeted chimeric receptor (e.g., a CLEC12A-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 87. In certain embodiments, the extracellular antigen-binding domain of the CLEC12A-targeted chimeric receptor comprises a V_{L} comprising the amino acid sequence set forth in SEQ ID NO: 87. SEQ ID NO: 87 is provided in Table 9 below.

In certain embodiments, the extracellular antigen-binding domain of the CLEC12A-targeted chimeric receptor (e.g., a CLEC12A-targeted scFv) comprises a V_{H} comprising the amino acid sequence set forth in SEQ ID NO: 86, and a V_{L} comprising the amino acid sequence set forth in SEQ ID NO: 87. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}. In certain embodiments, the CLEC12A-targeted scFv comprises or consists of the amino acid sequence set forth in SEQ ID NO: 88, which is provided in Table 9. In certain embodiments, the CLEC12A-targeted scFv is designated as “CLEC12A-B”. The CDRs provided in Table 9 are identified according to the IM GT numbering system.

TABLE 9
(CLEC12A-B)

| CDRs | 1 | 2 | 3 |
|---|---|---|---|
| V_{H} | GGSISTYY [SEQ ID | IYYSGST [SEQ ID | AREDYYGSGSPFDY |
| | NO: 81] | NO: 82] | [SEQ ID NO: 83] |
| V_{L} | QGIRYD [SEQ ID NO: | AAS [SEQ ID NO: | LQDYNFPRT [SEQ ID |
| | 84] | 73] | NO: 85] |

| | |
|---|---|
| Full V_{H} | QVQLQESGPGLVKPSETLSLTCTVSGGSISTYYWSWIRQPPGKGLEWIGYIYYSGSTKY |
| | NPSLKSRVTISVDTSKNLFSLKLSSVTAADTAVYYCAREDYYGSGSPFDYWGQGTLVTV |
| | SS [SEQ ID NO: 86] |
| Full V_{L} | AIQMTQSPSSLSASVGDRVTITCRASQGIRYDLGWYQQKPGKAPKLLIYAASSLQSGVP |
| | SRFSGSGSGTDFTLTISSLQPEDFATYYCLQDYNFPRTFGQGTKVEIK [SEQ ID |
| | NO: 87] |
| V_{H}-V_{L} | QVQLQESGPGLVKPSETLSLTCTVSGGSISTYYWSWIRQPPGKGLEWIGYIYYSGSTKY |
| scFv | NPSLKSRVTISVDTSKNLFSLKLSSVTAADTAVYYCAREDYYGSGSPFDYWGQGTLVTV |
| | SSASTGGGGSGGGGSGGGGSAIQMTQSPSSLSASVGDRVTITCRASQGIRYDLGWYQQK |
| | PGKAPKLLIYAASSLQSGVPSRFSGSGSGTDFTLTISSLQPEDFATYYCLQDYNFPRTF |
| | GQGTKVEIK [SEQ ID NO: 88] |

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 89 or a conservative modification thereof, a V_{H} CDR2 comprising the amino acid sequence set forth in SEQ ID NO: 90 or a conservative modification thereof, and a V_{H} CDR3 comprising the amino acid sequence set forth in SEQ ID NO: 91 or a conservative modification thereof. SEQ ID NOs: 89-91 are provided in Table 10.

In certain embodiments, the extracellular antigen-binding domain of the CLEC12A-targeted chimeric receptor (e.g., a CLEC12A-targeted scFv) comprises a V_{L} CDR1 comprising the amino acid sequence set forth in SEQ ID NO: 92 or a conservative modification thereof, a V_{L} CDR2 comprising the amino acid sequence set forth in SEQ ID NO: 93 or a conservative modification thereof, and a V_{L} CDR3 comprising the amino acid sequence set forth in SEQ ID NO: 94 or a conservative modification thereof. SEQ ID NOs: 92-94 are provided in Table 10.

In certain embodiments, the extracellular antigen-binding domain of the CLEC12A-targeted chimeric receptor (e.g., an scFv) comprises a V_{H} CDR1 comprising the amino acid sequence set forth in SEQ ID NO: 89 or a conservative modification thereof, a V_{H} CDR2 comprising the amino acid sequence set forth in SEQ ID NO: 90 or a conservative modification thereof, a V_{H} CDR3 comprising the amino acid sequence set forth in SEQ ID NO: 91 or a conservative modification thereof, a V_{L} CDR1 comprising the amino acid sequence set forth in SEQ ID NO: 92 or a conservative modification thereof, a V_{L} CDR2 comprising the amino acid sequence set forth in SEQ ID NO: 93 or a conservative modification, and a V_{L} CDR3 comprising the amino acid sequence set forth in SEQ ID NO: 94 or a conservative modification thereof.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 89, a V_{H} CDR2 comprising the amino acid sequence set forth in SEQ ID NO: 90, a V_{H} CDR3 comprising the amino acid sequence set forth in SEQ ID NO: 91, a V_{L} CDR1 comprising the amino acid sequence set forth in SEQ ID NO: 92, a V_{L} CDR2 comprising the amino acid sequence set forth in SEQ ID NO: 93, and a V_{L} CDR3 comprising the amino acid sequence set forth in SEQ ID NO: 94.

In certain embodiments, the extracellular antigen-binding domain of the CLEC12A-targeted chimeric receptor (e.g., a CLEC12A-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 95. For example, the extracellular antigen-binding domain of the CLEC12A-targeted chimeric receptor (e.g., a CLEC12A-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 95. In certain embodiments, the extracellular antigen-binding domain of the CLEC12A-targeted chimeric receptor comprises a V_{H} comprising the amino acid sequence set forth in SEQ ID NO: 95. SEQ ID NO: 95 is provided in Table 10 below.

In certain embodiments, the extracellular antigen-binding domain of the CLEC12A-targeted chimeric receptor (e.g., a CLEC12A-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 96. For example, the extracellular antigen-binding domain of the CLEC12A-targeted chimeric receptor (e.g., a CLEC12A-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 96. In certain embodiments, the extracellular antigen-binding domain of the CLEC12A-targeted chimeric receptor comprises a V_{L} comprising the amino acid sequence set forth in SEQ ID NO: 96. SEQ ID NO: 96 is provided in Table 10 below.

In certain embodiments, the extracellular antigen-binding domain of the CLEC12A-targeted chimeric receptor (e.g., a CLEC12A-targeted scFv) comprises a V_{H} comprising the amino acid sequence set forth in SEQ ID NO: 96, and a V_{L} comprising the amino acid sequence set forth in SEQ ID NO: 96. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}. In certain embodiments, the CLEC12A-targeted scFv comprises the amino acid sequence set forth in SEQ ID NO: 97, which is provided in Table 10. In certain embodiments, the CLEC12A-targeted scFv is designated as “CLEC12A-C”. The CDRs provided in Table 10 are identified according to the IM GT numbering system.

TABLE 10
(CLEC12A-C)

| CDRs | 1 | 2 | 3 |
|---|---|---|---|
| V_{H} | GFTFSSYG [SEQ ID | ISYDGSDK [SEQ ID | ARDKGYYFDY [SEQ ID |
| | NO: 89] | NO: 90] | NO: 91] |
| V_{L} | QSVGNRY [SEQ ID NO: | GAS [SEQ ID NO: | QQDYNLPLT [SEQ ID |
| | 92] | 93] | NO: 94] |

| | |
|---|---|
| Full V_{H} | QVQLVESGGGWQPGRSLRLSCAASGFTFSSYGMHWVRQAPGKGLEWVAVISYDGSDKY |
| | YVDSVKGRFTISRDNSKNTLYLHMNSLRAEDTAVYYCARDKGYYFDYWGQGTLVTVSS |
| | [SEQ ID NO: 95] |
| Full V_{L} | EIVMTQSPATLSLSPGERATLSCRASQSVGNRYLSWYQQKPGQAPRLLIYGASTRATGI |
| | PARFSGSGSGTDFTLTISSLQPEDFAVYYCQQDYNLPLTFGGGTKVEIK [SEQ ID |
| | NO: 96] |
| V_{H}-V_{L} | QVQLVESGGGVVQPGRSLRLSCAASGFTFSSYGMHWVRQAPGKGLEWVAVISYDGSDKY |
| scFv | YVDSVKGRFTISRDNSKNTLYLHMNSLRAEDTAVYYCARDKGYYFDYWGQGTLVTVSSA |
| | STGGGGSGGGGSGGGGSEIVMTQSPATLSLSPGERATLSCRASQSVGNRYLSWYQQKPG |
| | QAPRLLIYGASTRATGIPARFSGSGSGTDFTLTISSLQPEDFAVYYCQQDYNLPLTFGG |
| | GTKVEIK [SEQ ID NO: 97] |

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 89 or a conservative modification thereof, a V_{H} CDR2 comprising the amino acid sequence set forth in SEQ ID NO: 90 or a conservative modification thereof, and a V_{H} CDR3 comprising the amino acid sequence set forth in SEQ ID NO: 98 or a conservative modification thereof. SEQ ID NOs: 89, 90, and 98 are provided in Table 11.

In certain embodiments, the extracellular antigen-binding domain of the CLEC12A-targeted chimeric receptor (e.g., a CLEC12A-targeted scFv) comprises a V_{L} CDR1 comprising the amino acid sequence set forth in SEQ ID NO: 99 or a conservative modification thereof, a V_{L} CDR2 comprising the amino acid sequence set forth in SEQ ID NO: 93 or a conservative modification thereof, and a V_{L} CDR3 comprising the amino acid sequence set forth in SEQ ID NO: 151 or a conservative modification thereof. SEQ ID NOs: 93, 99, and 151 are provided in Table 11.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 89 or a conservative modification thereof, a V_{H} CDR2 comprising the amino acid sequence set forth in SEQ ID NO: 90 or a conservative modification thereof, a V_{H} CDR3 comprising the amino acid sequence set forth in SEQ ID NO: 98 or a conservative modification thereof, a V_{L} CDR1 comprising the amino acid sequence set forth in SEQ ID NO: 99 or a conservative modification thereof, a V_{L} CDR2 comprising the amino acid sequence set forth in SEQ ID NO: 93 or a conservative modification, and a V_{L} CDR3 comprising the amino acid sequence set forth in SEQ ID NO: 151 or a conservative modification thereof.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 89, a V_{H} CDR2 comprising the amino acid sequence set forth in SEQ ID NO: 90, a V_{H} CDR3 comprising the amino acid sequence set forth in SEQ ID NO: 98, a V_{L} CDR1 comprising the amino acid sequence set forth in SEQ ID NO: 99, a V_{L} CDR2 comprising the amino acid sequence set forth in SEQ ID NO: 93, and a V_{L} CDR3 comprising the amino acid sequence set forth in SEQ ID NO: 151.

In certain embodiments, the extracellular antigen-binding domain of the CLEC12A-targeted chimeric receptor (e.g., a CLEC12A-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 100. For example, the extracellular antigen-binding domain of the CLEC12A-targeted chimeric receptor (e.g., a CLEC12A-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 100. In certain embodiments, the extracellular antigen-binding domain of the CLEC12A-targeted chimeric receptor comprises a V_{H} comprising the amino acid sequence set forth in SEQ ID NO: 100. SEQ ID NO: 100 is provided in Table 11 below.

In certain embodiments, the extracellular antigen-binding domain of the CLEC12A-targeted chimeric receptor (e.g., a CLEC12A-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 101. For example, the extracellular antigen-binding domain of the CLEC12A-targeted chimeric receptor (e.g., a CLEC12A-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 101. In certain embodiments, the extracellular antigen-binding domain of the CLEC12A-targeted chimeric receptor comprises a V_{L} comprising the amino acid sequence set forth in SEQ ID NO: 101. SEQ ID NO: 101 is provided in Table 11 below.

In certain embodiments, the extracellular antigen-binding domain of the CLEC12A-targeted chimeric receptor (e.g., a CLEC12A-targeted scFv) comprises a V_{H} comprising the amino acid sequence set forth in SEQ ID NO: 100, and a V_{L} comprising the amino acid sequence set forth in SEQ ID NO: 101. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}. In certain embodiments, the CLEC12A-targeted scFv comprises the amino acid sequence set forth in SEQ ID NO: 102, which is provided in Table 11. In certain embodiments, the CLEC12A-targeted scFv is designated as “CLEC12A-D”. The CDRs provided in Table 11 are identified according to the IM GT numbering system.

TABLE 11
(CLEC12A-D)

| CDRs | 1 | 2 | 3 |
|---|---|---|---|
| V_{H} | GFTFSSYG [SEQ ID | ISYDGSDK [SEQ ID | ARDGSRYFDY [SEQ ID |
| | NO: 89] | NO: 90] | NO: 98] |
| V_{L} | QSVHSKY [SEQ ID NO: | GAS [SEQ ID NO: | QQDYNLPIT [SEQ ID |
| | 99] | 93] | NO: 151] |

| | |
|---|---|
| Full V_{H} | QVQLVESGGGWQPGRSLRLSCAASGFTFSSYGMHWVRQAPGKGLEWVAVISYDGSDKY |
| | SADSVKGRFNISRDNSKNTLYLQMNSLRAEDTAVYYCARDGSRYFDYWGQGTLVTVSS |
| | [SEQ ID NO: 100] |
| Full V_{L} | EIFMTQSPATLSLSPGERATLSCRASQSVHSKYLSWYQQKPGQAPSLLIYGASTRATGI |
| | PARFSGSGSGTDFTLTISSLQPEDFAVYYCQQDYNLPITFGQGTRLEIK [SEQ ID |
| | NO: 101] |
| V_{H}-V_{L} | QVQLVESGGGVVQPGRSLRLSCAASGFTFSSYGMHWVRQAPGKGLEWVAVISYDGSDKY |
| scFv | SADSVKGRFNISRDNSKNTLYLQMNSLRAEDTAVYYCARDGSRYFDYWGQGTLVTVSSA |
| | STGGGGSGGGGSGGGGSEIFMTQSPATLSLSPGERATLSCRASQSVHSKYLSWYQQKPG |
| | QAPSLLIYGASTRATGIPARFSGSGSGTDFTLTISSLQPEDFAVYYCQQDYNLPITFGQ |
| | GTRLEIK [SEQ ID NO: 102] |

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 81 or a conservative modification thereof, a V_{H} CDR2 comprising the amino acid sequence set forth in SEQ ID NO: 103 or a conservative modification thereof, and a V_{H} CDR3 comprising the amino acid sequence set forth in SEQ ID NO: 83 or a conservative modification thereof. SEQ ID NOs: 81, 83, and 103 are provided in Table 12.

In certain embodiments, the extracellular antigen-binding domain of the CLEC12A-targeted chimeric receptor (e.g., a CLEC12A-targeted scFv) comprises a V_{L} CDR1 comprising the amino acid sequence set forth in SEQ ID NO: 104 or a conservative modification thereof, a V_{L} CDR2 comprising the amino acid sequence set forth in SEQ ID NO: 73 or a conservative modification thereof, and a V_{L} CDR3 comprising the amino acid sequence set forth in SEQ ID NO: 105 or a conservative modification thereof. SEQ ID NOs: 73, 104, and 105 are provided in Table 12.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 81 or a conservative modification thereof, a V_{H} CDR2 comprising the amino acid sequence set forth in SEQ ID NO: 103 or a conservative modification thereof, a V_{H} CDR3 comprising the amino acid sequence set forth in SEQ ID NO: 83 or a conservative modification thereof, a V_{L} CDR1 comprising the amino acid sequence set forth in SEQ ID NO: 104 or a conservative modification thereof, a V_{L} CDR2 comprising the amino acid sequence set forth in SEQ ID NO: 73 or a conservative modification, and a V_{L} CDR3 comprising the amino acid sequence set forth in SEQ ID NO: 105 or a conservative modification thereof.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 81, a V_{H} CDR2 comprising the amino acid sequence set forth in SEQ ID NO: 103, a V_{H} CDR3 comprising the amino acid sequence set forth in SEQ ID NO: 83, a V_{L} CDR1 comprising the amino acid sequence set forth in SEQ ID NO: 104, a V_{L} CDR2 comprising the amino acid sequence set forth in SEQ ID NO: 73, and a V_{L} CDR3 comprising the amino acid sequence set forth in SEQ ID NO: 105.

In certain embodiments, the extracellular antigen-binding domain of the CLEC12A-targeted chimeric receptor (e.g., a CLEC12A-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 106. For example, the extracellular antigen-binding domain of the CLEC12A-targeted chimeric receptor (e.g., a CLEC12A-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 106. In certain embodiments, the extracellular antigen-binding domain of the CLEC12A-targeted chimeric receptor comprises a V_{H} comprising the amino acid sequence set forth in SEQ ID NO: 106. SEQ ID NO: 106 is provided in Table 12 below.

In certain embodiments, the extracellular antigen-binding domain of the CLEC12A-targeted chimeric receptor (e.g., a CLEC12A-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 107. For example, the extracellular antigen-binding domain of the CLEC12A-targeted chimeric receptor (e.g., a CLEC12A-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 107. In certain embodiments, the extracellular antigen-binding domain of the CLEC12A-targeted chimeric receptor comprises a V_{L} comprising the amino acid sequence set forth in SEQ ID NO: 107. SEQ ID NO: 107 is provided in Table 12 below.

In certain embodiments, the extracellular antigen-binding domain of the CLEC12A-targeted chimeric receptor (e.g., a CLEC12A-targeted scFv) comprises a V_{H} comprising the amino acid sequence set forth in SEQ ID NO: 106, and a V_{L} comprising the amino acid sequence set forth in SEQ ID NO: 107. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}. In certain embodiments, the CLEC12A-targeted scFv comprises the amino acid sequence set forth in SEQ ID NO: 108, which is provided in Table 12. In certain embodiments, the CLEC12A-targeted scFv is designated as “CLEC12A-E”. The CDRs provided in Table 12 are identified according to the IM GT numbering system.

TABLE 12
(CLEC12A-E)

| CDRs | 1 | 2 | 3 |
|---|---|---|---|
| V_{H} | GGSISTYY [SEQ ID | IYFSGST [SEQ ID | AREDYYGSGSPFDY |
| | NO: 81] | NO: 103] | [SEQ ID NO: 83] |
| V_{L} | QGIRND [SEQ ID NO: | AAS [SEQ ID NO: | LQDYNYPRT [SEQ ID |
| | 104] | 73] | NO: 105] |

| | |
|---|---|
| Full V_{H} | QVQLQESGPGLVKPSETLSLTCTVSGGSISTYYWSWIRQPPGKGLEWLGYIYFSGSTNY |
| | NPSLKSRLTISVAASKSQFSLKLSSVTAADTAVYYCAREDYYGSGSPFDYWGQGTLVTV |
| | SS [SEQ ID NO: 106] |
| Full V_{L} | AIQMTQSPSSLSASVGDRVTITCRASQGIRNDLGWFQQKPGKAPKLLIYAASSLQSGVP |
| | SRFSGSGSGTYFTLTISSLQPEDSATYYCLQDYNYPRTFGQGTKVEIK [SEQ ID |
| | NO: 107] |
| V_{H}-V_{L} | QVQLQESGPGLVKPSETLSLTCTVSGGSISTYYWSWIRQPPGKGLEWLGYIYFSGSTNY |
| scFv | NPSLKSRLTISVAASKSQFSLKLSSVTAADTAVYYCAREDYYGSGSPFDYWGQGTLVTV |
| | SSASTGGGGSGGGGSGGGGSAIQMTQSPSSLSASVGDRVTITCRASQGIRNDLGWFQQK |
| | PGKAPKLLIYAASSLQSGVPSRFSGSGSGTYFTLTISSLQPEDSATYYCLQDYNYPRTF |
| | GQGTKVEIK [SEQ ID NO: 108] |

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 109 or a conservative modification thereof, a V_{H} CDR2 comprising the amino acid sequence set forth in SEQ ID NO: 103 or a conservative modification thereof, and a V_{H} CDR3 comprising the amino acid sequence set forth in SEQ ID NO: 83 or a conservative modification thereof. SEQ ID NOs: 83, 103, and 109 are provided in Table 13.

In certain embodiments, the extracellular antigen-binding domain of the CLEC12A-targeted chimeric receptor (e.g., a CLEC12A-targeted scFv) comprises a V_{L} CDR1 comprising the amino acid sequence set forth in SEQ ID NO: 110 or a conservative modification thereof, a V_{L} CDR2 comprising the amino acid sequence set forth in SEQ ID NO: 73 or a conservative modification thereof, and a V_{L} CDR3 comprising the amino acid sequence set forth in SEQ ID NO: 85 or a conservative modification thereof. SEQ ID NOs: 73, 85, and 110 are provided in Table 13.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 109 or a conservative modification thereof, a V_{H} CDR2 comprising the amino acid sequence set forth in SEQ ID NO: 103 or a conservative modification thereof, a V_{H} CDR3 comprising the amino acid sequence set forth in SEQ ID NO: 83 or a conservative modification thereof, a V_{L} CDR1 comprising the amino acid sequence set forth in SEQ ID NO: 110 or a conservative modification thereof, a V_{L} CDR2 comprising the amino acid sequence set forth in SEQ ID NO: 73 or a conservative modification, and a V_{L} CDR3 comprising the amino acid sequence set forth in SEQ ID NO: 85 or a conservative modification thereof.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 109, a V_{H} CDR2 comprising the amino acid sequence set forth in SEQ ID NO: 103, a V_{H} CDR3 comprising the amino acid sequence set forth in SEQ ID NO: 83, a V_{L} CDR1 comprising the amino acid sequence set forth in SEQ ID NO: 110, a V_{L} CDR2 comprising the amino acid sequence set forth in SEQ ID NO: 73, and a V_{L} CDR3 comprising the amino acid sequence set forth in SEQ ID NO: 85.

In certain embodiments, the extracellular antigen-binding domain of the CLEC12A-targeted chimeric receptor (e.g., a CLEC12A-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 111. For example, the extracellular antigen-binding domain of the CLEC12A-targeted chimeric receptor (e.g., a CLEC12A-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 111. In certain embodiments, the extracellular antigen-binding domain of the CLEC12A-targeted chimeric receptor comprises a V_{H} comprising the amino acid sequence set forth in SEQ ID NO: 111. SEQ ID NO: 111 is provided in Table 13 below.

In certain embodiments, the extracellular antigen-binding domain of the CLEC12A-targeted chimeric receptor (e.g., a CLEC12A-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 112. For example, the extracellular antigen-binding domain of the CLEC12A-targeted chimeric receptor (e.g., a CLEC12A-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 112. In certain embodiments, the extracellular antigen-binding domain of the CLEC12A-targeted chimeric receptor comprises a V_{L} comprising the amino acid sequence set forth in SEQ ID NO: 112. SEQ ID NO: 112 is provided in Table 13 below.

In certain embodiments, the extracellular antigen-binding domain of the CLEC12A-targeted chimeric receptor (e.g., a CLEC12A-targeted scFv) comprises a V_{H} comprising the amino acid sequence set forth in SEQ ID NO: 111, and a V_{L} comprising the amino acid sequence set forth in SEQ ID NO: 112. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}. In certain embodiments, the CLEC12A-targeted scFv comprises the amino acid sequence set forth in SEQ ID NO: 113, which is provided in Table 13. In certain embodiments, the CLEC12A-targeted scFv is designated as “CLEC12A-F”. The CDRs provided in Table 13 are identified according to the IM GT numbering system.

TABLE 13
(CLEC12A-F)

| CDRs | 1 | 2 | 3 |
|---|---|---|---|
| V_{H} | GGSISTDY [SEQ ID | IYFSGST [SEQ ID | AREDYYGSGSPFDY |
| | NO: 109] | NO: 103] | [SEQ ID NO: 83] |
| V_{L} | QDIRND [SEQ ID NO: | AAS [SEQ ID NO: | LQDYNFPRT [SEQ ID |
| | 110] | 73] | NO: 85] |

| | |
|---|---|
| Full V_{H} | QVQLQESGPGLVKPSETLSLTCTVSGGSISTDYWSWIRQPPGKGLEWIGYIYFSGSTKY |
| | NPSLKSRVTISVDTSKNQFSLKLSSVTAADTAVYYCAREDYYGSGSPFDYWGQGTLVTV |
| | SS [SEQ ID NO: 111] |
| Full V_{L} | AIQMTQSPSSLSASVGDRVTITCRASQDIRNDLGWFQQKPGKAPKLLIYAASSLQSGVP |
| | SRFSGSGSGTDFTLTISSLQPEDFATYYCLQDYNFPRTFGQGTKVEIK [SEQ ID |
| | NO: 112] |
| V_{H}-V_{L} | QVQLQESGPGLVKPSETLSLTCTVSGGSISTDYWSWIRQPPGKGLEWIGYIYFSGSTKY |
| scFv | NPSLKSRVTISVDTSKNQFSLKLSSVTAADTAVYYCAREDYYGSGSPFDYWGQGTLVTV |
| | SSASTGGGGSGGGGSGGGGSAIQMTQSPSSLSASVGDRVTITCRASQDIRNDLGWFQQK |
| | PGKAPKLLIYAASSLQSGVPSRFSGSGSGTDFTLTISSLQPEDFATYYCLQDYNFPRTF |
| | GQGTKVEIK [SEQ ID NO: 113] |

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 89 or a conservative modification thereof, a V_{H} CDR2 comprising the amino acid sequence set forth in SEQ ID NO: 90 or a conservative modification thereof, and a V_{H} CDR3 comprising the amino acid sequence set forth in SEQ ID NO: 114 or a conservative modification thereof. SEQ ID NOs: 89, 90, and 114 are provided in Table 14.

In certain embodiments, the extracellular antigen-binding domain of the CLEC12A-targeted chimeric receptor (e.g., a CLEC12A-targeted scFv) comprises a V_{L} CDR1 comprising the amino acid sequence set forth in SEQ ID NO: 115 or a conservative modification thereof, a V_{L} CDR2 comprising the amino acid sequence set forth in SEQ ID NO: 93 or a conservative modification thereof, and a V_{L} CDR3 comprising the amino acid sequence set forth in SEQ ID NO: 116 or a conservative modification thereof. SEQ ID NOs: 93, 115, and 116 are provided in Table 14.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 89 or a conservative modification thereof, a V_{H} CDR2 comprising the amino acid sequence set forth in SEQ ID NO: 90 or a conservative modification thereof, a V_{H} CDR3 comprising the amino acid sequence set forth in SEQ ID NO: 114 or a conservative modification thereof, a V_{L} CDR1 comprising the amino acid sequence set forth in SEQ ID NO: 115 or a conservative modification thereof, a V_{L} CDR2 comprising the amino acid sequence set forth in SEQ ID NO: 93 or a conservative modification, and a V_{L} CDR3 comprising the amino acid sequence set forth in SEQ ID NO: 116 or a conservative modification thereof.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 89, a V_{H} CDR2 comprising the amino acid sequence set forth in SEQ ID NO: 90, a V_{H} CDR3 comprising the amino acid sequence set forth in SEQ ID NO: 114, a V_{L} CDR1 comprising the amino acid sequence set forth in SEQ ID NO: 115, a V_{L} CDR2 comprising the amino acid sequence set forth in SEQ ID NO: 93, and a V_{L} CDR3 comprising the amino acid sequence set forth in SEQ ID NO: 116.

In certain embodiments, the extracellular antigen-binding domain of the CLEC12A-targeted chimeric receptor (e.g., a CLEC12A-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 117. For example, the extracellular antigen-binding domain of the CLEC12A-targeted chimeric receptor (e.g., a CLEC12A-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 117. In certain embodiments, the extracellular antigen-binding domain of the CLEC12A-targeted chimeric receptor comprises a V_{H} comprising the amino acid sequence set forth in SEQ ID NO: 117. SEQ ID NO: 117 is provided in Table 14 below.

In certain embodiments, the extracellular antigen-binding domain of the CLEC12A-targeted chimeric receptor (e.g., a CLEC12A-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 118. For example, the extracellular antigen-binding domain of the CLEC12A-targeted chimeric receptor (e.g., a CLEC12A-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 118. In certain embodiments, the extracellular antigen-binding domain of the CLEC12A-targeted chimeric receptor comprises a V_{L} comprising the amino acid sequence set forth in SEQ ID NO: 118. SEQ ID NO: 118 is provided in Table 14 below.

In certain embodiments, the extracellular antigen-binding domain of the CLEC12A-targeted chimeric receptor (e.g., a CLEC12A-targeted scFv) comprises a V_{H} comprising the amino acid sequence set forth in SEQ ID NO: 117, and a V_{L} comprising the amino acid sequence set forth in SEQ ID NO: 118. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}. In certain embodiments, the CLEC12A-targeted scFv comprises the amino acid sequence set forth in SEQ ID NO: 119, which is provided in Table 14. In certain embodiments, the CLEC12A-targeted scFv is designated as “CLEC12A-G”. The CDRs provided in Table 14 are identified according to the IM GT numbering system.

TABLE 14
(CLEC12A-G)

| CDRs | 1 | 2 | 3 |
|---|---|---|---|
| V_{H} | GFTFSSYG [SEQ ID | ISYDGSDK [SEQ ID | ARDGQFYFDY [SEQ ID |
| | NO: 89] | NO: 90] | NO: 114] |
| V_{L} | QSVTSRY [SEQ ID NO: | GAS [SEQ ID NO: | QQDYNLPLT [SEQ ID |
| | 115] | 93] | NO: 116] |

| | |
|---|---|
| Full V_{H} | QVQLVESGGGWQPGRSLRLSCAASGFTFSSYGMHWVRQAPGEGLEWVTVISYDGSDKY |
| | YADSVKGRFTISRDNSKSTLFLQMNSLRAEDTAVYYCARDGQFYFDYWGQGTLVTVSS |
| | [SEQ ID NO: 117] |
| Full V_{L} | EIVMTQSPATLSLSPGESATLSCRASQSVTSRYLSWYQQKPGQAPRLLMYGASTRPTGI |
| | PARFSGSGSGTDFTLTISSLQPEDFAVYYCQQDYNLPLTFGGGTKVEIK [SEQ ID |
| | NO: 118] |
| V_{H}-V_{L} | QVQLVESGGGVVQPGRSLRLSCAASGFTFSSYGMHWVRQAPGEGLEWVTVISYDGSDKY |
| scFv | YADSVKGRFTISRDNSKSTLFLQMNSLRAEDTAVYYCARDGQFYFDYWGQGTLVTVSSA |
| | STGGGGSGGGGSGGGGSEIVMTQSPATLSLSPGESATLSCRASQSVTSRYLSWYQQKPG |
| | QAPRLLMYGASTRPTGIPARFSGSGSGTDFTLTISSLQPEDFAVYYCQQDYNLPLTFGG |
| | GTKVEIK [SEQ ID NO: 119] |

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 120 or a conservative modification thereof, a V_{H} CDR2 comprising the amino acid sequence set forth in SEQ ID NO: 121 or a conservative modification thereof, and a V_{H} CDR3 comprising the amino acid sequence set forth in SEQ ID NO: 122 or a conservative modification thereof. SEQ ID NOs: 120-122 are provided in Table 15.

In certain embodiments, the extracellular antigen-binding domain of the CLEC12A-targeted chimeric receptor (e.g., a CLEC12A-targeted scFv) comprises a V_{L} CDR1 comprising the amino acid sequence set forth in SEQ ID NO: 123 or a conservative modification thereof, a V_{L} CDR2 comprising the amino acid sequence set forth in SEQ ID NO: 124 or a conservative modification thereof, and a V_{L} CDR3 comprising the amino acid sequence set forth in SEQ ID NO: 125 or a conservative modification thereof. SEQ ID NOs: 123-125 are provided in Table 15.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 120 or a conservative modification thereof, a V_{H} CDR2 comprising the amino acid sequence set forth in SEQ ID NO: 121 or a conservative modification thereof, a V_{H} CDR3 comprising the amino acid sequence set forth in SEQ ID NO: 122 or a conservative modification thereof, a V_{L} CDR1 comprising the amino acid sequence set forth in SEQ ID NO: 123 or a conservative modification thereof, a V_{L} CDR2 comprising the amino acid sequence set forth in SEQ ID NO: 124 or a conservative modification, and a V_{L} CDR3 comprising the amino acid sequence set forth in SEQ ID NO: 125 or a conservative modification thereof.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 120, a V_{H} CDR2 comprising the amino acid sequence set forth in SEQ ID NO: 121, a V_{H} CDR3 comprising the amino acid sequence set forth in SEQ ID NO: 122, a V_{L} CDR1 comprising the amino acid sequence set forth in SEQ ID NO: 123, a V_{L} CDR2 comprising the amino acid sequence set forth in SEQ ID NO: 124, and a V_{L} CDR3 comprising the amino acid sequence set forth in SEQ ID NO: 125.

In certain embodiments, the extracellular antigen-binding domain of the chimeric receptor (e.g., an scFv) comprises a V_{H} comprising an amino acid sequence that is at least about 80% (e.g., at least about 85%, at least about 90%, or at least about 95%) homologous or identical to the amino acid sequence set forth in SEQ ID NO: 126. For example, the extracellular antigen-binding domain of the chimeric receptor (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 126. In certain embodiments, the extracellular antigen-binding domain comprises a V_{H} comprising the amino acid sequence set forth in SEQ ID NO: 126. SEQ ID NO: 126 is provided in Table 15 below.

In certain embodiments, the extracellular antigen-binding domain of the chimeric receptor (e.g., an scFv) comprises a V_{L} comprising an amino acid sequence that is at least about 80% (e.g., at least about 85%, at least about 90%, or at least about 95%) homologous or identical to the amino acid sequence set forth in SEQ ID NO: 127. For example, the extracellular antigen-binding domain of the chimeric receptor (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 127. In certain embodiments, the extracellular antigen-binding domain comprises a V_{L} comprising the amino acid sequence set forth in SEQ ID NO: 127. SEQ ID NO: 127 is provided in Table 15 below.

In certain embodiments, the extracellular antigen-binding domain of the CLEC12A-targeted chimeric receptor (e.g., a CLEC12A-targeted scFv) comprises a V_{H} comprising the amino acid sequence set forth in SEQ ID NO: 126, and a V_{L} comprising the amino acid sequence set forth in SEQ ID NO: 127. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}. In certain embodiments, the CLEC12A-targeted scFv comprises the amino acid sequence set forth in SEQ ID NO: 128, which is provided in Table 15. In certain embodiments, the CLEC12A-targeted scFv is designated as “CLEC12A-H”. The CDRs provided in Table 15 are identified according to the IM GT numbering system.

TABLE 15
(CLEC12A-H)

| CDRs | 1 | 2 | 3 |
|---|---|---|---|
| V_{H} | GFTFSNYG [SEQ ID | ISYDGSDK [SEQ ID | ARDSGRYFFDY [SEQ |
| | NO: 120] | NO: 121] | ID NO: 122] |
| V_{L} | QSVSSRS [SEQ ID NO: | GPS [SEQ ID NO: | HQDYNLPLT [SEQ ID |
| | 123] | 124] | NO: 125] |

| | |
|---|---|
| Full V_{H} | QVQLVESGGGWQPGRSLRLSCAASGFTFSNYGMHWVRQAPGKGLEWVAVISYDGSDKS |
| | YKDSVKGRFTIARDNSKNTLYLQMNSLRAEDTAVYYCARDSGRYFFDYWGQGTLVTVSS |
| | [SEQ ID NO: 126] |
| Full V_{L} | EIIMTQSPATLSLSPGERATLSCRASQSVSSRSLSWYQHKPGQAPRLLIYGPSTRATGI |
| | PARFSGSGSGTDFTLTISSLQPEDFAVYYCHQDYNLPLTFGGGTKVEIK [SEQ ID |
| | NO: 127] |
| V_{H}-V_{L} | QVQLVESGGGVVQPGRSLRLSCAASGFTFSNYGMHWVRQAPGKGLEWVAVISYDGSDKS |
| scFv | YKDSVKGRFTIARDNSKNTLYLQMNSLRAEDTAVYYCARDSGRYFFDYWGQGTLVTVSS |
| | ASTGGGGSGGGGSGGGGSEIIMTQSPATLSLSPGERATLSCRASQSVSSRSLSWYQHKP |
| | GQAPRLLIYGPSTRATGIPARFSGSGSGTDFTLTISSLQPEDFAVYYCHQDYNLPLTFG |
| | GGTKVEIK [SEQ ID NO: 128] |

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 129 or a conservative modification thereof, a V_{H} CDR2 comprising the amino acid sequence set forth in SEQ ID NO: 130 or a conservative modification thereof, and a V_{H} CDR3 comprising the amino acid sequence set forth in SEQ ID NO: 131 or a conservative modification thereof. SEQ ID NOs: 129-131 are provided in Table 16.

In certain embodiments, the extracellular antigen-binding domain of the CLEC12A-targeted chimeric receptor (e.g., a CLEC12A-targeted scFv) comprises a V_{L} CDR1 comprising the amino acid sequence set forth in SEQ ID NO: 132 or a conservative modification thereof, a V_{L} CDR2 comprising the amino acid sequence set forth in SEQ ID NO: 133 or a conservative modification thereof, and a V_{L} CDR3 comprising the amino acid sequence set forth in SEQ ID NO: 134 or a conservative modification thereof. SEQ ID NOs: 132-134 are provided in Table 16.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 129 or a conservative modification thereof, a V_{H} CDR2 comprising the amino acid sequence set forth in SEQ ID NO: 130 or a conservative modification thereof, a V_{H} CDR3 comprising the amino acid sequence set forth in SEQ ID NO: 131 or a conservative modification thereof, a V_{L} CDR1 comprising the amino acid sequence set forth in SEQ ID NO: 132 or a conservative modification thereof, a V_{L} CDR2 comprising the amino acid sequence set forth in SEQ ID NO: 133 or a conservative modification, and a V_{L} CDR3 comprising the amino acid sequence set forth in SEQ ID NO: 134 or a conservative modification thereof.

In certain embodiments, the extracellular antigen-binding domain of the CLEC12A-targeted chimeric receptor (e.g., a CLEC12A-targeted scFv) comprises a V_{H} CDR1 comprising the amino acid sequence set forth in SEQ ID NO: 129, a V_{H} CDR2 comprising the amino acid sequence set forth in SEQ ID NO: 130, a V_{H} CDR3 comprising the amino acid sequence set forth in SEQ ID NO: 131, a V_{L} CDR1 comprising the amino acid sequence set forth in SEQ ID NO: 132, a V_{L} CDR2 comprising the amino acid sequence set forth in SEQ ID NO: 133, and a V_{L} CDR3 comprising the amino acid sequence set forth in SEQ ID NO: 134.

In certain embodiments, the extracellular antigen-binding domain of the CLEC12A-targeted chimeric receptor (e.g., a CLEC12A-targeted scFv) comprises a V_{H} comprising an amino acid sequence that is at least about 80% (e.g., at least about 85%, at least about 90%, or at least about 95%) homologous or identical to the amino acid sequence set forth in SEQ ID NO: 135. For example, the extracellular antigen-binding domain of the CLEC12A-targeted chimeric receptor (e.g., a CLEC12A-targeted scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 135. In certain embodiments, the extracellular antigen-binding domain of the CLEC12A-targeted chimeric receptor comprises a V_{H} comprising the amino acid sequence set forth in SEQ ID NO: 135. SEQ ID NO: 135 is provided in Table 16 below.

In certain embodiments, the extracellular antigen-binding domain of the CLEC12A-targeted chimeric receptor (e.g., a CLEC12A-targeted scFv) comprises a V_{L} comprising an amino acid sequence that is at least about 80% (e.g., at least about 85%, at least about 90%, or at least about 95%) homologous or identical to the amino acid sequence set forth in SEQ ID NO: 136. For example, the extracellular antigen-binding domain of the CLEC12A-targeted chimeric receptor (e.g., a CLEC12A-targeted scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 136. In certain embodiments, the extracellular antigen-binding domain of the CLEC12A-targeted chimeric receptor comprises a V_{L} comprising the amino acid sequence set forth in SEQ ID NO: 136. SEQ ID NO: 136 is provided in Table 16 below.

In certain embodiments, the extracellular antigen-binding domain of the CLEC12A-targeted chimeric receptor (e.g., a CLEC12A-targeted scFv) comprises a V_{H} comprising the amino acid sequence set forth in SEQ ID NO: 135, and a V_{L} comprising the amino acid sequence set forth in SEQ ID NO: 136. In certain embodiments, the V_{H} and V_{L} are linked via a linker. In certain embodiments, the linker comprises the amino acid sequence set forth in SEQ ID NO: 149. In certain embodiments, the linker comprises the amino acid sequence set forth in SEQ ID NO: 2.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}. In certain embodiments, the scFv comprises the amino acid sequence set forth in SEQ ID NO: 137, which is provided in Table 16. In certain embodiments, the CLEC12A-targeted scFv is designated as “CLEC12A-J”. The CDRs provided in Table 16 are identified according to the IM GT numbering system.

TABLE 16
(CLEC12A-J)

| CDRs | 1 | 2 | 3 |
|---|---|---|---|
| V_{H} | GFTFSKYG [SEQ ID | IWYDGSIK [SEQ ID | ARGSLWFGEFYFDY |
| | NO: 129] | NO: 130] | [SEQ ID NO: 131] |
| V_{L} | QGISSA [SEQ ID NO: | DAS [SEQ ID NO: | QQFNNYPRT [SEQ ID |
| | 132] | 133] | NO: 134] |

| | |
|---|---|
| Full V_{H} | QVKLVESGGGVVQPGRSLRLSCAASGFTFSKYGMHWVRQAPGKGLEWVAFIWYDGSIKN |
| | YADSVKGRFTTSRDNSKNTLYLQMNSLRAEDTAVYYCARGSLWFGEFYFDYWGQGTLVT |
| | VSS [SEQ ID NO: 135] |
| Full V_{L} | AIQLTQSPSSLSASVGDRVTITCRTSQGISSALAWYQQKPGKTPKLLIYDASSLESGVP |
| | SRFSGSGSGTDFTLTISSLQPEDFATYYCQQFNNYPRTFGQGTKVEIK [SEQ ID |
| | NO: 136] |
| V_{H}-V_{L} | QVKLVESGGGVVQPGRSLRLSCAASGFTFSKYGMHWVRQAPGKGLEWVAFIWYDGSIKN |
| scFv | YADSVKGRFTTSRDNSKNTLYLQMNSLRAEDTAVYYCARGSLWFGEFYFDYWGQGTLVT |
| | VSSASTGGGGSGGGGSGGGGSAIQLTQSPSSLSASVGDRVTITCRTSQGISSALAWYQQ |
| | KPGKTPKLLIYDASSLESGVPSRFSGSGSGTDFTLTISSLQPEDFATYYCQQFNNYPRT |
| | FGQGTKVEIK [SEQ ID NO: 137] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 75, SEQ ID NO: 76, SEQ ID NO: 86, SEQ ID NO: 87, SEQ ID NO: 95, SEQ ID NO: 96, SEQ ID NO: 100, SEQ ID NO: 101, SEQ ID NO: 106, SEQ ID NO: 107, SEQ ID NO: 111, SEQ ID NO: 112, SEQ ID NO: 117, SEQ ID NO: 118, SEQ ID NO: 126, SEQ ID NO: 127, SEQ ID NO: 135, or SEQ ID NO: 136) may contain substitutions (e.g., conservative substitutions), insertions, or deletions relative to the specified sequence(s), but retain the ability to bind to a target antigen (e.g., CLEC12A). In certain embodiments, a total of 1 to 10 amino acids are substituted, inserted and/or deleted in a specific sequence (e.g., SEQ ID NO: 75, SEQ ID NO: 76, SEQ ID NO: 86, SEQ ID NO: 87, SEQ ID NO: 95, SEQ ID NO: 96, SEQ ID NO: 100, SEQ ID NO: 101, SEQ ID NO: 106, SEQ ID NO: 107, SEQ ID NO: 111, SEQ ID NO: 112, SEQ ID NO: 117, SEQ ID NO: 118, SEQ ID NO: 126, SEQ ID NO: 127, SEQ ID NO: 135, or SEQ ID NO: 136). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NO: 75, SEQ ID NO: 76, SEQ ID NO: 86, SEQ ID NO: 87, SEQ ID NO: 95, SEQ ID NO: 96, SEQ ID NO: 100, SEQ ID NO: 101, SEQ ID NO: 106, SEQ ID NO: 107, SEQ ID NO: 111, SEQ ID NO: 112, SEQ ID NO: 117, SEQ ID NO: 118, SEQ ID NO: 126, SEQ ID NO: 127, SEQ ID NO: 135, or SEQ ID NO: 136, including post-translational modifications of that sequence (SEQ ID NO: 75, SEQ ID NO: 76, SEQ ID NO: 86, SEQ ID NO: 87, SEQ ID NO: 95, SEQ ID NO: 96, SEQ ID NO: 100, SEQ ID NO: 101, SEQ ID NO: 106, SEQ ID NO: 107, SEQ ID NO: 111, SEQ ID NO: 112, SEQ ID NO: 117, SEQ ID NO: 118, SEQ ID NO: 126, SEQ ID NO: 127, SEQ ID NO: 135, or SEQ ID NO: 136).

In certain embodiments, the extracellular antigen-binding domain of a presently disclosed CLEC12A-targeted chimeric receptor cross-competes for binding to CLEC12A with a reference antibody or an antigen-binding fragment thereof comprising the V_{H} CDR1, CDR2, and CDR3 sequences and the V_{L} CDR1, CDR2, and CDR3 sequences of, for example, any one of the presently disclosed scFvs (e.g., CLEC12A-A, CLEC12A-B, CLEC12A-C, CLEC12A-D, CLEC12A-E, CLEC12A-F, CLEC12A-G, CLEC12A-H, and CLEC12A-J). In certain embodiments, the extracellular antigen-binding domain of a presently disclosed chimeric receptor cross-competes for binding to CLEC12A with a reference antibody or an antigen-binding portion thereof comprising the V_{H} and V_{L} sequences of, for example, any one of the presently disclosed scFvs (e.g., CLEC12A-A, CLEC12A-B, CLEC12A-C, CLEC12A-D, CLEC12A-E, CLEC12A-F, CLEC12A-G, CLEC12A-H, and CLEC12A-J).

In certain embodiments, the extracellular antigen-binding domain of a presently disclosed CLEC12A-targeted chimeric receptor cross-competes for binding to CLEC12A with a reference antibody or an antigen-binding portion thereof comprising the V_{H} CDR1, CDR2, and CDR3 sequences and the V_{L} CDR1, CDR2, and CDR3 sequences of scFv CLEC12A-A. For example, the extracellular antigen-binding domain of a presently disclosed CLEC12A-targeted chimeric receptor cross-competes for binding to CLEC12A with a reference antibody or an antigen-binding portion thereof comprising a V_{H} CDR1 comprising the amino acid sequence set forth in SEQ ID NO: 69, a V_{H} CDR2 comprising the amino acid sequence set forth in SEQ ID NO: 70; a V_{H} CDR3 comprising the amino acid sequence set forth in SEQ ID NO: 71; a V_{L} CDR1 comprising the amino acid sequence set forth in SEQ ID NO: 72; a V_{L} CDR2 comprising amino acids having the sequence set forth in SEQ ID NO: 73; and a V_{L} CDR3 comprising amino acids having the sequence set forth in SEQ ID NO: 74. In certain embodiments, the extracellular antigen-binding domain of a presently disclosed CLEC12A-targeted chimeric receptor cross-competes for binding to CLEC12A with a reference antibody or an antigen-binding portion thereof comprising the V_{H} and V_{L} sequences of scFv CLEC12A-A. For example, the extracellular antigen-binding domain of a presently disclosed CLEC12A-targeted chimeric receptor cross-competes for binding to CLEC12A with a reference antibody or an antigen-binding portion thereof comprising a V_{H} comprising amino acids having the sequence set forth in SEQ ID NO: 75, and a V_{L} comprising amino acids having the sequence set forth in SEQ ID NO: 76.

In certain embodiments, the extracellular antigen-binding domain of the presently disclosed CLEC12A-targeted chimeric receptors binds to the same epitope on CLEC12A as the reference antibody or antigen-binding portion thereof. For example, the extracellular antigen-binding domain of a presently disclosed CLEC12A-targeted chimeric receptor binds to the same epitope on CLEC12A as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., CLEC12A-A, CLEC12A-B, CLEC12A-C, CLEC12A-D, CLEC12A-E, CLEC12A-F, CLEC12A-G, CLEC12A-H, and CLEC12A-J). In certain embodiments, the extracellular antigen-binding domain of a presently disclosed CLEC12A-targeted chimeric receptor binds to the same epitope on CLEC12A as a reference antibody or an antigen-binding portion thereof comprising the V_{H} and V_{L} sequences of, for example, any one of the presently disclosed scFvs (e.g., CLEC12A-A, CLEC12A-B, CLEC12A-C, CLEC12A-D, CLEC12A-E, CLEC12A-F, CLEC12A-G, CLEC12A-H, and CLEC12A-J).

In certain embodiments, the extracellular antigen-binding domain of a presently disclosed CLEC12A-targeted chimeric receptor binds to the same epitope on CLEC12A as a reference antibody or an antigen-binding fragment thereof comprising the V_{H} CDR1, CDR2, and CDR3 sequences and the V_{L} CDR1, CDR2, and CDR3 sequences of scFv CLEC12A-A. For example, the extracellular antigen-binding domain of a presently disclosed CLEC12A-targeted chimeric receptor binds to the same epitope on CLEC12A as a reference antibody or an antigen-binding fragment thereof comprising a V_{H} CDR1 comprising the amino acid sequence set forth in SEQ ID NO: 69; a V_{H} CDR2 comprising the amino acid sequence set forth in SEQ ID NO: 70; a V_{H} CDR3 comprising the amino acid sequence set forth in SEQ ID NO: 71; a V_{L} CDR1 comprising the amino acid sequence set forth in SEQ ID NO: 72; a V_{L} CDR2 comprising the amino acid sequence set forth in SEQ ID NO: 73; and a V_{L} CDR3 comprising the amino acid sequence set forth in SEQ ID NO: 74. In certain embodiments, the extracellular antigen-binding domain of a presently disclosed CLEC12A-targeted chimeric receptor binds to the same or substantially the same epitope on CLEC12A as a reference antibody or an antigen-binding fragment thereof comprising the V_{H} and V_{L} sequences of scFv CLEC12A-A. For example, the extracellular antigen-binding domain of a presently disclosed CLEC12A-targeted chimeric receptor binds to the same epitope on CLEC12A as a reference antibody or an antigen-binding fragment thereof comprising a V_{H} comprising the amino acid sequence set forth in SEQ ID NO: 75, and a V_{L} comprising the amino acid sequence set forth in SEQ ID NO: 76.

Extracellular antigen-binding domains of the presently disclosed CLEC12A-targeted chimeric receptors that cross-compete or compete with the reference antibody or antigen-binding portions thereof for binding to CLEC12A can be identified by using routine methods known in the art, e.g., those disclosed in Section 5.3.

In certain embodiments, the extracellular antigen-binding domain of the presently disclosed CLEC12A-targeted chimeric comprises a linker connecting the heavy chain variable region and light chain variable region of the extracellular antigen-binding domain. In certain embodiments, the linker comprises the amino acid sequence set forth in SEQ ID NO: 1. In certain embodiments, the linker comprises the amino acid sequence set forth in SEQ ID NO: 2.

In certain embodiments, the linker comprises the amino acid sequence set forth in SEQ ID NO: 3. In certain embodiments, the linker comprises the amino acid sequence set forth in SEQ ID NO: 4. In certain embodiments, the linker comprises the amino acid sequence set forth in SEQ ID NO: 5. In certain embodiments, the linker comprises the amino acid sequence set forth in SEQ ID NO: 6. In certain embodiments, the linker comprises the amino acid sequence set forth in SEQ ID NO: 149.

In certain embodiments, the variable regions within the extracellular antigen-binding domain of the CLEC12A-targeted chimeric receptor have to be linked one after another such that at the N-terminus of the extracellular antigen-binding domain, a heavy chain variable region (V_{H}) is positioned. In certain embodiments, if the extracellular antigen-binding domain of the CLEC12A-targeted chimeric receptor is an scFv, the variable regions are positioned from the N- to the C-terminus: V_{H}-V_{L}.

In certain embodiments, the variable regions within the extracellular antigen-binding domain of the CLEC12A-targeted chimeric receptor have to be linked one after another such that at the N-terminus of the extracellular antigen-binding domain, a light chain variable region (V_{L}) is positioned. In certain embodiments, if the extracellular antigen-binding domain of the CLEC12A-targeted chimeric receptor is an scFv, the variable regions are positioned from the N- to the C-terminus: V_{L}-V_{H}.

In addition, the CLEC12A-targeted chimeric receptor can comprise a leader or a signal peptide that directs the nascent protein into the endoplasmic reticulum. In certain embodiments, the leader or signal peptide is positioned at (e.g., covalently joined to) the N-terminus of the extracellular antigen-binding domain of the CLEC12A-targeted chimeric receptor. In certain embodiments, the CLEC12A-targeted chimeric receptor comprises a leader or signal peptides disclosed in Section 5.3. In certain embodiments, the CLEC12A-targeted chimeric receptor comprises a signal peptide that comprises a CD8 polypeptide. In certain embodiments, the CLEC12A-targeted chimeric receptor comprises a signal peptide that comprises a CD8 polypeptide comprising or consisting of the amino acid sequence set forth in SEQ ID NO: 63.

### 5.6. Exemplified CLEC12A-Targeted Chimeric Receptor

In certain embodiments, the CLEC12A-targeted chimeric receptor is a CCR. In certain embodiments, the CLEC12A-targeted CCR comprises (a) an extracellular antigen-binding domain comprising (i) a V_{H} that comprises a CDR1 comprising the amino acid sequence set forth in SEQ ID NO: 69, a CDR2 comprising the amino acid sequence set forth in SEQ ID NO: 70, and a V_{H} CDR3 comprising the amino acid sequence set forth in SEQ ID NO: 71, and (ii) a V_{L} that comprises a CDR1 comprising the amino acid sequence set forth in SEQ ID NO: 72, a CDR2 comprising the amino acid sequence set forth in SEQ ID NO: 73, and a V_{L} CDR3 comprising the amino acid sequence set forth in SEQ ID NO: 74; (b) an intracellular domain comprising a 4-1BB polypeptide (e.g., a human 4-1BB polypeptide, e.g., an intracellular domain of 4-1BB (e.g., human 4-1BB) of a portion thereof). In certain embodiments, the CLEC12A-targeted CCR further comprises a transmembrane domain comprising a CD8 polypeptide (e.g., a human CD8 polypeptide, e.g., a transmembrane domain of CD8 (e.g., human CD8) or a portion thereof). In certain embodiments, the intracellular domain comprises a 4-1BB polypeptide comprising or consisting of the amino acids 214 to 255 of SEQ ID NO: 30. In certain embodiments, the transmembrane domain comprises a CD8 polypeptide comprising or consisting of the amino acids 137 to 207 of SEQ ID NO: 7. In certain embodiments, the V_{H} and V_{L} are linked via a linker comprising or consisting of the amino acid sequence set forth in SEQ ID NO: 149. In certain embodiments, the V_{H} and V_{L} are positioned from the N- to the C-terminus: V_{H}-V_{L}. In certain embodiments, the extracellular antigen-binding domain and transmembrane domain are linked via a linker. In certain embodiments, the linker consists of the amino acid sequence set forth in SEQ ID NO: 150. In certain embodiments, the CLEC12A-targeted CCR comprises the amino acid sequence set forth in SEQ ID NO: 138, which is provided below.

[SEQ ID NO: 138]
QLQLQESGPGLVKPSETLSLTCTVSGGSISSSTYYWGWIRQPPRKGLEWI
GSTHYRGSTYYNPSLKSRVTISVDTSKNQFSLKVSSVTAADTAVYYCARE
LTGEVFDYWGQGTLVTVSSASTGGGGSGGGGSGGGGSDIQMTQSPSSLSA
SVGDRVTITCRASQSISSYLNWYQQKPGKAPKLLIYAASSLQSGVPSRFS
GSGSGTDFTLTISSLQPEDFATYYCQQSYSTPFTFGPGTKVDIKRAAAPT
TTPAPRPPTPAPTIASQPLSLRPEACRPAAGGAVHTRGLDFACDIYIWAP
LAGTCGVLLLSLVITLYCNKRGRKKLLYIFKQPFMRPVQTTQEEDGCSCR
FPEEEEGGCEL

An exemplary nucleic acid sequence the amino acid sequence of SEQ ID NO: 138 is set forth in SEO ID NO: 139, which is provided below.

[SEQ ID NO: 139]
cagctccagctccaagagtcagggccaggtctcgtgaaaccgagtgagac
cctgtccctgacctgcacagtgagtggtggatcaatctcaagctctacct
actattgggggtggattcggcagccccctagaaaggggcttgagtggatt
ggcagcactcattatcgaggatctacctattataatccttctctgaaaag
cagagttaccatctctgtggatacgtccaaaaatcagttcagtctgaagg
tatcatccgtgactgctgccgacacggccgtgtactattgcgcgagggag
ctgacaggtgaggtctttgactactggggccagggcacactcgtgaccgt
gtcttctgcctcaacaggagggggtgggagtggaggcggtggatcagggg
gaggagggagtgacatccagatgacgcagtccccttccagcttgtccgca
tctgtgggtgatagggtcacgattacatgtagggctagtcagagtatttc
tagttacctgaattggtaccagcagaaaccaggcaaggcaccaaagttgc
tcatctatgcggcctcctctctgcaatctggcgtgccgtccagatttagt
ggatcaggctccggaaccgatttcacccttacgatctcctcacttcaacc
cgaggatttcgccacatattactgtcaacaaagctattctacaccgttca
ccttcggaccggggacaaaagtggatattaaacgggcggccgcccccacc
acgacgccagcgccgcgaccaccaaccccggcgcccacgatcgcgtcgca
gcccctgtccctgcgcccagaggcgtgccggccagcggcggggggcgcag
tgcacacgagggggctggacttcgcctgtgatatctacatctgggcgccc
ctggccgggacttgtggggtccttctcctgtcactggttatcacccttta
ctgcaacaaacggggcagaaagaagctcctgtatatattcaaacaaccat
ttatgagaccagtacaaactactcaagaggaagatggctgtagctgccga
tttccagaagaagaagaaggaggatgtgaactg

In certain embodiments, the CLEC12A-targeted chimeric receptor comprises a signal peptide at the N-terminus of the extracellular antigen-binding domain. In certain embodiments, the signal peptide comprises a CD8 polypeptide comprising or consisting of the amino acid sequence set forth in SEQ ID NO: 63.

### 5.7. Cells

The presently disclosed subject matter provides cells comprising a presently disclosed ADGRE2-targeted chimeric receptor (e.g., one disclosed in Sections 5.3 and 5.4). In addition, the presently disclosed subject matter provides cells comprising a presently disclosed CLEC12A-targeted chimeric receptor (e.g., one disclosed in Sections 5.5 and 5.6).

In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell. In certain embodiments, the pluripotent stem cell is an embryonic stem cell (ESC) or an induced pluripotent stem cell (iPSC).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an chimeric receptor, e.g., a CAR or a CCR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells. In certain embodiments, the cell is a genetically modified NK cell. In certain embodiments, the cell is an edited NK cell. In certain embodiments, the cell is a NK cell derived from a stem cell. In certain embodiments, the cell is a NK cell derived from a pluripotent stem cell. In certain embodiments, the cell is an induced pluripotent stem cell (iPSC)-derived NK cell.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells or NK cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAM s), immune suppressive cytokines including TGF-B, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred modified T cells upon encounter with targeted tumor cells.

In certain embodiments, the cells can be transduced with the presently disclosed ADGRE2-targeted chimeric receptor and/or the presently disclosed CLEC12A-targeted chimeric receptor such that the cells express the chimeric receptor(s).

Furthermore, the presently disclosed subject matter provides cells comprising a presently disclosed ADGRE2-targeted chimeric receptor (e.g., one disclosed in Section 5.3) and a presently disclosed CLEC12A-targeted chimeric receptor (e.g., one disclosed in Section 5.4). In certain embodiments, the ADGRE2-targeted chimeric receptor is a CAR. In certain embodiments, the CLEC12-targeted chimeric receptor is a CCR. Thus, in certain embodiments, the presently disclosed subject matter provides cells comprising a presently disclosed CLEC12A-targeted CAR and a presently disclosed CLEC12A-targeted CCR.

In certain embodiments, the presently disclosed cells exhibit a greater degree of cytolytic activity against cells that are positive for both ADGRE2 and CLEC12A as compared to against cells that are singly positive for ADGRE2. In certain embodiments, the ADGRE2-targeted CAR binds to the ADGRE2 with a low binding affinity or a low binding avidity. In certain embodiments, the ADGRE2-targeted CAR binds to ADGRE2 at an epitope of low accessibility. In certain embodiments, the ADGRE2-targeted CAR binds to ADGRE2 with a binding affinity that is lower compared to the binding affinity with which the CLEC12A-targeted CCR binds to CLEC12A. In certain embodiments, the ADGRE2-targeted CAR binds to ADGRE2 with a binding affinity that is at least 5-fold lower compared to the binding affinity with which the CLEC12A-targeted CCR binds to CLEC12A. In certain embodiments, the ADGRE2-targeted CAR binds to ADGRE2 with a binding affinity that is at least 10 fold, 20 fold, 30 fold, 40 fold, 50 fold, 60 fold, 70 fold, 80 fold, 90 fold, 100 fold, 200 fold, 5000 fold, 1000 fold, 5000 fold, or 10000 fold lower compared to the binding affinity with which the CLEC12A-targeted CCR binds to CLEC12A.

### 5.5.1. Exemplified Cells

In certain embodiments, the cell comprises an ADGRE2-targeted CAR and a CLEC12A-targeted CCR.

In certain embodiments, the ADGRE2-targeted CAR comprises (a) an extracellular antigen-binding domain comprising (i) a V_{H} that comprises a CDR1 comprising the amino acid sequence set forth in SEQ ID NO: 33, a CDR2 comprising the amino acid sequence set forth in SEQ ID NO: 34, and a V_{H} CDR3 comprising the amino acid sequence set forth in SEQ ID NO: 35, and (ii) a V_{L} that comprises a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; (b) a hinge/spacer region comprising a CD28 polypeptide, (c) a transmembrane domain comprising a CD28 polypeptide (e.g., a transmembrane domain of human CD28 or a fragment thereof), and (d) an intracellular signaling domain comprising (i) a CD3% polypeptide, and (ii) a co-stimulatory signaling region comprising a CD28 polypeptide (e.g., an intracellular domain of human CD28 or a fragment thereof). In certain embodiments, the V_{H} and V_{L} are linked via a linker comprising or consisting of the amino acid sequence set forth in SEQ ID NO: 2. In certain embodiments, the V_{H} and V_{L} are positioned from the N- to the C-terminus: V_{H}-V_{L}. In certain embodiments, the transmembrane domain comprises a CD28 polypeptide comprising or consisting of the amino acids 153 to 179 of SEQ ID NO: 10. In certain embodiments, the co-stimulatory signaling region comprises a CD28 polypeptide comprising or consisting of the amino acids 180 to 220 of SEQ ID NO: 10. In certain embodiments, the hinge/spacer region comprises a CD28 polypeptide comprising or consisting of the amino acids 114 to 152 of SEQ ID NO: 10. In certain embodiments, the ADGRE2-targeted CAR comprises or consists of the amino acid sequence set forth in SEQ ID NO: 66.

In certain embodiments, the CLEC12A-targeted CCR comprises (a) an extracellular antigen-binding domain comprising (i) a V_{H} that comprises a CDR1 comprising the amino acid sequence set forth in SEQ ID NO: 69, a CDR2 comprising the amino acid sequence set forth in SEQ ID NO: 70, and a V_{H} CDR3 comprising the amino acid sequence set forth in SEQ ID NO: 71, and (ii) a V_{L} that comprises a CDR1 comprising the amino acid sequence set forth in SEQ ID NO: 72, a CDR2 comprising the amino acid sequence set forth in SEQ ID NO: 73, and a V_{L} CDR3 comprising the amino acid sequence set forth in SEQ ID NO: 74; (b) an intracellular domain comprising a 4-1BB polypeptide (e.g., a human 4-1BB polypeptide, e.g., an intracellular domain of 4-1BB (e.g., human 4-1BB) of a portion thereof). In certain embodiments, the CLEC12A-targeted CCR further comprises a transmembrane domain comprising a CD8 polypeptide (e.g., a human CD8 polypeptide, e.g., a transmembrane domain of CD8 (e.g., human CD8) or a portion thereof). In certain embodiments, the intracellular domain comprises a 4-1BB polypeptide comprising or consisting of the amino acids 214 to 255 of SEQ ID NO: 30. In certain embodiments, the transmembrane domain comprises a CD8 polypeptide comprising or consisting of the amino acids 137 to 207 of SEQ ID NO: 7. In certain embodiments, the V_{H} and V_{L} are linked via a linker comprising or consisting of the amino acid sequence set forth in SEQ ID NO: 2. In certain embodiments, the V_{H} and V_{L} are positioned from the N- to the C-terminus: V_{H}-V_{L}. In certain embodiments, the CLEC12A-targeted CCR comprises the amino acid sequence set forth in SEQ ID NO: 136.

### 5.8. Nucleic Acid Molecules, Vector and Genetic Modifications

The presently disclosed subject matter provides nucleic acid molecules encoding the presently disclosed ADGRE2-targeted chimeric receptors (e.g., those disclosed in Sections 5.3 and 5.4). In certain embodiments, the nucleic acid molecule further comprises a promoter that is operably linked to the presently disclosed ADGRE2-targeted CAR. Also provided are cells comprising such nucleic acid molecules.

In addition, the presently disclosed subject matter provides nucleic acid molecules encoding the presently disclosed CLEC12A-targeted chimeric receptors (e.g., those disclosed in Sections 5.5 and 5.6). In certain embodiments, the nucleic acid molecule further comprises a promoter that is operably linked to the presently disclosed CLEC12A-targeted CAR. Also provided are cells comprising such nucleic acid molecules.

Furthermore, the presently disclosed subject matter provides nucleic acid compositions comprising a nucleic acid molecule encoding an ADGRE2-targeted chimeric receptor and a nucleic acid molecule encoding a CLEC12A-targeted chimeric receptor. Also provided are cells comprising such nucleic acid compositions.

In certain embodiments, the promoter is endogenous or exogenous. In certain embodiments, the exogenous promoter is selected from the group consisting of an elongation factor (EF)-1 promoter, a cytomegalovirus immediate-early promoter (CM V) promoter, a simian virus 40 early promoter (SV40) promoter, a phosphoglycerate kinase (PGK) promoter, a metallothionein promoter, and Ubiquitin C promoter. In certain embodiments, the endogenous promoter is selected from a TCR alpha promoter, a TCR beta promoter, and a beta 2-microglobulin promoter. In certain embodiments, the promoter is an inducible promoter. In certain embodiment, the inducible promoter is selected from the group consisting of a NFAT transcriptional response element (TRE) promoter, a CD69 promoter, a CD25 promoter, an IL-2 promoter, a 4-1BB promoter, a PD 1 promoter, and a LA G3 promoter.

The presently disclosed subject matter also provides vectors comprising the presently disclosed nucleic acid molecules. In certain embodiments, the vector is a viral vector. In certain embodiments, the viral vector is a retroviral vector. In certain embodiments, the retroviral vector is a gamma retroviral vector or lentiviral vector.

In certain embodiments, the vector comprises a nucleic acid molecule encoding a presently disclosed ADGRE2-targeted CAR and a presently disclosed CLEC12A-targeted CCR. In certain embodiments, the nucleic acid molecule encodes a polypeptide comprising the amino acid sequence set forth in SEQ ID NO: 140. In certain embodiments, the nucleic acid molecule comprises the nucleotide sequence set forth in SEQ ID NO: 141. SEQ ID NOS: 140 and 141 are provided below.

[SEQ ID NO: 140]
MALPVTALLLPLALLLHAQVQLQQSGAEVAKPGASVKLSCKASGYTETNYWMQWIKQAPGQGLEWIGAVYPGDGDTR
HTQKFKGKATLTADKSTSTAYMEVSSLRSEDTAVYYCARGFTAYGMDYWGQGTTVTVSSGGGGSGGGGSGGGGSEIV
LTQSPATMSASPGERVTMSCSASSSVSYMHWYQQKSGQSPKRWIYDTSKLASGVPARFSGSGSGTDYTFTISSMEPE
DFATYYCQQWSSNPLTFGGGTKLEIKRAAAIEVMYPPPYLDNEKSNGTIIHVKGKHLCPSPLFPGPSKPFWVLVVVG
GVLACYSLLVTVAFIIFWVRSKRSRLLHSDYMNMTPRRPGPTRKHYQPYAPPRDFAAYRSRVKFSRSADAPAYQQGQ
NQLYNELNLGRREEYDVLDKRRGRDPEMGGKPRRKNPQEGLFNELQKDKMAEAFSEIGMKGERRRGKGHDGLFQGLS
TATKDTFDALHMQALPPRGSGATNFSLLKQAGDVEENPGPMALPVTALLLPLALLLHAQLQLQESGPGLVKPSETLS
LTCTVSGGSISSSTYYWGWIRQPPRKGLEWIGSTHYRGSTYYNPSLKSRVTISVDTSKNQFSLKVSSVTAADTAVYY
CARELTGEVFDYWGQGTLVTVSSASTGGGGSGGGGSGGGGSDIQMTQSPSSLSASVGDRVTITCRASQSISSYLNWY
QQKPGKAPKLLIYAASSLQSGVPSRFSGSGSGTDFTLTISSLQPEDFATYYCQQSYSTPFTFGPGTKVDIKRAAAPT
TTPAPRPPTPAPTIASQPLSLRPEACRPAAGGAVHTRGLDFACDIYIWAPLAGTCGVLLLSLVITLYCNKRGRKKLL
YIFKQPFMRPVQTTQEEDGCSCRFPEEEEGGCEL
[SEQ ID NO: 141]
GGATTAGTCCAATTTGTTAAAGACAGGATATCAGTGGTCCAGGCTCTAGTTTTGACTCAACAATATCACCAGCTGAA
GCCTATAGAGTACGAGCCATAGATAAAATAAAAGATTTTATTTAGTCTCCAGAAAAAGGGGGGAATGAAAGACCCCA
CCTGTAGGTTTGGCAAGCTAGCTTAAGTAACGCCATTTTGCAAGGCATGGAAAAATACATAACTGAGAATAGAGAAG
TTCAGATCAAGGTCAGGAACAGATGGAACAGCTGAATATGGGCCAAACAGGATATCTGTGGTAAGCAGTTCCTGCCC
CGGCTCAGGGCCAAGAACAGATGGAACAGCTGAATATGGGCCAAACAGGATATCTGTGGTAAGCAGTTCCTGCCCCG
GCTCAGGGCCAAGAACAGATGGTCCCCAGATGCGGTCCAGCCCTCAGCAGTTTCTAGAGAACCATCAGATGTTTCCA
GGGTGCCCCAAGGACCTGAAATGACCCTGTGCCTTATTTGAACTAACCAATCAGTTCGCTTCTCGCTTCTGTTCGCG
CGCTTCTGCTCCCCGAGCTCAATAAAAGAGCCCACAACCCCTCACTCGGGGCGCCAGTCCTCCGATTGACTGAGTCG
CCCGGGTACCCGTGTATCCAATAAACCCTCTTGCAGTTGCATCCGACTTGTGGTCTCGCTGTTCCTTGGGAGGGTCT
CCTCTGAGTGATTGACTACCCGTCAGCGGGGGTCTTTCACATGCAGCATGTATCAAAATTAATTTGGTTTTTTTTCT
TAAGTATTTACATTAAATGGCCATAGTACTTAAAGTTACATTGGCTTCCTTGAAATAAACATGGAGTATTCAGAATG
TGTCATAAATATTTCTAATTTTAAGATAGTATCTCCATTGGCTTTCTACTTTTTCTTTTATTTTTTTTTGTCCTCTG
TCTTCCATTTGTTGTTGTTGTTGTTTGTTTGTTTGTTTGTTGGTTGGTTGGTTAATTTTTTTTTAAAGATCCTACAC
TATAGTTCAAGCTAGACTATTAGCTACTCTGTAACCCAGGGTGACCTTGAAGTCATGGGTAGCCTGCTGTTTTAGCC
TTCCCACATCTAAGATTACAGGTATGAGCTATCATTTTTGGTATATTGATTGATTGATTGATTGATGTGTGTGTGTG
TGATTGTGTTTGTGTGTGTGATTGTGTATATGTGTGTATGGTTGTGTGTGATTGTGTGTATGTATGTTTGTGTGTGA
TTGTGTGTGTGTGATTGTGCATGTGTGTGTGTGTGATTGTGTTTATGTGTATGATTGTGTGTGTGTGTGTGTGTGTG
TGTGTGTGTGTGTGTGTGTGTGTGTTGTGTATATATATTTATGGTAGTGAGAGGCAACGCTCCGGCTCAGGTGTCAG
GTTGGTTTTTGAGACAGAGTCTTTCACTTAGCTTGGAATTCACTGGCCGTCGTTTTACAACGTCGTGACTGGGAAAA
CCCTGGCGTTACCCAACTTAATCGCCTTGCAGCACATCCCCCTTTCGCCAGCTGGCGTAATAGCGAAGAGGCCCGCA
CCGATCGCCCTTCCCAACAGTTGCGCAGCCTGAATGGCGAATGGCGCCTGATGCGGTATTTTCTCCTTACGCATCTG
TGCGGTATTTCACACCGCATATGGTGCACTCTCAGTACAATCTGCTCTGATGCCGCATAGTTAAGCCAGCCCCGACA
CCCGCCAACACCCGCTGACGCGCCCTGACGGGCTTGTCTGCTCCCGGCATCCGCTTACAGACAAGCTGTGACCGTCT
CCGGGAGCTGCATGTGTCAGAGGTTTTCACCGTCATCACCGAAACGCGCGAGACGAAAGGGCCTCGTGATACGCCTA
TTTTTATAGGTTAATGTCATGATAATAATGGTTTCTTAGACGTCAGGTGGCACTTTTCGGGGAAATGTGCGCGGAAC
CCCTATTTGTTTATTTTTTTAAATACATTCAAATATGTATCCGCTCATGAGACAATAACCCTGATAAATGCTTCAAT
AATATTGAAAAAGGAAGAGTATGAGCCATATTCAACGGGAAACGTCGAGGCCGCGATTAAATTCCAACATGGATGCT
GATTTATATGGGTATAAATGGGCTCGCGATAATGTCGGGCAATCAGGTGCGACAATCTATCGCTTGTATGGGAAGCC
CGATGCGCCAGAGTTGTTTCTGAAACATGGCAAAGGTAGCGTTGCCAATGATGTTACAGATGAGATGGTCAGACTAA
ACTGGCTGACGGAATTTATGCCTCTTCCGACCATCAAGCATTTTATCCGTACTCCTGATGATGCATGGTTACTCACC
ACTGCGATCCCCGGAAAAACAGCATTCCAGGTATTAGAAGAATATCCTGATTCAGGTGAAAATATTGTTGATGCGCT
GGCAGTGTTCCTGCGCCGGTTGCATTCGATTCCTGTTTGTAATTGTCCTTTTAACAGCGATCGCGTATTTCGTCTCG
CTCAGGCGCAATCACGAATGAATAACGGTTTGGTTGATGCGAGTGATTTTGATGACGAGCGTAATGGCTGGCCTGTT
GAACAAGTCTGGAAAGAAATGCATAAACTTTTGCCATTCTCACCGGATTCAGTCGTCACTCATGGTGATTTCTCACT
TGATAACCTTATTTTTGACGAGGGGAAATTAATAGGTTGTATTGATGTTGGACGAGTCGGAATCGCAGACCGATACC
AGGATCTTGCCATCCTATGGAACTGCCTCGGTGAGTTTTCTCCTTCATTACAGAAACGGCTTTTTCAAAAATATGGT
ATTGATAATCCTGATATGAATAAATTGCAGTTTCATTTGATGCTCGATGAGTTTTTCTAACTGTCAGACCAAGTTTA
CTCATATATACTTTAGATTGATTTAAAACTTCATTTTTAATTTAAAAGGATCTAGGTGAAGATCCTTTTTGATAATC
TCATGACCAAAATCCCTTAACGTGAGTTTTCGTTCCACTGAGCGTCAGACCCCGTAGAAAAGATCAAAGGATCTTCT
TGAGATCCTTTTTTTCTGCGCGTAATCTGCTGCTTGCAAACAAAAAAACCACCGCTACCAGCGGTGGTTTGTTTGCC
GGATCAAGAGCTACCAACTCTTTTTCCGAAGGTAACTGGCTTCAGCAGAGCGCAGATACCAAATACTGTTCTTCTAG
TGTAGCCGTAGTTAGGCCACCACTTCAAGAACTCTGTAGCACCGCCTACATACCTCGCTCTGCTAATCCTGTTACCA
GTGGCTGCTGCCAGTGGCGATAAGTCGTGTCTTACCGGGTTGGACTCAAGACGATAGTTACCGGATAAGGCGCAGCG
GTCGGGCTGAACGGGGGGTTCGTGCACACAGCCCAGCTTGGAGCGAACGACCTACACCGAACTGAGATACCTACAGC
GTGAGCTATGAGAAAGCGCCACGCTTCCCGAAGGGAGAAAGGCGGACAGGTATCCGGTAAGCGGCAGGGTCGGAACA
GGAGAGCGCACGAGGGAGCTTCCAGGGGGAAACGCCTGGTATCTTTATAGTCCTGTCGGGTTTCGCCACCTCTGACT
TGAGCGTCGATTTTTGTGATGCTCGTCAGGGGGGCGGAGCCTATGGAAAAACGCCAGCAACGCGGCCTTTTTACGGT
TCCTGGCCTTTTGCTGGCCTTTTGCTCACATGTTCTTTCCTGCGTTATCCCCTGATTCTGTGGATAACCGTATTACC
GCCTTTGAGTGAGCTGATACCGCTCGCCGCAGCCGAACGACCGAGCGCAGCGAGTCAGTGAGCGAGGAAGCGGAAGA
GCGCCCAATACGCAAACCGCCTCTCCCCGCGCGTTGGCCGATTCATTAATGCAGCTGGCACGACAGGTTTCCCGACT
GGAAAGCGGGCAGTGAGCGCAACGCAATTAATGTGAGTTAGCTCACTCATTAGGCACCCCAGGCTTTACACTTTATG
CTTCCGGCTCGTATGTTGTGTGGAATTGTGAGCGGATAACAATTTCACACAGGAAACAGCTATGACCATGATTACGC
CAAGCTTTGCTCTTAGGAGTTTCCTAATACATCCCAAACTCAAATATATAAAGCATTTGACTTGTTCTATGCCCTAG
GGGGCGGGGGGAAGCTAAGCCAGCTTTTTTTAACATTTAAAATGTTAATTCCATTTTAAATGCACAGATGTTTTTAT
TTCATAAGGGTTTCAATGTGCATGAATGCTGCAATATTCCTGTTACCAAAGCTAGTATAAATAAAAATAGATAAACG
TGGAAATTACTTAGAGTTTCTGTCATTAACGTTTCCTTCCTCAGTTGACAACATAAATGCGCTGCTGAGAAGCCAGT
TTGCATCTGTCAGGATCAATTTCCCATTATGCCAGTCATATTAATTACTAGTCAATTAGTTGATTTTTATTTTTGAC
ATATACATGTGAAAGACCCCACCTGTAGGTTTGGCAAGCTAGCTTAAGTAACGCCATTTTGCAAGGCATGGAAAAAT
ACATAACTGAGAATAGAAAAGTTCAGATCAAGGTCAGGAACAGATGGAACAGCTGAATATGGGCCAAACAGGATATC
TGTGGTAAGCAGTTCCTGCCCCGGCTCAGGGCCAAGAACAGATGGAACAGCTGAATATGGGCCAAACAGGATATCTG
TGGTAAGCAGTTCCTGCCCCGGCTCAGGGCCAAGAACAGATGGTCCCCAGATGCGGTCCAGCCCTCAGCAGTTTCTA
GAGAACCATCAGATGTTTCCAGGGTGCCCCAAGGACCTGAAATGACCCTGTGCCTTATTTGAACTAACCAATCAGTT
CGCTTCTCGCTTCTGTTCGCGCGCTTCTGCTCCCCGAGCTCAATAAAAGAGCCCACAACCCCTCACTCGGCGCGCCA
GTCCTCCGATTGACTGAGTCGCCCGGGTACCCGTGTATCCAATAAACCCTCTTGCAGTTGCATCCGACTTGTGGTCT
CGCTGTTCCTTGGGAGGGTCTCCTCTGAGTGATTGACTACCCGTCAGCGGGGGTCTTTCATTTGGGGGCTCGTCCGG
GATCGGGAGACCCCTGCCCAGGGACCACCGACCCACCACCGGGAGGTAAGCTGGCCAGCAACTTATCTGTGTCTGTC
CGATTGTCTAGTGTCTATGACTGATTTTATGCGCCTGCGTCGGTACTAGTTAGCTAACTAGCTCTGTATCTGGCGGA
CCCGTGGTGGAACTGACGAGTTCGGAACACCCGGCCGCAACCCTGGGAGACGTCCCAGGGACTTCGGGGGCCGTTTT
TGTGGCCCGACCTGAGTCCTAAAATCCCGATCGTTTAGGACTCTTTGGTGCACCCCCCTTAGAGGAGGGATATGTGG
TTCTGGTAGGAGACGAGAACCTAAAACAGTTCCCGCCTCCGTCTGAATTTTTGCTTTCGGTTTGGGACCGAAGCCGC
GCCGCGCGTCTTGTCTGCTGCAGCATCGTTCTGTGTTGTCTCTGTCTGACTGTGTTTCTGTATTTGTCTGAAAATAT
GGGCCCGGGCTAGACTGTTACCACTCCCTTAAGTTTGACCTTAGGTCACTGGAAAGATGTCGAGCGGATCGCTCACA
ACCAGTCGGTAGATGTCAAGAAGAGACGTTGGGTTACCTTCTGCTCTGCAGAATGGCCAACCTTTAACGTCGGATGG
CCGCGAGACGGCACCTTTAACCGAGACCTCATCACCCAGGTTAAGATCAAGGTCTTTTCACCTGGCCCGCATGGACA
CCCAGACCAGGTCCCCTACATCGTGACCTGGGAAGCCTTGGCTTTTGACCCCCCTCCCTGGGTCAAGCCCTTTGTAC
ACCCTAAGCCTCCGCCTCCTCTTCCTCCATCCGCCCCGTCTCTCCCCCTTGAACCTCCTCGTTCGACCCCGCCTCGA
TCCTCCCTTTATCCAGCCCTCACTCCTTCTCTAGGCGCCCCCATATGGCCATATGAGATCTTATATGGGGCACCCCC
GCCCCTTGTAAACTTCCCTGACCCTGACATGACAAGAGTTACTAACAGCCCCTCTCTCCAAGCTCACTTACAGGCTC
TCTACTTAGTCCAGCACGAAGTCTGGAGACCTCTGGCGGCAGCCTACCAAGAACAACTGGACCGACCGGTGGTACCT
CACCCTTACCGAGTCGGCGACACAGTGTGGGTCCGCCGACACCAGACTAAGAACCTAGAACCTCGCTGGAAAGGACC
TTACACAGTCCTGCTGACCACCCCCACCGCCCTCAAAGTAGACGGCATCGCAGCTTGGATACACGCCGCCCACGTGA
AGGCTGCCGACCCCGGGGGTGGACCATCCTCTAGACTGCCatggctctcccagtgactgccctactgcttcccctag
cgcttctcctgcatgcacaagttcagctccagcagagcggcgccgaagtggcaaagcctggagcgtcagtcaagctg
tcctgcaaagcgagtggctatacgttcacgaactactggatgcagtggataaagcaggctcccgggcagggtctgga
gtggattggagccgtctacccaggggacggcgacacccggcacactcaaaagttcaagggcaaggccaccctgaccg
ctgacaagagcacaagcacagcgtacatggaggtgtcctctttgagatccgaagataccgctgtgtattattgtgcc
cggggcttcactgcatacgggatggattactggggacaaggcactaccgtgactgtcagctccgggggtggaggctc
aggcggggggggttcaggaggggggggatctgaaattgtgctgacacagagccctgccacaatgtctgctagccctg
gcgagcgcgtgaccatgtcttgtagcgccagcagcagcgtgtcctacatgcattggtatcaacagaagtccggccag
tctcccaagcggtggatctacgatacaagcaagctggcctccggcgtgcccgccagattttctggcagcggctctgg
aacagattacaccttcaccatctctagcatggaacctgaggattttgccacctactattgccagcagtggtccagca
atcccctgacatttggaggaggcaccaagctggaaattaagagagcggccgcaattgaagttatgtatcctcctcct
tacctagacaatgagaagagcaatggaaccattatccatgtgaaagggaaacacctttgtccaagtcccctatttcc
cggaccttctaagcccttttgggtgctggtggtggttggtggagtcctggcttgctatagcttgctagtaacagtgg
cctttattattttctgggtgaggagtaagaggagcaggctcctgcacagtgactacatgaacatgactccccgccgc
cccgggcccacccgcaagcattaccagccctatgccccaccacgcgacttcgcagcctatcgctccagagtgaagtt
cagcaggagcgcagacgcccccgcgtaccagcagggccagaaccagctctataacgagctcaatctaggacgaagag
aggagtacgatgttttggacaagagacgtggccgggaccctgagatggggggaaagccgagaaggaagaaccctcag
gaaggcctgttcaatgaactgcagaaagataagatggcggaggccttcagtgagattgggatgaaaggcgagcgccg
gaggggcaaggggcacgatggccttttccagggtctcagtacagccaccaaggacaccttcgacgcccttcacatgc
aggccctgccccctcgcggaagcggagctactaacttcagcctgctgaagcaggctggagacgtggaggagaaccct
ggacccatggccctgcccgtcaccgctttgcttctgccactggccttgctgctccacgctcagctccagctccaaga
gtcagggccaggtctcgtgaaaccgagtgagaccctgtccctgacctgcacagtgagtggtggatcaatctcaagct
ctacctactattgggggtggattcggcagccccctagaaaggggcttgagtggattggcagcactcattatcgagga
tctacctattataatccttctctgaaaagcagagttaccatctctgtggatacgtccaaaaatcagttcagtctgaa
ggtatcatccgtgactgctgccgacacggccgtgtactattgcgcgagggagctgacaggtgaggtctttgactact
ggggccagggcacactcgtgaccgtgtcttctgcctcaacaggagggggtgggagtggaggcggtggatcaggggga
ggagggagtgacatccagatgacgcagtccccttccagcttgtccgcatctgtgggtgatagggtcacgattacatg
tagggctagtcagagtatttctagttacctgaattggtaccagcagaaaccaggcaaggcaccaaagttgctcatct
atgcggcctcctctctgcaatctggcgtgccgtccagatttagtggatcaggctccggaaccgatttcacccttacg
atctcctcacttcaacccgaggatttcgccacatattactgtcaacaaagctattctacaccgttcaccttcggacc
ggggacaaaagtggatattaaacgggcggccgcccccaccacgacgccagcgccgcgaccaccaaccccggcgccca
cgatcgcgtcgcagcccctgtccctgcgcccagaggcgtgccggccagcggcggggggcgcagtgcacacgaggggg
ctggacttcgcctgtgatatctacatctgggcgcccctggccgggacttgtggggtccttctcctgtcactggttat
caccctttactgcaacaaacggggcagaaagaagctcctgtatatattcaaacaaccatttatgagaccagtacaaa
ctactcaagaggaagatggctgtagctgccgatttccagaagaagaagaaggaggatgtgaactgtaaCAGCCACTC
GAGGATCC

The nucleic acid molecules can be delivered into cells by art-known methods or as described herein. Genetic modification of a cell can be accomplished by transducing a substantially homogeneous cell composition with a recombinant DNA construct. In certain embodiments, a retroviral vector (e.g., gammaretroviral vector or lentiviral vector) is employed for the introduction of the DNA construct into the cell. For example, a polynucleotide encoding a presently disclosed chimeric receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include at least one presently disclosed chimeric receptor (e.g., two presently disclosed chimeric receptors, e.g., a presently disclosed ADGRE2-targeted chimeric receptor and a presently disclosed CLEC12A-targeted chimeric receptor), a retroviral vector can be employed for transduction, however any other suitable viral vector or non-viral delivery system can be used. The chimeric receptor(s) can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In certain embodiments, the P2A peptide comprises or consists of the amino acid sequence set forth in SEO ID NO: 142, which is provided below:

[SEQ ID NO: 142]
GSGATNFSLLKQAGDVEENPGP

An exemplary nucleotide sequence encoding the amino acid sequence of SEQ ID NO: 142 is set forth in 143, which is provided below:

[SEQ ID NO: 143]
ggaagcggagctactaacttcagcctgctgaagcaggctggagacgtgga
ggagaaccctggaccc

Combinations of retroviral vector and an appropriate packaging line are also suitable, where the capsid proteins will be functional for infecting human cells. Various amphotropic virus-producing cell lines are known, including, but not limited to, PA 12 (Miller et al., (1985) Mol Cell Biol (1985); 5:431-437); PA 317 (Miller., et al., Mol Cell Biol (1986); 6:2895-2902); and CRIP (Danos et al., Proc Natl Acad Sci USA (1988); 85:6460-6464). Non-amphotropic particles are suitable too, e.g., particles pseudotyped with VSVG, RD114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. A cad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol (1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995) 107: 77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEA E dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALEN, CRISPR). Transient expression may be obtained by RNA electroporation.

Any targeted genome editing methods can also be used to deliver at least one presently disclosed chimeric receptor (e.g., two presently disclosed chimeric receptors, e.g., a presently disclosed ADGRE2-targeted chimeric receptor and a presently disclosed CLEC12A-targeted chimeric receptor) to a cell. In certain embodiments, a CRISPR system is used to deliver at least one presently disclosed chimeric receptor (e.g., two presently disclosed chimeric receptors, e.g., a presently disclosed ADGRE2-targeted chimeric receptor and a presently disclosed CLEC12A-targeted chimeric receptor). In certain embodiments, zinc-finger nucleases are used to deliver at least one presently disclosed chimeric receptor (e.g., two presently disclosed chimeric receptors, e.g., a presently disclosed ADGRE2-targeted chimeric receptor and a presently disclosed CLEC12A-targeted chimeric receptor). In certain embodiments, a TALEN system is used to deliver at least one presently disclosed chimeric receptor (e.g., two presently disclosed chimeric receptors, e.g., a presently disclosed ADGRE2-targeted chimeric receptor and a presently disclosed CLEC12A-targeted chimeric receptor).

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of base pairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), metallothionein promoters, or Ubiquitin C promoter), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 5.9. Formulations and Administration

The presently disclosed subject matter also provides compositions comprising the presently disclosed cells (e.g., those disclosed in Section 5.5). In certain embodiments, the composition is a pharmaceutical composition that further comprises a pharmaceutically acceptable carrier.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 104 and about 1010, between about 104 and about 107, between about 105 and about 107, between about 105 and about 109, or between about 106 and about 108 of the presently disclosed cells are administered to a subject. In certain embodiments, between about 10×10⁶ and about 150×10⁶ of the presently disclosed cells are administered to a subject. In certain embodiments, between about 25×10⁶ and about 150×10⁶ of the presently disclosed cells are administered to a subject. In certain embodiments, between about 25×10⁶ and about 50×10⁶ of the presently disclosed cells are administered to a subject. In certain embodiments, at least about 10×10⁶, about 25×10⁶, about 50×10⁶, about 100×10⁶, or about 150×10⁶ of the presently disclosed cells are administered to a subject. In certain embodiments, about 25×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus).

### 5.10. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor in a subject. The presently disclosed cells and compositions comprising thereof can be used for prolonging the survival of a subject suffering from a tumor. In certain embodiments, each of the above-noted method comprises administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

In certain embodiments, the tumor is associated with ADGRE2 and/or CLEC12A. In certain embodiments, the tumor cell expresses both ADGRE2 and CLEC12A. In certain embodiments, the tumor cell overexpresses both ADGRE2 and CLEC12A.

In certain embodiments, the tumor is a cancer. In certain embodiments, the tumor is blood cancer. In certain embodiments, the tumor is selected from the group consisting of multiple myeloma, leukemia, lymphomas, and myeloid malignancies. Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. Non-limiting examples of lymphoma include AIDS-related lymphoma, ALK-positive large B-cell lymphoma, Burkitt's lymphoma, Diffuse large B-cell lymphoma (DLBCL), Follicular lymphoma, Intravascular large B-cell lymphoma, Large B-cell lymphoma arising in HHV8-associated multicentric Castleman's disease, Lymphomatoid granulomatosis, Lymphoplasmacytic lymphoma, Mantle cell lymphoma (MCL), Marginal zone B-cell lymphoma (MZL), Mucosa-Associated Lymphatic Tissue lymphoma (MALT), Nodal marginal zone B cell lymphoma (NMZL), Nodular lymphocyte predominant Hodgkin's lymphoma, Non-Hodgkin's lymphoma, Plasmablastic lymphoma, Primary central nervous system lymphoma, Primary effusion lymphoma, Splenic marginal zone lymphoma (SMZL), and Waldenstrom's macroglobulinemia. The lymphoma can be Hodgkin's lymphoma or non-Hodgkin's lymphoma. Non-limiting examples of myeloid malignancies include myelodysplastic syndromes (MDS), myeloproliferative neoplasms (MPN), myeloid/lymphoid neoplasms (e.g., myeloid/lymphoid neoplasms with eosinophilia and rearrangement of Platelet Derived Growth Factor Receptor Alpha (PDGFRA), Platelet Derived Growth Factor Receptor Beta (PDGFRB), or Fibroblast Growth Factor Receptor 1 (FGFR1), or with PCM 1-JAK 2), acute myeloid leukemia (AML), blastic plasmacytoid dendritic cell neoplasm, B-lymphoblastic leukemia/lymphoma, and T-lymphoblastic leukemia/lymphoma. In certain embodiments, the myeloid malignancies comprise myelodysplastic syndromes.

In certain embodiments, the tumor is acute myeloid leukemia (AML). In certain embodiments, the tumor is relapsed/refractory acute myeloid leukemia (R/RAML).

In certain embodiments, the subject is a human subject. The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

As a consequence of surface expression of at least one presently disclosed chimeric receptor (e.g., two presently disclosed chimeric receptors, e.g., a presently disclosed ADGRE2-targeted CAR and a presently disclosed CLEC12A-targeted CCR), adoptively transferred cells are endowed with augmented and selective cytolytic activity at the tumor site. Furthermore, subsequent to their localization to tumor and their proliferation, the cells turn the tumor site into a highly conductive environment for a wide range of cells involved in the physiological anti-tumor response.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the presently disclosed chimeric receptor(s). The suicide gene can be included within the vector comprising a nucleic acid encoding a presently disclosed chimeric receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed chimeric receptor. The incorporation of a suicide gene into the a presently disclosed chimeric receptor gives an added level of safety with the ability to eliminate the majority of receptor-expressing cells within a very short time period. A presently disclosed cell incorporated with a suicide gene can be pre-emptively eliminated at a given timepoint post the cell infusion, or eradicated at the earliest signs of toxicity.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—Targeting ADGRE2 and CLEC12A

The presently disclosed subject matter evaluates a novel combinatorial CAR format for R/RAML that has the potential to provide improved safety and efficacy relative to alternative CAR therapies currently under clinical investigation.

Application of CAR therapy in AML is challenged by its intra- and interindividual phenotypic heterogeneity, and the ideal CAR target needs to be validated. To identify potential CAR targets, the available transcriptomics and proteomics datasets from malignant and normal tissues were analyzed and flow cytometric analysis of primary AML patient samples, healthy bone marrow hematopoietic stem/progenitor cells (HSPCs), and healthy primary T cells were carried out (Perna et al., Cancer Cell 2 (4): 506-519.e5. (2017)). None of the targets showed a surface expression profile comparable with that of CD19, which was expressed at high levels in virtually all B cell leukemia cells, completely absent from HSPCs and T cells, and undetectable systematically outside B cell areas. However, the analysis identified the adhesion G protein-coupled receptor E2 (ADGRE2), a novel potential CAR target for AML. Pair-wise analyses of the potential CAR targets suggested that, consistent with AML clonal heterogeneity, a combinatorial CAR targeting approach can be potentially efficacious in eradicating AML tumors while minimizing toxicity.

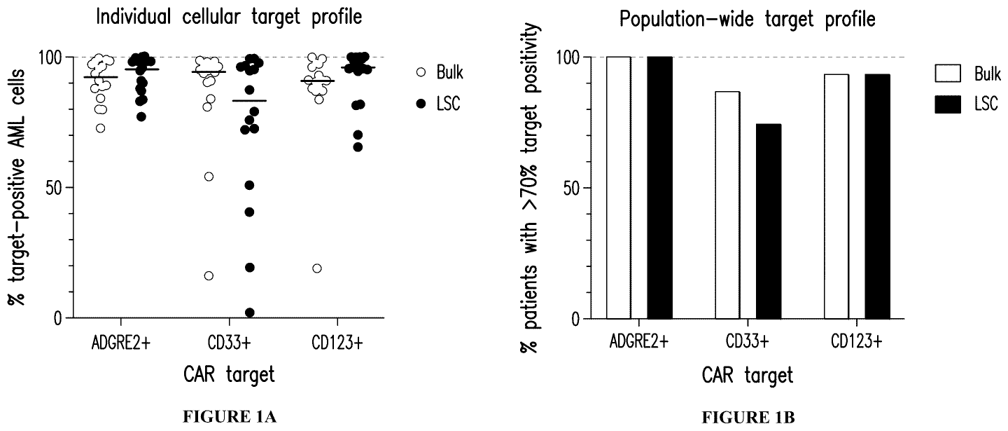

ADGRE2 (EMR2) is a member of the epidermal growth factor (EGF)-TM 7 family of proteins (Lin et al., Genomics 41.3 (1997): 301-308). Its expression is restricted to monocytes/macrophages and is not upregulated in activated T and B cells (Lin et al., Genomics 67.2 (2000): 188-200). Flow cytometric analyses revealed ADGRE2 positivity on >90% of AML cells in the majority of the analyzed R/RAML patient population (FIG. 1). ADGRE2 was found to be positive both in the total AML bulk population and, most importantly, in the therapeutically relevant fraction of leukemic stem cells (LSC). Other candidate targets such as CD33 and CD123 were also found to be positive in the majority of patients, however not as consistently as ADGRE2, especially in LSCs. Therefore, ADGRE2 was selected as the CAR target as it can offer a higher chance for achieving complete remission in R/RAML patients.

C-type lectin domain family 12 member A (CLEC12A/CD371) is a well-described candidate target in AML, and it is expressed in the majority of AML patients (Perna et al., Cancer cell 32.4 (2017): 506-519; Haubner et al., Leukemia 33.1 (2019): 64-74; Bakker et al., Cancer research 64.22 (2004): 8443-8450). Other groups are already pursuing it as a CAR target in ongoing Phase 1 clinical trials in the US (Tashiro et al., Molecular Therapy 25.9 (2017): 2202-2213).

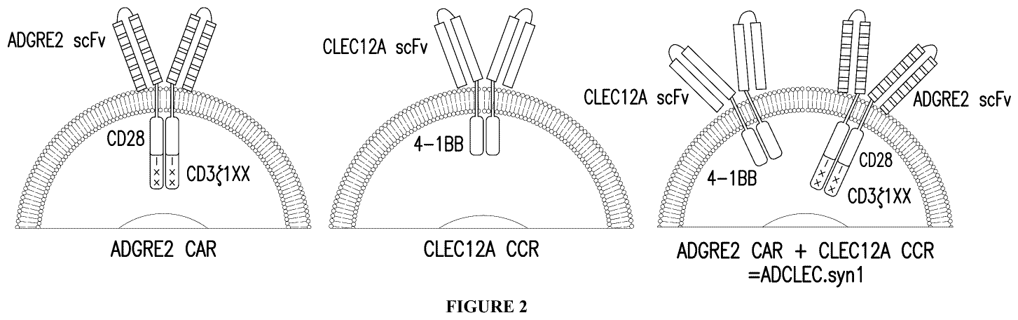

The presently disclosed subject matter proposes using a combinatorial CAR-CCR vector, termed ADCLEC.syn1 (FIG. 2), which encodes a CAR specific for ADGRE2 and a CCR specific for CLEC12A to treat R/RAML. The CAR comprises a CD28 costimulatory domain and an attenuated CD3ζ1XX activation domain. It was previously demonstrated that genetic modification of the ITAM motifs of the CD3ζ signaling domain provided an enhanced therapeutic benefit by achieving a favorable balance of effector and memory signatures, thereby enhancing the persistence of functional CAR T cells in a pre-B acute lymphoblastic leukemia NALM 6 mouse model (Feucht et al., Nature medicine 25.1 (2019): 82-88). The CCR provides 4-1BB co-stimulation to enhance T cell persistence and prevents antigen-low AML escape; it does not comprise a CD3ζ domain and thus does not mediate cell killing like a CAR.

ADGRE2 expression is commonly upregulated in AML cells as compared to non-malignant cells. Considering the ADGRE2 expression in some normal cell types, including HSCs, a targeting strategy that would preferentially target AML cells was designed. In this approach, a first step was decreasing the affinity and activation potential of the ADGRE2 CAR to improve the safety of the CAR therapy, and a second step was rescuing ADGRE2 CAR engagement against LSCs by co-targeting CLEC12A (CD371), a second molecule expressed on LSCs but not HSCs, to prevent AML escape. CLEC12A was targeted through a chimeric costimulatory receptor (CCR), which provides increased avidity and co-stimulation but does not initiate cytolytic activity. Thus, the CCR assists the CAR to detect ADGRE2 in AML cells.

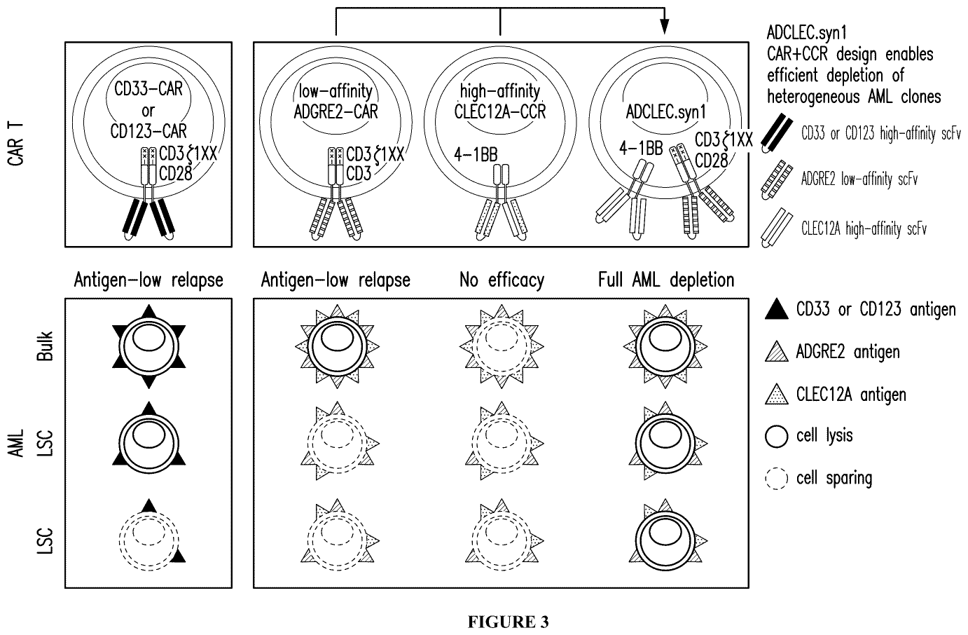

CAR signaling is triggered upon target engagement of the scFv portion of the CAR, which depends on CAR scFv affinity and CAR target antigen density. The ADGRE2 CAR scFv affinity were optimized so that healthy cells, which have lower ADGRE2 antigen levels than AML tumor cells, did not trigger CAR activation and were therefore spared, adding an important safety feature but at the same time increasing the risk of AML escape via downregulation of ADGRE2 antigen levels (FIG. 3). To mitigate the risk of ADGRE2-low AML escape, the addition of the CLEC12A CCR increased the overall avidity to AML cells and thereby “compensated” for the lower-affinity ADGRE2 CAR, while it does not initiate the elimination of ADGRE2-negative/CLEC12a-positive cells. As the CLEC12A CCR lacks a CD3ζ domain, it did not function as a CAR. Isolated CCR activation in the absence of simultaneous CAR activation did not trigger T cell activation or cytotoxicity (FIG. 3).

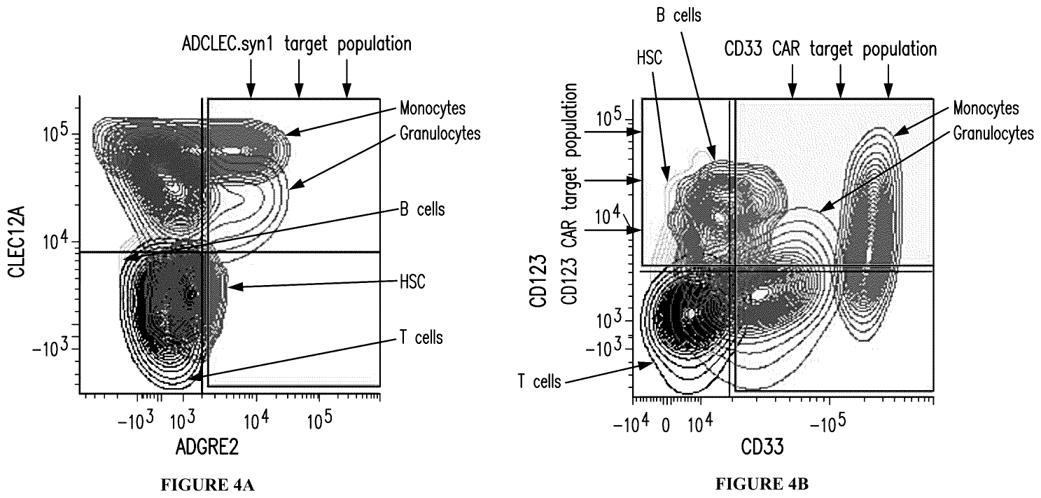

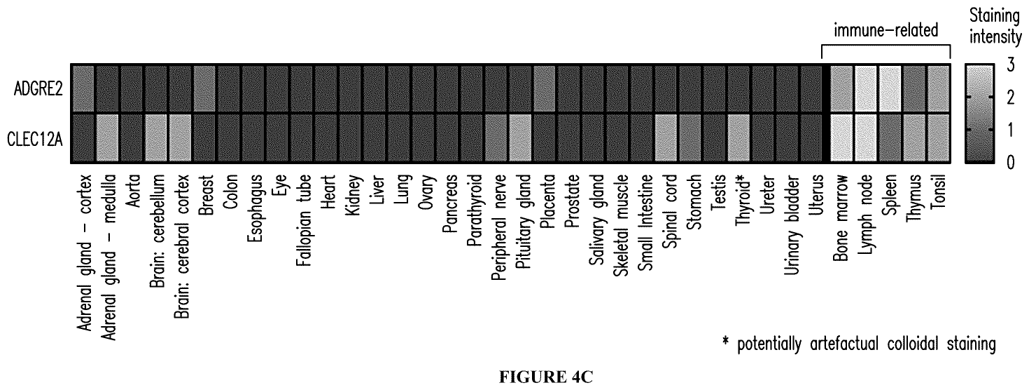

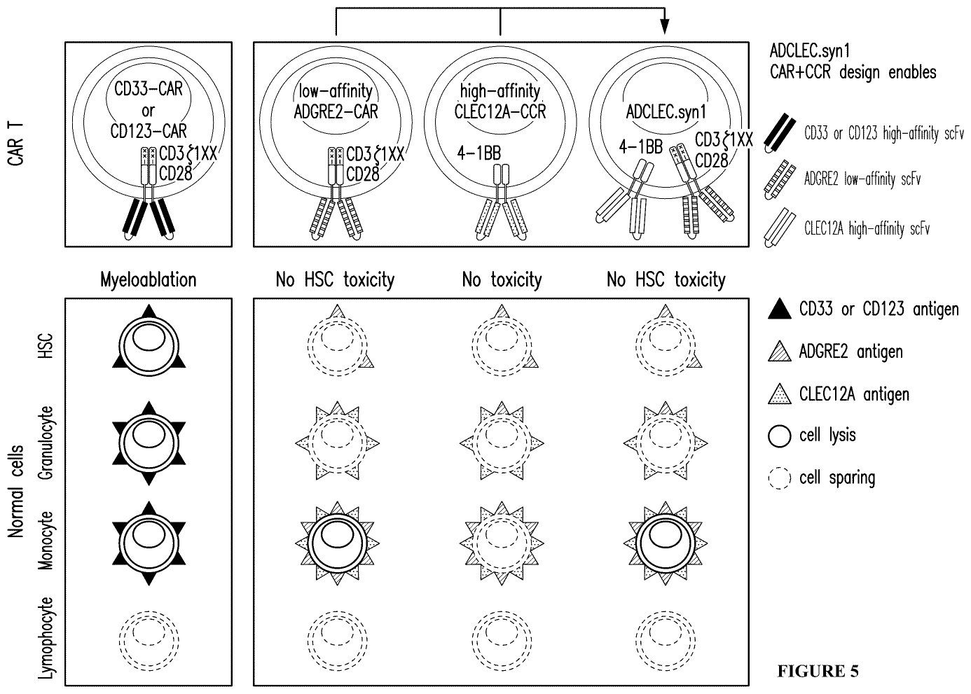

The choice of targets and chimeric receptor design was made under consideration of safety features linked to the target expression in normal tissues and normal hematopoiesis. Co-expression of ADGRE2 and CLEC12A was limited to the monocyte subset, while all other major lineages had either a low or undetectable expression of ADGRE2 by flow cytometry (FIG. 4A) suggesting that A DCLEC.syn1 would display diminished activity against HSCs and granulocytes (FIG. 5). In comparison, the alternative targets CD33 and CD123, which were co-expressed in HSCs, led to greater CAR-mediated hematologic toxicity (FIGS. 4B and 5). Using commercially available antibodies for ADGRE2 and CLEC12A for IHC, the presently disclosed subject matter demonstrated that non-hematopoietic tissues had a largely non-overlapping expression profile with the majority of the detected signal associated with tissue-resident myeloid-derived cells such as macrophages (FIG. 4C). Taken together, these target expression data indicate that combinatorial targeting of ADGRE2 with a CAR and CLEC12A with a CCR via ADCLEC.syn1 vector can minimize the toxicity of the CAR-CCR T cells to normal hematopoietic and non-hematopoietic tissues relative to A DGRE2 targeting alone and provides a higher chance of sparing HSC compared to CD33 or CD123 CAR T cells.

In summary, the ADGRE2 CAR and CLEC12A CCR combined in the ADCLEC.syn1 vector mitigated the risk of ADGRE2 antigen escape and enhanced overall CAR T cell efficacy via optimized delivery of CD28 and 4-1BB costimulatory signaling in the context of fine-tuned CD3ζ1XX CAR signaling, but without cumulating potential on-target/off-tumor toxicity as would arise by combining two CARs. This rational combinatorial choice of target and chimeric receptor design can reduce the risk of unwanted toxicity while enhancing the targeting of R/R AML, including LSCs.

### Example 2—ADGRE2 scFv

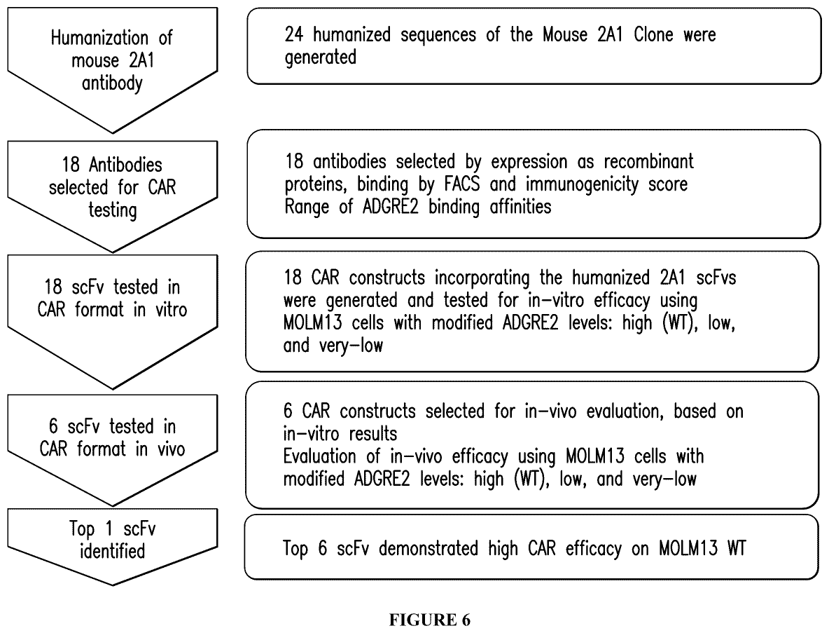

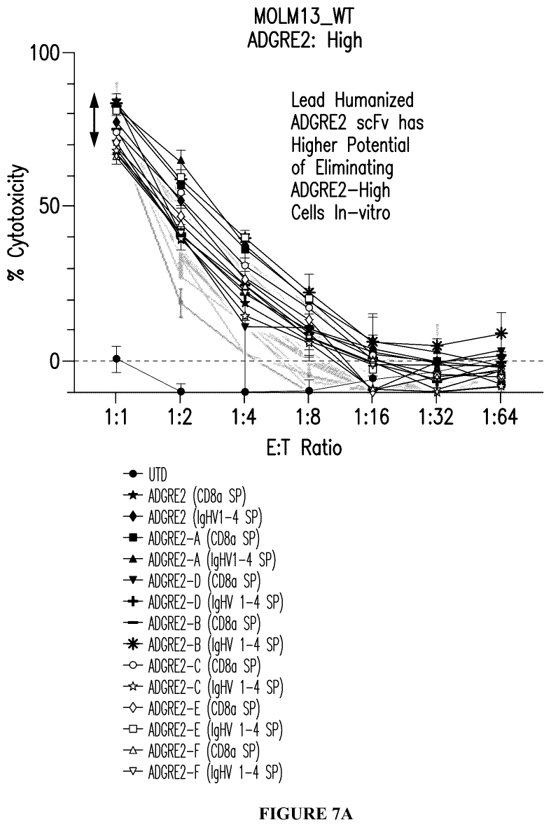

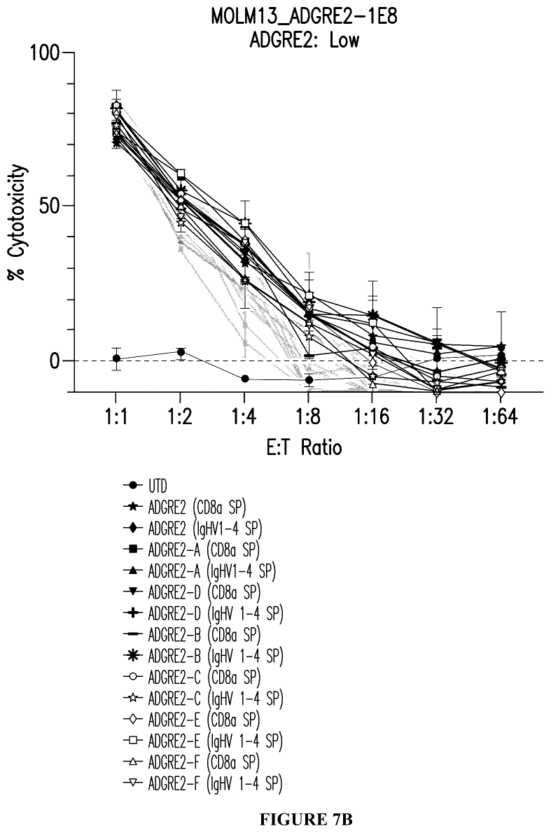

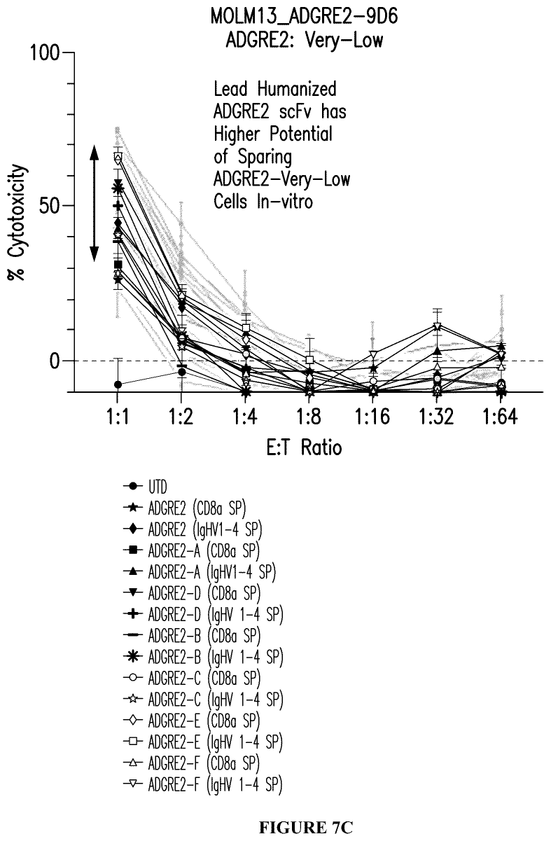

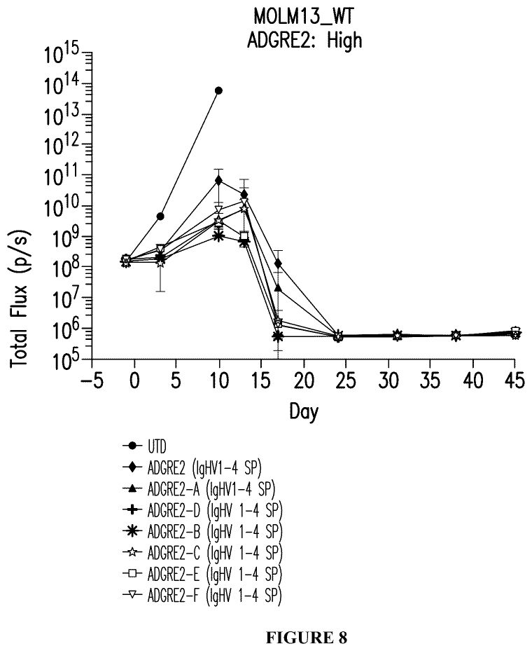

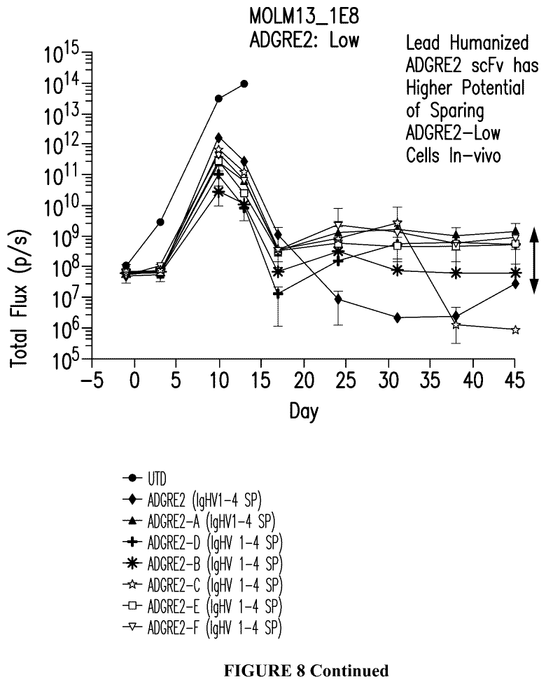

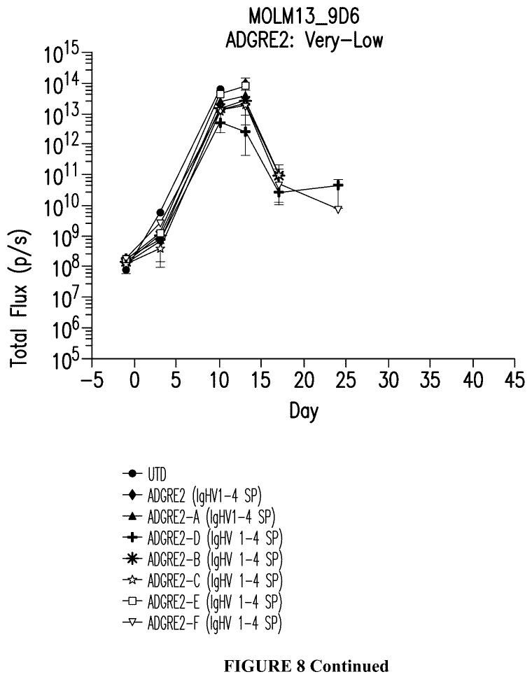

The ADGRE2 single-chain fragment variable (scFv) in the ADCLEC.syn1 vector was selected from 24 versions of the humanized recombinant antibodies generated using the peptide sequence of the monoclonal anti-human ADGRE2 clone 2A 1 antibody. The binder selection scheme is present in FIG. 6. The mouse monoclonal anti-human ADGRE2 clone 2A 1 is a widely used antibody to detect ADGRE2 expression in human samples (Boyden et al., N Engl J Med. 374 (7): 656-63. (2016)). Based on (1) the expression of the humanized 2A 1 antibody variants, (2) the binding of the recombinant antibodies to ADGRE2-overexpressing murine lymphoma EL4 cells as measured by flow cytometry, and (3) the immunogenicity score, 18 humanized recombinant antibodies were selected from the pool of candidates. Next, VH and VL domains were identified to generate 18 scFv candidates that were subsequently integrated into the SFG based gammaretroviral CAR vectors and tested in functional CAR assays (FIGS. 7A-7C and 8).

ADGRE2-specific CAR T cells were produced by transducing T cells with the SFG-gammaretroviral vector expressing each of the humanized 2A 1 scFv in the CAR cassette, and CAR T cell cytotoxicity was measured using the AML cell line M OLM 13. Cognizant of ADGRE2 expression in some normal cell types, including HSCs, which express lower levels of ADGRE2 than AML cells, MOLM 13 cell lines were genetically modified for high (WT), low, or very low levels of ADGRE2 expression, with coexpression of low (WT) or high levels of CLEC12A (see Table 17 below).

TABLE 17
MOLM13 cell lines for CAR studies used for identification of
lead ADGRE2 and CLEC12A scFvs

| | ADGRE2 level | CLEC12A level |
|---|---|---|
| Target cell line | (MFI relative to WT) | (MFI relative to WT) |
| MOLM13-WT | high (1×) | low (1×) |
| MOLM13_ADGRE2-1E8 | low (0.2×) | low (1×) |
| MOLM13_ADGRE2-9D6 | very-low (0.1×) | low (1×) |
| MOLM13_ADGRE2- | low (0.2×) | high (25.9×) |
| E6_CLEC12A-C6 | | |
| MOLM13_ADGRE2- | very-low (0.1×) | high (15.7×) |
| 9D6_CLEC12A-B6 | | |

These modified MOL M 13 cell lines allowed the identification of the humanized ADGRE2 scFv with the highest potential of efficiently depleting ADGRE2-high AML cells while sparing ADGRE2-low normal cells and thereby maintaining a favorable safety profile (FIGS. 7A-7C and 8). The in vitro CAR screening included 18 different ADGRE2 scFv candidates in the context of two different signal peptides (CD8a or IgHV 1-4). A set of 6 scFvs that mediated the most suitable pattern of in vitro target cell lysis (maximum lysis of ADGRE2-high cells and minimum lysis of ADGRE2-very-low cells) were identified with a high similarity between the two tested signal peptides (FIGS. 7A-7C). Then, these 6 scFvs were tested in in vivo model using the same AML cell line M OLM 13 with high (WT), low or very low levels of ADGRE2. Here, the ADGRE2 scFv ADGRE2-A showed the most suitable in vivo response pattern, with maximum efficacy against target cells with high ADGRE2 levels and reduced/absent efficacy against target cells with low/very-low ADGRE2 levels, respectively. Therefore, ADGRE2-A was selected as ADGRE2 scFv and with the CD8a signal peptide.

### Example 3—CLEC12A scFv

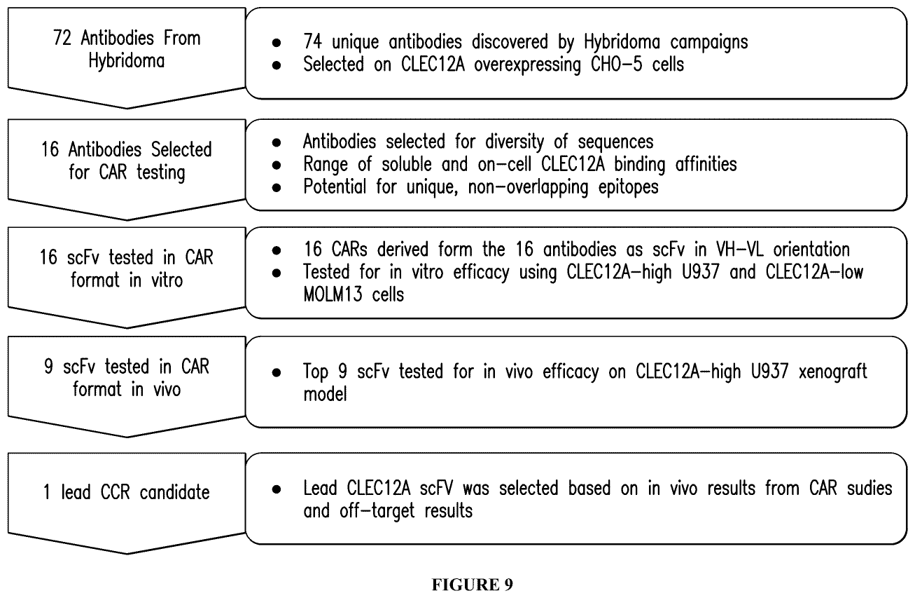

The human CLEC12A scFv was selected from 74 unique human antibodies produced by hybridomas generated by fusing P3X 63A g8U.1 cell with lymphocytes isolated from mice immunized with recombinant human CLEC12A protein. Positive clones were selected by using on-cell binding to CLEC12A-expressing CHO-S cells and surface plasmon resonance assay. The selection scheme of the scFv selection is shown in FIG. 9.

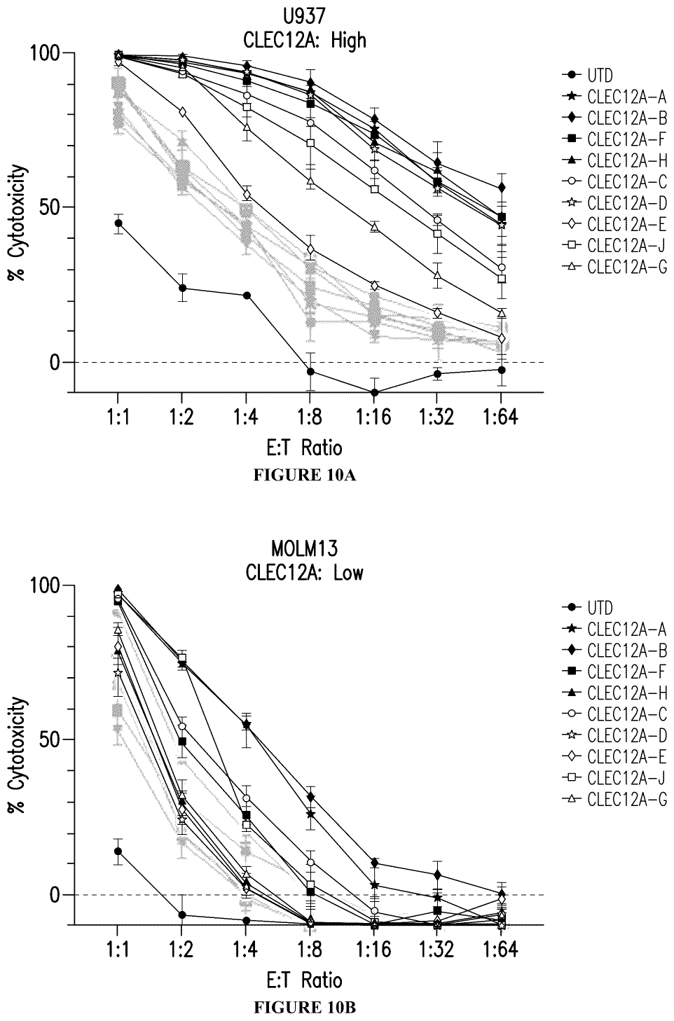

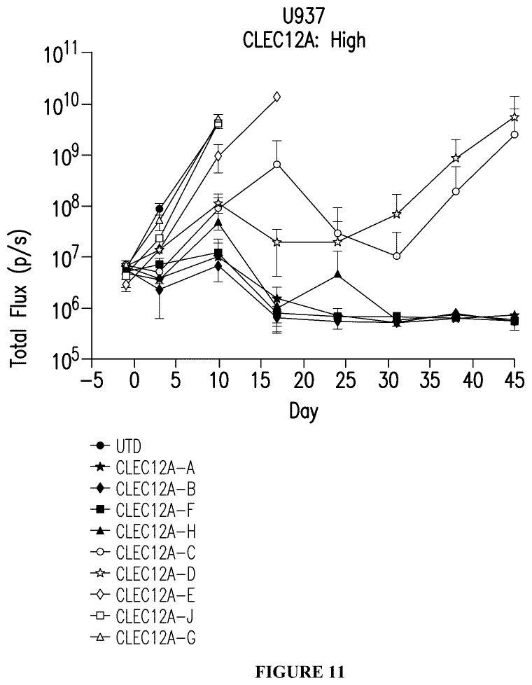

16 different human CLEC12A scFv candidates were tested in a 28z1XX TRAC-CAR format using in vitro 18 h CAR cytotoxicity assays and in vivo xenograft models. Different from the strategy for A DGRE2 scFv identification, here the efficacious scFv were prioritized in the context of both high and low target antigen levels, considering that the eventual application of the scFv would be in a CCR, not CAR, and high target sensitivity of the CLEC12A scFv would therefore not be associated with increased toxicity. In vitro screening of 16 scFvs in the context of U937 (CLEC12A-high) and MOLM 13 (CLEC12A-low) cell lines identified 9 highly potent CLEC12A scFvs (FIGS. 10A and 10B). Subsequent in vivo screening of these 9 scFvs identified the lead CLEC12A scFv (CLEC12A-A) (FIG. 11).

### Example 4—Off-Target Binding of ADGRE2 scFv and CLEC12A scFv

The off-target binding interactions of ADGRE2 scFv (ADGRE2-A) and CLEC12A scFv (CLEC12A-A) to human plasma membrane proteins were carried out in a 4-phase approach: (1) pre-screening to determine the optimal concentrations of purified recombinant ADGRE2 scFv-Fc and CLEC12A scFv-Fc test proteins for the screening; (2) screening for binding of the test proteins against fixed HEK 293 cells overexpressing 5,474 full-length human plasma membrane proteins and cell surface tethered human secreted proteins and 371 human cell surface heterodimers, which identified library hits; (3) confirmatory/specific screens using fixed-cell and live-cell microarrays; (4) flow cytometry-based follow-on study to investigate further identified test protein-specific interactions on live cells; and (5) cell-to-cell binding assays to confirm positive hits. The table below summarizes the off-binding data for ADGRE2 scFv and CLEC12A scFv.

TABLE 18
Binding of ADGRE2 scFv and CLEC12A scFv to proteins identified in
the fixed and live-cell screenings

| | Interacting | | | |
|---|---|---|---|---|
| | Proteins | | | |
| | identified | | Flow | Cell-to- |
| Test | from | | Cytometry | Cell |
| Article | the screens | Protein Name | EC50 | Binding |
| ADGRE2 | ADGRE2 | Adhesion G | Not tested | Not tested |
| | | protein-coupled | | |
| scFv-Fc | | receptor E2 | | |
| CLEC12A | CLEC12A | C-type lectin | 0.081 μg/mL | medium to |
| | | domain family | | |
| scFv-Fc | | 12 member A | | strong |
| | SECTM1 | Secreted and | 799.5 μg/mL | weak to |
| | | transmembrane | | very weak |
| | | protein 1 | | |
| | RTN4R | Reticulon-4 | >300 μg/mL | Not tested |
| | | receptor | | |

From the 5,845 plasma membrane proteins tested, the purified ADGRE2-scFv-Fc protein bound only its primary target cell surface protein, ADGRE2, both in fixed-cell and live-cell microarrays. In addition, ADGRE2-scFv-Fc did not bind to other ADGRE proteins ADGRE1, ADGRE3, and ADGRE5.

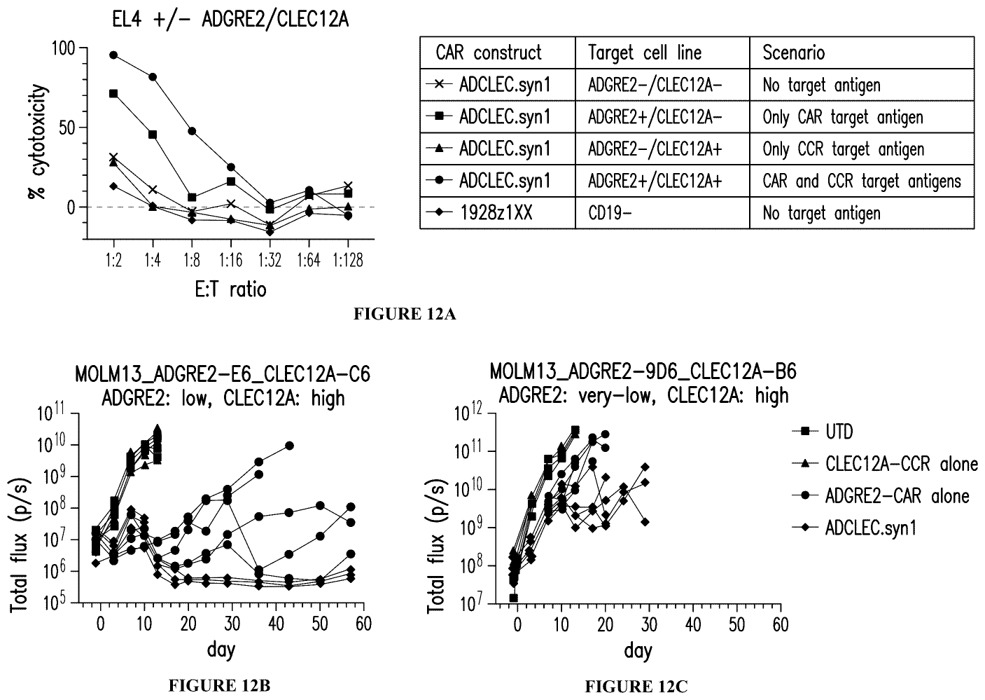

CLEC12A scFv-Fc showed a strong specific interaction with its primary target, CLEC12A. It also had a low but detectable interaction with SECTM1 in both fixed-cell and live-cell microarrays and with RTN3R in live-cell microarray only. Follow-on studies using dose titration and flow cytometry experiments using purified CLEC12A scFv-Fc demonstrated that CLEC12A scFv EC50s against SECTM1 and RTN4R were 800-fold and >3,700-fold greater than against the primary target CLEC12A, respectively. Furthermore, although the CLEC12A CCR-expressing Jurkat cells bound CLEC12A-overexpressing HEK 293 cells, CLEC12A CCR-expressing Jurkat cells had very weak to weak binding to SECTM1-overexpressing HEK 293 cells. Together, these data suggest that the binding of CLEC12A scFv-Fc to SECTM1 and RTN4R may not be physiologically relevant. Any potential off-target binding of the CLEC12A CCR does not lead to cytotoxicity without simultaneous ADGRE2 CAR activation, adding another safety layer (FIG. 12).

### Example 5—Validation of Combinatorial CAR+CCR Targeting Concept of ADCLE C.Syn1

As outlined in the schematics depicted in FIGS. 3 and 5, the presently disclosed subject matter hypothesized that the combinatorial CAR+CCR design of ADCLEC.syn1 would allow efficient elimination of AML while sparing normal cells with low CAR target levels. Importantly, the CCR alone should assist and enhance CAR-mediated cytolysis, however, not trigger cytolysis independently. This approach was validated in in vitro and in vivo models (FIG. 12). First, an in vitro model was established with a target cell line expressing either no target (ADGRE2−/CLEC12A−), CAR target alone (ADGRE2+/CLEC12A−), CCR target alone (ADGRE2−/CLEC12A+), or both CAR and CCR targets (ADGRE2+/CLEC12A+). The CCR target alone did not trigger in vitro killing, however, the CAR target alone triggered killing, which was further enhanced when the CCR target was co-expressed on the target cell line (FIG. 12A). Next, it was demonstrated in vivo that ADGRE2 CAR signaling alone was not sufficient to fully eradicate ADGRE2-low cells (modeling normal cells), however, combined ADGRE2 CAR and CLEC12A CCR signaling allowed for complete and durable AML remission (FIGS. 12B and 12C). Overall, these results indicate the potential of ADCLEC.syn1 to spare normal cells with low levels of CAR target while efficiently eradicating AML.

### Example 6—SF G-ADCLEC.syn1 Retroviral Vector

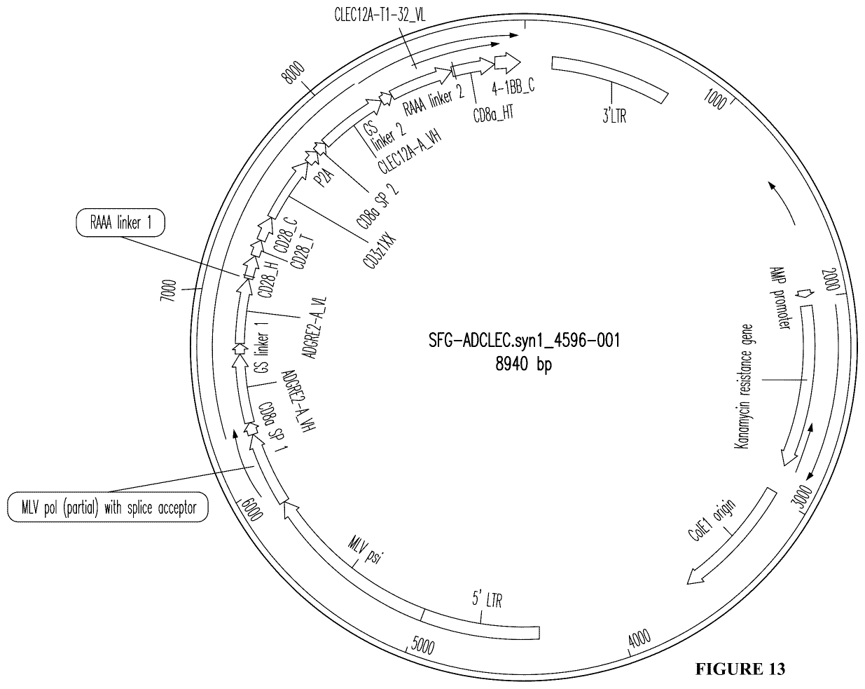

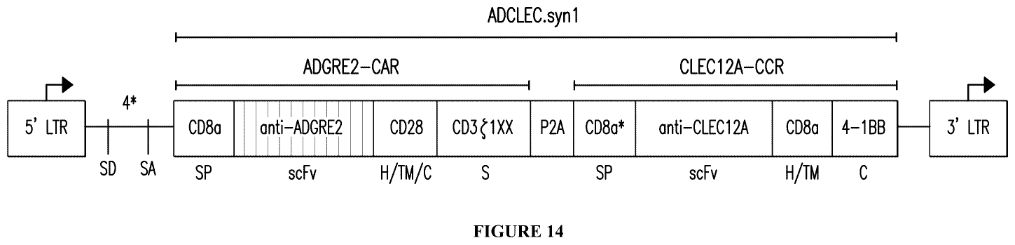

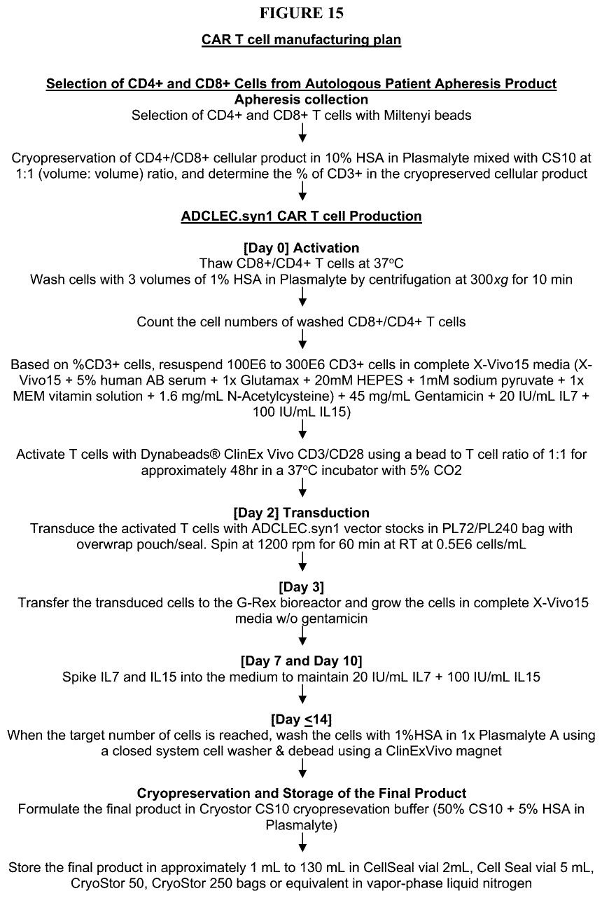

The gammaretroviral plasmid SFG-ADCLEC.syn1 (FIG. 13) was constructed and the identity of the plasmid was verified by Sanger sequencing analysis. The gammaretroviral vector SFG-ADCLEC.syn1 expresses both the ADGRE2-specific CAR and CLEC12A-specific CCR linked through a P2A element, as shown in FIG. 14.

The SFG plasmid is used to transiently transfect 293Vec-RD 114 cells, a HEK 293-based packaging cell line that produces retroviral vectors pseudotyped with feline RD 114 envelope. The vector supernatant that is harvested from the 293Vec-RD 114™ packaging cells is subsequently used to transduce the 293Vec-GalV™, a second HEK 293-based packaging cell line pseudotyped with the gibbon ape leukemia virus envelope protein, to generate a stable producer cell population expressing the 293Vec-GalV-SFG-ADCLEC.syn1. Single clones are isolated from the 293Vec-GalV™ SFG-ADCLEC.syn1 vector producer population, and several seed banks are generated and best titer clones are characterized. The best producer cell line, the lead clone, is used to generate the Master Cell Bank (MCB) from the seed banks. Retroviral Vector stock (VS) is manufactured from one vial of the MCB.

### Example 7—Ex Vivo Transduction of Autologous T Cells with SFG-ADCLEC.Syn1

Activated, autologous patient-derived peripheral blood T cells are transduced ex vivo with Gal V pseudotyped SFG-ADCLEC.syn1 retroviral vector stocks. CD8+ and CD4+ T cells are positively isolated using CD8 and CD4 Microbeads using the Prodigy CliniMACS plus instrument (Miltenyi Biotec) and activated with Dynabeads (ThermoFisher). The initial positive selection of CD8+ and CD4+ T cells mainly minimizes the number of B lymphocytes, plasma cells, and monocytes in the transduced T cell product. Fluorescence-activated cell sorting (FA CS) assays using dye-labeled purified ADGRE2 or CLEC12A polypeptides are carried out to measure the expression of the ADGRE2 CAR and CLEC12A CCR to determine transduction efficiency. Products for patient administration have transduction efficiency greater than or equal to 4%.

### Example 8—Dosage Form, Route of Administration, and Dosing Regimen (Frequency and Duration)

ADCLEC.syn1 CAR T cells are provided in a cryopreserved bag, thawed at the facility, e.g., hospital facility, and administered as an intravenous (IV) infusion via gravity. The planned dose levels to be evaluated during dose escalation are described in Table 19 below.

TABLE 19
Planned ADCLEC.syn1 CAR T cells Dose Levels

| Dose Level (DL) | ADCLEC.syn1 CAR T cell dose (flat) |
|---|---|
| −1 | 10 × 10⁶ |
| 1 | 25 × 10⁶ |
| 2 | 50 × 10⁶ |
| 3 | 100 × 10⁶ |
| 4 | 150 × 10⁶ |

The starting dose for ADCLEC.syn1 CAR T cells is 25×10⁶ CAR T cells. This dose is significantly lower than the approved dosing of CD19 CARs (2×10⁶ CAR T cells/kg for axicabtagene and 6-60×10⁷ CAR T cells for tisagenlecleucel), and also lower than the doses of BCMA CARs currently being investigated in multiple myeloma (50-800×10⁶ CAR T cells). Dose escalation follows a standard 3+3 design. Following identification of the RP2D, the RP2D dose cohort can be expanded.

### Example 9—Clinical Study

The present example is a phase 1, open-label, dose-escalation study to evaluate safety and activity of ADCLEC.syn1 CAR T cells disclosed herein in adult patients with R/RAML. A standard 3+3 dose escalation scheme is used to determine the maximum tolerated dose (MTD) or maximal administered dose (MAD) if the MTD is not reached. Once the MTD or MAD is identified, the cohort is expanded to include additional patients to further evaluate the safety and activity of ADCLEC.syn1 CAR T cells.

Cohorts of 3-6 patients are infused with escalating doses of ADCLEC.syn1 CAR T cells to establish the MTD. There are 4 planned dose levels: 25×10⁶, 50×10⁶, 100×10⁶, 150×10⁶ CAR T cells. A standard 3+3 dose escalation design is implemented starting from dose 25×10⁶. 3-6 patients are treated in each cohort and dose escalation proceeds to the next cohort if less than 33% of patients in a cohort experience unanticipated dose-limiting toxicity (DLT). If unacceptable toxicity is seen in 1 of 3 patients in any given cohort, up to 6 patients are treated in that cohort using a conventional dose escalation scheme. If 2 of 6 patients in any given cohort experience unacceptable toxicity, the MTD of T cells has been exceeded, and established at the previous cohort dose level. If the first dose level exceeds the MTD, a subsequent cohort of 3-6 patients is treated at the −1 dose level of 10×10⁶ ADCLEC.syn1 CAR T cells.

Conditioning or lymphodepleting chemotherapy. Patients receive conditioning chemotherapy consisting of fludarabine 25 mg/m² on days −4, −3, −2, and cyclophosphamide 500 mg/m² on days −3, −2, followed by CAR T cell infusion on day 0.

Bridging therapy. At the discretion of the treating physician, bridging therapy may be considered. Bridging therapy may be administered after leukapheresis and must be discontinued at least 1 week prior to administration of conditioning chemotherapy, with the exception of hydroxyurea which can be continued until at least 24 hours prior to the start of conditioning chemotherapy. Subjects should be restaged after the end of the bridging therapy and prior to start of conditioning chemotherapy or within 48 hours prior to administration of conditioning chemotherapy.

Disease response assessment. Bone marrow biopsy is obtained at day 28-35 of CAR T cell infusion for response assessment and is assessed by the ELN criteria.

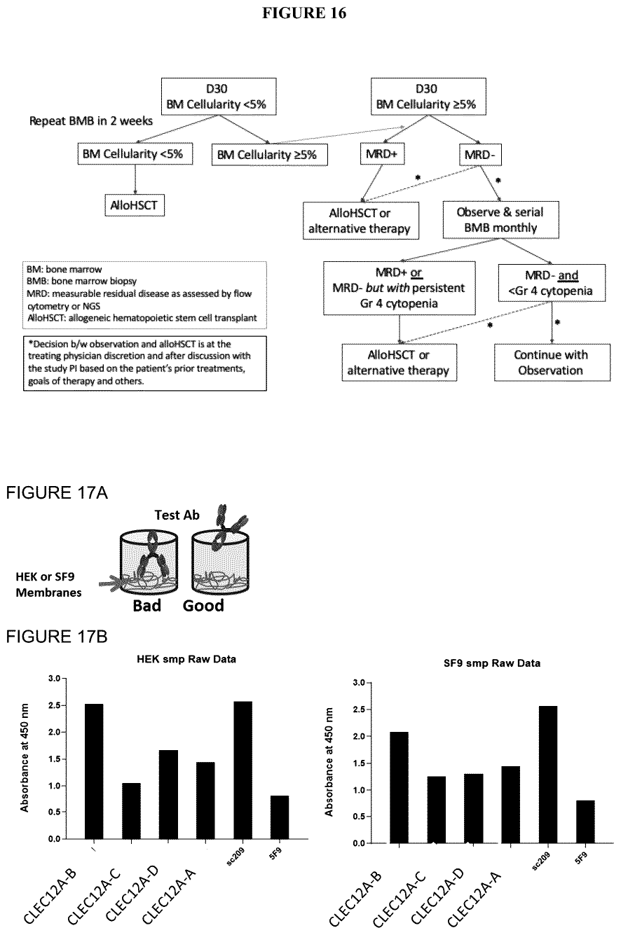

Treatment course following D 28-35 disease response assessment. Following the disease response assessment, subsequent care is guided per the suggested algorithm as shown in FIG. 16.

Toxicity assessment. At each study visit, toxicities are graded according to the National Cancer Institute (NCI) Common Terminology Criteria for Adverse Events (CTCAE) Version 5.0 and ASTCT consensus grading for cytokine release syndrome (CRS) and immune effector cell-associated neurotoxicity syndrome (ICA NS).

Pharmacokinetic assessment. Blood tests for pharmacokinetic measurements are performed prior to dosing, on day 1, 3, 5, 7, 14, 21, and 28-30. Subsequent blood samples are collected on months 2, 3, 6, 9, 12 and 24 months.

ADCLEC.syn1 CAR T cell Dose Levels. The starting dose for ADCLEC.syn1 CAR T cells is 25×10⁶ CAR T cells. This dose is significantly lower than the approved dosing of CD19 CARs (2×10⁶ CAR T cells/kg for axicabtagene and 6-60×10⁷ CAR T cells for tisagenlecleucel), and also lower than the doses of BCMA CARs currently being investigated in multiple myeloma (50-800×10⁶ CAR T cells). Assuming a body weight of 70 kg, the proposed starting dose translates to 2×10⁵ CAR T cells/kg and is comparable (or slightly lower) compared to other autologous AML CAR starting doses.

Dose Limiting Toxicity (DLT). A DLT is defined as any of the following adverse event (AE) that occurs within 28 days of the ADCLEC.syn1 CAR T cell infusion, based on Common Terminology Criteria for Adverse Events (CTCAE) v5.0, or the ASTCT Consensus Grading guidelines for Cytokine Release Syndrome (CRS) and Immune Effector Cell Associated Neurotoxicity Syndrome (ICA NS): (a) any Grade ≥3 non-hematologic A E to vital organs (excluding CRS and ICANS) which fail to resolve to grade 2 or below within 7 days; (b) Grade 4 neutropenia or thrombocytopenia and day 28 bone marrow cellularity of <5% in the absence of persistent AML. Persistent cytopenias that were present at baseline (pre-conditioning chemotherapy and T cell infusion) that persist until day 30 in the absence of active disease is not considered a DLT; (c) ≥ Grade 3 infusion reaction; (d) Grade 3 or 4 ICANS of any duration; (e) Grade 4 CRS of any duration; (f) Grade 4 tumor lysis syndrome; (g) Grade 4 capillary leak syndrome; and (h) any Grade 5 event related to ADCLEC.syn1 CAR T cells.

Dose Escalation. The objective of Dose Escalation is to determine the MTD/MAD of ADCLEC.syn1 CAR T cells. Dose escalation follows 3+3 dose escalation rules as follows: (a) subjects are enrolled in cohorts of three subjects per dose level with a minimum of 10 days between ADCLEC.syn1 CAR T cells dosing of the first and the second subject within a cohort; (b) if no DLT is reported in the initial cohort of three subjects, dose escalation can proceed to the next higher dose level; (c) if one subject has a DLT, the cohort is expanded up to six subjects. If no additional DLTs are observed, that dose level is considered tolerable and dose escalation can proceed to the next higher dose level; (d) a subject who discontinues study treatment or withdraws from the study prior to the end of the DLT observation period for reasons other than DLT is considered unevaluable for DLT and is replaced; (e) if more than one subject out of six enrolled subjects enrolled at the same dose level cohort has a DLT, the MTD has been exceeded. Further enrollment in that dose cohort stops; six subjects at a lower dose level are enrolled to determine the MTD per 3+3 dose escalation rules; (f) if the dose level that exceeded the MTD is ≥2 times higher than the previously highest tolerable dose level, another dose level approximately midway between these two levels can be enrolled to determine the MTD per 3+3 dose escalation rules; (g) the MTD is the highest dose level cohort with at most one DLT in six evaluable subjects.

Dose Expansion. The objectives of Dose Expansion are to further assess safety and tolerability and to identify activity signals to guide and support future development. The dose of ADCLEC.syn1 CAR T cells in the Dose Expansion is the MTD/MAD that was established during Dose Escalation. During the Dose Expansion phase, subjects with R/RAML are treated in the same manner as in the Dose Escalation phase and includes up to approximal 12 subjects.

Investigational Medicinal Product. ADCLEC.syn1 CAR T cells are provided as a cryopreserved bag, thawed at the facility, and administered as an intravenous (IV) infusion via gravity. The planned dose levels is evaluated during dose escalation described herein. ADCLEC.syn1 CAR T cells are administered on Day 0.

Non-Investigational Medicinal Products. Conditioning chemotherapy includes (a) cyclophosphamide (CY), 500 mg/m² IV on Days −3 and −2 of each treatment cycle; and (b) fludarabine (FLU), 25 mg/m² IV on Days −4, −3 and −2 of each treatment cycle. The dose and schedule of CY and FLU may be modified based on cytopenia and medical comorbidities.

Study Endpoints. Primary endpoints include the incidence and nature of DLTs when ADCLEC.syn1 CAR T cells are administered in patients with R/RAML. Secondary endpoints include (a) incidence, nature and severity of adverse events of ADCLEC.syn1 CAR T cells; (b) investigator-assessed objective-response rate (ORR), defined as the proportion of subjects who achieve a Complete Response (CR), CR with incomplete hematologic recovery (CRi), morphologic leukemia free state (MLFS) and partial response (PR), as per the ELN response criteria; (c) progression-free survival (PFS), defined as the duration from first dose of ADCLEC.syn1 CAR T cells (Day 0) to progressive disease (PD), or to the day of death for any reason, whichever occurs earlier; (d) one-year overall survival (OS), defined as the percentage of subjects who are alive at one year from initiation of study treatment; and (e) cellular kinetics of ADCLEC.syn1 CAR T cell, peak expansion and persistence defined as duration from Day 1 to the last assessment of detectable levels of ADCLEC.syn1 CAR T cells, as assessed by PCR and flow cytometry. Exploratory endpoints include (a) association of ORR and/or PFS with expansion and duration of ADCLEC.syn1 CAR T cells, with ADGRE2 and CLEC12A antigen expression, and CAR T cell phenotypes; (b) MRD assessment in those patients who achieve CR or CRi as assessed by multiparameter flow cytometry and/or NGS; (c) analysis of serum cytokines following CAR T cell infusion; (d) analysis for immunogenicity towards the CAR-CCR construct; and (e) association of relapse with ADGRE2 and CLEC12A expression levels, ADCLEC.syn1 CAR T cell persistence and phenotype and changes in tumor microenvironment.

Tumor Response Assessment Criteria. Antitumor responses is assessed according to the ELN response criteria in AML. Eligibility/Inclusion criteria include (a) being ≥18 years of age at the time of signing the ICF; (b) have been refractory or relapsed AML who have exhausted or are not eligible or intolerant to, standard therapeutic options (the following disease status is eligible for the study: (1) primary refractory disease after two courses of induction chemotherapy or after one course of hypomethylating agent or low dose cytarabine in combination with venetoclax that has not achieved CR/CRi or MLFS by the ELN criteria; or (2) recurrent A M L at any time after achieving a response (CR or CRi) during or after the course of treatment, including HSCT); (c) any degree of detectable disease is eligible; (d) ECOG performance status 0 or 1; (e) having a suitable stem cell donor identified who may donate cells in the event that the subject needs to undergo an allogeneic HSCT for rescue from prolonged marrow aplasia (donor can be from related or unrelated matched source, haplo or cord, and must be found to be suitable according to standard criteria); and (f) having adequate organ function defined as serum creatinine <2.0 mg/100 ml, direct bilirubin <2.0 mg/100 ml, AST and/or ALT ≤5×ULN, unless considered due to leukemic organ involvement. Exclusion Criteria include (a) diagnosis of acute promyelocytic leukemia; (b) radiologically-detected or symptomatic CNS disease or CNS 3 disease, i.e., presence of ≥5/ul WBCs in CSF (subjects with adequately treated CNS leukemia are eligible); (c) oxygen saturation <90% on room air; (d) prior allogeneic HSCT within 3 months of signing ICF or with ongoing requirement for systemic graft-versus-host therapy; (e) clinically significant cardiovascular disease, including stroke or myocardial infarction within 6 months prior to first study medication; or the presence of unstable angina or congestive heart failure of New York Heart Association grade 2 or higher, or cardiac ejection fraction <40%; (f) uncontrolled clinically significant infections; (g) positive serologic test results for HIV; (h) acute or chronic HBV or HCV infection as assessed by serologic (HBVs Ag or HCV ab) or PCR results; and (i) active second malignancy that requires systemic treatments, with the exception of malignancy treated with curative intent and without evidence of disease for >2 years before screening. Stopping Rules include (a) for Dose Escalation, the stopping rules are as per the dose escalation rules; (b) for Dose Expansion, a temporary halt to enrollment in the study occurs when any patient death deemed probably or possibly related to ADCLEC.syn1 CAR T cells, or ≥30% subjects in an expansion cohort, where at least 6 subjects have been enrolled, experience an ADCLEC.syn1 CAR T cell treatment-related toxicity at any time on study that would have otherwise qualified as a DLT.

Statistical Methods. In general, clinical data are summarized by cohort, separately by each regimen, using descriptive statistics (n, mean, standard deviation, standard error, median, first quartile (Q1), third quartile (Q3), minimum, and maximum for continuous variables, and frequencies and percentages for categorical variables). When categorical data are presented, the percentages are suppressed when the frequency count is zero. Non-zero percentages are rounded to one decimal place, except 100% is displayed without any decimal places. For selected assessments, confidence intervals (CIs) is displayed. Time-to-event variables are summarized using Kaplan-Meier methods.

### Example 10—Identification and Characterization of Anti-ADGRE2 Antibodies and scFvs

The present example demonstrates derivation and characterization of binding affinities of anti-ADGRE2 antibodies and scFvs. Antibodies were developed using hybridoma technology, comprising 24 humanized sequences of the mouse Reference 1 Clone. Antibodies were selected based on expression as recombinant protein variants, binding to ADGRE2-overexpressing murine lymphoma EL4 cells as measured by FA CS, and immunogenicity score, 18 humanized recombinant antibodies were selected representing a range of A DGRE2 binding affinities. The amino acid sequence of the Reference 1 anti-ADGRE2 antibody was determined by endoprotease digestion and subsequent analysis of peptide pools by LC-MS/MS. Briefly, the heavy and light chains of the antibody were separated by SDS-PAGE under reducing conditions. After staining with Coomassie Blue, respective bands were cut from the gel and digested with A sp N, chymotrypsin, trypsin and elastase endopeptidases. In addition, antibody was digested in solution by pepsin. The pool of peptides generated from digestion was analyzed on an Orbitrap analyzer (LC-MS/MS Q-Exactive, ThermoFisher). LC-MS/MS data was processed using the PEAKS AB antibody sequencing software. The Reference 1 anti-ADGRE2 V_{H} and V_{L} coding sequences were derived from the respective antibody chain sequences, and cloned with an lgG2 constant region. Recombinant Reference 1 antibody was expressed in HEK 293 cells, and purified antibody was compared with commercially available Reference 1 antibody by surface plasmon resonance (SPR) K_{D} analysis using a recombinantly produced protein comprising the extracellular domain of ADGRE2 as well as EC50 determination was carried out for binding to cells expressing ADGRE2. For subsequent antibody screening work, purified recombinant Reference 1 antibody was used as a reference antibody.

### Example 11—On-Cell Binding of Anti-ADGRE2 scFvs

This example illustrates the on-cell binding for the anti-ADGRE2 scFvs as measured by flow cytometry. The on-cell binding for the anti-ADGRE2 scFvs was assessed by flow cytometry on the E4 cells overexpressing ADGRE2. Each of the scFvs were tested for binding to ADGRE2 and compared to the Reference 1 mAb. Briefly, 100,000 cells per well, were plated in 96 well V bottom plate, scFvs were diluted to 200 nM scFv then 1:4 serially down to 0.01 nM. A dose dependent titration of the scFvs validated recombinant scFvs folding and binding to ADGRE2. EC₅₀ affinities were compared in order to better interpret any differences seen in-vivo that might be caused by variations in affinities resulting from the humanization process Data was analyzed using Prism software, using a four parameter regression. The approximate EC₅₀ values were determined using equation: Y=Bottom+(Top−Bottom)/(1+10\hat{ }((LogEC₅₀−X)*HillSlope)) where the fitted parameters are defined as follows: Bottom, the lower plateau describing minimum binding achievable; Top, the upper plateau describing the maximum binding achievable; LogEC50, the inflection point of the dose response curve also known as the concentration producing a half-maximal response; and Hill-Slope, the slope of the dose response curve.

TABLE 20
Binding Affinity of anti-ADGRE2 scFv

| scFv | Affinity (EC₅₀ nm) |
|---|---|
| ADGRE2-D | 16.4 |
| ADGRE2-B | 55.3 |
| ADGRE2-E | 10.6 |
| ADGRE2-A | 93.8 |
| ADGRE2-F | 16.4 |
| ADGRE2-C | 53.2 |
| Reference 1 | 10.1 |

As shown in Table 20, the EC₅₀ values calculated from the curves show that each of the antibodies binds cells at affinities from 10.2 to 93.8. Overall, the results showed that the tested humanized scFvs had EC₅₀ values comparable to the Reference 1 standard.

### Example 12—in Silico Immunogenicity Analysis of Anti-ADGRE2 scFv

This example illustrates in silico immunogenicity analysis. Briefly, mouse Reference 1 scFv sequences was analyzed with the humanized scFvs by a human MHCI and MHCII presentation prediction software, based on various prediction databases IEDB, SMN-Align, NN-Align.

TABLE 21
Immunogenicity of anti-ADGRE2 scFvs

| | Immunogenicity | | |
|---|---|---|---|
| scFv | MHC I | MHC II | MHC I + MHC II |
| ADGRE2-D | 533 | 645 | 1178 |
| ADGRE2-B | 686 | 876 | 1562 |
| ADGRE2-E | 500 | 685 | 1185 |
| ADGRE2-A | 647 | 995 | 1642 |
| ADGRE2-F | 628 | 844 | 1472 |
| ADGRE2-C | 643 | 941 | 1584 |
| Reference 1 | 886 | 1183 | 2069 |

As shown in Table 21, the immunogenicity of the antibody was characterized based on MHC I or MHC II binding or binding to both MHC I and MHC II. Overall, the data predict low immunogenicity for all tested humanized antibodies.

### Example 13—Off-Target Screening Panel Assay of Anti-ADGRE2 scFv

This example illustrates the specificity of anti-ADGRE2 scFvs in an off-target binding assay. Briefly, humanized scFv variants were tested for off-target binding. Three exemplary ADGRE2 scFv clones were run in the “Cut-down Assay” to screen for binding of to over 3000 human receptors. In the cut-down assay, the higher the binding, the higher the likelihood of the interaction being real. Generally, hits labelled “V. weak” are unlikely to be real interactions. The results showed that all the clones tested did not show any off-target binding. Thus, these clones were found to be highly specific for ADGRE2.

### Example 14—Characterization of Clec12A binding of anti-CLEC12A scFv

The present example demonstrates derivation and characterization of binding affinities of anti-CLEC12A antibodies. In order to select and screen for Clec12A antibodies, hybridoma technology was used. Selections were carried out on Clec12A overexpressing CHO-S cells. Antibodies were selected for a diversity of sequences. 16 antibodies were selected out of 74 antibodies for a range of soluble and on-cell Clec12A binding affinities. The binding affinities of Clec12A antibodies were determined by Biacore analysis.

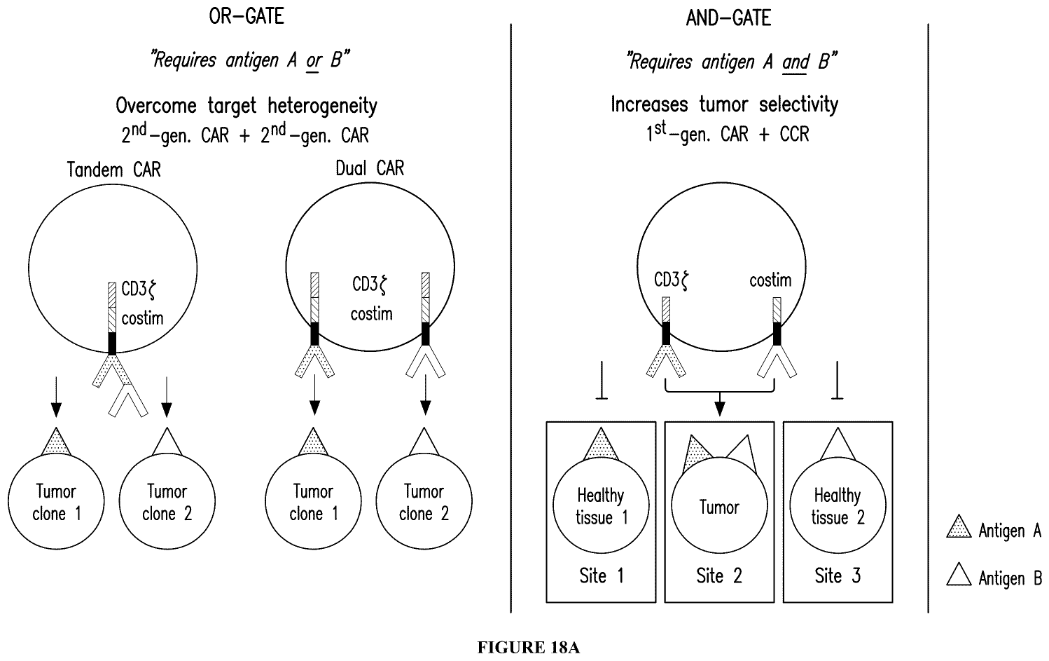

Further, the present example evaluates non-specific binding of the anti-CLEC12A antibodies. To assess the potential for the anti-CLEC12A antibodies to bind non-specific membrane proteins, scFvs derived from the 4 lead antibodies were evaluated in a surface membrane protein (SM P) assay (FIG. 17A). The SM P assay used was an ELISA based assay with human HEK-293 or insect SF9 cell membranes coated on the plate to test for non-specific binding to these membranes by the test antibodies. Internal control high and low non-specific binding antibodies were included. The high non-specific binding control, sc209, used was an antibody that has off target tox in clinic while the low non-specific binding control, 5f9, has not demonstrated off target tox in clinic (FIG. 17B).

Overall, the results showed low non-specific binding by anti-CLEC12A antibodies CLEC12-C, CLEC12-D, and CLEC12A (FIG. 17B)

### Example 15—Off-Target Screening Panel Assay of Anti-CLEC12A scFvs

This example illustrates the specificity of anti-CLEC12A scFvs in an off-target binding assay. Briefly, to test anti-CLEC12A scFvs for specificity to Clec12A as well as other membrane proteins, an off-target binding assay was conducted. Two selected Clec12A scFv clones were run in a “cut-down assay” to screen for binding of over 3000 human receptors. In the cut-down assay, the higher the binding, the higher the likelihood of the interaction being real. Generally, hits labelled “V. weak” are unlikely to be real interactions. scFvCLEC12A-A did not show any non-specific interactions to any receptor other than Clec12A or the receptors which come up as artifacts in this assay. The cut-down assay was used to screen clones for off-target assays. Selected scFv-Fc clones are being tested more comprehensively in additional screening and confirmation assays.

### Example 16—Preclinical Study

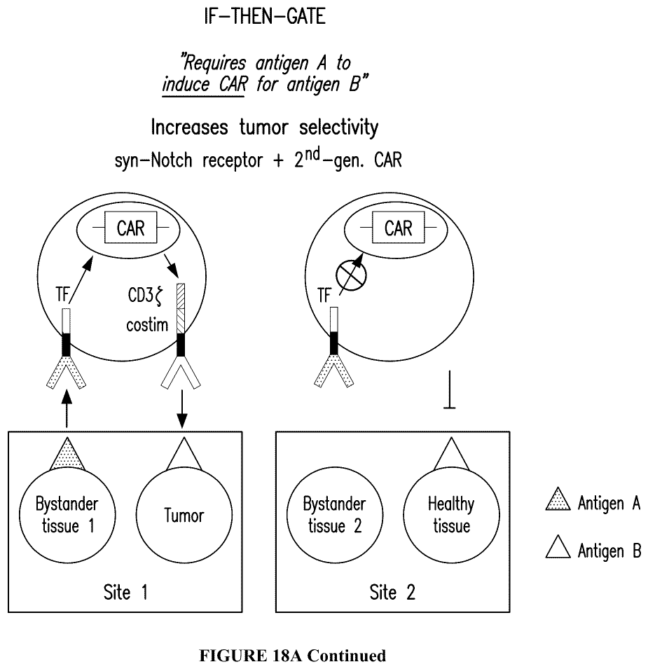

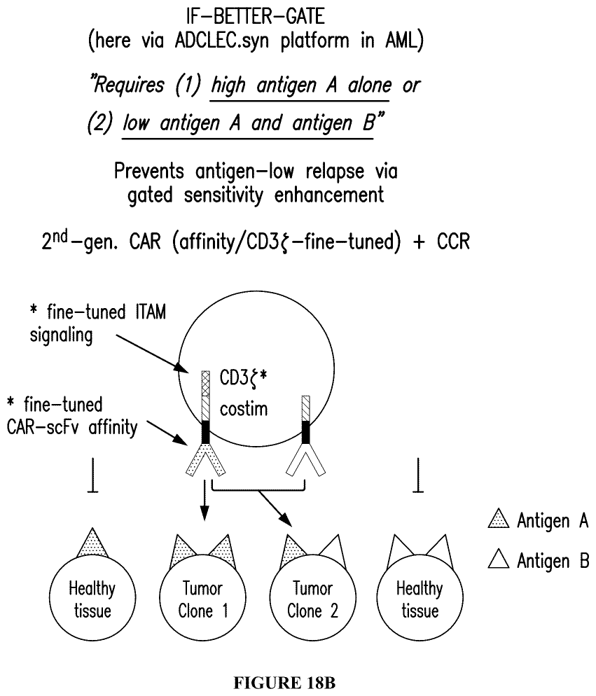

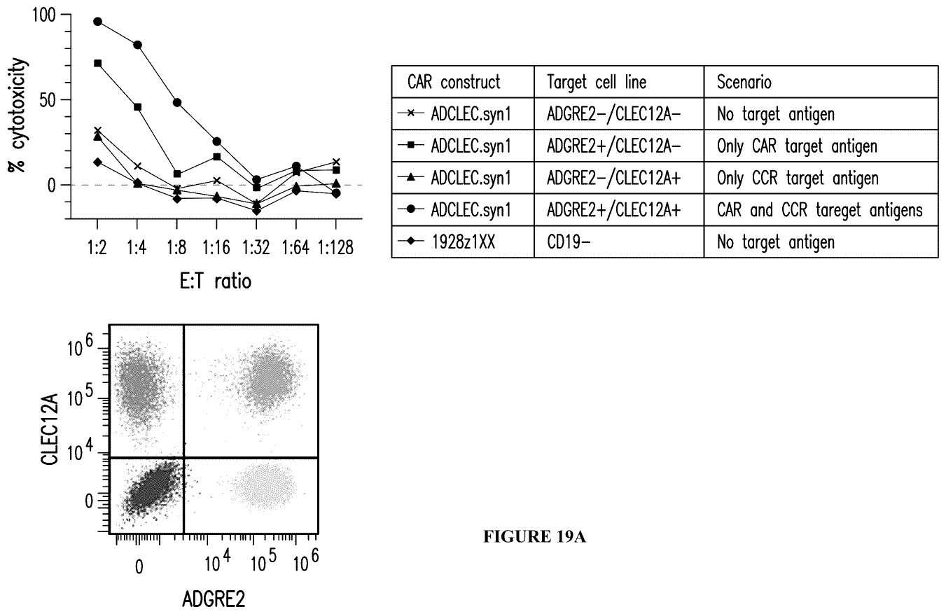

CAR T cell therapy provides a potent therapeutic option in various B cell-related hematologic malignancies. One of the major efficacy challenges is escape of tumor cells with low antigen density, which has been clinically observed in several malignancies treated with CAR therapy. Novel concepts of CAR design are needed to address phenotypic heterogeneity including clonal variability of target antigen expression. FIG. 18A illustrates previously reported combinatorial CAR concepts including Boolean-logic OR- and AND-gate concepts as well as IF-THEN-gated CAR expression.

The inventors developed a combinatorial CAR concept that can overcome AML resistance due to target heterogeneity and antigen-low escape. Antigen-low relapse can be prevented via a novel chimeric receptor design with combinatorial signaling that is synergistic and adjusted to the respective target choice.

Flow cytometric antigen expression profiling in AML versus normal hematopoiesis was performed for several previously discovered CAR target candidates in AML. To provide a platform for identification of the ideal combinatorial CAR design, in-vitro and in-vivo models based on human AML cell lines with up- or down-regulated antigen levels of ADGRE2 and CLEC12A were established to mimic AML target heterogeneity and antigen-low escape. Using a bicistronic γ-retroviral vector, different combinatorial CAR formats targeting ADGRE2 and CLEC12A were screened.

ADGRE2 was selected as a CAR target due to its high rate of positivity on AML bulk and leukemic stem cells (LSC) in a molecularly heterogeneous AML patient population (FIGS. 1A and 1B). The affinity of an ADGRE2-targeted CAR comprising an extracellular antigen-binding domain comprising the ADGRE2-A scFv was measured and CD3zeta signaling was fine-tuned to achieve an ideal killing threshold that would allow for sparing of ADGRE2-low normal cells. Next, the inventors investigated the potential of co-targeting of a second AML-related antigen to mitigate potential CAR target antigen-low AML escape. CLEC12A was identified as a suitable co-target due to its non-overlapping expression profiles in normal hematopoiesis and other vital tissues (FIGS. 4A-4C).

ADCLEC.syn1, a novel combinatorial CAR construct including an ADGRE2-targeted 28z1XX-CAR that comprises an extracellular antigen-binding domain comprising the ADGRE2-A scFv and a CLEC12A-targeted chimeric costimulatory receptor (CCR) that comprises an extracellular antigen-binding domain comprising the CLEC12A-A scFv was developed. In addition, a construct including an ADGRE2-targeted 28z1X X-CAR that comprises an extracellular antigen-binding domain comprising the ADGRE2-A scFv and a CLEC12A-targeted BBz-CAR that comprises an extracellular antigen-binding domain comprising the CLEC12A-A scFv was also developed.

ADCLEC.syn1 operates based on a gating strategy described as “IF-BETTER” (FIG. 18B): high CAR target expression alone triggers killing, whereas low CAR target expression does not, unless a CCR target is present. Additional CCR interaction lowers the threshold for CAR-mediated killing through increased avidity and co-stimulation, allowing for higher CAR sensitivity that is purposefully limited to target cells expressing both antigens.

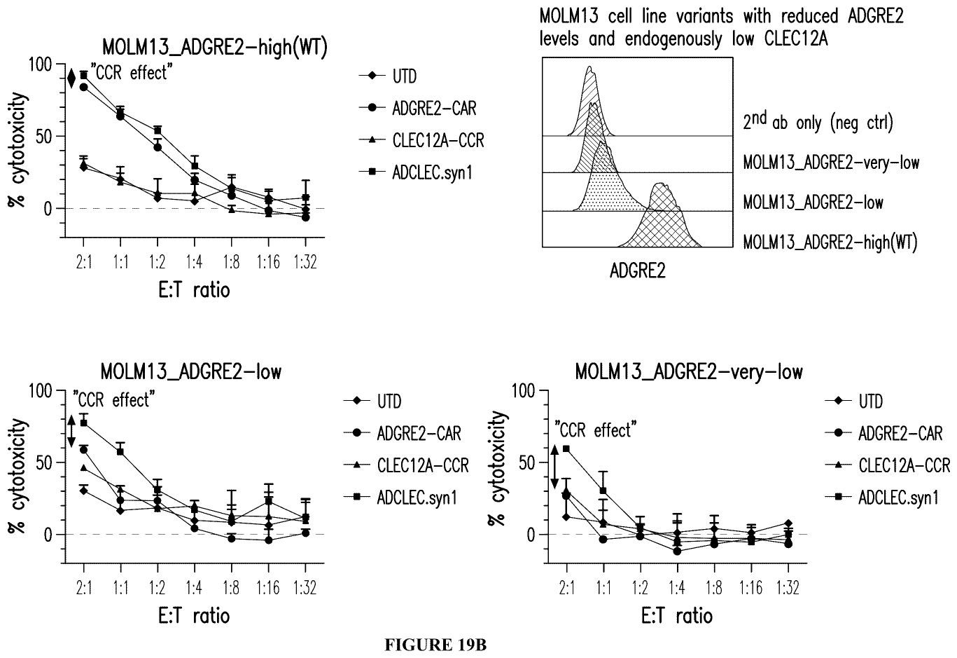

In the context of ADCLEC.syn1, ADGRE2-positive/CLEC12A-negative and ADGRE2-positive/CLEC12A-positive cells triggered cell lysis while ADGRE2-negative/CLEC12A-positive cells and ADGRE2-negative/CLEC12A-negative cells were spared. Importantly, ADCLEC.syn1 mediated more efficient killing of ADGRE2-positive/CLEC12A-positive cells as compared to ADGRE2-positive/CLEC12A-negative cells (FIG. 19A).

Using AML cell lines with varying levels of ADGRE2 to model antigen escape, ADCLEC.syn1 had superior killing capacity against ADGRE2-low/CLEC12A-low and ADGRE2-very-low/CLEC12A-low AML target cells as compared to ADGRE2-CAR (FIG. 19B).

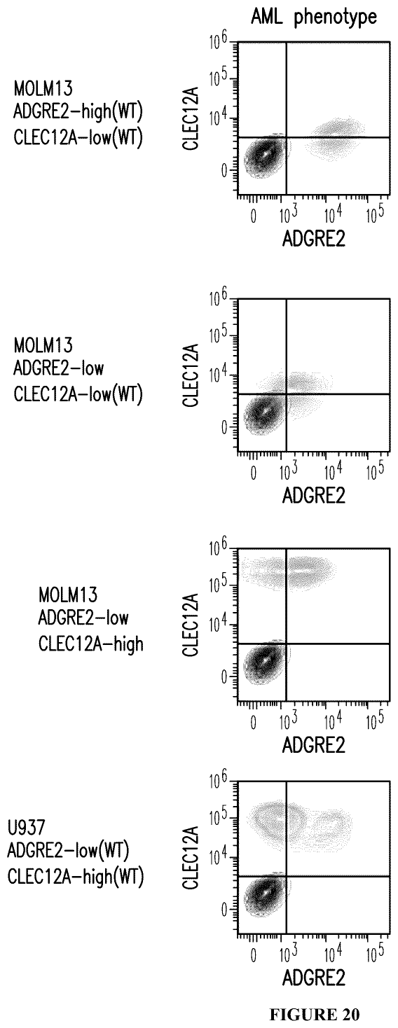

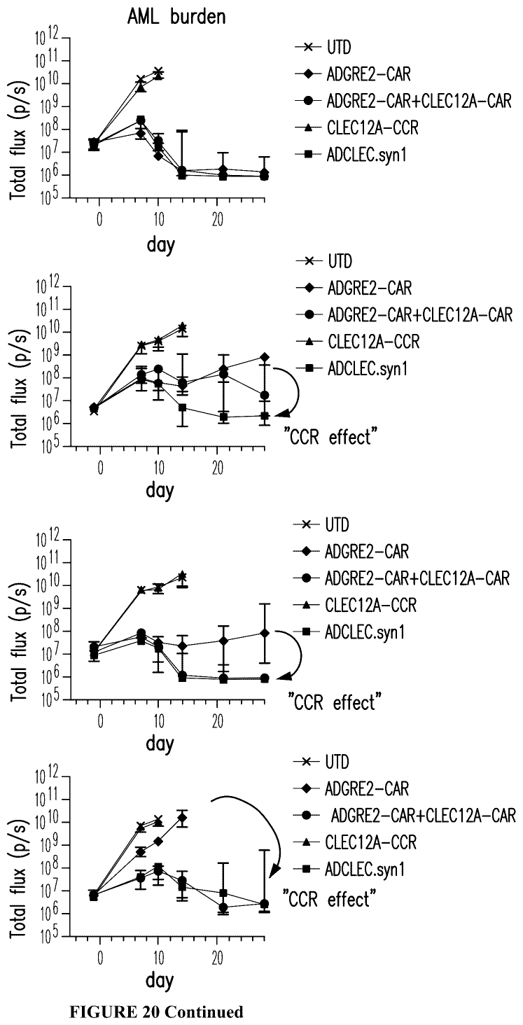

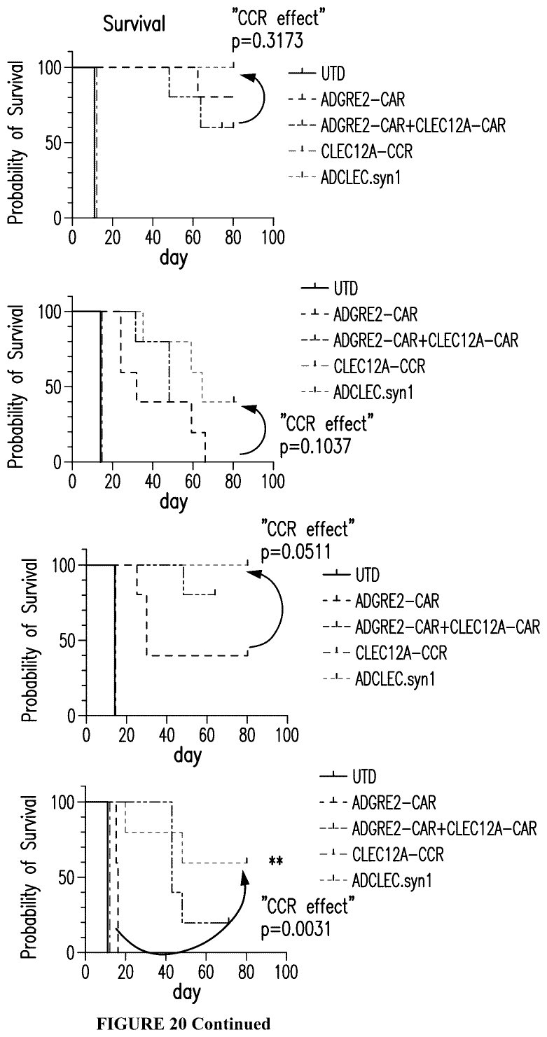

Using NSG in-vivo xenograft models of engineered MOL M 13 AML cell line variants with low levels of ADGRE2 to model antigen escape, it was found that ADCLEC.syn1 outperformed an ADGRE2-targeted CAR comprising an extracellular antigen-binding domain comprising the ADGRE2-A scFv alone without a CLEC12A-targeted CCR lacking assistance via CLEC12A-CCR. Importantly, ADCLEC.syn1 also outperformed an otherwise identical alternative dual-CAR version (OR-gated ADGRE2-CAR+CLEC12A-CAR) in the setting of ADGRE2-low MOLM 13, further underlining the importance of fine-tuned overall signaling (FIG. 20). High in-vivo potency was also confirmed against diverse AML cell lines with a wide range of ADGRE2 and CLEC12A levels reflecting population-wide AML heterogeneity (FIG. 20).

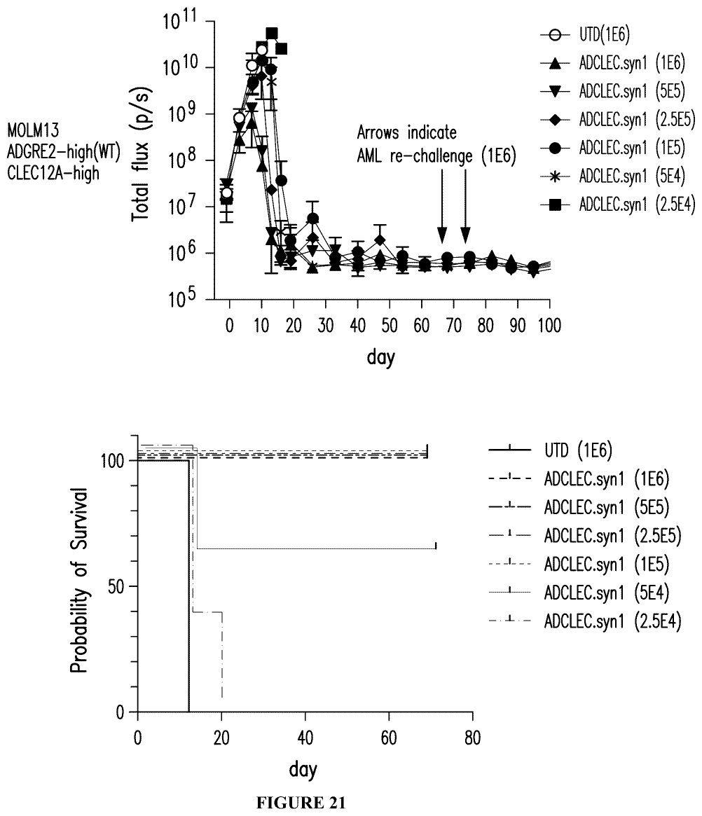

At clinically relevant CAR T cell doses, ADCLEC.syn1 induced complete and durable remissions in xenograft models of MOLM 13 (ADGRE2-high/CLEC12A-low) and U937 (ADGRE2-low/CLEC12A-high). Specifically, in NSG xenograft models using a MOL M 13 AML cell line variant with ADGRE2-high (WT) and CLEC12A-high antigen levels, ADCLEC.syn1 CAR T cells were titrated to low doses, establishing 1×10⁵ ADCLEC.syn1 T cells as minimum efficacious dose to induce complete and durable remission (FIG. 20). ADCLEC.syn1 CAR T cells were found to be functionally persistent for >70 days, with a single CAR T cell dose potently averting relapse modeled via AML re-challenges (FIG. 21).

In summary, these data provide pre-clinical evidence that an “IF-BETTER”-gated CAR+CCR T cell (ADCLEC.syn1) can outperform a single-CAR T cell (ADGRE2-CAR) and a dual-CAR T cell (ADGRE2-CAR+CLEC12A-CAR). ADCLEC.syn1 enhanced antileukemic efficacy and prevented antigen-low AML escape via detection of a rationally selected combinatorial target antigen signature that is commonly found in AML but limited in vital normal cells. Using phenotypically representative A M L xenograft models and clinically relevant T cell doses, it was demonstrated the high therapeutic potential of ADCLEC.syn1 CAR T cells, further supporting clinical translation of an “IF-BETTER”-gated CAR concept into a phase 1 trial.

Although the presently disclosed subject matter and certain of its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. A nucleic acid composition comprising
(a) a first nucleic acid molecule encoding a chimeric receptor that binds to ADGRE2 comprising an extracellular antigen-binding domain comprising a heavy chain variable region comprising a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and a light chain variable region comprising a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; and
(b) a second nucleic acid molecule encoding a chimeric receptor that binds to CLEC12A, comprising an extracellular antigen-binding domain comprising:
(i) a heavy chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and a light chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
(ii) a heavy chain variable region comprising a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and a light chain variable region comprising a CDR1 comprising the amino acid sequence set forth in SEQ ID NO: 84, a CDR2 comprising the amino acid sequence set forth in SEQ ID NO: 73, and a CDR3 comprising the amino acid sequence set forth in SEQ ID NO: 85;
(iii) a heavy chain variable region comprising a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 91; and a light chain variable region comprising a CDR1 comprising the amino acid sequence set forth in SEQ ID NO: 92, a CDR2 comprising the amino acid sequence set forth in SEQ ID NO: 93, and a CDR3 comprising the amino acid sequence set forth in SEQ ID NO: 94;
(iv) a heavy chain variable region comprising a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 93, and a CDR3 comprising the amino acid sequence set forth in SEQ ID NO: 151;
(v) a heavy chain variable region comprising a CDR1 comprising the amino acid sequence set forth in SEQ ID NO: 81, a CDR2 comprising the amino acid sequence set forth in SEQ ID NO: 103, and a CDR3 comprising the amino acid sequence set forth in SEQ ID NO: 83; and a light chain variable region comprising a CDR1 comprising the amino acid sequence set forth in SEQ ID NO: 104, a CDR2 comprising the amino acid sequence set forth in SEQ ID NO: 73, and a CDR3 comprising the amino acid sequence set forth in SEQ ID NO: 105;
(vi) a heavy chain variable region comprising a CDR1 comprising the amino acid sequence set forth in SEQ ID NO: 109, a CDR2 comprising the amino acid sequence set forth in SEQ ID NO: 103, and a CDR3 comprising the amino acid sequence set forth in SEQ ID NO: 83; and a light chain variable region comprising a CDR1 comprising the amino acid sequence set forth in SEQ ID NO: 110, a CDR2 comprising the amino acid sequence set forth in SEQ ID NO: 73, and a CDR3 comprising the amino acid sequence set forth in SEQ ID NO: 85;
(vii) a heavy chain variable region comprising a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 114; and a light chain variable region comprising a CDR1 comprising the amino acid sequence set forth in SEQ ID NO: 115, a CDR2 comprising the amino acid sequence set forth in SEQ ID NO: 93, and a CDR3 comprising the amino acid sequence set forth in SEQ ID NO: 116;
(viii) a heavy chain variable region comprising a CDR1 comprising the amino acid sequence set forth in SEQ ID NO: 120, a CDR2 comprising the amino acid sequence set forth in SEQ ID NO: 121, and a CDR3 comprising the amino acid sequence set forth in SEQ ID NO: 122; and a light chain variable region comprising a CDR1 comprising the amino acid sequence set forth in SEQ ID NO: 123, a CDR2 comprising the amino acid sequence set forth in SEQ ID NO: 124, and a CDR3 comprising the amino acid sequence set forth in SEQ ID NO: 125; or
(ix) a heavy chain variable region comprising a CDR1 comprising the amino acid sequence set forth in SEQ ID NO: 129, a CDR2 comprising the amino acid sequence set forth in SEQ ID NO: 130, and a CDR3 comprising the amino acid sequence set forth in SEQ ID NO: 131; and a light chain variable region comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134.

2. The nucleic acid composition of claim 1, wherein each extracellular antigen-binding domain comprises a single-chain variable fragment (scFv), a Fab, or an F(ab)₂.

3. The nucleic acid composition of claim 1, wherein
(a) the first chimeric receptor comprises a humanized scFv; and/or
(b) the second chimeric receptor comprises a human scFv.

4. The nucleic acid composition of claim 1, wherein the first chimeric receptor comprises:
(a) the heavy chain variable region comprises the amino acid sequence set forth in SEQ ID NO: 39; and the light chain variable region comprises the amino acid sequence set forth in SEQ ID NO: 40;
(b) the heavy chain variable region comprises the amino acid sequence set forth in SEQ ID NO: 43; and the light chain variable region comprises the amino acid sequence set forth in SEQ ID NO: 44;
(c) the heavy chain variable region comprises the amino acid sequence set forth in SEQ ID NO: 46; and the light chain variable region comprises the amino acid sequence set forth in SEQ ID NO: 47;
(d) the heavy chain variable region comprises the amino acid sequence set forth in SEQ ID NO: 49; and the light chain variable region comprises the amino acid sequence set forth in SEQ ID NO: 50;
(e) the heavy chain variable region comprises the amino acid sequence set forth in SEQ ID NO: 52; and the light chain variable region comprises the amino acid sequence set forth in SEQ ID NO: 53;
(f) the heavy chain variable region comprises the amino acid sequence set forth in SEQ ID NO: 55; and the light chain variable region comprises the amino acid sequence set forth in SEQ ID NO: 56; or
(g) the heavy chain variable region comprises the amino acid sequence set forth in SEQ ID NO: 146; and the light chain variable region comprises the amino acid sequence set forth in SEQ ID NO: 147.

5. The nucleic acid composition of claim 1, wherein the extracellular antigen-binding domain of the first chimeric receptor comprises or is an scFv, which comprises or consists of the amino acid sequence set forth in SEQ ID NO: 41, SEQ ID NO: 45, SEQ ID NO: 48, SEQ ID NO: 51, SEQ ID NO: 54, SEQ ID NO: 57, or SEQ ID NO: 148.

6. The nucleic acid composition of claim 1, wherein the first chimeric receptor comprises a transmembrane domain comprising a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LA G-3 polypeptide, a 2B 4 polypeptide, or a BTLA polypeptide.

7. The nucleic acid composition of claim 1, wherein the first chimeric receptor comprises an intracellular domain comprising a native CD3ζ polypeptide or a modified CD3ζ polypeptide.

8. The nucleic acid composition of claim 7, wherein the modified CD3ζ polypeptide comprises a native ITAM1, an ITAM2 variant consisting of two loss-of-function mutations, and an ITAM3 variant consisting of two loss-of-function mutations.

9. The nucleic acid composition of claim 8, wherein the modified CD3ζ polypeptide comprises or consists of the amino acid sequence set forth in SEQ ID NO: 27.

10. The nucleic acid composition of claim 9, wherein the intracellular domain further comprises at least one co-stimulatory signaling region.

11. The nucleic acid composition of claim 10, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

12. The nucleic acid composition of claim 1, wherein the second chimeric receptor comprises:
(a) the heavy chain variable region comprises the amino acid sequence set forth in SEQ ID NO: 75; and the light chain variable region comprises the amino acid sequence set forth in SEQ ID NO: 76;
(b) the heavy chain variable region comprises the amino acid sequence set forth in SEQ ID NO: 86; and the light chain variable region comprises the amino acid sequence set forth in SEQ ID NO: 87;
(c) the heavy chain variable region comprises the amino acid sequence set forth in SEQ ID NO: 95; and the light chain variable region comprises the amino acid sequence set forth in SEQ ID NO: 96;
d) the heavy chain variable region comprises the amino acid sequence set forth in SEQ ID NO: 100; and the light chain variable region comprises the amino acid sequence set forth in SEQ ID NO: 101;
(e) the heavy chain variable region comprises the amino acid sequence set forth in SEQ ID NO: 106; and the light chain variable region comprises the amino acid sequence set forth in SEQ ID NO: 107;
(f) the heavy chain variable region comprises the amino acid sequence set forth in SEQ ID NO: 111; and the light chain variable region comprises the amino acid sequence set forth in SEQ ID NO: 112;
(g) the heavy chain variable region comprises the amino acid sequence set forth in SEQ ID NO: 117; and the light chain variable region comprises the amino acid sequence set forth in SEQ ID NO: 118;
(h) the heavy chain variable region comprises the amino acid sequence set forth in SEQ ID NO: 126; and the light chain variable region comprises the amino acid sequence set forth in SEQ ID NO: 127; or
(i) the heavy chain variable region comprises the amino acid sequence set forth in SEQ ID NO: 135; and the light chain variable region comprises the amino acid sequence set forth in SEQ ID NO: 136.

13. The nucleic acid composition of claim 1, wherein the extracellular antigen-binding domain of the second chimeric receptor comprises or is an scFv, which comprises or consists of the amino acid sequence set forth in SEQ ID NO: 79, SEQ ID NO: 88, SEQ ID NO: 97, SEQ ID NO: 102, SEQ ID NO: 108, SEQ ID NO: 113, SEQ ID NO: 119, SEQ ID NO: 128, or SEQ ID NO: 137.

14. The nucleic acid composition of claim 1, wherein the second chimeric receptor comprises a transmembrane domain comprising a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LA G-3 polypeptide, a 2B 4 polypeptide, or a BTLA polypeptide.

15. The nucleic acid composition of claim 1, wherein the second chimeric receptor comprises an intracellular domain that does not comprise a CD3ζ polypeptide.

16. The nucleic acid composition of claim 15, wherein the intracellular domain comprises at least one co-stimulatory signaling region.

17. The nucleic acid composition of claim 16, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

18. The nucleic acid composition of claim 1, wherein
(a) the first chimeric receptor is a chimeric antigen receptor (CAR); and
(b) the second chimeric receptor is a chimeric co-stimulatory receptor (CCR).

19. The nucleic acid molecule of claim 1, further comprising a promoter that is operably linked to each chimeric receptor.

20. A vector comprising the nucleic acid composition of claim 1.

21. The vector of claim 20, wherein the vector is a viral vector.

22. The vector of claim 21, wherein the viral vector is as retroviral vector.

23. A cell comprising the nucleic acid composition of claim 1, wherein the cell is a T cell or a Natural Killer cell.

24. A nucleic acid molecule comprising
(a) a first polynucleotide encoding a chimeric receptor that binds to ADGRE2 comprising an extracellular antigen-binding domain comprising a heavy chain variable region comprising a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and a light chain variable region comprising a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; and
(b) a second polynucleotide encoding a chimeric receptor that binds to CLEC12A, comprising an extracellular antigen-binding domain, a transmembrane domain, and an intracellular domain wherein the extracellular antigen-binding domain comprises:
(i) a heavy chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and a light chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
(ii) a heavy chain variable region comprising a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and a light chain variable region comprising a CDR1 comprising the amino acid sequence set forth in SEQ ID NO: 84, a CDR2 comprising the amino acid sequence set forth in SEQ ID NO: 73, and a CDR3 comprising the amino acid sequence set forth in SEQ ID NO: 85;
(iii) a heavy chain variable region comprising a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 91; and a light chain variable region comprising a CDR1 comprising the amino acid sequence set forth in SEQ ID NO: 92, a CDR2 comprising the amino acid sequence set forth in SEQ ID NO: 93, and a CDR3 comprising the amino acid sequence set forth in SEQ ID NO: 94;
(iv) a heavy chain variable region comprising a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 93, and a CDR3 comprising the amino acid sequence set forth in SEQ ID NO: 151;
(v) a heavy chain variable region comprising a CDR1 comprising the amino acid sequence set forth in SEQ ID NO: 81, a CDR2 comprising the amino acid sequence set forth in SEQ ID NO: 103, and a CDR3 comprising the amino acid sequence set forth in SEQ ID NO: 83; and a light chain variable region comprising a CDR1 comprising the amino acid sequence set forth in SEQ ID NO: 104, a CDR2 comprising the amino acid sequence set forth in SEQ ID NO: 73, and a CDR3 comprising the amino acid sequence set forth in SEQ ID NO: 105;
(vi) a heavy chain variable region comprising a CDR1 comprising the amino acid sequence set forth in SEQ ID NO: 109, a CDR2 comprising the amino acid sequence set forth in SEQ ID NO: 103, and a CDR3 comprising the amino acid sequence set forth in SEQ ID NO: 83; and a light chain variable region comprising a CDR1 comprising the amino acid sequence set forth in SEQ ID NO: 110, a CDR2 comprising the amino acid sequence set forth in SEQ ID NO: 73, and a CDR3 comprising the amino acid sequence set forth in SEQ ID NO: 85;
(vii) a heavy chain variable region comprising a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 114; and a light chain variable region comprising a CDR1 comprising the amino acid sequence set forth in SEQ ID NO: 115, a CDR2 comprising the amino acid sequence set forth in SEQ ID NO: 93, and a CDR3 comprising the amino acid sequence set forth in SEQ ID NO: 116;
(viii) a heavy chain variable region comprising a CDR1 comprising the amino acid sequence set forth in SEQ ID NO: 120, a CDR2 comprising the amino acid sequence set forth in SEQ ID NO: 121, and a CDR3 comprising the amino acid sequence set forth in SEQ ID NO: 122; and a light chain variable region comprising a CDR1 comprising the amino acid sequence set forth in SEQ ID NO: 123, a CDR2 comprising the amino acid sequence set forth in SEQ ID NO: 124, and a CDR3 comprising the amino acid sequence set forth in SEQ ID NO: 125; or
(ix) a heavy chain variable region comprising a CDR1 comprising the amino acid sequence set forth in SEQ ID NO: 129, a CDR2 comprising the amino acid sequence set forth in SEQ ID NO: 130, and a CDR3 comprising the amino acid sequence set forth in SEQ ID NO: 131; and a light chain variable region comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134.

25. A vector comprising the nucleic acid molecule of claim 24.

26. A composition comprising
(a) a first vector encoding a chimeric receptor that binds to ADGRE2 comprising an extracellular antigen-binding domain comprising a heavy chain variable region comprising a CDR1 comprising the amino acid sequence set forth in SEQ ID NO: 33, a CDR2 comprising the amino acid sequence set forth in SEQ ID NO: 34, and a CDR3 comprising the amino acid sequence set forth in SEQ ID NO: 35; and a light chain variable region comprising a CDR1 comprising the amino acid sequence set forth in SEQ ID NO: 36, a CDR2 comprising the amino acid sequence set forth in SEQ ID NO: 37, and a CDR3 comprising the amino acid sequence set forth in SEQ ID NO: 38; and
(b) a second vector encoding a chimeric receptor that binds to CLEC12A, comprising an extracellular antigen-binding domain, a transmembrane domain, and an intracellular domain wherein the extracellular antigen-binding domain comprises:
(i) a heavy chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and a light chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
(ii) a heavy chain variable region comprising a CDR1 comprising the amino acid sequence set forth in SEQ ID NO: 81, a CDR2 comprising the amino acid sequence set forth in SEQ ID NO: 82, and a CDR3 comprising the amino acid sequence set forth in SEQ ID NO: 83; and a light chain variable region comprising a CDR1 comprising the amino acid sequence set forth in SEQ ID NO: 84, a CDR2 comprising the amino acid sequence set forth in SEQ ID NO: 73, and a CDR3 comprising the amino acid sequence set forth in SEQ ID NO: 85;
(iii) a heavy chain variable region comprising a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 91; and a light chain variable region comprising a CDR1 comprising the amino acid sequence set forth in SEQ ID NO: 92, a CDR2 comprising the amino acid sequence set forth in SEQ ID NO: 93, and a CDR3 comprising the amino acid sequence set forth in SEQ ID NO: 94;
(iv) a heavy chain variable region comprising a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 98; and a light chain variable region comprising a CDR1 comprising the amino acid sequence set forth in SEQ ID NO: 99, a CDR2 comprising the amino acid sequence set forth in SEQ ID NO: 93, and a CDR3 comprising the amino acid sequence set forth in SEQ ID NO: 151;
(v) a heavy chain variable region comprising a CDR1 comprising the amino acid sequence set forth in SEQ ID NO: 81, a CDR2 comprising the amino acid sequence set forth in SEQ ID NO: 103, and a CDR3 comprising the amino acid sequence set forth in SEQ ID NO: 83; and a light chain variable region comprising a CDR1 comprising the amino acid sequence set forth in SEQ ID NO: 104, a CDR2 comprising the amino acid sequence set forth in SEQ ID NO: 73, and a CDR3 comprising the amino acid sequence set forth in SEQ ID NO: 105;
(vi) a heavy chain variable region comprising a CDR1 comprising the amino acid sequence set forth in SEQ ID NO: 109, a CDR2 comprising the amino acid sequence set forth in SEQ ID NO: 103, and a CDR3 comprising the amino acid sequence set forth in SEQ ID NO: 83; and a light chain variable region comprising a CDR1 comprising the amino acid sequence set forth in SEQ ID NO: 110, a CDR2 comprising the amino acid sequence set forth in SEQ ID NO: 73, and a CDR3 comprising the amino acid sequence set forth in SEQ ID NO: 85;
(vii) a heavy chain variable region comprising a CDR1 comprising the amino acid sequence set forth in SEQ ID NO: 89, a CDR2 comprising the amino acid sequence set forth in SEQ ID NO: 90, and a CDR3 comprising the amino acid sequence set forth in SEQ ID NO: 114; and a light chain variable region comprising a CDR1 comprising the amino acid sequence set forth in SEQ ID NO: 115, a CDR2 comprising the amino acid sequence set forth in SEQ ID NO: 93, and a CDR3 comprising the amino acid sequence set forth in SEQ ID NO: 116;
(viii) a heavy chain variable region comprising a CDR1 comprising the amino acid sequence set forth in SEQ ID NO: 120, a CDR2 comprising the amino acid sequence set forth in SEQ ID NO: 121, and a CDR3 comprising the amino acid sequence set forth in SEQ ID NO: 122; and a light chain variable region comprising a CDR1 comprising the amino acid sequence set forth in SEQ ID NO: 123, a CDR2 comprising the amino acid sequence set forth in SEQ ID NO: 124, and a CDR3 comprising the amino acid sequence set forth in SEQ ID NO: 125; or
(ix) a heavy chain variable region comprising a CDR1 comprising the amino acid sequence set forth in SEQ ID NO: 129, a CDR2 comprising the amino acid sequence set forth in SEQ ID NO: 130, and a CDR3 comprising the amino acid sequence set forth in SEQ ID NO: 131; and a light chain variable region comprising a CDR1 comprising the amino acid sequence set forth in SEQ ID NO: 132, a CDR2 comprising the amino acid sequence set forth in SEQ ID NO: 133, and a CDR3 comprising the amino acid sequence set forth in SEQ ID NO: 134.
